SEQUENCE LISTING (28 sequences)

SEQ ID NO: 1 - DNA, 2670 bp, Homo sapiens
atggacgcgcgcgggggcggcgggcggcccggggagagcccgggcgcgagccccacgaccgggccgccgccgccgccgccgcccgcgcccccccaacagcagccgccgccgccgccgccgcccgcgccccccccgggccccgggcccgcgcccccccagcacccgccccgggccgaggcgttgcccccggaggcggcggatgagggcggcccgcggggccggctccgcagccgcgacagctcgtgcggccgccccggcaccccgggcgcggcgagcacggccaagggcagcccgaacggcgagtgcgggcgcggcgagccgcagtgcagccccgcggggcccgagggcccggcgcgggggcccaaggtgtcgttctcgtgccgcggggcggcctcggggcccgcgccggggccggggccggcggaggaggcgggcagcgaggaggcgggcccggcgggggagccgcgcggcagccaggccagcttcatgcagcgccagttcggcgcgctcctgcagccgggcgtcaacaagttctcgctgcggatgttcggcagccagaaggccgtggagcgcgagcaggagcgcgtcaagtcggcgggggcctggatcatccacccgtacagcgacttcaggttttactgggatttaataatgcttataatgatggttggaaatctagtcatcataccagttggaatcacattctttacagagcaaacaacaacaccatggattattttcaatgtggcatcagatacagttttcctattggacctgatcatgaattttaggactgggactgtcaatgaagacagttctgaaatcatcctggaccccaaagtgatcaagatgaattatttaaaaagctggtctgtggttgacttcatctcatccatcccagtggattatatctttcttattgtagaaaaaggaatggattctgaagtttacaagacagccagggcacttcgcattgtgaggtttacaaaaattctcagtctcttgcgtttattacgactttcaaggttaattagatacatacatcaatgggaagagatattccacatgacatatgatctcgccagtgcagtggtgagaatttttaatctcatcggcatgatgctgctcctgtgccactgggatggttgtcttcagttcttagtaccactactgcaggacttcccaccagattgctgggtgtctttaaatgaaatggttaatgattcttggggaaagcagtattcatacgcactcttcaaagctatgagtcacatgctgtgcattgggtatggagcccaagccccagtcagcatgtctgacctctggattaccatgctgagcatgatcgtcggggccacctgctatgccatgtttgtcggccatgccaccgctttaatccagtctctggactcctcgcggcgccagtaccaggagaagtacaagcaggtggagcagtacatgtccttccacaagctgccagctgacttccgccagaagatccacgactactatgagcaccgttaccagggcaagatgtttgacgaggacagcatcctgggcgagctcaacgggcccctgcgggaggagatcgtcaacttcaactgccggaagctggtggcctccatgccgctgttcgccaacgccgaccccaacttcgtcacggccatgctgaccaagctcaagttcgaggtcttccagccgggtgactacatcatccgcgaaggcaccatcgggaagaagatgtacttcatccagcacggcgtggtcagcgtgctcactaagggcaacaaggagatgaagctgtccgatggctcctacttcggggagatctgcctgctcacccggggccgccgcacggcgagcgtgcgggctgacacctactgccgcctctattcgctgagcgtggacaacttcaacgaggtgctggaggagtaccccatgatgcggcgcgccttcgagacggtggccatcgaccgcctggaccgcatcggcaagaagaattccatcctcctgcacaaggtgcagcatgacctcaactcgggcgtattcaacaaccaggagaacgccatcatccaggagatcgtcaagtacgaccgcgagatggtgcagcaggccgagctgggtcagcgcgtgggcctcttcccgccgccgccgccgccgccgcaggtcacctcggccatcgccacgctgcagcaggcggcggccatgagcttctgcccgcaggtggcgcggccgctcgtggggccgctggcgctcggctcgccgcgcctcgtgcgccgcccgcccccggggcccgcacctgccgccgcctcacccgggcccccgccccccgccagccccccgggcgcgcccgccagcccccgggcaccgcggacctcgccctacggcggcctgcccgccgccccccttgctgggcccgccctgcccgcgcgccgcctgagccgcgcgtcgcgcccactgtccgcctcgcagccctcgctgcctcacggcgcccccggccccgcggcctccacacgcccggccagcagctccacaccgcgcttggggcccacgcccgctgcccgggccgccgcgcccagcccggaccgcagggactcggcctcacccggcgccgccggcggcctggacccccaggactccgcgcgctcgcgcctctcgtccaacttgtga

SEQ ID NO: 2 - protein, 889 aa, Homo sapiens
MDARGGGGRPGESPGASPTTGPPPPPPPAPPQQQPPPPPPPAPPPGPGPAPPQHPPRAEALPPEAADEGGPRGRLRSRDSSCGRPGTPGAASTAKGSPNGECGRGEPQCSPAGPEGPARGPKVSFSCRGAASGPAPGPGPAEEAGSEEAGPAGEPRGSQASFMQRQFGALLQPGVNKFSLRMFGSQKAVEREQERVKSAGAWIIHPYSDFRFYWDLIMLIMMVGNLVIIPVGITFFTEQTTTPWIIFNVASDTVFLLDLIMNFRTGTVNEDSSEIILDPKVIKMNYLKSWSVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLNSGVFNNQENAIIQEIVKYDREMVQQAELGQRVGLFPPPPPPPQVTSAIATLQQAAAMSFCPQVARPLVGPLALGSPRLVRRPPPGPAPAAASPGPPPPASPPGAPASPRAPRTSPYGGLPAAPLAGPALPARRLSRASRPLSASQPSLPHGAPGPAASTRPASSSTPRLGPTPAARAAAPSPDRRDSASPGAAGGLDPQDSARSRLSSNL

SEQ ID NO: 3 - DNA, 2325 bp, Homo sapiens
atggaggcagagcagcggccggcggcgggggccagcgaaggggcgacccctggactggaggcggtgcctcccgttgctcccccgcctgcgaccgcggcctcaggtccgatccccaaatctgggcctgagcctaagaggaggcaccttgggacgctgctccagcctacggtcaacaagttctcccttcgggtgttcggcagccacaaagcagtggaaatcgagcaggagcgggtgaagtcagcgggggcctggatcatccacccctacagcgacttccggttttactgggacctgatcatgctgctgctgatggtggggaacctcatcgtcctgcctgtgggcatcaccttcttcaaggaggagaactccccgccttggatcgtcttcaacgtattgtctgatactttcttcctactggatctggtgctcaacttccgaacgggcatcgtggtggaggagggtgctgagatcctgctggcaccgcgggccatccgcacgcgctacctgcgcacctggttcctggttgacctcatctcttctatccctgtggattacatcttcctagtggtggagctggagccacggttggacgctgaggtctacaaaacggcacgggccctacgcatcgttcgcttcaccaagatcctaagcctgctgaggctgctccgcctctcccgcctcatccgctacatacaccagtgggaggagatctttcacatgacctatgacctggccagtgctgtggttcgcatcttcaacctcattgggatgatgctgctgctatgtcactgggatggctgtctgcagttcctggtgcccatgctgcaggacttccctcccgactgctgggtctccatcaaccacatggtgaaccactcgtggggccgccagtattcccatgccctgttcaaggccatgagccacatgctgtgcattggctatgggcagcaggcacctgtaggcatgcccgacgtctggctcaccatgctcagcatgatcgtaggtgccacatgctacgccatgttcatcggccatgccacggcactcatccagtccctggactcttcccggcgtcagtaccaggagaagtacaagcaggtggagcagtacatgtccttccacaagctgccagcagacacgcggcagcgcatccacgagtactatgagcaccgctaccagggcaagatgttcgatgaggaaagcatcctgggcgagctgagcgagccgcttcgcgaggagatcattaacttcacctgtcggggcctggtggcccacatgccgctgtttgcccatgccgaccccagcttcgtcactgcagttctcaccaagctgcgctttgaggtcttccagccgggggatctcgtggtgcgtgagggctccgtggggaggaagatgtacttcatccagcatgggctgctcagtgtgctggcccgcggcgcccgggacacacgcctcaccgatggatcctactttggggagatctgcctgctaactaggggccggcgcacagccagtgttcgggctgacacctactgccgcctttactcactcagcgtggaccatttcaatgctgtgcttgaggagttccccatgatgcgccgggcctttgagactgtggccatggatcggctgctccgcatcggcaagaagaattccatactgcagcggaagcgctccgagccaagtccaggcagcagtggtggcatcatggagcagcacttggtgcaacatgacagagacatggctcggggtgttcggggtcgggccccgagcacaggagctcagcttagtggaaagccagtactgtgggagccactggtacatgcgccccttcaggcagctgctgtgacctccaatgtggccattgccctgactcatcagcggggccctctgcccctctcccctgactctccagccaccctccttgctcgctctgcttggcgctcagcaggctctccagcttccccgctggtgcccgtccgagctggcccatgggcatccacctcccgcctgcccgccccacctgcccgaaccctgcacgccagcctatcccgggcagggcgctcccaggtctccctgctgggtccccctccaggaggaggtggacggcggctaggacctcggggccgcccactctcagcctcccaaccctctctgcctcagcgggcaacaggcgatggctctcctgggcgtaagggatcaggaagtgagcggctgcctccctcagggctcctggccaaacctccaaggacagcccagccccccaggccaccagtgcctgagccagccacaccccggggtctccagctttctgccaacatgtaa

SEQ ID NO: 4 - protein, 774 aa, Homo sapiens
MEAEQRPAAGASEGATPGLEAVPPVAPPPATAASGPIPKSGPEPKRRHLGTLLQPTVNKFSLRVFGSHKAVEIEQERVKSAGAWIIHPYSDFRFYWDLIMLLLMVGNLIVLPVGITFFKEENSPPWIVFNVLSDTFFLLDLVLNFRTGIVVEEGAEILLAPRAIRTRYLRTWFLVDLISSIPVDYIFLVVELEPRLDAEVYKTARALRIVRFTKILSLLRLLRLSRLIRYIHQWEEIFHMTYDLASAVVRIFNLIGMMLLLCHWDGCLQFLVPMLQDFPPDCWVSINHMVNHSWGRQYSHALFKAMSHMLCIGYGQQAPVGMPDVWLTMLSMIVGATCYAMFIGHATALIQSLDSSRRQYQEKYKQVEQYMSFHKLPADTRQRIHEYYEHRYQGKMFDEESILGELSEPLREEIINFTCRGLVAHMPLFAHADPSFVTAVLTKLRFEVFQPGDLVVREGSVGRKMYFIQHGLLSVLARGARDTRLTDGSYFGEICLLTRGRRTASVRADTYCRLYSLSVDHFNAVLEEFPMMRRAFETVAMDRLLRIGKKNSILQRKRSEPSPGSSGGIMEQHLVQHDRDMARGVRGRAPSTGAQLSGKPVLWEPLVHAPLQAAAVTSNVAIALTHQRGPLPLSPDSPATLLARSAWRSAGSPASPLVPVRAGPWASTSRLPAPPARTLHASLSRAGRSQVSLLGPPPGGGGRRLGPRGRPLSASQPSLPQRATGDGSPGRKGSGSERLPPSGLLAKPPRTAQPPRPPVPEPATPRGLQLSANM

SEQ ID NO: 5 - DNA, 2592 bp, Mus musculus
atggatgcgcgcgggggcggcgggcggccgggcgatagtccgggcacgacccctgcgccggggccgccgccaccgccgccgccgcccgcgccccctcagcctcagccaccacccgcgccacccccgaaccccacgaccccctcgcacccggagtcggcggacgagcccggcccgcgcgcccggctctgcagccgcgacagcgcctgcacccctggcgcggccaagggcggcgcgaatggcgagtgcgggcgcggggagccgcagtgcagccccgagggccccgcgcgcggccccaaggtttcgttctcatgccgcggggcggcctccgggccctcggcggccgaggaggcgggcagcgaggaggcgggcccggcgggtgagccgcgcggcagccaggctagcttcctgcagcgccaattcggggcgcttctgcagcccggcgtcaacaagttctccctgcggatgttcggcagccagaaggccgtggagcgcgagcaggaacgcgtgaagtcggcgggggcctggatcatccacccctacagcgacttcaggttttattggggattaatcatgcttataatgatggttggaaatttggtcatcataccagttggaatcacgttcttcacagagcagacgacaacaccgtggattattttcaacgtggcatccgatactgttttcctgttggacttaatcatgaattttaggactgggactgtcaatgaagacagctcggaaatcatcctggaccctaaagtgatcaagatgaattatttaaaaagctggtttgtggtggacttcatctcatcgatcccggtggattatatctttctcattgtagagaaagggatggactcagaagtttacaagacagccagagcacttcgtatcgtgaggtttacaaaaattctcagtctcttgcggttattacgcctttcaaggttaatcagatacatacaccagtgggaagagatattccacatgacctatgacctcgccagtgctgtggtgaggatcttcaacctcattggcatgatgctgcttctgtgccactgggatggctgtcttcagttcctggttcccctgctgcaggacttcccaccagattgctgggtttctctgaatgaaatggttaatgattcctggggaaaacaatattcctacgcactcttcaaagctatgagtcacatgctgtgcattggttatggcgcccaagcccctgtcagcatgtctgacctctggattaccatgctgagcatgattgtgggcgccacctgctacgcaatgtttgttggccatgccacagctttgatccagtctttggattcgtcacggcgccaataccaggagaagtacaagcaagtagagcaatacatgtccttccacaaactgcccgctgacttccgccagaagatccacgattactatgaacaccggtaccaagggaagatgtttgatgaggacagcatccttggggaactcaacgggccactgcgtgaggagattgtgaacttcaactgccggaagctggtggcttccatgccgctgtttgccaatgcagaccccaacttcgtcacagccatgctgacaaagctcaaatttgaggtcttccagcctggagattacatcatccgagaggggaccatcgggaagaagatgtacttcatccagcatggggtggtgagcgtgctcaccaagggcaacaaggagatgaagctgtcggatggctcctatttcggggagatctgcttgctcacgaggggccggcgtacggccagcgtgcgagctgacacctactgtcgcctctactcactgagtgtggacaatttcaacgaagtactggaggaataccccatgatgcggcgtgcctttgagactgtggctattgaccggctagatcgcataggcaagaagaactccatcttgctgcacaaggttcagcatgatctcagctcaggtgtgttcaacaaccaggagaatgccatcatccaggagattgtcaaatatgaccgtgagatggtgcagcaggcagagcttggacagcgtgtggggctcttcccaccaccgccaccaccgcaggtcacatcggccattgccaccctacagcaggctgtggccatgagcttctgcccgcaggtggcccgcccgctcgtggggcccctggcgctaggctccccacgcctagtgcgccgcgcgcccccagggcctctgcctcctgcagcctcgccagggccacccgcagcaagccccccggctgcaccctcgagccctcgggcaccgcggacctcaccctacggtgtgcctggctctccggcaacgcgtgtggggcccgcattgcccgcacgtcgcctgagccgcgcctcgcgcccactgtccgcctcgcagccctcgctgccccatggcgtgcccgcgcccagccccgcggcctctgcgcgcccggccagcagctccacgccgcgcctgggacccgcacccaccgcccggaccgccgcgcccagtccggaccgcagggactcagcctcgccgggcgctgccagtggcctcgacccactggactctgcgcgctcgcgcctctcttccaacttgtga

SEQ ID NO: 6 - protein, 863 aa, Mus musculus
MDARGGGGRPGDSPGTTPAPGPPPPPPPPAPPQPQPPPAPPPNPTTPSHPESADEPGPRARLCSRDSACTPGAAKGGANGECGRGEPQCSPEGPARGPKVSFSCRGAASGPSAAEEAGSEEAGPAGEPRGSQASFLQRQFGALLQPGVNKFSLRMFGSQKAVEREQERVKSAGAWIIHPYSDFRFYWGLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLSSGVFNNQENAIIQEIVKYDREMVQQAELGQRVGLFPPPPPPQVTSAIATLQQAVAMSFCPQVARPLVGPLALGSPRLVRRAPPGPLPPAASPGPPAASPPAAPSSPRAPRTSPYGVPGSPATRVGPALPARRLSRASRPLSASQPSLPHGVPAPSPAASARPASSSTPRLGPAPTARTAAPSPDRRDSASPGAASGLDPLDSARSRLSSNL

SEQ ID NO: 7 - DNA, 2340 bp, Mus musculus
atggaggaggaggcgcggccggcggcgggggccggcgaagcggcgacccctgcacgcgagacgcctcctgcggctccggcccaggcccgcgcggcctcaggtggggtgccggagtctgcgcccgagccgaagaggcggcagctcgggacgctgctgcagccgacggtcaacaagttctctctccgggtcttcggcagccacaaagcagtagaaatcgagcaggagagggtgaagtccgccggggcctggatcatccacccctacagcgacttccggttttactgggatctcatcatgctgctgctgatggtggggaacctcatagttctgcctgtgggtatcactttcttcaaggaggagaactctccaccctggatcgtcttcaatgtcctctctgacactttcttcctgctggatctggtgctcaacttccgaactggcatcgtggtggaggaaggtgccgagatcctgctggcgccaagggccatccgaacgcgttacctgcgcacctggttcctggttgatctgatctcctccatccctgtggattatatcttcctagtggtggagctggagccacgactagatgctgaggtctacaaaacggcacgggccctgcgcatcgttagattcaccaagatccttagcctgctgcggctgctccgcctctcccgcctcatccgctacatacaccagtgggaggagatctttcacatgacctacgacctggccagtgcagtggttcgcatcttcaacctcattggaatgatgttgctgctgtgtcactgggacggctgtctgcagtttctggtccctatgctgcaggacttcccgtccgactgctgggtctccatgaaccgcatggtgaaccactcgtggggccgccagtattcccacgccctgttcaaggccatgagtcacatgctatgcattggctatgggcagcaggcaccggtaggcatgcctgacgtctggctcaccatgctcagtatgattgtgggcgccacgtgttatgccatgttcatcggtcacgccaccgccctcatccagtccctggactcttcccggcgacagtaccaggagaagtacaagcaggtggagcagtacatgtccttccacaagctgcccgctgacacccggcagcgcatccacgagtactacgagcatcgctaccagggcaagatgtttgatgaagagagcatcctgggggagctgagcgagccacttcgggaggagattattaacttcacctgccggggcctggtggcccacatgccgctgtttgctcatgctgaccccagcttcgtcaccgcagtgctcaccaagctccgttttgaggtcttccaaccaggggacctggtggtgcgtgagggctccgtgggcaggaagatgtacttcatccagcacgggctgctgagtgtgctggcacgtggcgcccgcgacacccgcctcactgatggatcctactttggggagatctgcctgctgactcgaggtcggagaacagccagtgtaagggctgacacctattgtcgcctctactcgctcagcgtggaccacttcaatgcggtgcttgaggagttcccaatgatgcgcagggcttttgagacggtggccatggaccggcttcggcgcatcggcaaaaagaattcgatactgcagcggaaacgctctgagccgagtccaggcagcagcggtggcgtcatggagcagcatttggtacaacacgacagagacatggctcgtggtgttcggggcctggctcctggtacaggagctcgactcagtggaaagccagtgctgtgggaaccactggtgcacgcccctctgcaggcagctgctgtgacctccaacgtggccatagccttgactcaccagcgaggccctctgcccctctcccctgattctccagccaccctcctagctcgatctgctagacgctcagcaggctccccagcctccccactggtgcctgtccgagcaggtcctctgctggcccggggaccctgggcgtccacttctcgcctgcctgctccacctgcccgaaccctccatgccagcctatcccggacagggcgttcccaggtatctctgttgggccctcccccaggaggaggtgctcggaggctaggacctcggggccgcccactttctgcctcgcaaccctctctgcctcagcgagcaacaggggatggctctcctaggcgtaaaggctctggaagtgagcgcctgcccccctctgggctcttggccaaacctccagggacagtccagccacccaggtcatcagtgcctgagccagttacccccagaggtccccaaatttctgccaacatgtga

SEQ ID NO: 8 - protein, 779 aa, Mus musculus
MEEEARPAAGAGEAATPARETPPAAPAQARAASGGVPESAPEPKRRQLGTLLQPTVNKFSLRVFGSHKAVEIEQERVKSAGAWIIHPYSDFRFYWDLIMLLLMVGNLIVLPVGITFFKEENSPPWIVFNVLSDTFFLLDLVLNFRTGIVVEEGAEILLAPRAIRTRYLRTWFLVDLISSIPVDYIFLVVELEPRLDAEVYKTARALRIVRFTKILSLLRLLRLSRLIRYIHQWEEIFHMTYDLASAVVRIFNLIGMMLLLCHWDGCLQFLVPMLQDFPSDCWVSMNRMVNHSWGRQYSHALFKAMSHMLCIGYGQQAPVGMPDVWLTMLSMIVGATCYAMFIGHATALIQSLDSSRRQYQEKYKQVEQYMSFHKLPADTRQRIHEYYEHRYQGKMFDEESILGELSEPLREEIINFTCRGLVAHMPLFAHADPSFVTAVLTKLRFEVFQPGDLVVREGSVGRKMYFIQHGLLSVLARGARDTRLTDGSYFGEICLLTRGRRTASVRADTYCRLYSLSVDHFNAVLEEFPMMRRAFETVAMDRLRRIGKKNSILQRKRSEPSPGSSGGVMEQHLVQHDRDMARGVRGLAPGTGARLSGKPVLWEPLVHAPLQAAAVTSNVAIALTHQRGPLPLSPDSPATLLARSARRSAGSPASPLVPVRAGPLLARGPWASTSRLPAPPARTLHASLSRTGRSQVSLLGPPPGGGARRLGPRGRPLSASQPSLPQRATGDGSPRRKGSGSERLPPSGLLAKPPGTVQPPRSSVPEPVTPRGPQISANM

SEQ ID NO: 9 - protein, 910 aa, Mus musculus
MEGGGKPNSASNSRDDGNSVFPSKAPATGPVAADKRLGTPPRGGAAGKEHGNSVCFKVDGGGGEEPAGSFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILSELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIPPINYPQMTALNCTSSTTTPTSRMRTQSPPVYTATSLSHSNLHSPSPSTQTPQPSAILSPCSYTTAVCSPPIQSPLATRTFHYASPTASQLSLMQQPQQQLPQSQVQQTQTQTQQQQQQQQQQQQQQQQQQQQQQQQQQQQQQQQQQQQQPQTPGSSTPKNEVHKSTQALHNTNLTKEVRPLSASQPSLPHEVSTLISRPHPTVGESLASIPQPVAAVHSTGLQAGSRSTVPQRVTLFRQMSSGAIPPNRGVPPAPPPPAAVQRESPSVLNTDPDAEKPRFASNL

SEQ ID NO: 10 - protein, 910 aa, Rattus norvegicus
MEGGGKPNSASNSRDDGNSVYPSKAPATGPAAADKRLGTPPGGGAAGKEHGNSVCFKVDGGGGEEPAGSFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILSELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIPPINYPQMTALNCTSSTTTPTSRMRTQSPPVYTATSLSHSNLHSPSPSTQTPQPSAILSPCSYTTAVCSPPIQSPLATRTFHYASPTASQLSLMQQPQPQLQQSQVQQTQTQTQQQQQQQQPQPQPQQPQQQQQQQQQQQQQQQQQQQQQPQTPGSSTPKNEVHKSTQALHNTHLTREVRPLSASQPSLPHEVSTMISRPHPTVGESLASIPQPVATVHSTGLQAGSRSTVPQRVTLFRQMSSGAIPPNRGVPPAPPPPAAVQRESPSVLNKDPDAEKPRFASNL

SEQ ID NO: 11 - protein, 890 aa, Homo sapiens
MEGGGKPNSSSNSRDDGNSVFPAKASATGAGPAAAEKRLGTPPGGGGAGAKEHGNSVCFKVDGGGGGGGGGGGGEEPAGGFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWSVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILNELNDPLRGEIVNFNCRKLVATMPLFANADPNFVTAMLSKLRFEVFQPGDYIVREGAVGKKMYFIQHGVAGVITKSSKEMKLTDGSYFGEICLLTKGRRTASVRADTYCRLYSLSVDNFNEVPEEYPMMRRAFETVAIDRLDRIGKKNSILLQKFQKDLNTGVFNNQENEILKQIVKHDREMVQAIAPINYPQMTTLNSASSTTTPTSRMRTQSPPVYTATSLSHSNLHSPSPSTQTPQPSAILSPCSYTTAVCSPPVQSPLAARTFHYASPTASQLSLMQQQPQQQVQQSQPPQTQPQQPSPQPQTPGSSTPKNEVHKSTQALHNTNLTREVRPLSASQPSLPHEVPTLISRPHPTVGESLASIPQPVTAVPGTGLQAGGRSTVPQRVTLFRQMSSGAIPPNRGVPPAPPPPAAALPRESSSVLNTDPDAEKPRFASNL

SEQ ID NO: 12 - protein, 822 aa, Oryctolagus cuniculus
MATASSPPRRPRRARGLEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILNELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIAPISYPQMTALNSTSSTATPTSRMRTQSPPVYTATSLSHSNLHSPSPSTQTPQPSAILSPCSYTTAVCSPPVQSPLATRTFHYASPTASQLSLMPQQQQQPQAPQTQPQQPPQQPQTPGSATPKNEVHRSTQALPNTSLTREVRPLSASQPSLPHEVSTLISRPHPTVGESLASIPQPVAAVHSAGLQAAGRSTVPQRVTLFRQMSSGAIPPNRGVPPAPPPPAAPLQREASSVLNTDPEAEKPRFASNL

SEQ ID NO: 13 - protein, 202 aa, Cavia porcellus
IMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNKMVNVSWGQQYSYALFKA

SEQ ID NO: 14 - protein, 863 aa, Mus musculus
MDARGGGGRPGDSPGTTPAPGPPPPPPPPAPPQPQPPPAPPPNPTTPSHPESADEPGPRARLCSRDSACTPGAAKGGANGECGRGEPQCSPEGPARGPKVSFSCRGAASGPSAAEEAGSEEAGPAGEPRGSQASFLQRQFGALLQPGVNKFSLRMFGSQKAVEREQERVKSAGAWIIHPYSDFRFYWDFTMLLFMVGNLIIIPVGITFFKDETTAPWIVFNVVSDTFFLMDLVLNFRTGIVIEDNTEIILDPEKIKKKYLRTWFVVDFVSSIPVDYIFLIVEKGIDSEVYKTARALRIVRFTKILSLLRLLRLSRLIRYIHQWEEIFHMTYDLASAVMRICNLISMMLLLCHWDGCLQFLVPMLQDFPSDCWVSINNMVNHSWSELYSFALFKAMSHMLCIGYGRQAPESMTDIWLTMLSMIVGATCYAMFIGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLSSGVFNNQENAIIQEIVKYDREMVQQAELGQRVGLFPPPPPPQVTSAIATLQQAVAMSFCPQVARPLVGPLALGSPRLVRRAPPGPLPPAASPGPPAASPPAAPSSPRAPRTSPYGVPGSPATRVGPALPARRLSRASRPLSASQPSLPHGVPAPSPAASARPASSSTPRLGPAPTARTAAPSPDRRDSASPGAASGLDPLDSARSRLSSNL

SEQ ID NO: 15 - protein, 863 aa, Rattus norvegicus
MDARGGGGRPGDSPGATPAPGPPPPPPPPAPPQPQPPPAPPPNPTTPSHPESADEPGPRSRLCSRDSSCTPGAAKGGANGECGRGEPQCSPEGPARGPKVSFSCRGAASGPAAAEEAGSEEAGPAGEPRGSQASFLQRQFGALLQPGVNKFSLRMFGSQKAVEREQERVKSAGAWIIHPYSDFRFYWDFTMLLFMVGNLIIIPVGITFFKDETTAPWIVFNVVSDTFFLMDLVLNFRTGIVIEDNTEIILDPEKIKKKYLRTWFVVDFVSSIPVDYIFLIVEKGIDSEVYKTARALRIVRFTKILSLLRLLRLSRLIRYIHQWEEIFHMTYDLASAVMRICNLISMMLLLCHWDGCLQFLVPMLQDFPSDCWVSINNMVNHSWSELYSFALFKAMSHMLCIGYGRQAPESMTDIWLTMLSMIVGATCYAMFIGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLSSGVFNNQENAIIQEIVKYDREMVQQAELGQRVGLFPPPPPPQVTSAIATLQQAVAMSFCPQVARPLVGPLALGSPRLVRRAPPGPLPPAASPGPPAASPPAAPSSPRAPRTSPYGVPGSPATRVGPALPARRLSRASRPLSASQPSLPHGAPAPSPAASARPASSSTPRLGPAPTTRTAAPSPDRRDSASPGAASGLDPLDSARSRLSSNL

SEQ ID NO: 16 - protein, 889 aa, Homo sapiens
MDARGGGGRPGESPGATPAPGPPPPPPPAPPQQQPPPPPPPAPPPGPGPAPPQHPPRAEALPPEAADEGGPRGRLRSRDSSCGRPGTPGAASTAKGSPNGECGRGEPQCSPAGPEGPARGPKVSFSCRGAASGPAPGPGPAEEAGSEEAGPAGEPRGSQASFMQRQFGALLQPGVNKFSLRMFGSQKAVEREQERVKSAGAWIIHPYSDFRFYWDFTMLLFMVGNLIIIPVGITFFKDETTAPWIVFNVVSDTFFLMDLVLNFRTGIVIEDNTEIILDPEKIKKKYLRTWFVVDFVSSIPVDYIFLIVEKGIDSEVYKTARALRIVRFTKILSLLRLLRLSRLIRYIHQWEEIFHMTYDLASAVMRICNLISMMLLLCHWDGCLQFLVPMLQDFPRNCWVSINGMVNHSWSELYSFALFKAMSHMLCIGYGRQAPESMTDIWLTMLSMIVGATCYAMFIGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLNSGVFNNQENAIIQEIVKYDREMVQQAELGQRVGLFPPPPPPPQVTSAIATLQQAAAMSFCPQVARPLVGPLALGSPRLVRRPPPGPAPAAASPGPPPPASPPGAPASPRAPRTSPYGGLPAAPLAGPALPARRLSRASRPLSASQPSLPHGAPGPAASTRPASSSTPRLRPTPAARAAAPSPDRRDSASPGAAGGLDPQDSARSRLSSNL

SEQ ID NO: 17 - protein, 97 aa, Canis familiaris
AMSHMLCIGYGRQAPESMTDIWLTMLSMIVGATCYAMFIGHATALIQSLDSSRRQYQEKYKQVEQYMSFHKLPADFRQKIHDYYEHRYQGKMFDEES

SEQ ID NO: 18 - protein, 1186 aa, Mus musculus
MDKLPPSMRKRLYSLPQQVGAKAWIMDEEEDGEEEGAGGRQDPSRRSIRLRPLPSPSPSVAAGCSESRGAALGATESEGPGRSAGKSSTNGDCRRFRGSLASLGSRGGGSGGAGGGSSLGHLHDSAEERRLIAAEGDASPGEDRTPPGLATEPERPATAAQPAASPPPQQPPQPASASCEQPSADTAIKVEGGAAAIDHILPEAEVRLGQSGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFVVDFISSIPVEYIFLIVETRIDSEVYKTARAVRIVRFTKILSLLRLLRLSRLIRYIHQWEEIFHMTYDLASAVVRIVNLIGMMLLLCHWDGCLQFLVPMLQDFPHDCWVSINGMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETRLADGSYFGEICLLTRGRRTASVRADTYCRLYSLSVDNFNEVLEEYPMMRKKNSILLHKVQHDLNSGVFNYQENEIIQQIVRHDREMAHCAHRVQAAASATPTPTPVIWTPLIQAPLQAAAATTSVAIALTHHPRLPAAIFRPPPGPGLGNLGAGQTPRHPRRLQSLIPSALGSASPASSPSQVDTPSSSSFHIQQLAGFSAPPGLSPLLPSSSSSPPPGACGSPPAPTPSTSTAAAASTTGFGHFHKALGGSLSSSDSPLLTPLQPGARSPQAAQPPPPLPGARGGLGLLEHFLPPPPSSRSPSSSPGQLGQPPGELSLGLAAGPSSTPETPPRPERPSFMAGASGGASPVAFTPRGGLSPPGHSPGPPRTFPSAPPRASGSHGSLLLPPASSPPPPQVPQRRGTPPLTPGRLTQDLKLISASQPALPQDGAQTLRRASPHSSGESVAAFSLYPRAGGGSGSSGGLGPPGRPYGAIPGQHVTLPRKTSSGSLPPPLSLFGARAASSGGPPLTTAAPQREPGARSEPVRSKLPSNL

SEQ ID NO: 19 - protein, 1198 aa, Rattus norvegicus
MDKLPPSMRKRLYSLPQQVGAKAWIMDEEEDGEEEGAGGLQDPSRRSIRLRPLPSPSPSVAAGCSESRGAALGAADSEGPGRSAGKSSTNGDCRRFRGSLASLGSRGGGSGGAGGGSSLGHLHDSAEERRLIAAEGDASPGEDRTPPGLATEPERPGAAAQPAASPPPQQPPQPASASCEQPSADTAIKVEGGAAASDQILPEAEVRLGQSGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFVVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPHDCWVSINGMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLNSGVFNYQENEIIQQIVRHDREMAHCAHRVQAAASATPTPTPVIWTPLIQAPLQAAAATTSVAIALTHHPRLPAAIFRPPPGPGLGNLGAGQTPRHPRRLQSLIPSALGSASPASSPSQVDTPSSSSFHIQQLAGFSAPPGLSPLLPSSSSSPPPGACSSPPAPTPSTSTAATTTGFGHFHKALGGSLSSSDSPLLTPLQPGARSPQAAQPPPPLPGARGGLGLLEHFLPPPPSSRSPSSSPGQLGQPPGELSPGLAAGPPSTPETPPRPERPSFMAGASGGASPVAFTPRGGLSPPGHSPGPPRTFPSAPPRASGSHGSLLLPPASSPPPPQVPQRRGTPPLTPGRLTQDLKLISASQPALPQDGAQTLRRASPHSSGESMAAFSLYPRAGGGSGSSGGLGPPGRPYGAIPGQHVTLPRKTSSGSLPPPLSLFGARAASSGGPPLTAAPQREPGARSEPVRSKLPSNL

SEQ ID NO: 20 - protein, 1203 aa, Homo sapiens
MDKLPPSMRKRLYSLPQQVGAKAWIMDEEEDAEEEGAGGRQDPSRRSIRLRPLPSPSPSAAAGGTESRSSALGAADSEGPARGAGKSSTNGDCRRFRGSLASLGSRGGGSGGTGSGSSHGHLHDSAEERRLIAEGDASPGEDRTPPGLAAEPERPGASAQPAASPPPPQQPPQPASASCEQPSVDTAIKVEGGAAAGDQILPEAEVRLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTEIILDPQRIKMKYLKSWFMVDFISSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSINNMVNNSWGKQYSYALFKAMSHMLCIGYGRQAPVGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLNSGVFNYQENEIIQQIVQHDREMAHCAHRVQAAASATPTPTPVIWTPLIQAPLQAAAATTSVAIALTHHPRLPAAIFRPPPGSGLGNLGAGQTPRHLKRLQSLIPSALGSASPASSPSQVDTPSSSSFHIQQLAGFSAPAGLSPLLPSSSSSPPPGACGSPSAPTPSAGVAATTIAGFGHFHKALGGSLSSSDSPLLTPLQPGARSPQAAQPSPAPPGARGGLGLPEHFLPPPPSSRSPSSSPGQLGQPPGELSLGLATGPLSTPETPPRQPEPPSLVAGASGGASPVGFTPRGGLSPPGHSPGPPRTFPSAPPRASGSHGSLLLPPASSPPPPQVPQRRGTPPLTPGRLTQDLKLISASQPALPQDGAQTLRRASPHSSGESMAAFPLFPRAGGGSGGSGSSGGLGPPGRPYGAIPGQHVTLPRKTSSGSLPPPLSLFGARATSSGGPPLTAGPQREPGARPEPVRSKLPSNL

SEQ ID NO: 21 - protein, 1175 aa, Oryctolagus cuniculus
MDKLPPSMRKRLYSLPQQVGAKAWIMDEEEDAEEEGAGGRQDPRRRSIRLRPLPSPSPSPSAAAAAAGGAESRGAALGGAADGEGPARGAAKSSTNGDCRRFRGSLASLGSRGGGGGGGSTGGGSHGHLHDSAEERRLIAEGDASPGEDRTPPGLAAEPERPGAPAPPAASPPQVPSSCGEQRPADAAVKVEGGAAAGDQILPEAEARLGQAGFMQRQFGAMLQPGVNKFSLRMFGSQKAVEREQERVKSAGFWIIHPYSDFRFYWDLTMLLLMVGNLIIIPVGITFFKDENTTPWIVFNVVSDTFFLIDLVLNFRTGIVVEDNTDIILDPRRIKMKYLKSWFVVDFVSSIPVDYIFLIVETRIDSEVYKTARALRIVRFTKILSLLRLLRLSRLIRYIHQWEEIFHMTYDLASAVVRIVNLIGMMLLLCHWDGCLQFLVPMLQDFPDDCWVSLNNMVNNSWGKQYSYALFKAMSHMLCIGYGRQAPMGMSDVWLTMLSMIVGATCYAMFIGHATALIQSLDSSRRQYQEKYKQVEQYMSFHKLPPDTRQRIHDYYEHRYQGKMFDEESILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDNFNEVLEEYPMMRRAFETVALDRLDRIGKKNSILLHKVQHDLSSGVSNYQENAIVQRIVQHDREMAHCARRAQATTPVAPAIWTPLIQAPLQAAAATTSVAIALTHHPRLPAAIFRPPPGPTTLGSLGAGQTPRHLRRLQSLAPSAPSPASPASSPSQPDTPSSASLHVQPLPGCSTPAGLGSLLPTAGSPPAPTPPTTAGAAGFSHFHRALGGSLSSSDSPLLTPMQSAARSPQQPPPPPGAPAGLGLLEHFLPPPARSPTSSPGQLGQPPGELSPGLGSGPPGTPETPPRQPERLPFAAGASAGASPVAFSPRGGPSPPGHSPGTPRTFPSAPPRASGSHGSLLLPPASSPPPPPPPPAPQRRATPPLAPGRLSQDLKLISASQPALPQDGAQTLRRASPHSSSGESVAALPPFPRAPGRPPGAGPGQHVTLTLPRKASSGSLPPPLSLFGPRAAPAGGPRLTAAPQREPGAKSEPVRSKLPSNL

SEQ ID NO: 22 - protein, 124 aa, Canis familiaris
DEDSILGELSEPLREEIINFNCRKLVASMPLFANADPNFVTSMLTKLRFEVFQPGDYIIREGTIGKKMYFIQHGVVSVLTKGNKETKLADGSYFGEICLLTRGRRTASVRADTYCRLYSLSVDN

SEQ ID NO: 23 - DNA, 1528 bp, Homo sapiens
gaggcagttcacctccatgctgcagcccggggtcaacaaattctccctccgcatgtttgggagccagaaggcggtggaaaaggagcaggaaagggttaaaactgcaggcttctggattatccacccttacagtgatttcaggttttactgggatttaataatgcttataatgatggttggaaatctagtcatcataccagttggaatcacattctttacagagcaaacaacaacaccatggattattttcaatgtggcatcagatacagttttcctattggacctgatcatgaattttaggactgggactgtcaatgaagacagttctgaaatcatcctggaccccaaagtgatcaagatgaattatttaaaaagctggtctgtggttgacttcatctcatccatcccagtggattatatctttcttattgtagaaaaaggaatggattctgaagtttacaagacagccagggcacttcgcattgtgaggtttacaaaaattctcagtctcttgcgtttattacgactttcaaggttaattagatacatacatcaatgggaagagatattccacatgacatatgatctcgccagtgcagtggtgagaatttttaatctcatcggcatgatgctgctcctgtgccactgggatggttgtcttcagttcttagtaccactactgcaggacttcccaccagattgctgggtgtctttaaatgaaatggttaatgattcttggggaaagcagtattcatacgcactcttcaaagctatgagtcacatgctgtgcattgggtatggagcccaagccccagtcagcatgtctgacctctggattaccatgctgagcatgatcgtcggggccacctgctatgccatgtttgtcggccatgccaccgctttaatccagtctctggattcttcgaggcggcagtatcaagagaagtataagcaagtggaacaatacatgtcattccataagttaccagctgatatgcgtcagaagatacatgattactatgaacacagataccaaggcaaaatctttgatgaggaaaatattctcaatgaactcaatgatcctctgagaggggagatagtcaacttcaactgtcggaaactggtggctacaatgcctttatttgctaatgcggatcctaattttgtgactgccatgctgagcaagttgagatttgaggtgtttcaacctggagattatatcgtacgagaaggagccgtgggtaaaaaaatgtatttcattcaacacggtgttgctggtgtcattacaaaatccagtaaagaaatgaagctgacagatggctcttactttggagagatttgcctgctgaccaaaggacgtcgtactgccagtgttcgagctgatacatattgtcgtctttactcactttccgtggacaatttcaacgaggtcccggaggaatatccaatgatgaggagagcctttgagacagttgccattgaccgactagatcgaataggaaagaaaaattcaattcttctgcaaaag

SEQ ID NO: 24 - DNA, 1528 bp, Homo sapiens
gcgccagttcggcgcgctcctgcagccgggcgtcaacaagttctcgctgcggatgttcggcagccagaaggccgtggagcgcgagcaggagcgcgtcaagtcggcgggggcctggatcatccacccgtacagcgacttcaggttctactgggacttcaccatgctgctgttcatggtgggaaacctcatcatcatcccagtgggcatcaccttcttcaaggatgagaccactgccccgtggatcgtgttcaacgtggtctcggacaccttcttcctcatggacctggtgttgaacttccgcaccggcattgtgatcgaggacaacacggagatcatcctggaccccgagaagatcaagaagaagtatctgcgcacgtggttcgtggtggacttcgtgtcctccatccccgtggactacatcttccttattgtggagaagggcattgactccgaggtctacaagacggcacgcgccctgcgcatcgtgcgcttcaccaagatcctcagcctcctgcggctgctgcgcctctcacgcctgatccgctacatccatcagtgggaggagatcttccacatgacctatgacctggccagcgcggtgatgaggatctgcaatctcatcagcatgatgctgctgctctgccactgggacggctgcctgcagttcctggtgcctatgctgcaggacttcccgcgcaactgctgggtgtccatcaatggcatggtgaaccactcgtggagtgaactgtactccttcgcactcttcaaggccatgagccacatgctgtgcatcgggtacggccggcaggcgcccgagagcatgacggacatctggctgaccatgctcagcatgattgtgggtgccacctgctacgccatgttcatcggccacgccactgccctcatccagtcgctggactcctcgcggcgccagtaccaggagaagtacaagcaggtggagcagtacatgtccttccacaagctgccagctgacttccgccagaagatccacgactactatgagcaccgttaccagggcaagatgtttgacgaggacagcatcctgggcgagctcaacgggcccctgcgggaggagatcgtcaacttcaactgccggaagctggtggcctccatgccgctgttcgccaacgccgaccccaacttcgtcacggccatgctgaccaagctcaagttcgaggtcttccagccgggtgactacatcatccgcgaaggcaccatcgggaagaagatgtacttcatccagcacggcgtggtcagcgtgctcactaagggcaacaaggagatgaagctgtccgatggctcctacttcggggagatctgcctgctcacccggggccgccgcacggcgagcgtgcgggccgacacctactgccgcctctattcgctgagcgtggacaacttcaacgaggtgctggaggagtaccccatgatgcggcgcgccttcgagacggtggccatcgaccgcctggaccgcatcggcaagaagaattccatcctcctgcacaag

SEQ ID NO: 25 - DNA, 1520 bp, Homo sapiens
gcgccagttcggggccatgctccaacccggggtcaacaaattctccctaaggatgttcggcagccagaaagccgtggagcgcgaacaggagagggtcaagtcggccggattttggattatccacccctacagtgacttcagattttactgggacctgaccatgctgctgctgatggtgggaaacctgattatcattcctgtgggcatcaccttcttcaaggatgagaacaccacaccctggattgtcttcaatgtggtgtcagacacattcttcctcatcgacttggtcctcaacttccgcacagggatcgtggtggaggacaacacagagatcatcctggacccgcagcggattaaaatgaagtacctgaaaagctggttcatggtagatttcatttcctccatccccgtggactacatcttcctcattgtggagacacgcatcgactcggaggtctacaagactgcccgggccctgcgcattgtccgcttcacgaagatcctcagcctcttacgcctgttacgcctctcccgcctcattcgatatattcaccagtgggaagagatcttccacatgacctacgacctggccagcgccgtggtgcgcatcgtgaacctcatcggcatgatgctcctgctctgccactgggacggctgcctgcagttcctggtacccatgctacaggacttccctgacgactgctgggtgtccatcaacaacatggtgaacaactcctgggggaagcagtactcctacgcgctcttcaaggccatgagccacatgctgtgcatcggctacgggcggcaggcgcccgtgggcatgtccgacgtctggctcaccatgctcagcatgatcgtgggtgccacctgctacgccatgttcattggccacgccactgccctcatccagtccctggactcctcccggcgccagtaccaggaaaagtacaagcaggtggagcagtacatgtcctttcacaagctcccgcccgacacccggcagcgcatccacgactactacgagcaccgctaccagggcaagatgttcgacgaggagagcatcctgggcgagctaagcgagcccctgcgggaggagatcatcaactttaactgtcggaagctggtggcctccatgccactgtttgccaatgcggaccccaacttcgtgacgtccatgctgaccaagctgcgtttcgaggtcttccagcctggggactacatcatccgggaaggcaccattggcaagaagatgtacttcatccagcatggcgtggtcagcgtgctcaccaagggcaacaaggagaccaagctggccgacggctcctactttggagagatctgcctgctgacccggggccggcgcacagccagcgtgagggccgacacctactgccgcctctactcgctgagcgtggacaacttcaatgaggtgctggaggagtaccccatgatgcgaagggccttcgagaccgtggcgctggaccgcctggaccgcattggcaagaagaactccatcctcc

SEQ ID NO: 26 - DNA, 1527 bp, Mus musculus
gaggcagttcacctccatgctgcagcctggggtcaacaaattctccctccgcatgtttgggagccagaaggcggtggagaaggagcaggaaagggttaaaactgcaggcttctggattatccatccgtacagtgacttcaggttttattgggatttaatcatgcttataatgatggttggaaatttggtcatcataccagttggaatcacgttcttcacagagcagacgacaacaccgtggattattttcaacgtggcatccgatactgttttcctgttggacttaatcatgaattttaggactgggactgtcaatgaagacagctcggaaatcatcctggaccctaaagtgatcaagatgaattatttaaaaagctggtttgtggtggacttcatctcatcgatcccggtggattatatctttctcattgtagagaaagggatggactcagaagtttacaagacagccagagcacttcgtatcgtgaggtttacaaaaattctcagtctcttgcggttattacgcctttcaaggttaatcagatacatacaccagtgggaagagatattccacatgacctatgacctcgccagtgctgtggtgaggatcttcaacctcattggcatgatgctgcttctgtgccactgggatggctgtcttcagttcctggttcccctgctgcaggacttcccaccagattgctgggtttctctgaatgaaatggttaatgattcctggggaaaacaatattcctacgcactcttcaaagctatgagtcacatgctgtgcattggttatggcgcccaagcccctgtcagcatgtctgacctctggattaccatgctgagcatgattgtgggcgccacctgctacgcaatgtttgttggccatgccacagctttgatccagtctttggactcttcaaggaggcagtatcaagagaagtataagcaagtagagcaatacatgtcattccacaagttaccagctgacatgcgccagaagatacatgattactatgagcaccgataccaaggcaagatcttcgatgaagaaaatattctcagtgagcttaatgatcctctgagagaggaaatagtcaacttcaactgccggaaactggtggctactatgcctctttttgctaacgccgatcccaatttcgtgacggccatgctgagcaagctgagatttgaggtgttccagcccggagactatatcattcgagaaggagctgtggggaagaaaatgtatttcatccagcacggtgttgctggcgttatcaccaagtccagtaaagaaatgaagctgacagatggctcttacttcggagagatatgcctgctgaccaagggccggcgcactgccagtgtccgagctgatacctactgtcgtctttactccctttcggtggacaatttcaatgaggtcttggaggaatatccaatgatgagaagagcctttgagacagttgctattgaccgactcgatcggataggcaagaaaaactctattctcctgcagaa

SEQ ID NO: 27 - DNA, 1527 bp, Mus musculus
gcgccaattcggggcgcttctgcagcccggcgtcaacaagttctccctgcggatgttcggcagccagaaggccgtggagcgcgagcaggaacgcgtgaagtcggcgggggcctggatcatccacccctacagcgacttcaggttctactgggacttcaccatgctgttgttcatggtgggaaatctcattatcattcccgtgggcatcactttcttcaaggacgagaccaccgcgccctggatcgtcttcaacgtggtctcggacactttcttcctcatggacttggtgttgaacttccgcaccggcattgttattgaggacaacacggagatcatcctggaccccgagaagataaagaagaagtacttgcgtacgtggttcgtggtggacttcgtgtcatccatcccggtggactacatcttcctcatagtggagaagggaatcgactccgaggtctacaagacagcgcgtgctctgcgcatcgtgcgcttcaccaagatcctcagtctgctgcggctgctgcggctatcacggctcatccgatatatccaccagtgggaagagattttccacatgacctacgacctggcaagtgcagtgatgcgcatctgtaacctgatcagcatgatgctactgctctgccactgggacggttgcctgcagttcctggtgcccatgctgcaagacttccccagcgactgctgggtgtccatcaacaacatggtgaaccactcgtggagcgagctctactcgttcgcgctcttcaaggccatgagccacatgctgtgcatcggctacgggcggcaggcgcccgagagcatgacagacatctggctgaccatgctcagcatgatcgtaggcgccacctgctatgccatgttcattgggcacgccactgcgctcatccagtccctggattcgtcacggcgccaataccaggagaagtacaagcaagtagagcaatacatgtccttccacaaactgcccgctgacttccgccagaagatccacgattactatgaacaccggtaccaagggaagatgtttgatgaggacagcatccttggggaactcaacgggccactgcgtgaggagattgtgaacttcaactgccggaagctggtggcttccatgccgctgtttgccaatgcagaccccaacttcgtcacagccatgctgacaaagctcaaatttgaggtcttccagcctggagattacatcatccgagaggggaccatcgggaagaagatgtacttcatccagcatggggtggtgagcgtgctcaccaagggcaacaaggagatgaagctgtcggatggctcctatttcggggagatctgcttgctcacgaggggccggcgtacggccagcgtgcgagctgacacctactgtcgcctctactcactgagtgtggacaatttcaacgaggtgctggaggaataccccatgatgcggcgtgcctttgagactgtggctattgaccggctagatcgcataggcaagaagaactccatcttgctgcacaa

SEQ ID NO: 28 - DNA, 1547 bp, Mus musculus
gcgcctgggccagagcggcttcatgcagcgccagttcggtgccatgctgcaacctggggtcaacaaattctccctaaggatgttcggcagccagaaagcggtggagcgcgagcaggagagggttaagtcagcagggttttggattatccacccctacagtgacttcagattttactgggacctgacgatgctgttgctgatggtggggaatctgatcatcatacccgtgggcatcaccttcttcaaggatgagaacaccacaccctggatcgtcttcaatgtggtgtcagacacattcttcctcattgacttggtcctcaacttccgcacggggatcgtggtggaggacaacacagaaatcatccttgacccgcagaggatcaagatgaagtacctgaaaagctggtttgtggtagatttcatctcctccatccctgtcgactacatcttccttatagtggagactcgcattgactcggaggtctacaaaaccgctagggctctgcgcattgtccgtttcactaagatcctcagcctcctgcgcctcttgaggctttcccgcctcattcgatacattcatcagtgggaagagatcttccacatgacctatgacctggccagcgccgtggtacgcatcgtgaacctcattggcatgatgcttctgctgtgtcactgggatggctgcctgcagttcctagtgcccatgctgcaggacttcccccatgactgctgggtgtccatcaatggcatggtgaataactcctgggggaagcagtattcctacgccctcttcaaggccatgagccacatgctgtgcattgggtatggacggcaggcacccgtaggcatgtctgacgtctggctcaccatgctcagcatgatcgtgggggccacctgctatgccatgttcatcggccacgccactgccctcatccagtcgctagactcctcccggcgccagtaccaggagaagtataaacaggtggagcagtacatgtccttccacaagctcccgcctgacacccgacagcgcatccatgactactatgaacaccgctaccaaggcaagatgtttgatgaggaaagcatcctgggtgagctgagtgagccacttcgagaggagatcatcaactttaactgccgaaagctggtggcatccatgccactgtttgccaacgcagatcccaactttgtgacatccatgctgaccaagttgcgtttcgaggtcttccagcctggggattacatcatccgcgaaggcaccatcggcaagaagatgtactttatccagcacggcgtggtcagcgtgctcactaagggcaacaaagagaccaagctggctgatggctcctattttggagagatctgcttgctgacccggggtcggcgcacagccagcgtcagagcggatacttattgccgcctctactcactgagcgtggacaacttcaatgaggtgctggaggagtatcccatgatgcggagggccttcgagacggttgcgctggaccgcctggaccgcataggcaagaagaactccatcctcc

(19) United States
(12) Patent Application Publication
(10) Pub. No.: US 2007/0099268 A1
(43) Pub. Date: May 3, 2007

(54) Chimeric HCN channels

(76) Inventors: Ira S. Cohen, Stony Brook, NY (US); Michael R. Rosen, New York, NY (US); Peter R. Brink, Setauket, NY (US); Richard B. Robinson, Cresskill, NJ (US)

Correspondence Address: KENYON & KENYON LLP, ONE BROADWAY, NEW YORK, NY, 10004, US

(21) Appl. No.: 11/519,399

(22) Filed: Sep. 11, 2006

Related U.S. Application Data
(60) Provisional application No. 60/832,515, filed on Jul. 21, 2006.
(60) Provisional application No. 60/781,723, filed on Mar. 14, 2006.
(60) Provisional application No. 60/715,934, filed on Sep. 9, 2005.

(51) Int. Cl. C07K 14/705; C07H 21/04; C12P 21/06

(52) U.S. Cl. 435/69.1; 435/320.1; 435/325; 530/350; 536/23.5

(57) ABSTRACT

This invention provides a chimeric hyperpolarization-activated, cyclic nucleotide-gated (HCN) polypeptide comprising portions of more than one type of HCN channel. The invention also provides methods of treating a subject afflicted with a cardiac rhythm disorder comprising expression of the chimeric HCN polypeptide in a selected region of the heart so as to induce a pacemaker current in the heart and thereby treat the subject.

This application claims the benefit of U.S. Provisional Application Nos. 60/832,515, filed Jul. 21, 2006, 60/781,723, filed Mar. 14, 2006, and 60/715,934, filed Sep. 9, 2005, and U.S. Ser. No. 11/490,997, filed Jul. 21, 2006, the entire contents of which are incorporated herein by reference.

The invention disclosed herein was made with United States Government support under NIH Grant No. HL-28958 from the National Institutes of Health. Accordingly, the United States Government has certain rights in this invention.

Throughout this application, various publications are referenced in parentheses by author name and date, patent number, or patent publication number. Full citations for these publications may be found at the end of the specification immediately preceding the claims. The disclosures of these publications in their entireties are hereby incorporated by reference into this application in order to more fully describe the state of the art to those skilled therein as of the date of the invention described and claimed herein. However, the citation of a reference herein should not be construed as an acknowledgement that such reference is prior art to the present invention.

### FIELD OF THE INVENTION

The present invention relates to chimeric hyperpolarization-activated, cyclic nucleotide-gated (HCN) polypeptides comprising portions derived from more than one HCN isoform, and the expression of these chimeric polypeptides in the heart to induce a pacemaker current therein and thereby treat cardiac rhythm disorders.

### BACKGROUND OF THE INVENTION

The mammalian heart generates a rhythm that is myogenic in origin. All the channels and transporters that are necessary to generate the rhythm of the heart reside in the myocytes. Regional variations in the abundance or characteristics of these elements are such that the rhythm originates in a specific anatomic location, the sinoatrial node. The sinoatrial node consists of only a few thousand electrically active pacemaker cells that generate spontaneous rhythmic action potentials that subsequently propagate to induce coordinated muscle contractions of the atria and ventricles. The rhythm is modulated, but not initiated, by the autonomic nervous system.

Malfunction or loss of pacemaker cells can occur due to disease or aging. For example, acute myocardial infarction kills millions of people each year and generally induces in survivors marked reductions in myocyte number and cardiac pump function. Adult cardiac myocytes divide only rarely, and the usual responses to myocyte cell loss include compensatory hypertrophy and/or congestive heart failure, a disease with a significant annual mortality.

Electronic pacemakers are lifesaving devices that provide a regular heartbeat in settings where the sinoatrial node, atrioventricular conduction, or both, have failed. They also have been adapted to the therapy of congestive heart failure. One of the major indications for electronic pacemaker therapy is high degree heart block, such that a normally functioning sinus node impulse cannot propagate to the ventricle. The result is ventricular arrest and/or fibrillation, and death. Another major indication for electronic pacemaker therapy is sinoatrial node dysfunction, in which the sinus node fails to initiate a normal heartbeat, thereby compromising cardiac output.

Despite their utility in treating heart block and/or sinoatrial node dysfunction, electronic pacemakers have certain disadvantages, including their requirement for regular monitoring and maintenance, and their inadequate response to the demands of exercise or emotion (Rosen et al., 2004; Rosen, 2005; Cohen et al., 2005). Thus, although electronic pacemakers represent superb medical palliation, they are not a cure (Rosen et al., 2004). There is therefore a need for the development of alternatives that more completely reproduce normal function, e.g., by exhibiting autonomic responsiveness, and can ultimately provide a cure (Rosen et al., 2004).

As a therapeutic solution, a biological pacemaker, based on the expression of an ion channel in the heart, can be used to generate a spontaneous rate within the physiologically acceptable range. One of the key issues in advancing the field of biological pacemaking is identification of an ion channel(s) that (1) optimize(s) heart rhythm such that excessively long pauses do not occur following sudden failure of endogenous rhythms, and (2) induce(s) rhythms having physiologically low basal rates while maintaining an appropriate response to catecholamines and acetylcholine. Previous studies have focused for two reasons on the hyperpolarization-activated, cyclic nucleotide-gated (HCN) ion channels responsible for the I_{f} pacemaker current (Biel et al., 2002): first, the HCN ion current channels initiate pacemaker activity in the mammalian heart; and second, activation of these channels is increased by catecholamines and slowed by acetylcholine, making them autonomically responsive. Autonomic responsiveness should clearly be a cornerstone of pacemaker activity in the heart; yet, lack of this is a key shortcoming of electronic pacemakers.

The present invention relates to the production of chimeric HCN channels which exhibit improved characteristics, as compared to wild-type HCN channels, and the use of these chimeric channels for biological pacemaking and treating cardiac rhythm disorders.

### SUMMARY OF THE INVENTION

The invention disclosed herein provides a chimeric HCN polypeptide comprising portions derived from more than one HCN channel isoform. In preferred embodiments, these portions are an amino terminal portion, an intramembranous portion, and a carboxy terminal portion. In certain embodiments, at least one portion of the HCN chimera is derived from an animal species which is different from the animal species from which at least one of the other two portions is derived. In some embodiments, the intramembranous portion is derived from an HCN1 channel, or is D140-L400 of hHCN1 having the sequence set forth in SEQ ID NO:______, or is D129-L389 of mHCN1 having the sequence set forth in SEQ ID NO:______. In certain embodiments, the amino terminal portion is derived from HCN2, HCN3 or HCN4 and the carboxy terminal portion is derived from HCN2, HCN3 or HCN4. In a preferred embodiment, the amino terminal portion is derived from HCN2 and the carboxy terminal portion is derived from HCN2. Preferably, the chimeric HCN polypeptide provides an improved characteristic, as compared to a wild-type HCN channel, selected from the group consisting of faster kinetics, more positive activation, increased expression, increased stability, enhanced cAMP responsiveness, and enhanced neurohumoral response.

Exemplary chimeras include mHCN112, mHCN212, mHCN312, mHCN412, mHCN114, mHCN214, mHCN314, mHCN414, hHCN112, hHCN212, hHCN312, hHCN412, hHCN114, hHCN214, hHCN314, or hHCN414.

In certain embodiments, portions of the chimeras further comprise a mutant HCN channel. For instance, the mutant HCN channel may contain a mutation in a region of the channel selected from the group consisting of the S3-S4 linker, S4 voltage sensor, S4-S5 linker, S5, S6 and S5-S6 linker, C-linker, and the C-terminal cyclic nucleotide binding domain (“CNBD”). Exemplary mutant channels are derived from mHCN2 having the sequence set forth in SEQ ID NO:______, and include E324A-mHCN2, Y331A-mHCN2, R339A-mHCN2, and Y331A,E324A-mHCN2. Exemplary mutant channels, derived from mHCN1 having the sequence set forth in SEQ ID NO:______, include mHCN1-ΔΔΔ.

The present invention also provides a nucleic acid encoding any of the chimeric HCN polypeptides described herein and a vector comprising said nucleic acid. The invention further provides a cell comprising the instant nucleic acid, wherein the cell expresses the chimeric HCN polypeptide. In certain embodiments, the cell is an human adult mesenchymal stem cell (hMSC) that (a) has been passaged at least 9 times, (b) expresses CD29, CD44, CD54, and HLA class I surface markers; and (c) does not express CD14, CD34, CD45, and HLA class II surface markers.

The invention still further provides a pharmaceutical composition comprising the instant nucleic acid, vector or cell.

In addition, the invention provides a method of treating a subject afflicted with a cardiac rhythm disorder comprising administering to a region of the subject's heart any of the cells expressing a chimeric HCN polypeptide described herein, wherein expression of the chimeric HCN polypeptide in said region of the heart is effective to induce a pacemaker current in the heart and thereby treat the subject.

This invention also provides a method of treating a subject afflicted with a cardiac rhythm disorder comprising transfecting a cell of the subject's heart with a nucleic acid encoding a chimeric HCN polypeptide so as to functionally express the chimeric HCN polypeptide in the heart, wherein expression of the polypeptide is effective to induce a pacemaker current in the heart and thereby treat the subject.

The present invention further provides a method of producing a chimeric HCN polypeptide comprising (a) generating a recombinant nucleic acid by joining a nucleic acid encoding an amino terminal portion of a HCN polypeptide to a nucleic acid encoding an intramembranous portion of a HCN polypeptide and joining said nucleic acid encoding the intramembranous portion to a nucleic acid encoding a carboxy terminal portion of a HCN polypeptide, wherein the encoded portions of the HCN polypeptide are derived from more than one HCN isoform or mutant thereof, and (b) functionally expressing the recombinant nucleic acid in a cell so as to produce the chimeric HCN polypeptide.

This invention still further provides a tandem pacemaker system comprising (1) an electronic pacemaker, and (2) a biological pacemaker, wherein the biological pacemaker comprises an implantable cell that functionally expresses a chimeric HCN ion channel, and wherein the expressed chimeric HCN channel generates an effective pacemaker current when the cell is implanted into a subject's heart, and wherein the chimeric HCN comprises portions of more than one type of HCN channel. In preferred embodiments, the implantable cell is capable of gap junction-mediated communication with cardiomyocytes. In other embodiments, the cell is selected from the group consisting of a stem cell, a cardiomyocyte, a fibroblast or skeletal muscle cell engineered to express cardiac connexins, and an endothelial cell. In more preferred embodiments, the cell is a HMSC.

In preferred embodiments, the biological pacemaker of the tandem system comprises at least about 200,000 hMSCs and more preferably comprises at least about 700,000 hMSCs.

### BRIEF DESCRIPTION OF THE FIGURES

FIG. 1. Schematic representation of possible chimeric HCN channels. Illustrated are examples of channels constructed from elements of HCN2 (shown in light lines) and HCN1 (shown in dark lines), and designed to combine the rapid activation kinetics of HCN1 with the strong cAMP response of HCN2. The approach derives from the fact that the C-terminal cytoplasmic domain of the HCN channel contains the cyclic nucleotide binding domain and contributes significantly to cAMP responsiveness, whereas the transmembrane domain contributes significantly to the gating characteristics such as activation kinetics. Shown from top to bottom are: HCN2, HCN212 (in which the middle, transmembrane portion of HCN2 is replaced by the corresponding portion of HCN1), HCN112 (in which the C-terminal cytoplasmic portion of HCN1 is replaced by the corresponding portion of HCN2), and HCN1.

FIG. 2. Initiation of spontaneous rhythms by wild-type or genetically engineered pacemaker cells as well as by genetically engineered stem cell pacemakers. Top, In a native pacemaker cell or in a myocyte engineered to incorporate pacemaker current via gene transfer, action potentials (inset) are initiated via inward current flowing through transmembrane HCN channels. These open when the membrane repolarizes to its maximum diastolic potential and close when the membrane has depolarized during the action potential. Current flowing via gap junctions to adjacent myocytes results in their excitation and the propagation of impulses through the conducting system. Bottom, A stem cell has been engineered to incorporate HCN channels in its membrane. These channels can only open, and current can only flow through them (inset) when the membrane is hyperpolarized; such hyperpolarization can only be delivered if an adjacent myocyte is tightly coupled to the stem cell via gap junctions. In the presence of such coupling and the opening of the HCN channels to induce local current flow, the adjacent myocyte will be excited and initiate an action potential that then propagates through the conducting system. The depolarization of the action potential will result in the closing of the HCN channels until the next repolarization restores a high negative membrane potential. Thus, wild-type and genetically engineered pacemaker cells incorporate in each cell all the machinery needed to initiate and propagate action potentials. In contrast, in the stem cell-myocyte pairing, two cells together work as a single functional unit whose operation is critically dependent on the gap junctions that form between the two disparate cell types.

FIG. 3. The role of I_{f} in generation of pacemaker potentials in the sinoatrial node (SAN) (from Biel et al., 2002). A, Pacemaker potentials in the SAN under control conditions, and after β-adrenergic stimulation with norepinephrine (NE). The four major currents that control the generation of the pacemaker potential are indicated: I_{f} current (produced by hyperpolarization-activated cyclic nucleotide-gated [HCN] channels), T-type (I_{CaT}) and L-type (I_{CaL}) calcium currents, and repolarizing K currents (I_{K}). B, Scheme of an SAN cell showing the regulation of the HCN channel by up- or downregulation of cellular cyclic adenosine monophosphate (cAMP). M2, type-2 muscarinic receptor; ACh, acetylcholine; AC, adenylyl cyclase; Gαi, G-protein α subunit (inhibits AC); Gβγ, G-protein βγ subunit; β1-AR, type-1 β-adrenergic receptor; Gαs, G-protein α subunit (stimulates AC); ΔV, shift of the voltage dependence of HCN channel activation induced by increase or decrease of cAMP.

FIG. 4. Alignment of mammalian HCN1 polypeptide sequences. The mouse (SEQ ID NO:______, rat (SEQ ID NO:______), human (SEQ ID NO:______), rabbit (SEQ ID NO:______) and guinea pig (partial sequence; SEQ ID NO:______) HCN1 polypeptide sequences are aligned for maximum correspondence.

FIG. 5. Amino acid sequence of the human HCN212 chimeric channel. The shaded N-terminal portion of the sequence is derived from hHCN2; the underlined intramembranous portion from hHCN1; and the C-terminal portion (without shading or underline) from hHCN2. The amino acid sequence of the hHCN212 chimeric channel is set forth in SEQ ID NO:______. This 889-amino acid long chimeric hHCN212 sequence shows 91.2% identity with the 863-amino acid long mHCN212 sequence in 893 residues overlap when aligned for maximum correspondence.

FIG. 6. Amino acid sequence of the mouse HCN212 chimeric channel. The shaded N-terminal portion of the sequence is derived from mouse HCN2; the underlined intramembranous portion from mouse HCN1; and the C-terminal portion (without shading or underline) from mouse HCN2. The amino acid sequence of the mouse HCN212 chimeric channel is set forth in SEQ ID NO:______. This 863-amino acid long chimeric mHCN212 sequence shows 91.2% identity with the 889-amino acid long hHCN212 sequence in 893 residues overlap when aligned for maximum correspondence.

FIG. 7. Alignment of mammalian HCN2 polypeptide sequences. The mouse (SEQ ID NO:______), rat (SEQ ID NO:______), human (SEQ ID NO:______) and dog (partial sequence; SEQ ID NO:______) HCN2 polypeptide sequences are aligned for maximum correspondence.

FIG. 8. Alignment of mammalian HCN3 polypeptide sequences. The mouse (SEQ ID NO:______) and human (SEQ ID NO:______) HCN3 polypeptide sequences are aligned for maximum correspondence and exhibit 94.6% identity in 780 residues overlap. Asterisks indicate identical residues and periods indicate non-identical residues.

FIG. 9. Alignment of mammalian HCN4 polypeptide sequences. The mouse (SEQ ID NO:______), rat (SEQ ID NO:______), human (SEQ ID NO:______), rabbit (SEQ ID NO:______) and dog (partial sequence; SEQ ID NO:______) HCN4 polypeptide sequences are aligned for maximum correspondence.

FIG. 10. Functional expression of mHCN2 and mE324A in newborn ventricular myocytes. Representative whole-cell current traces of ventricular myocytes infected with AdmHCN2 (A) or AdmE324A (B). Currents were evoked by stepping from a holding potential of −10 mV to different hyperpolarizing voltage steps ranging from −25 to −125 mV with increments of −10 mV. Insets at right shown the current traces recorded at −35, −45 and −55 mV at an expanded scale for both mHCN2 and mE324A. C, For illustrative purposes the mean activation data of mHCN2 (squares) and mE324A (circles) currents were fitted to the Boltzmann equation (lines). D, Voltage-dependence of activation (filled symbols) and deactivation (unfilled symbols) time constants of mHCN2 (squares) and mE324A (circles). Mean activation values were obtained from 14 cells for both mHCN2 and mE324A; mean deactivation time constants values were obtained from 8 and 7 cells for mHCN2 and mE324A respectively.

FIG. 11. Modulation of mHCN2 and mE324A by cAMP. Mean fractional activation curves of mHCN2 (squares) and mE324A (circles) obtained in the absence (unfilled symbols) and in the presence (filled symbols) of 10 μM cAMP in the pipette solution. The average data were fit to the Boltzmann equation for experiments in the absence (solid lines) and in the presence (dashed lines) of cAMP. Calculated values for mHCN2 were V_{1/2}=−69.6 mV and −59.9 (9.7 mV shift) and s=10.8 and 11.0 mV in the absence and in the presence of cAMP respectively. Calculated values for mE324A were V_{1/2}=−46.3 mV and −40.7 mV (5.6 mV) and s=9.1 mV and 8.7 mV in the absence and in the presence of cAMP respectively.

FIG. 12. Activation of expressed wild-type mHCN2 or mutant mE324A in oocytes. A and B, Activation of the expressed mHCN2 (A) or mE324A (B). Upper panels: Typical recordings of the activation of expressed mHCN2 and mE324A. The inset shows the pulse protocol used. For mHCN2, currents were elicited by 2-s long hyperpolarizing pulses between −30 mV and −160 mV with 10 mV increments, followed by a 1-s depolarizing pulse to +15 mV. The holding potential was −30 mV. For mE324A, currents were elicited by 3-s long hyperpolarizing pulses between +20 mV and −130 mV with 10 mV increments, followed by a 1-s depolarizing pulse to +50 mV. The holding potential was +20 mV. Middle panels: The corresponding tail currents used for the construction of steady state activation curves. Lower panels: The activation curves for mHCN2 or mE324A. The data were fit to the Boltzmann equation (1/[1+exp((V_{1/2}−Vₜₑₛₜ)/s)])). The half maximal activation (Vₕ) for mHCN2 was −92.7 mV±1.1 mV (n=9 cells), and currents saturated around −130 mV. A more positive activation threshold was noticed for mE324A (around −30 mV) and the Vₕ was −57.3 mV±1.6 mV (n=9 cells). C and D, Activation time constants of mHCN2 and mE324A. Note both a positive shift in voltage dependence and faster activation kinetics for mE324A.

FIG. 13. cAMP modulation of I_{HCN2} in oocytes injected with mHCN2 or mE324A. The Boltzmann fit of normalized ionic conductance showed that extracellular application of 8-Br-cAMP (cAMP, 1 mM) positively shifted the potential of half-maximal activation (Vₕ) of I_{HCN2} for both mHCN2 (left panel) and mE324A (right panel) by 7-8 mV.

FIG. 14. The pharmacological evaluation and the reversal potential of I_{HCN2} for mHCN2 and mE324A. A and B, The current/voltage relationships of I_{HCN2} for mHCN2 (A) and mE324A (B). Upper panels: The voltage protocols for the recording of the current/voltage relationship of I_{f}. For mHCN2, the cell was held at −30 mV, current was elicited by a 2-s hyperpolarizing voltage step to −140 mV to saturate activation, and followed by 2-s depolarizing voltage steps between −80 mV and +50 mV in 10 mV increments. For mE324A, the cell was held at +20 mV, current was elicited by a 1.5-s hyperpolarizing voltage step to −110 mV to saturate activation, and then followed by 1.5-s depolarizing voltage steps between −80 mV and +50 mV in 10 mV increments for the recording of tail currents. Lower panels: The representative traces used to construct the fully activated current/voltage relationship of I_{HCN2} in the presence of control, Cs⁺ (5 mM) and washout conditions, respectively. Note a large inhibition of the I_{f} by Cs⁺ for both mHCN2 and mE324A. C and D, The fully activated current/voltage curves of for mHCN2 (C) and mE324A (D) in the presence of control, Cs⁺ and washout conditions. The fully activated current/voltage relations were constructed by dividing the tail current magnitudes by the change in gating variable which occurred between the two test voltages (obtained from FIGS. 15A and B). The calculated reversal potential of I_{HCN2} is −41 mV for mHCN2 and −40 mV for mE324A.

FIG. 15. Comparison of current magnitude of I_{HCN2} in oocytes injected with mHCN2 or mE324A. The I_{HCN2} was measured at −120 mV for mHCN2 (n=10 cells) and mE324A (n=10 cells). Note the smaller current magnitude for the expressed mE324A (t-test, P<0.01). Voltage protocols are shown in the insets. For mHCN2, the current was evoked by applying a 3-s hyperpolarizing voltage pulse to −120 mV from a holding potential of −30 mV. For mE324A, the current was evoked by applying a 3-s hyperpolarizing voltage pulse to −120 mV from a holding potential of +20 mV.

FIG. 16. Current traces in neonatal ventricular culture of native I_{f} and I_{f} expressed HCN2 or HCN4. A, Records from a control (non-transfected) myocyte. B, Records from a myocyte co-transfected with pCI-mHCN2 and pEGFP-C1 using lipofectin. C, Records from a myocyte co-transfected with pCI-mHCN4 and pEGFP-C1 using lipofectin. In all panels, the test voltage varied from −55 to −125 V in 10 mV increments. Note that selected traces are omitted from panel (A) for clarity.

FIG. 17. Activation-voltage relation and kinetics of expressed HCN2 and HCN4 in neonatal ventricle. A, I-V curves converted to activation relation using a Boltzmann relation. Activation relation for native current (dashed line) is taken from Qu et al. (2000). B, Time constant of current activation for native I_{f} and for expressed I_{HCN2} and I_{HCN4}.

FIG. 18. Current traces from adult ventricular myocytes. A, Records from an acutely isolated myocyte. B, Records from an adult myocyte maintained in culture for 48 hours. C, records from an adult myocyte infected with AdHCN2 and then maintained in culture for 48 hours. D, Illustration of voltage protocol. Note the different vertical scale in (C).

FIG. 19. Effect of HCN2 overexpression on spontaneous activity of neonatal ventricle culture. Monolayer culture was infested with AdHCN2 or AdGFP and spontaneous action potentials subsequently recorded with whole-cell patch electrodes. A, Spontaneous action potentials from a control monolayer culture. B, Spontaneous action potentials from an AdHCN2 infected monolayer culture. C, Summary data comparing control, AdHCN2 infected and AdGFP infected cultures with respect to spontaneous rate, slope of phase 4 depolarization and maximum diastolic potential (MDP). Asterisk indicates significant difference relative to control culture; n values for control were 16-17, for AdHCN2 infected were 12-16, and for AdGFP infected were 6.

FIG. 20. Modulation of rate by isoproterenol in an AdHCN2 infected culture. A, Action potential recordings of spontaneous rate during control superfusion. B, Recording from the same culture during superfusion with isoproterenol, demonstrating an increase in spontaneous rate from 48 beats/min during the control record to 63 beats/min during drug exposure.

FIG. 21. Modulation of rate by carbachol in an AdHCN2 infected culture. A, Action potential recordings of spontaneous rate during control superfusion. B, Recording from the same culture during superfusion with carbachol, demonstrating a decrease in spontaneous rate from 54 beats/min during the control record to 45 beats/min during drug exposure.

FIG. 22. Modulation of rate by ZD-7288 in an AdHCN2 infected culture. A, Action potential recordings of spontaneous rate during control superfusion. B, Recording from the same culture during superfusion with ZD-7288, demonstrating a decrease in spontaneous rate from 96 beats/min during the control record to 78 beats/min during drug exposure.

FIG. 23. Effect of threshold concentration of isoproterenol on expressed HCN2 current in a neonatal ventricular myocyte. Exposure to isoproterenol increased current for a voltage step to the midpoint of the activation curve without increasing the maximal current attained with a second voltage step to the maximum of activation curve, demonstrating that the nature of the effect was to shift activation curve positive on the voltage axis. Separate measurements indicated the magnitude of the shift in this cell was approximately 5 mV.

FIG. 24. Activation relation and kinetics of native I_{f} in adult myocytes. A, Activation relation for I_{f} in acutely dissociated and cultured adult ventricular myocytes. B, Time constant of current activation for native I_{f} in acutely isolated and cultured adult ventricular myocytes. Neonatal data from FIG. 17 is superimposed as dashed line for comparison.

FIG. 25. Activation relation and kinetics of I_{HCN2} expressed with AdHCN2 in neonatal and adult ventricle. A, Activation relations for neonatal and adult ventricle cultures as measured by tail currents. B, Time constant of activation (squares) and deactivation (circles) for neonatal and adult myocytes. Lines are generated by a best fit to the equation.

FIG. 26. Regression relation for V_{1/2} of Boltzmann relation as a function of expressed HCN2 current density in neonatal and adult myocytes. Cultures were infected with AdHCN2. Lines are calculated linear regressions. The vertical and horizontal error bars represent S.E.M. of V_{1/2} and I_{HCN2}, respectively. Inset shows expanded time scale for current densities <60 pA/pF.

FIG. 27. Effect of intracellular cAMP on activation relation of expressed HCN2 current in neonate and adult myocytes. Earlier data with control pipette solution (FIG. 25A) are shown as dashed (neonate) and dotted (adult) lines.

FIG. 28. Effect of HCN2 overexpression in adult ventricular myocytes. A, Representative anode break excitation tracings from a control myocyte (left, including stimulus time course) and an AdHCN2 infected myocyte (right). Resting potential in the two examples is −66 and −60 mV, respectively. Only selected traces are shown for clarity. B, Graph of relation between maximal negative potential achieved during anodal stimulation as a function of I_{f} or I_{HCN2} current density (measured at the end of a 2-s step to −125 mV). Inset shows current density range of 0-1.2 pA/pF on an expanded time base, with calculated linear regression as solid line.

FIG. 29. Functional expression of HCN1 and HCN2 channels with and without minK and MiRP1 in Xenopus oocytes. The holding potential is −35 mV, and the voltage increment is always 10 mV. A, 5 ng HCN1 cRNA injection and test pulses 3-s long from −65 mV to a maximum voltage of −115 mV. B, 5 ng HCN1 plus 0.2 ng minK injection with test pulses 3-s long from a minimum voltage of −55 mV to a maximum voltage of −115 mV. C, 5 ng HCN1 plus 0.2 ng MiRP1 injection with test pulses 3-s long from −55 mV to −115 mV. D, 5 ng HCN2 cRNA injection with test pulses 8-s long from −55 mV to −95 mV. E, 5 ng HCN2 plus 0.2 ng minK injection with test pulses 8-s long from −65 mV to −105 mV. F, 5 ng HCN2 plus 0.2 ng MiRP1 injection with test pulses 8-s long from −55 mV to −95 mV. G, The maximum conductance of the tail current was obtained by dividing its amplitude by the driving force at that potential.

FIG. 30. Gating properties of the expressed channels. A, Activation curves of HCN1 alone and HCN1 coexpressed with MiRP1. The inset shows the representative tail currents used to construct the activation curve. B, Activation curves of HCN2 alone and HCN2 coexpressed with MiRP1. C, Sample data illustrating activation kinetics of HCN1 alone and HCN1 coexpressed with MiRP1. D, Sample data illustrating activation kinetics of HCN2 alone and HCN2 coexpressed with MiRP1. E, Plot of activation and deactivation (in box) time constants for HCN1 alone and HCN1+MiRP1. F, Same as (E) but for HCN2 and HCN2+MiRP1.

FIG. 31. MiRP1 mRNA expression in rabbit as determined by RNase protection assays. A, An example of a representative RPA performed on 2 μg of total RNA isolated from left ventricle, right atrium, SA node and whole brain. B, Histogram showing the relative abundance of MiRP1. Data are normalized to the cyclophilin protected fragment; values are the means of three independent mRNA samples and the error bars are SEM.

FIG. 32. Western blots showing protein expression of HCN1 channel subunits with and without MiRP1 in Xenopus oocytes following immunoprecipitation with the HCN1 ion channel subunit. A, Proteins in oocyte membranes fractionated and probed with anti-HCN1 antibody. B, Oocyte membrane protein probed with anti-HA antibody. C, Products of IP reactions by anti-HCN1 antibody from membrane protein from oocytes injected with HCN1, MiRP1 or by both cRNAs probed with anti-HA antibody.

FIG. 33. Identification of connexins in gap junctions of human mesenchymal stem cells (hMSCs). Immunostaining of Cx43 (A), Cx40 (B) and Cx45 (C). D, Immunoblot analysis of Cx43 in canine ventricle myocytes and hMSCs. Whole cell lysates (120 μg) from ventricle cells or hMSCs were resolved by SDS, transferred to membranes, and blotted with Cx43 antibodies. Molecular weight markers are indicated.

FIG. 34. Macroscopic and single channel properties of gap junctions between hMSC pairs. Gap junction currents (Iⱼ) elicited from hMSCs using a symmetrical bipolar pulse protocol (10 s, from±10 mV to ±10 mV, Vₕ=0 mV) showed two types of voltage-dependent current deactivation: symmetrical (A) and asymmetrical (B). C, summary plots of normalized instantaneous (∘) and steady-state (●) gⱼ versus Vⱼ. Left panel, quasi-symetrical relationship from 5 pairs; continuous line, Boltzmann fit: V_{j,0}=−70/65 mV, g_{j,min}=0.29/0.34, g_{j,max}=0.99/1.00, z=2.2/2.3 for negative/positive Vⱼ. Right panel, asymmetrical relationship from 6 pairs; Boltzmann fit for negative Vⱼ: V_{j,0}=−72 mV, g_{j,min}=0.25, g_{j,max}=0.99, z=1.5. D and E, single channel recordings from pairs of hMSCs. Pulse protocol (V₁ and V₂) and associated multichannel currents (I₂) recorded from a cell pair during maintained Vⱼ of ±80 mV. The discrete current steps indicate the opening and closing of single channels. Dashed line: zero current level. The all points current histograms on the right-hand side reveal a conductance of ˜50 pS.

FIG. 35. Macroscopic properties of junctions in cell pairs between a hMSC and HeLa cell expressing only Cx40, Cx43 or Cx45. In all cases hMSC to Hela cell coupling was tested 6 to 12 after hours initiating co-culture. A, Iⱼ elicited in response to a series of 5-s voltage steps (Vⱼ) in hMSC-HeLaCx43 pairs. Top, symmetrical current deactivation; bottom, asymmetrical current voltage dependence. B, Macroscopic Iⱼ recordings from hMSC-HelaCx40 pairs exhibit symmetrical (top panel) and asymmetrical (bottom panel) voltage dependent deactivation. C, Asymmetric Iⱼ from hMSC-HeLaCx43 pair exhibits voltage dependent gating when Cx45 side is relatively negative. Iⱼ recorded from hMSC. D,g_{j,ss} plots versus Vⱼ from pairs between hMSC and transfected HeLa cells. Left panel, hMSC-HeLaCx43 pairs, quasi-symmetrical relationship (●) and asymmetrical relationship (∘); continuous and dashed lines are Boltzmann fits (see text for details). Middle panel, symmetrical (●) and asymmetrical (∘) relationships from hMSC-HeLaCx 40 pairs; the continuous and dashed lines correspond to Boltzmann fits (see text for details). Right panel, asymmetrical relationship from hMSC-HeLaCx45 cell pairs; continuous line, Boltzmann fit for positive Vⱼ (see text for details). E, Cell-to-cell Lucifer Yellow (LY) spread in cell pairs: from an hMSC to an hMSC (upper panel), from a HeLaCx43 to an hMSC (middle panel), and from an hMSC to a HeLaCx43 (bottom panel). In all cases a pipette containing 2 mM LY was attached to the left-hand cell in the whole-cell configuration. Epifluorescent micrographs taken at 12 min after dye injection show LY spread to the adjacent (right-hand) cell. The simultaneously measured junctional conductance revealed gⱼ of ˜13 nS, ˜16 nS, and ˜18 nS of the pairs, respectively. Cell Tracker green was used to distinguish hMSCs from HeLa cells or vice versa in all experiments.

FIG. 36. Macroscopic and single channel properties of gap junctions between hMSC-canine ventricle cell pairs. Myocytes were plated between 12 and 72 h and co-cultured with hMSCs for 6 to 12 h before measuring coupling. A, Localization of Cx43 for hMSC-canine ventricle cell pairs. Most of Cx43 was localized to the ventricular cell ends and a small amount of Cx43 was present along the lateral borders. The intensive Cx43 staining was detected between the end of the rod-shaped ventricular cell (middle cell) and the hMSC (right cell). There is no detectable Cx43 staining between the ventricular cell and the hMSC on the left side. B, Top, phase-contrast micrograph of a hMSC-canine ventricular myocyte pair. Bottom, monopolar pulse protocol (V₁ and V₂) and associated macroscopic junctional currents (I₂) exhibiting asymmetrical voltage dependence. C, Top, multichannel current elicited by symmetrical biphasic 60 mV pulse. Dashed line, zero current level; dotted lines, represent discrete current steps indicative of opening and closing of channels. The current histograms yielded a conductance of ˜40-50 pS. Bottom, multichannel recording during maintained Vⱼ of 60 mV. The current histograms revealed several conductances of 48-64 pS with several events with conductance of 84 pS to 99 pS (arrows) which resemble operation of Cx43, heterotypic Cx40-Cx43 and/or homotypic Cx40 channels.

FIG. 37. Comparison of gating kinetics of mHCN2 and chimeric mHCN212 channels when expressed in neonatal rat ventricular myocytes. Results using mHCN2 (solid squares) and a chimeric mHCN212 channel (solid circles) are shown. Left, Activation kinetics, determined by fitting the early portion of the current traces (after omitting the initial delay) to a single exponential, for hyperpolarizing test potentials to the voltages indicated on the X-axis. Right, Deactivation kinetics, determined by fitting the current trace from depolarizing test potentials to the indicated voltages following a pre-pulse to a negative potential to fully activate the channels. The time constant of the single exponential fit is plotted on the y-axis in each case, illustrating faster kinetics at all voltages for mHCN212 compared to mHCN2.

FIG. 38. Comparison of expression efficiency of mHCN2 and chimeric mHCN212 channels in neonatal rat ventricular myocytes. Left, Mean current density of expressed current for a step to a negative voltage that maximally activates the channels. Right, Plot of voltage dependence of activation.

FIG. 39. Comparison of mHCN212 characteristics expressed in myocytes and stem cells. The current generated from expression of murine HCN212 in neonatal rat ventricular myocytes and human adult mesenchymal stem cells was measured. Left, voltage dependence of activation; Right, kinetics of activation.

FIG. 40. Properties of wildtype mHCN2 and mHCN112 expressed in oocytes. The steady state activation curve (A), activation kinetics (B) and cAMP modulation (C) are depicted.

FIG. 41. Comparison of gating characteristics of HCN2 and chimeric HCN212 channels when expressed in adult human mesenchvmal stem cells. Left, Voltage dependence of activation is shifted significantly positive for mHCN212 (solid circles) compared to HCN2 (solid squares). Right, Kinetics of activation at any measured voltage are significantly faster for mHCN212 compared to HCN2.

FIG. 42. Comparison of performance of biological-electronic tandem pacemaker versus electronic-only pacemaker. A, Percent of electronically paced beats occurring in hearts injected with saline and implanted with an electronic pacemaker or injected with mHCN2 in tandem with an electronic pacemaker. In both groups the electronic pacemaker was set at VVI 45 bpm. Throughout the 14 day period the number of beats initiated electronically was higher in the saline-injected group than in the HCN2-injected group (P<0.05) for comparisons at each time point). B, Mean basal heart rate over days 1-7 and 8-14 of groups injected with saline, mHCN2 or mE324A. Rate in the latter two groups was significantly faster than in the saline group (P<0.05).

FIG. 43. Representative trace of interaction between biological and electronic pacemaker components of tandem unit. This animal had been administered mHCN2. There is a smooth transition from biological to electronic pacemaker activity and from electronic back to biological.

FIG. 44. Effects of epinephrine infusion on biological-electronic tandem pacemaker versus electronic-only pacemaker. IV infusions of 1.0, 1.5 and 2.0 ug/kg/min were given on day 14 until there was either a 50% increase in non-electrically driven pacemaker rate, an arrhythmia occurred, or a maximal dose of 2 μg/kg/min was administered for 10 min. A, Effects of epinephrine, 1 μg/kg/min, on ECGs in three representative animals. Note the greatest rate increase in the mE324A-administered animal. B, A 50% increase in heart rate resulting from idioventricular pacemaker function is indicated in grey. In the saline group, the protocol terminated with all animals having either <50% increase at the highest dose (75% of animals) or an arrhythmia (25% of animals). In the mHCN2 group, 50% of animals had less than a 50% increase in rate: in one animal infusion was terminated because the highest dose was achieved whereas two animals developed ventricular arrhythmias. Of the other 50%, one achieved the 50% rate increase at the lowest epinephrine dose and the other two required 1.5 or 2 μg/kg/min. In contrast, in the mE324A group, 100% achieved a 50% increase in rate at the lowest epinephrine dose and no arrhythmias were seen.

FIG. 45. Comparison of mHCN2 and chimeric mHCN212 provided to rat myocytes in an adenoviral vector. mHCN212 demonstrated a higher basal signal frequency than HCN2, and a less negative maximum diastolic potential.

FIG. 46. Autonomic responsiveness of mHCN2 and HCN212 in newborn rat myocytes. mHCN212 exhibits autonomic responsiveness, demonstrated by an increased signal frequency after exposure to isoproterenol (a beta adrenergic receptor agonist).

FIG. 47. Expression of mHCN212 in human mesencymal stem cells. Panel A shows that hMSCs are expressing GFP, which was co-expressed with mHCN212. GFP is seen in the slides. An electrical potential was applied to the cells following the voltage protocol shown in Panel B. Panel C shows that the current response was blocked, as expected, by cesium.

FIG. 48. Activation of expressed mHCN212 in human mesenchymal stem cells (MSCs). Panel A shows that the amount of current varies with the amount of electrical potential applied. Panel B shows the relationship between the voltage applied and the current generated.

FIG. 49. cAMP modulation of expressed mHCN212 in human mesenchymal stem cells. For a given electrical potential, cAMP will increase the current response. A positive shift for voltage dependence is seen in the presence of cAMP, which indicates a good autonomic responsiveness.

FIG. 50. Expression of mHCN212 in human mesenchymal stem cells provides a higher current density than mHCN2. “n” equals the number of cells tested.

FIG. 51. Characteristics of a biological pacemaker. mHCN2 and mHCN212 express current density(Panel A and B, respectively). Panel C shows that mHCN212 has a more positive current response to an applied electrical potential than mHCN2. Panels D and E show kinetics and demonstrate that HCN212 has faster kinetics than HCN2.

FIG. 52. hMSCs expressing HCN2 provide pacemaker current to generate a stable heart beating rate by day 12-14 after implant. As the number of hMSCs loaded with HCN2 increases, so does the rate. A steady state is reached above roughly 500,000 hMSCs

FIG. 53. Percent of beats triggered by a electronic pacemaker decreased as a function of biological pacemaking by hMSCs on days 12-42 after implant. Dogs were implanted with hMSCs expressing mHCN2. The electronic pacemaker was set to fire when the heart rate fell below 35 beats per minute. As demonstrated in the figure, the number of beats triggered by the electronic pacemaker decreased with implantation of a biological pacemaker comprising about 700,000 hMSCs engineered to express mHCN2.

### DETAILED DESCRIPTION OF THE INVENTION

Hyperpolarization-activated cation currents, termed I_{f}, Iₕ, or I_{q}, were initially discovered in heart and nerve cells over 20 years ago (for review, see DiFrancesco, 1993; Pape, 1996). These currents, carried by Na⁺ and K⁺ ions, contribute to a wide range of physiological functions, including cardiac and neuronal pacemaker activity, the setting of resting potentials, input conductance and length constants, and dendritic integration (see Robinson and Siegelbaum, 2003; Biel et al., 2002). The hyperpolarization-activated, cyclic nucleotide-gated (HCN) family of ion channel subunits has been identified by molecular cloning (for review, see Clapham, 1998; Santoro and Tibbs, 1999; Biel et al., 2002), and when heterologously expressed, each of the four different HCN isoforms (HCN1-4) generates channels with the principal properties of native I_{f}, confirming that HCN channels are the molecular correlate of this current. The molecular components of the channels thus present a natural target for modulating heart rate.

In general terms, HCN polypeptides can be divided into three major portions: (1) a cytoplasmic amino terminal domain; (2) an intramembranous portion comprising the membrane-spanning domains and their linking regions; and (3) a cytoplasmic carboxy-terminal domain. The N-terminal domain does not appear to play a major role in channel activation (Biel et al., 2002). However, the membrane-spanning domains with their linking regions play an important role in determining the kinetics of gating, whereas the C-terminal CNBD is largely responsible for the ability of the channel to respond to the sympathetic and parasympathetic nervous systems that respectively raise and lower cellular cAMP levels.

Chimeric HCN Channels

Wang et al. (2001b) used chimeras between HCN1 and HCN2 to investigate the molecular bases for the modulatory action of cAMP and for the differences in the functional properties of the two channels. The present invention encompasses manipulation of the properties of HCN channels by in vitro recombination of nucleotide sequences encoding portions of all four HCN isoforms to produce chimeric HCN channels. As detailed in the Examples, certain of these chimeric channels exhibit characteristics which are advantageous, compared to wild type channels, for generating pacemaker currents for use in treating heart disorders.

As such, the present invention provides a chimeric HCN polypeptide comprising portions derived from more than one HCN isoform. There are four HCN isoforms: HCN1, HCN2, HCN3 and HCN4. All four isoforms are expressed in brain; HCN1, HCN2 and HCN4 are also prominently expressed in heart, with HCN4 and HCN1 predominating in sinoatrial node and HCN2 in the ventricular specialized conducting system. “mHCN” designates murine or mouse HCN; “hHCN” designates human HCN. The HCN channel may be any HCN channel that is capable of inducing biological pacemaker activity.

In preferred embodiments, the portions are an amino terminal portion, an intramembranous portion, and a carboxy terminal portion. In other preferred embodiments, the portions are derived from human HCN isoforms. As used herein, a “chimeric HCN polypeptide” or “HCN chimera” shall mean a HCN polypeptide comprising portions of more than one HCN channel isoform. Thus, a chimera may comprise portions of HCN1 and HCN2 or HCN3 or HCN4, and so forth. For example, this invention also provides a human chimeric HCN polypeptide comprising an amino terminal portion of a human HCN1 channel or a human HCN2 channel contiguous with an intramembranous portion of a human HCN channel contiguous with a carboxy terminus portion of a human HCN channel, wherein one portion is derived from an HCN channel which is different from the HCN channel from which at least one of the other two portions is derived.

In certain embodiments, at least one portion of the HCN chimera is derived from an animal species which is different from the animal species from which at least one of the other two portions is derived. For example, one portion of the channel may be derived from a human and another portion may be derived from a non-human.

The term “HCNXYZ” (wherein X, Y and Z are any one of the integers 1, 2, 3 or 4, with the proviso that at least one of X, Y and Z is a different number from at least one of the other numbers) shall mean a chimeric HCN polypeptide comprising three contiguous portions in the order XYZ, wherein X is an N-terminal portion, Y is an intramembranous portion, and Z is a C-terminal portion, and wherein the number X, Y or Z designates the HCN channel from which that portion is derived. For example, HCN112 is an HCN chimera with a N-terminal portion and intramembranous portion from HCN1 and a C-terminal portion from HCN2.

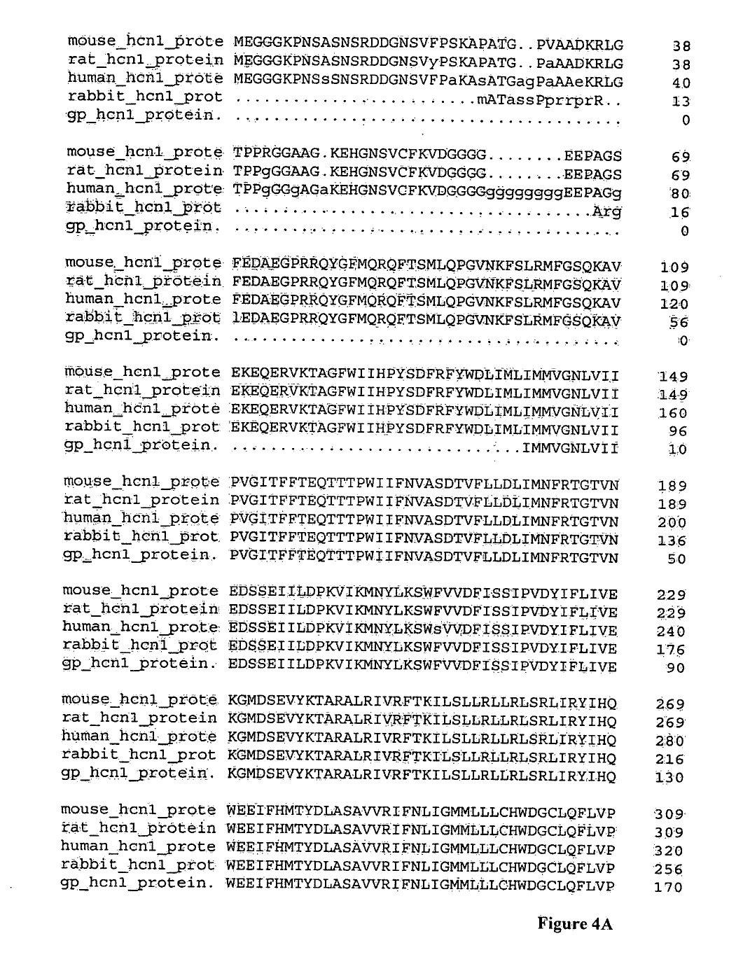

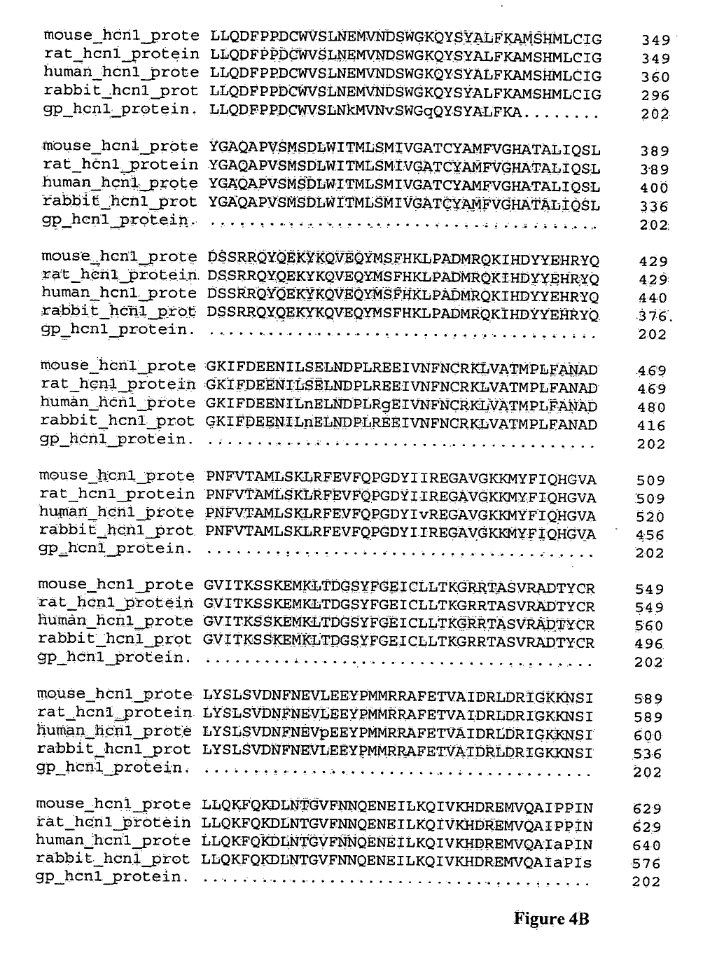

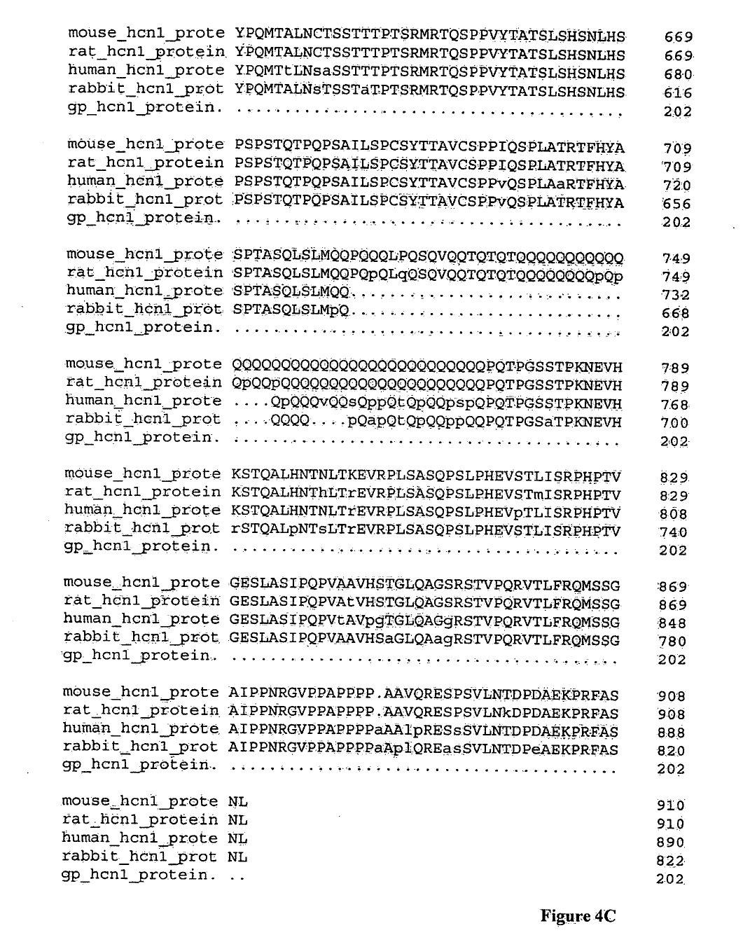

In other embodiments of the instant chimeric HCN polypeptide, the intramembranous portion is derived from an HCN1 channel. In further embodiments, the intramembranous portion is D140-L400 of hHCN1 having the sequence set forth in SEQ ID NO:______ (see FIG. 4). In still further embodiments, the intramembranous portion is D129-L389 of mHCN1 having the sequence set forth in SEQ ID NO:______ (see FIG. 4). In different embodiments, the amino terminal portion is derived from HCN2, HCN3 or HCN4 and the carboxy terminal portion is derived from HCN2, HCN3 or HCN4. In other embodiments, the amino terminal portion is derived from HCN2 and the carboxy terminal portion is derived from HCN2.

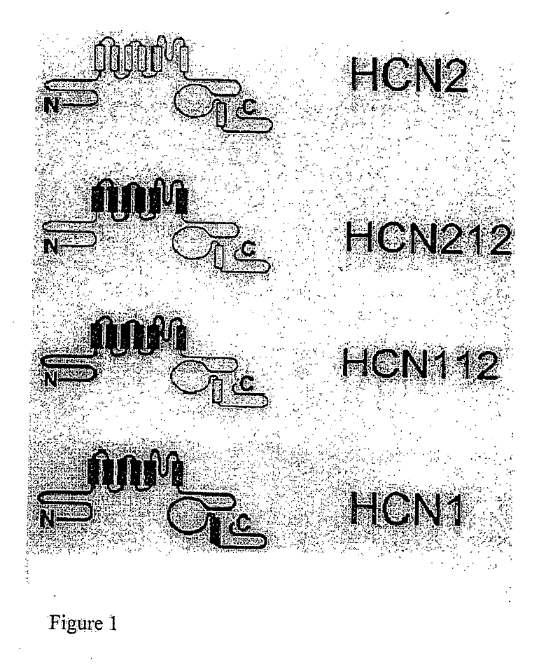

Preferred embodiments of the present invention provide a chimeric HCN polypeptide that exhibits an improved characteristic, as compared to a wild-type HCN channel, selected from the group consisting of faster kinetics, more positive activation, increased expression, increased stability, enhanced cAMP responsiveness, and enhanced neurohumoral response. HCN1 has the fastest kinetics but poor cAMP responsiveness. HCN2 has slower kinetics and good cAMP responsiveness. Accordingly, chimeras of HCN1 and HCN2 were studied experimentally and the invention provides pacemaker systems comprising cells expressing these and other chimeras. A schematic representation of HCN1/HCN2 chimeras is shown in FIG. 1.

In other embodiments, the instant chimeric HCN polypeptide comprises mHCN112, mHCN212, mHCN312, mHCN412, mHCN114, mHCN214, mHCN314, mHCN414, hHCN112, hHCN212, hHCN312, hHCN412, hHCN114, hHCN214, hHCN314, or hHCN414.

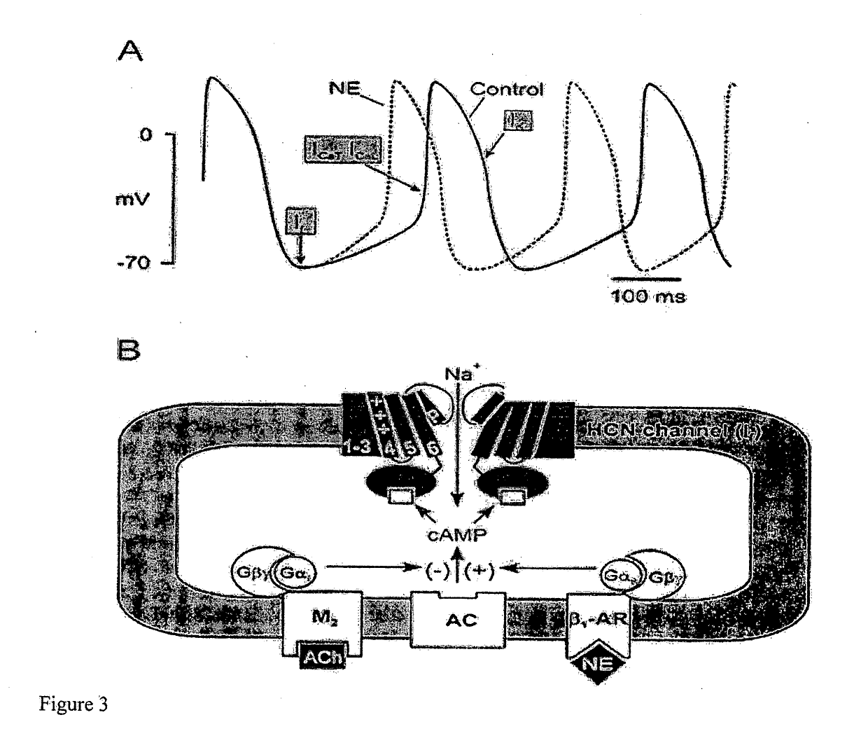

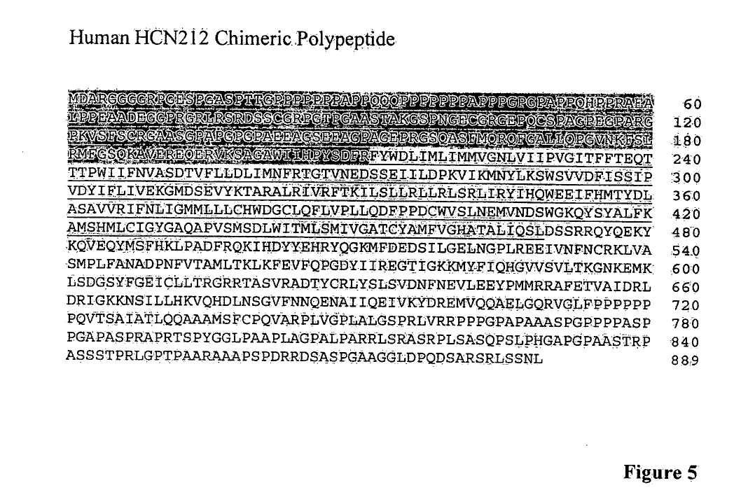

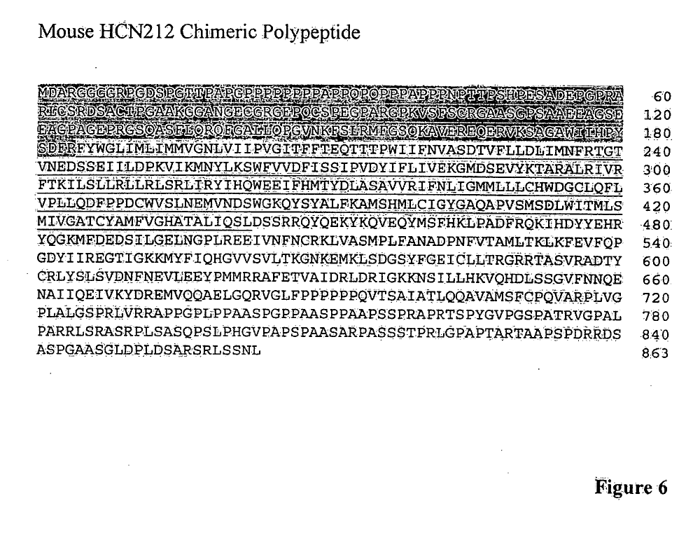

The HCN112 chimera (containing the N-terminal domain of HCN1, membrane spanning domains of HCN1, and C-terminal domain of HCN2; see FIG. 1) is a preferred chimeric channel for biological pacemaking because it contains the relevant membrane spanning domains of HCN1 (exhibiting fast kinetics) and the C-terminal domain of HCN2 (exhibiting good cAMP responsiveness). Since the contribution of the N-terminal domain to channel gating and cAMP responsiveness is not defined, HCN212 (see FIG. 3) is also a preferred candidate. Thus, in a preferred embodiment, the chimeric HCN polypeptide is hHCN212 having the sequence set forth in SEQ ID NO:______ (see FIG. 5). In yet another preferred embodiment, the chimeric HCN polypeptide is mHCN212 having the sequence set forth in SEQ ID NO:______ (see FIG. 6). Other preferred chimeras are HCN312 and HCN412. HCN4 also exhibits slow kinetics and good cAMP responsiveness; thus, HCN114, HCN214, HCN314 and HCN414 are also preferred chimeras.

Whereas the HCN channels are defined above in terms of three broad functional domains, there are multiple locations at which the borders between these domains in a chimeric channel could be set. The present invention also encompasses variants of HCN chimeras created using domains with differently defined boundaries that also serve to recombine the desirable biochemical and biophysical characteristics of individual HCN channels.

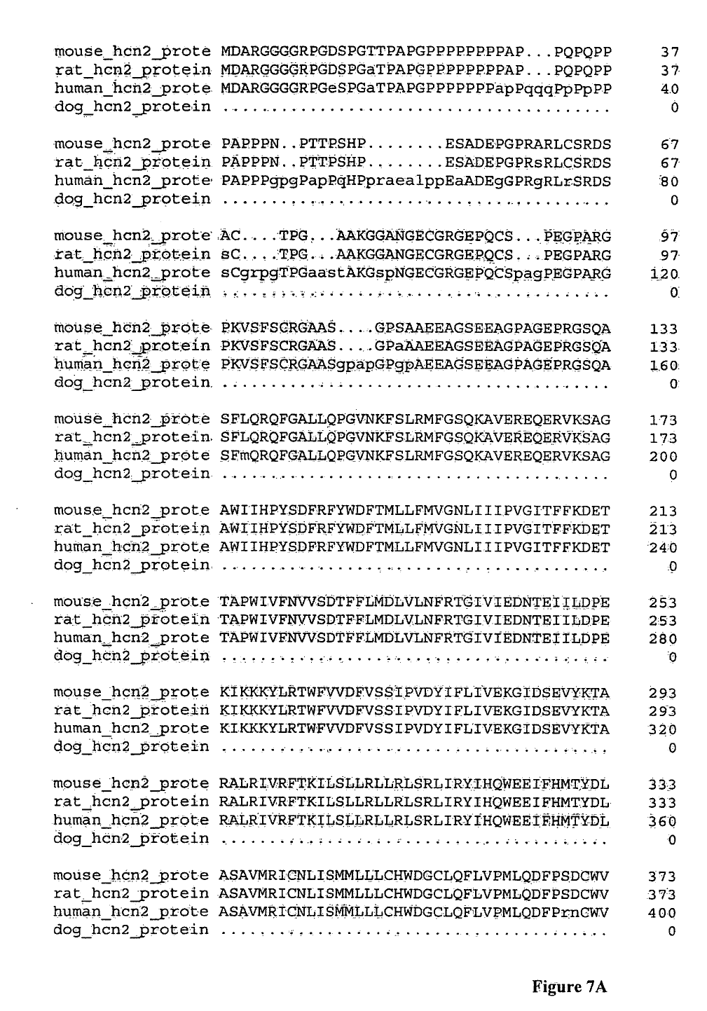

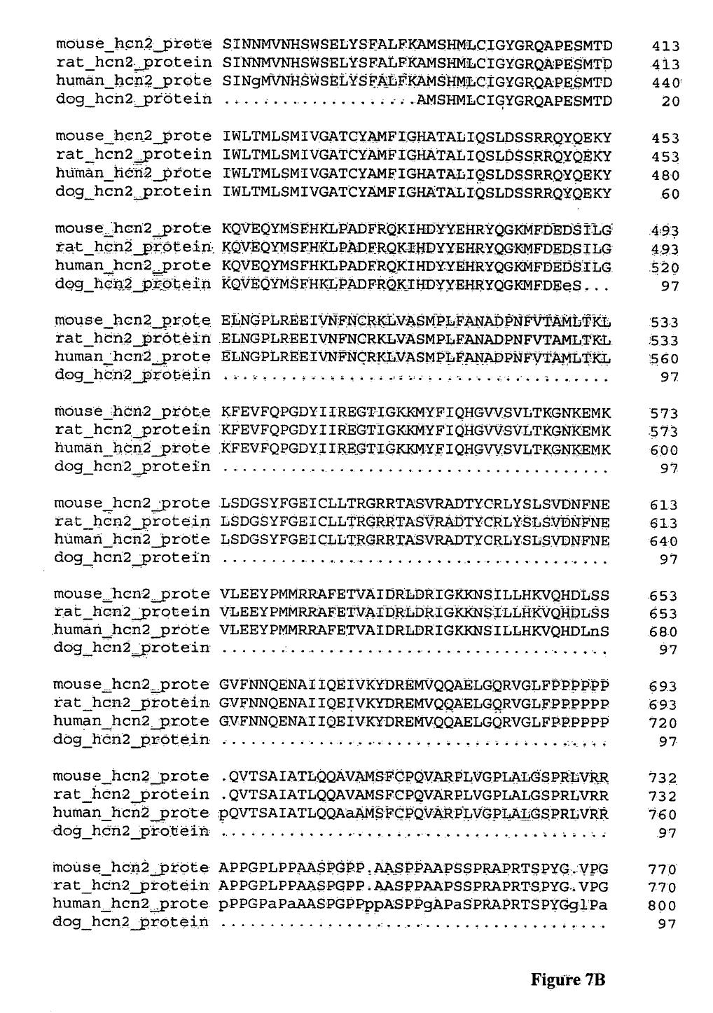

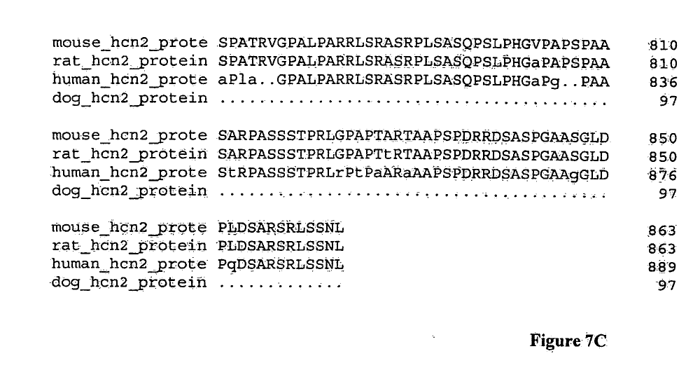

In certain embodiments, the HCN chimera comprises an amino terminal portion contiguous with an intramembrane portion contiguous with a carboxy terminal portion, wherein each portion is a portion of an HCN channel or a portion of a mutant thereof, and wherein one portion derives from an HCN channel or a mutant thereof which is different from the HCN channel or mutant thereof from which at least one of the other two portions derive. In various embodiments, at least one portion of the polypeptide is derived from a HCN channel containing a mutation which provides an improved characteristic, as compared to a portion from a wild-type HCN channel, selected from the group consisting of faster kinetics, more positive activation, increased expression, increased stability, enhanced cAMP responsiveness, and enhanced neurohumoral response. In certain embodiments, the mutant HCN channel contains a mutation in a region of the channel selected from the group consisting of the S3-S4 linker, S4 voltage sensor, S4-S5 linker, S5, S6 and S5-S6 linker, C-linker, and the C-terminal CNBD. In other embodiments, the mutant portion is derived from mHCN2 having the sequence set forth in SEQ ID NO:______ (see FIG. 7) and comprises E324A-mHCN2, Y331A-mHCN2, R339A-mHCN2, or Y331A,E324A-mHCN2. In preferred embodiments, the mutant portion comprises E324A-mHCN2. In certain other embodiments, the mutant portion comprises HCN1-Δ229-231, HCN1-Δ233-237, HCN1-Δ234-237, HCN1-Δ235-237, HCN1-Δ229-231/Δ233-237, HCN1-Δ229-231/Δ234-237, and HCN1-Δ229-231/Δ235-237 (see Tsang et al., 2004). In preferred embodiments, the mutant portion comprises HCN1-Δ235-237 (also referred to herein as HCN1-ΔΔΔ; see Tse et al., 2006), the S3-S4 linker of which has been systematically shortened by deleting residues 235-237 to favor channel opening.

Polypeptide mutations involving amino acid substitutions are identified herein by a designation with provides the single letter abbreviation of the amino acid residue that underwent mutation, the position of that residue within a polypeptide, and the single letter abbreviation of the amino acid residue to which the residue was mutated. Thus, for example, E324A identifies a mutant polypeptide in which the glutamate residue (E) at position 324 was mutated to alanine (A). Y331A, E324A-HCN2 indicates a mouse HCN2 having a double mutation, one in which tyrosine (Y) at position 331 was mutated to alanine (A), and the other in which the glutamate residue at position 324 was mutated to alanine.

HCN mutants resulting from deletions within the S3-S4 linker are identified herein by a “Δ” designation, wherein the amino acid residues deleted are indicated by their numbered positions within the polypeptide chain. Thus, for example, HCN1-Δ229-231/Δ235-237 identifies a mutant HCN1 polypeptide in which the residues at positions 229-231 and 235-237 were deleted.

Nucleic Acids Encoding Chimeric HCN Channels and Vectors Comprising Same

This invention also provides a nucleic acid encoding any of the chimeric HCN polypeptides described herein. The nucleic acid may be a DNA, an RNA, or a mixture thereof. The DNA may be a cDNA or a genomic DNA. This invention also provides a nucleic acid capable of specifically hybridizing under high stringency conditions (0.5×SSC or SSPE buffer, 1% SDS, at 68° C.) to the instant nucleic acids. The invention further provides a vector comprising any of the instant nucleic acids. As used herein, a “vector” shall mean any nucleic acid vector known in the art. The vector may be a recombinant vector comprising an expression vector with the nucleic acid inserted therein. Such vectors include, but are not limited to, plasmid vectors, cosmid vectors and viral vectors. In different embodiments, the viral vector is an adenoviral, adeno-associated viral (AAV), or retroviral vector. Several eukaryotic expression plasmids, including pCI, pCMS-EGFP, pHygEGFP, pEGFP-C1, and shuttle plasmids for Cre-1ox Ad vector construction, pDC515 and pDC516, are used in constructs described herein. However, the invention is not limited to these plasmid vectors or their derivatives, and may include other vectors known to those skilled in the art.

Cells Expressing Chimeric HCN Channels

The invention also provides a cell comprising any of the nucleic acids or recombinant vectors described herein, wherein the cell functionally expresses the nucleic acid and thereby expresses the encoded chimeric HCN polypeptide. In preferred embodiments, the cell expresses the chimeric HCN polypeptide at a level effective to induce a pacemaker current in the cell. A “cell” shall include a biological cell, e.g., a HeLa cell, a stem cell, or a myocyte, and a non-biological cell, e.g., a phospholipid vesicle (liposome) or virion. Preferably biological pacemakers of the present invention comprise a biological cell capable of gap junction-mediated communication with cardiomyocytes. Exemplary cells include, but are not limited to, a stem cell, a cardiomyocyte, a fibroblast or skeletal muscle cell engineered to express at least one cardiac connexin, or an endothelial cell. In preferred embodiments, the stem cell is an adult mesenchymal stem cell or an embryonic stem cell, wherein the stem cell is substantially incapable of differentiation. In more preferred embodiments, the stem cell is a human adult mesenchymal stem cell (hMSC) or a human embryonic stem cell (hESC), wherein the stem cell is substantially incapable of differentiation. In other preferred embodiments, the hMSC (a) has been passaged at least nine times, more preferably 9-12 times, (b) expresses CD29, CD44, CD54, and HLA class I surface markers, and (c) does not express CD14, CD34, CD45, and HLA class II surface markers. In further embodiments, the cell further expresses at least one cardiac connexin. In still further embodiments, the at least one cardiac connexin is Cx43, Cx40, or Cx45.

As used herein, to “functionally express” or to “express” a nucleic acid shall mean to introduce the nucleic acid into a cell or other biological system in such a manner as to permit the production of a functional polypeptide encoded by the nucleic acid, so as to thereby produce the functional polypeptide. The encoded polypeptide itself may also be said to be functionally expressed.

In different embodiments of this invention, the nucleic acid is introduced into the cell by infection with a viral vector, plasmid transformation, cosmid transformation, electroporation, lipofection, transfection using a chemical transfection reagent, heat shock transfection, or microinjection. In further embodiments, the viral vector is an adenoviral, an AAV, or a retroviral vector.

There have been recent reports of the delivery of bone marrow-derived and/or circulating hMSCs to the hearts of post-myocardial infarct patients resulting in some improvement of mechanical performance (Strauer et al., 2002; Perin et al., 2003) in the absence of overt toxicity. The presumption in these and other animal studies (Orlic et al., 2001) is that the hMSCs integrate into the cardiac syncytium and then differentiate into new heart cells restoring mechanical function. However, no differentiation of hMSCs was seen over a 42-day period following injection of mHCN2-transfected hMSCs into LV subepicardium of 6 non-immunosuppressed adult dogs (Plotnikov et al., 2005b). Moreover, it has been shown that hMSCs passaged at least 9 times, and preferably 9-12 times, are substantially incapable of differentiation while retaining hMSC surface markers including CD29, CD44, CD54, and HLA class I surface markers, but not expressing CD14, CD34, CD45, and HLA class II surface markers. See U.S. Provisional Application No. 60/832,518, filed Jul. 21, 2006.

Pharmaceutical Compositions

The invention further provides a pharmaceutical composition comprising any of the nucleic acids, vectors, cells, stem cells, HCN polypeptides and mutants and chimeras thereof described herein and a pharmaceutically acceptable carrier. Pharmaceutically acceptable carriers are well known to those skilled in the art and include, but are not limited to, 0.01-0.1M and preferably 0.05M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Preservatives and other additives, such as, for example, antimicrobials, antioxidants and chelating agents may also be included with all the above carriers.

Biological Pacemakers Comprising Biological Material Expressing HCN Channels

The present invention also provides a biological pacemaker comprising an implantable cell that functionally expresses a nucleic acid encoding a HCN polypeptide, or a mutant or chimera thereof, at a level effective to induce a pacemaker current in the cell, and the use of these biological pacemakers to treat cardiac conditions.

A “biological pacemaker” shall mean a biological material that expresses or is capable of causing the expression of a gene such as an HCN ion channel gene, wherein introduction of this biological material into a heart induces biological pacemaker activity in the heart. “Biological pacemaker activity” shall mean the rhythmic generation of an action potential originating from the introduction of biological material in a cell or a syncytial structure comprising the cell. A “syncytium” or “syncytial structure” shall mean a tissue in which there is gap junction-mediated continuity between the constituent cells. Such a syncytium permits electrotonic propagation of electrical signals. “Inducing a current in a cell” shall mean causing a cell to produce an electric current. An “ion channel” shall mean a channel in a cell membrane created by polypeptide or a combination of polypeptides that localizes to a cell membrane and facilitates the movement of ions across the membrane, thereby generating a transmembrane electric current. An “ion channel gene” shall mean a polynucleotide that encodes a subunit of an ion channel, or more than one subunit(s) thereof or an entire ion channel. A “pacemaker current” shall mean a rhythmic electric current generated by a biological material or electronic device.

A “HCN channel” shall mean a hyperpolarization-activated, cyclic nucleotide-gated ion channel responsible for the hyperpolarization-activated cation currents that are directly regulated by cAMP and contribute to pacemaker activity in heart and brain.

“Inducing biological pacemaker activity” in a heart or selected site therein shall mean causing the heart or site therein to rhythmically generate an action potential. The HCN channel may include, but is not limited to, a wild type homologous or heterologous HCN channel, a chimeric HCN channel, a mutant HCN channel, and a chimeric-mutant HCN channel, i.e., a chimeric HCN channel in which one or more portions is derived from a mutant HCN channel.

As a therapeutic solution, a biological pacemaker can be used to generate a spontaneous beating rate within the physiologically acceptable range that originates from its site of implantation in the heart. “Beating rate” shall mean (1) the contraction rate of heart/myocardium, a portion thereof, or an individual myocyte contraction or contractions over a given time period by a cell (e.g., number of contractions or beats per minute), or (2) the rate of production of an electrical pulse or electrical pulses over a given time period by a cell. A biological pacemaker may be used to either increase the beating rate of a normally spontaneous, but too slowly firing, locus of cardiac cells or to initiate spontaneous activity in a normally quiescent region. Since impulse initiation by a native biological pacemaker relies on the balance between a number of ion channels and transporters, many of which are hormonally modulated, there are several possible approaches to creating a biological pacemaker.

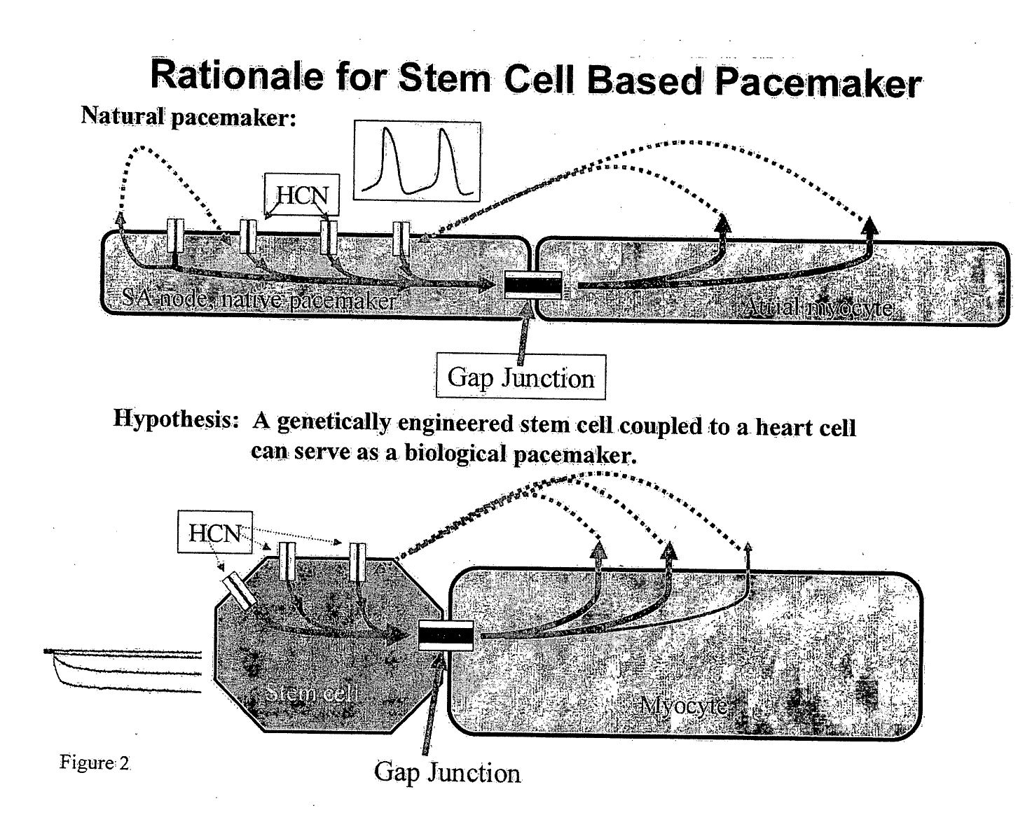

These approaches include, but are not limited to, over-expression of beta-2 adrenergic receptors to increase endogenous atrial rates (Edelberg et al., 1998; 2001), expression of dominant negative Kir2.1AAA constructs together with the wild-type Kir2.1 gene to suppress the inward rectifier current, I_{K1} (Miake et al., 2002; 2003), overexpression of HCN2 channels to increase hyperpolarization-activated, inward pacemaker current (I_{f}) and hence the rate of impulse initiation (Qu et al., 2003; Plotnikov et al., 2004; Potapova et al., 2004), and creating new pacemaker cells from embryonic or mesenchymal stem cells (Kehat et al., 2004; Xue et al., 2005). These approaches seek to manipulate the basic determinants of native pacemaker function in normal hearts; that is, any intervention that increases sympathetic input, decreases repolarizing current, and/or increases depolarizing current during diastole should increase the rate of impulse initiation (Biel et al., 2002). Methods used to achieve these ends have involved gene transfer via viral infection or naked plasmid transfection (Edelberg et al., 1998; 2001), use of embryonic stem cells incorporating a complement of native genes (Kehat et al., 2004), or adult mesenchymal stem cells (MSCs) engineered as platforms to carry pacemaker genes (Potapova et al., 2004). The philosophy behind the latter approach is illustrated in FIG. 2. The production of pacemaker action potentials in non-cardiac cells, and/or inducing fusion of non-cardiac and cardiac cells, have also been recently attempted (Cho et al., 2005).

When choosing a strategy for biological pacemakers, the potential for arrhythmogenesis must be considered. The ideal approach would create or enhance spontaneous activity without undesired side effects. In this regard, enhancing autonomic responsiveness by the upregulation of β-adrenergic receptors poses the problem of specificity, since an increase in sympathetic tone is not specific to a single ion current. The targeting of specific ion currents, either by reducing the hyperpolarizing inward rectifier current I_{K1} or enhancing the inward pacemaker current I_{f} both result in increased net inward current in the pacemaker range of potentials. However, I_{K1} also contributes to terminal repolarization, and its down-regulation results in a prolonged action potential (Miake et al., 2002), which has attendant arrhythmic possibilities. By contrast, I_{f} flows only at diastolic potentials and should not affect action-potential duration. Consequently, I_{f} is an attractive molecular target and is preferred for developing biological pacemakers.

The generation of biological pacemakers based on expression of HCN genes has previously been described. See, e.g., U.S. Pat. Nos. 6,849,611 and 6,783,979. U.S. Pat. No. 6,849,611 teaches an HCN ion channel-containing composition administered to a subject that functions as a site of impulse initiation where sinus node activity is abnormal, thus acting as a biological pacemaker to account for the deficit in the sinus node. U.S. Pat. No. 6,783,979 teaches vectors comprising nucleic acids encoding HCN ion channels which can be applied to a heart tissue so as to provide an ion current in the heart biological tissue. Appropriate administration of such vectors to the heart can provide currents to act as pacemakers. Also described in U.S. Pat. No. 6,783,979 are biological pacemakers based on expression of HCN genes in combination with MiRP1. The entire contents of the above publications are incorporated herein by reference.

The different HCN isoforms show distinct biophysical properties. For example, in cell-free patches from Xenopus oocytes, the steady-state activation curve of HCN2 channels is 20 mV more hyperpolarized that that of HCN1. Also, whereas the binding of cAMP to the CNBD markedly shifts the activation curve of HCN2 by 17 mV to more positive potentials, the response of HCN1 is much less pronounced (4 mV shift). Experiments to generate biological pacemaker activity have been centered on HCN2 because its kinetics are more favorable than those of HCN4, and its cAMP responsiveness is greater than that of HCN1.

FIG. 3 provides a starting point for understanding the role of HCN channels and the I_{f} current they carry in initiating the pacemaker potential. In brief, phase 4 depolarization is initiated by inward sodium current activated on hyperpolarization of the cell membrane and is continued and sustained by other currents (Biel et al., 2002). The latter incorporate a balance between inward currents carried by the calcium channel and the sodium/calcium exchanger and outward currents carried by potassium. Activation of the pacemaker potential is increased by β-adrenergic catecholamines and reduced by acetylcholine through their respective G protein-coupled receptors and the adenylyl cyclase-cAMP second messenger system.

Full-length cDNAs encoding HCN1-4 isoforms have been cloned from different species and functionally characterized following expression in mammalian cell lines. See, for example, Santoro et al. (1998) and Ludwig et al. (1998) reporting the cloning and functional characterization of HCN1-3 from mouse brain; Ludwig et al. (1999) reporting the cloning and functional characterization of HCN2 and HCN4 from human heart; Ishii et al. (1999) reporting the cloning and functional characterization of HCN4 from rabbit heart; Monteggia et al. (2000) reporting the cloning of HCN1-4 in rat brain; and Steiber et al. (2005) reporting the cloning and functional characterization of HCN3 from human brain.

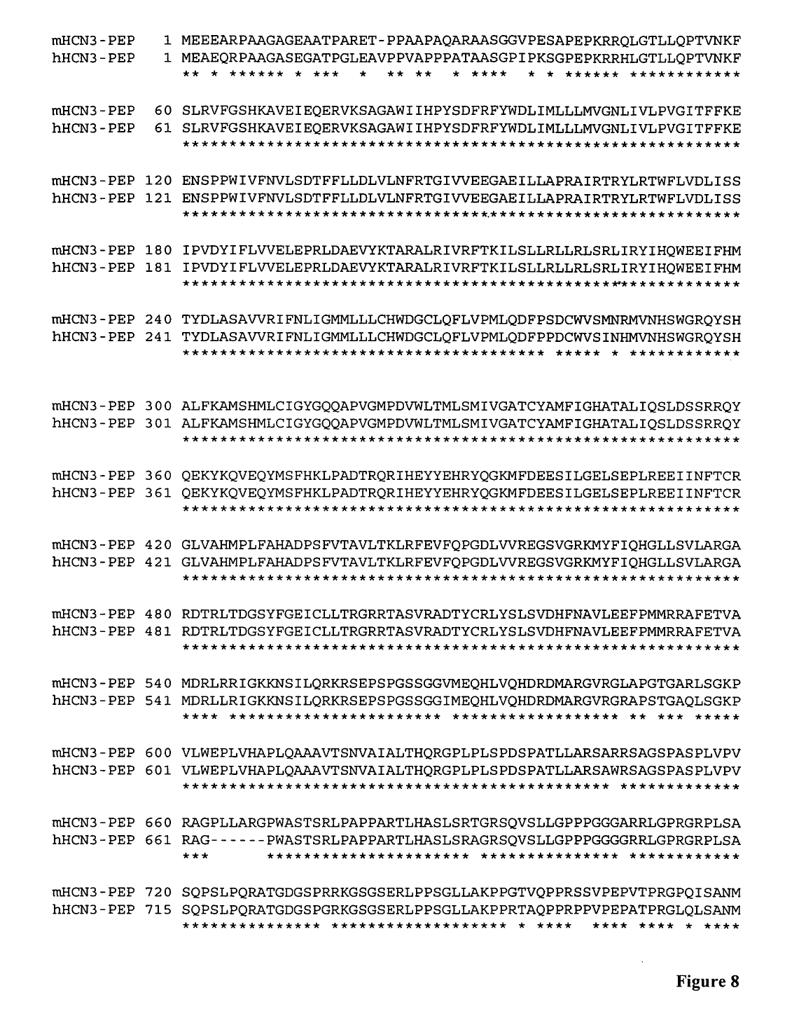

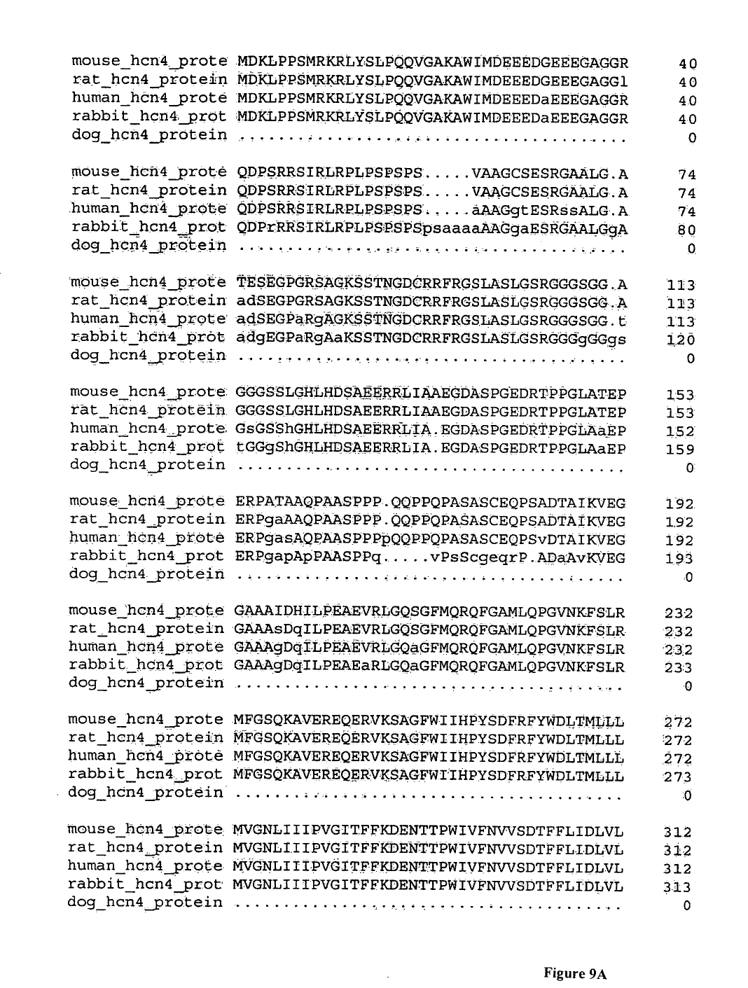

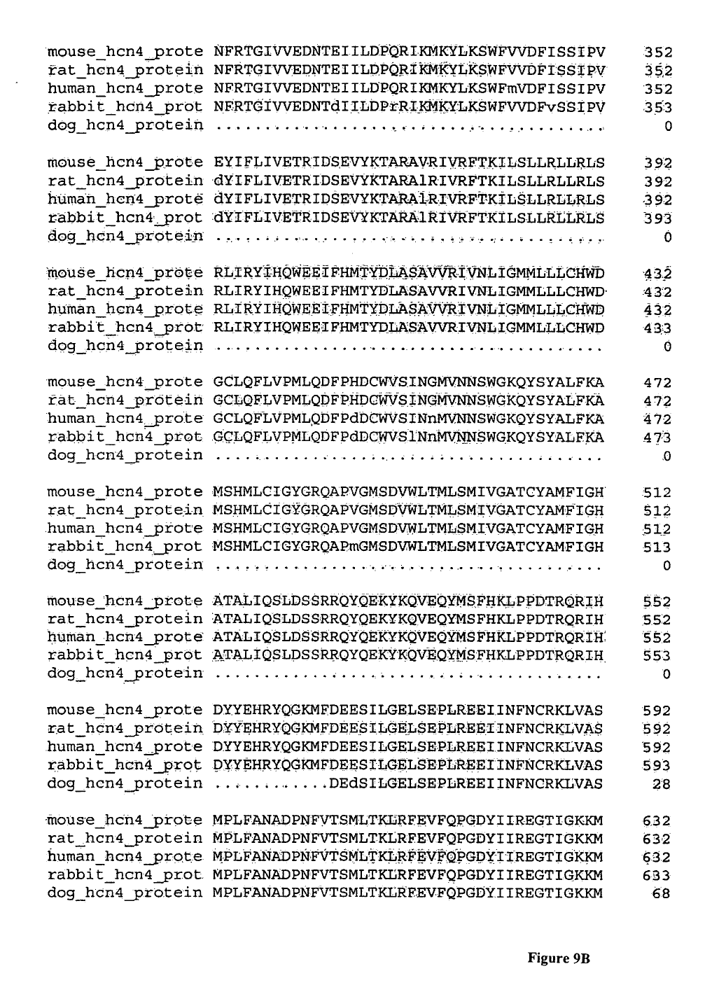

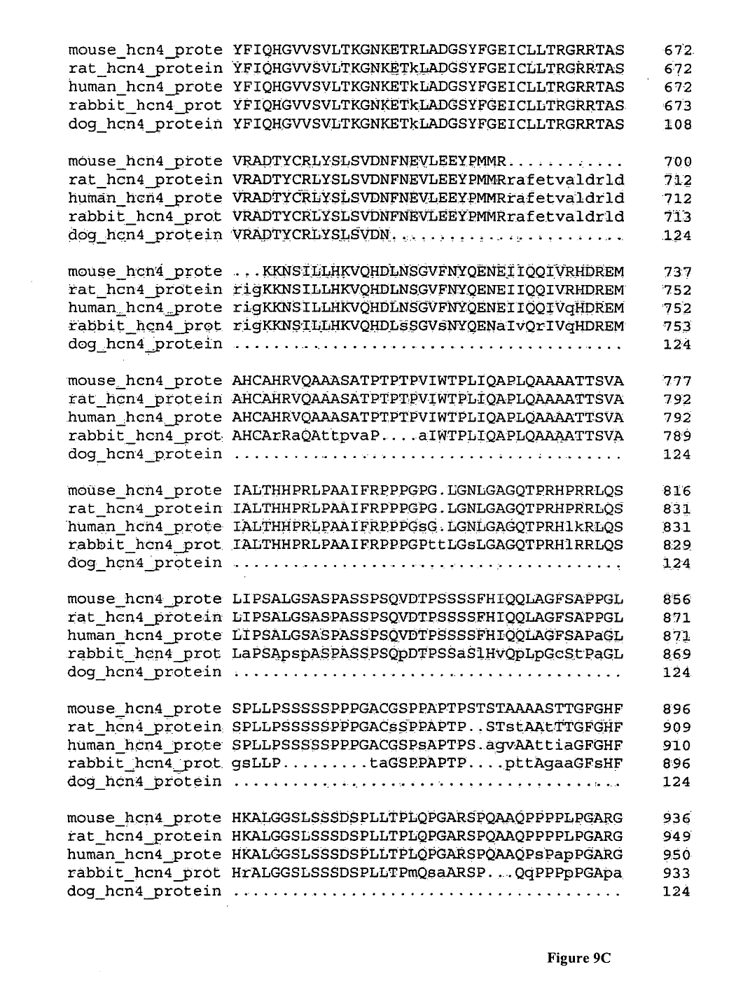

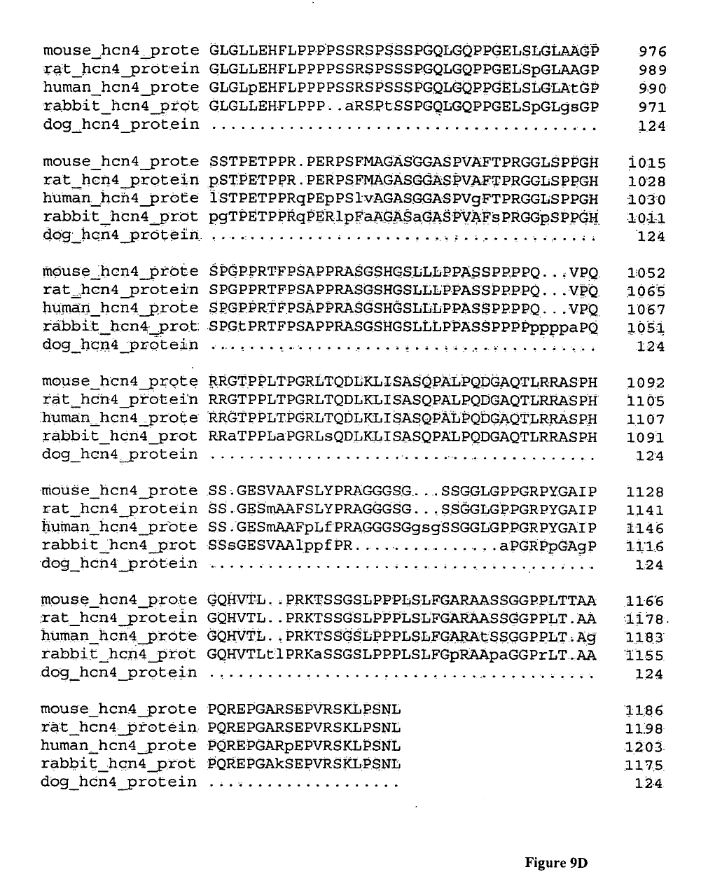

FIG. 4 shows the amino acid sequences of the HCN1 polypeptides from mouse (SEQ ID NO:______), rat (SEQ ID NO:______), human (SEQ ID NO:______), rabbit (SEQ ID NO:______) and guinea pig (partial sequence; SEQ ID NO:______), aligned for maximum correspondence. Similar alignments for HCN2, HCN3 and HCN4 from a variety of mammalian species are depicted in FIGS. 7, 8 and 9, respectively. The amino acid identity between different HCN isoforms in a species varies from about 45-60%, with differences primarily due to low sequence identity in the N- and C-terminal regions. For example, the primary sequences of mHCN1-3 have an overall amino acid identity of about 60% (Ludwig et al., 1999), and hHCN3 has 46-56% homology with the other hHCNs (Stieber et al., 2005). By comparison, significantly higher degrees of homology have been observed between cognate isoforms in different species. For example, Ludwig et al. (1999) report that the hHCN2 cDNA clone has 94% overall sequence identity with a mHCN2 clone; Stieber et al. (2005) report that hHCN3 has 94.5% amino acid homology with mHCN3; and in a review on HCN channels, Biel et al. (2002) disclose that the primary sequences of individual HCN channel types exhibit over 90% sequence identity in mammals.

Table 1, adapted from Stieber et al. (2005), Supplement Table S2, shows the amino acid homology of hHCN3 with the other hHCNs and with mHCN3. Particularly striking is the near-100% homology of the hHCN3 and mHCN3 sequences in the core transmembrane domains and the cyclic nucleotide binding domain. The N-terminal and C-terminal regions of hHCN3 and mHCN3 are 81 and 91% homologous, respectively, which are lower than the degree of homology in the transmembrane and CNDB regions, but still considerable higher than the 22-35% homology between the N-terminus of hHCN3 and the N-terminal regions of other hHCN isoforms, 17-27% homology in the C-terminal regions, and 46-56% overall homology between hHCN3 and other hHCN isoforms.

TABLE 1
Amino Acid Homology between hHCN3
and hHCN1, 2 and 4 and mHCN3

| Amino acid homology¹ | hHCN1 | hHCN2 | hHCN4 | mHCN3 |
|---|---|---|---|---|
| compared to hHCN3 | (%) | (%) | (%) | (%) |
| Overall | 53.0 | 55.8 | 45.7 | 94.5 |
| N-terminus | 34.6 | 28.4 | 22.2 | 80.7 |
| S1 | 78.3 | 78.3 | 87.0 | 100 |
| S1-S2 linker | 64.3 | 71.4 | 78.6 | 100 |
| S2 | 77.3 | 90.9 | 90.9 | 100 |
| S2-S3 linker | 41.7 | 54.2 | 50.0 | 100 |
| S3 | 84.2 | 79.0 | 84.2 | 100 |
| S3-S4 linker | 36.4 | 36.4 | 45.5 | 100 |
| S4 | 100 | 100 | 100 | 100 |
| S4-S5 linker | 100 | 94.4 | 100 | 100 |
| S5 | 96.0 | 92.0 | 96.0 | 100 |
| S5 linker-Pore-S6 linker | 82.0 | 77.6 | 85.7 | 93.9 |
| S6 | 89.7 | 96.6 | 100 | 100 |
| S6-CNBD linker | 82.9 | 85.4 | 91.5 | 100 |
| CNBD² | 78.3 | 80.0 | 80.8 | 99.2 |
| C-terminus | 17.4 | 26.5 | 19.1 | 90.7 |

¹For this comparison, identical and similar amino acids are considered homologous.
²Cyclic nucleotide binding domain

The above homology data suggest that cognate HCN isoforms from different species can be effectively substituted in the present invention; for example, hHCN2 or portions thereof can be substituted for mHCN2 or corresponding portions thereof. Accordingly, in the present invention, a biological pacemaker or method comprising the use of HCN2 or portions thereof from one species, for example mouse, encompasses the use of HCN2 or corresponding portions thereof from other species, preferably mammalian species, including, but not limited to, a human, rat, dog, rabbit, or guinea pig. Similarly, a biological pacemaker or method comprising the use of mouse HCN1, HCN3 or HCN4 or portions thereof encompasses the use of HCN1, HCN3, or HCN4, or corresponding portions thereof, respectively, from other species, preferably other mammalian species.

More generally, a biological pacemaker or method comprising the use of a particular HCN isoform encompasses the use of an HCN channel exhibiting at least 75%, preferably at least 85%, more preferably at least 90%, and most preferably at least 95% overall homology with that isoform. In embodiments of the invention comprising portions of an HCN isoform, the use of a N-terminal portion of a particular HCN isoform encompasses the use of a N-terminal portion of a HCN channel exhibiting at least 60%, preferably at least 70%, more preferably at least 80% homology with the N-terminus of that isoform. In addition, the use of a C-terminal portion of a particular HCN isoform encompasses the use of a C-terminal portion of a HCN channel exhibiting at least 60%, preferably at least 70%, more preferably at least 80%, and most preferably at least 90% homology with the C-terminus of that isoform.

Percentage “homology” between peptide sequences shall mean the degree, expressed as a percentage, to which the amino acid residues at equivalent positions in the peptides, when aligned for maximum correspondence, are identical or functionally similar. Examples of functionally similar amino acids include glutamine and asparagine; serine and threonine; and valine, leucine and isoleucine. Percentage “amino acid identity” or percentage “sequence identity” between peptide sequences shall mean the degree, expressed as a percentage, to which the amino acid residues at equivalent positions in the peptides, when aligned for maximum correspondence, are identical. For peptides, the percentage homology is usually greater than the percentage sequence identity. For nucleic acids, percentage “homology” shall mean the same as percentage “sequence identity,” which is the degree, expressed as a percentage, to which the nucleotides at equivalent positions in the nucleic acids, when aligned for maximum correspondence, are identical.

For the purpose of the invention, two sequences that share homology, i.e., a desired polynucleotide and a target sequence, may hybridize when they form a double-stranded complex in a hybridization solution of 6×SSC, 0.5% SDS, 5× Denhardt's solution and 100 g of non-specific carrier DNA. See section 2.9, supplement 27, of Ausubel et al. (1994), the entire contents of which are herein incorporated by reference. Such sequence may hybridize at “moderate stringency,” which is defined as a temperature of 60° C. in a hybridization solution of 6×SSC, 0.5% SDS, 5× Denhardt's solution and 100 μg of non-specific carrier DNA. For “high stringency” hybridization, the temperature is increased to 68° C. Following the moderate stringency hybridization reaction, the nucleotides are washed in a solution of 2×SSC plus 0.05% SDS for five times at room temperature, with subsequent washes with 0.1×SSC plus 0.1% SDS at 60° C. for 1 h. For high stringency, the wash temperature is increased to typically a temperature that is about 68° C. Hybridized nucleotides may be those that are detected using 1 ng of a radiolabeled probe having a specific radioactivity of 10,000 cpm/ng, where the hybridized nucleotides are clearly visible following exposure to X-ray film at −70° C. for no more than 72 hours.

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215:403-410 (1990); and, Altschul et al., Nucleic Acids Res. 25:3389-3402 (1997).

Software for performing BLAST analyses is publicly available, e.g., through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad Sci. USA 90:5873-5877 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences that may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

Biological Pacemakers Comprising Cells Expressing Chimeric HCN Channels

As disclosed above, the present invention provides cells that functionally express a variety of chimeric HCN polypeptides at a level effective to induce pacemaker current in the cells. Such cells constitute biological pacemakers, wherein the use of certain chimeras confers beneficial characteristics for biological pacemaking (see Example 6).

Biological Pacemakers Comprising Cells Expressing Mutant HCN Channels

This invention also provides biological pacemakers comprising a cell that functionally expresses a mutant HCN polypeptide at a level effective to induce pacemaker current in the cell.

Most of what is known about voltage activation of ion channels comes from studies of voltage-gated K⁺ (Kv) channels. Although HCN channels open in response to membrane hyperpolarization instead of depolarization as in Kv channels, HCN channels have a transmembrane topology that is highly similar to Kv channels. All of these ion channels have four subunits, each of which has six transmembrane segments, S1-S6: the positively charged S4 domain forms the major voltage sensor, whereas S5 and S6, together with the S5-S6 linker connecting the two, form the pore domain containing the ion permeation pathway and the gates that control the flow of ions (Larsson, 2002). The activation gate is formed by the crossing of the C-terminal end of the S6 helices (Decher et al., 2004). Much progress has been made, based on biophysical experiments and the recently described structures of bacterial K⁺ channels, in understanding the physical basis for the activation and inactivation of gates, selective ion permeability, and voltage sensing mechanisms of ion channels. However, the molecular mechanism whereby changes in voltage open and close these channels, and the mechanism between the voltage sensors and the gates, are still largely not understood. In particular, it remains unclear how the coupling mechanism results in opposite voltage dependence of activation for Kv and HCN channels.

Coupling of the movement of the voltage sensor to the opening and closing of the HCN channel pore could involve global rearrangements of the S4, S5 and S6 transmembrane domains without the need for specific amino acid interactions. However, recent studies suggest that physical coupling may include specific interactions between amino acids in the S4-S5 linker and the S6 domain (Chen et al., 2001a; Decher et al., 2004). These studies suggest that the S4-S5 linker is an important component of the coupling mechanism that mediates the hyperpolarization-activated opening of HCN channels.

Voltage sensing and activation of HCN channels can be altered by mutation. For example, alanine-scanning mutagenesis of the S4-S5 linker in HCN2 revealed that three amino acids were especially critical for normal gating (Chen et al., 2001a). Mutation of Y331 or R339, and to a lesser extent, E324, disrupted channel closure. Mutation of a basic residue in the S4 domain (R318Q) prevented channel opening. Conversely, channels with R318Q and Y331S double mutations were constitutively open.

Using alanine-scanning mutagenesis of the C-terminal end of S6 and the C-linker that connects S6 to the CNBD, Decher et al. (2004) identified five residues that were important for normal gating as mutations disrupted channel closure. Further mutation analyses suggested that a specific electrostatic interaction between R339 of the S4-S5 linker and D443 of the C-linker stabilizes the closed state and thus participates in the coupling of voltage sensing and activation gating in HCN channels. Interactions between residues in the S4-S5 linker and the C-terminal end of the S6 domain have also been shown to be critical for stabilizing hERG and ether-á-go-go channels in a closed state (Ferrer et al., 2006). These mutation studies indicate that mutations in the S4 voltage sensor, the S4-S5 linker implicated in the coupling of voltage sensing to pore opening and closing, the S5, S6 and S5-S6 linker which form the pore, the C-linker, and the CNBD, may be particularly important in affecting HCN channel activity.

The S3-S4 linker (residues ²²⁹EKGMDSEVY²³⁷ of HCN1) has also been shown to be important in influencing the activation phenotypes of HCN channels (Tsang et al., 2004). Specifically, complete deletion of the S3-S4 linker (Δ229-237), as well as the deletions Δ229-234, Δ232-234, and Δ232-237, abolished normal current activity. Conversely, Δ229-231, Δ233-237, Δ234-237, Δ235-237, Δ229-231/Δ233-237, Δ229-231/Δ234-237, and Δ229-231/Δ235-237 all yielded robust hyperpolarization-activated inward currents, suggesting that manipulations of the S3-S4 linker length may provide a flexible way to customize HCN gating for fine tuning the electrical activity of endogenous and engineered cells expressing HCN. Recently, expression of mHCN1-Δ235-237 (HCN1-ΔΔΔ) in the left atrium of pigs with sick-sinus syndrome was shown to reproducibly induce stable, catecholamine-responsive biological pacemaker activity in the transfected porcine heart in situ (Tse et al. 2006). This biological pacemaker exhibited a physiological heart rate and was capable of reliably pacing the myocardium, substantially reducing electronic pacing by an implanted dual-chamber electronic pacemaker (Tse et al. 2006).

Accordingly, the present invention provides a biological pacemaker, wherein the biological pacemaker comprises an implantable cell that functionally expresses a mutant HCN ion channel at a level effective to induce pacemaker current in the cell. In preferred embodiments the mutant HCN channel provides an improved characteristic, as compared to a wild-type HCN channel, including, but not limited to, faster kinetics, more positive activation, increased expression levels, increased stability, enhanced cAMP responsiveness, and enhanced neurohumoral response. In certain embodiments of the present invention, the mutant HCN channel carries at least one mutation in S4 voltage sensor, the S4-S5 linker, S5, S6, the S5-S6 linker, and/or the C-linker, and the CNBD which mutations result in one or more of the above discussed characteristics. In other embodiments, the HCN mutant is E324A-HCN2, Y331A-HCN2, R339A-HCN2, or Y331A,E324A-HCN2. In preferred embodiments, the mutant HCN channel is E324A-HCN2.

In addition to the mutations noted above, many mutations in different HCN isoforms have been reported. These include R318Q, W323A, E324A, E324D, E324K, E324Q, F327A, T330A and Y331A, Y331D, Y331F, Y331K, D332A, M338A, R339A, R339C, R339D, R339E and R339Q in HCN2 made by Chen et al. (2001a) to investigate in greater detail the role of the E324, Y331 and R339 residues in voltage sensing and activation. Chen et al. (2001b) have also reported the R538E and R591E mutations in mHCN1; Tsang et al. (2004) have reported G231A and M232A in mHCN1; Vemana et al (2004) have reported R247C, T249C, K250C, 1251C, L252C, S253C, L254C, L258C, R259C, L260C, S261C, C318S, S338C in mHCN2; Macri and Accili (2004) have reported S306Q, Y331D AND G404S in mHCN2; and Decher et al. (2004) have reported Y331A, Y331D, Y331S, R331FD, R339E, R339Q, 1439A, S441A, S441T, D443A, D443C, D443E, D443K, D443N, D443R, R447A, R447D, R447E, R447Y, Y449A, Y449D, Y449F, Y449G, Y449W, Y453A, Y453D, Y453F, Y453L, Y453W, P466Q, P466V, Y476A, Y477A and Y481A in mHCN2. The entire contents of all of the above publications are incorporated herein by reference. Certain of the reported mutations listed above may confer, singly or in combination, beneficial characteristics on the HCN channel with regard to creating a biological pacemaker. The invention disclosed herein encompasses all mutations in HCN channels, singly or in combinations, which improve pacemaker activity of the channel such as by providing faster kinetics, more positive activation, increased expression and/or stability, enhanced cAMP responsiveness, and enhanced neurohumoral response.

Experiments disclosed herein have explored the E324A mutation in mHCN2 that has been reported to exhibit both faster kinetics and a more positive activation relation (Chen et al., 2001a). Both these characteristics should enhance pacemaking. Details of the pacemaker activity of E324A compared to HCN2 when expressed in myocytes, Xenopus oocytes, and in situ in dog hearts are provided in Example 1.

Biological Pacemaker Comprising Expression of HCN Channels and MiRP1

Another approach to enhancing biological pacemaker activity of a HCN channel by increasing the magnitude of the current expressed and/or speeding its kinetics of activation is to co-express HCN with its beta subunit, MiRP1. As described in Example 3, infection of myocyte cultures with a HCN2 adenovirus and a second adenovirus comprising an HA-tagged form of MiRP1 results in a significant increase in current magnitude and acceleration of activation and deactivation kinetics. See also U.S. Pat. No. 6,783,979 and Qu et al. (2004), the entire contents of which are incorporated herein by reference. Many MiRP1 mutations have been reported (see, e.g., Mitcheson et al., 2000; Lu et al., 2003; Piper et al., 2005), and certain of these mutations, or combinations thereof, may be beneficial in increasing the magnitude and kinetics of activation of the current expressed by a HCN channel used to create a biological pacemaker. The invention disclosed herein encompasses all such mutations, or combinations thereof, in MiRP1.

Delivery of HCN Channel Genes to Cells to form Biological Pacemakers

The genes or mutants or chimeras thereof used for expressing ion channels and cardiac connexins in biological pacemakers must be delivered into the heart. Many methods are known in the art for introducing DNA into cells, and of these, there are at least three broad approaches to delivering DNA into hearts: use of naked DNA, viral vectors, and cells; among the latter are hMSCs or embryonic stem cells ESCs. Although current experimentation has employed hMSCs and ESCs, any cell type which expresses the HCN genes and cardiac connexin genes, or can be made to do so, could serve as a cellular delivery system. Examples of alternative cell types that could be used as delivery platforms for pacemaker genes include, but are not limited to, any late-passage stem cell, a cardiomyocyte, a fibroblast or skeletal muscle cell engineered to express connexins, or an endothelial cell.

Each method of gene delivery has unique difficulties. Naked DNA is poorly taken up and its effects only have a short duration. Viral vectors are far more efficient but the use of replication-deficient adenoviruses having little infectious potential raises the likelihood of only transient improvement in pacemaker function as well as potential inflammatory responses, whereas the use of retroviruses and other more persistent viral vectors carry the risk of carcinogenicity and infectivity.

Relying on cell therapy using, for example, hESCs or hMSCs, is one way to avoid the use of viral vectors. Several laboratories are exploring the use of ESCs that can be differentiated along a cardiac lineage and might provide a cell-based control of cardiac rhythm. Among the advantages of these cells is that they make functional gap junctions and generate spontaneous rhythms (Rosen et al., 2004). Such approaches are, however, still in their infancy and present problems, including the immunogenicity of the cells, identifying appropriate cell lineages, the possibility of stem cell differentiation into lines other than pacemaker cells, and potential for neoplasia (Rosen et al., 2004). Alternatively, employing genetically engineered adult hMSCs derived from bone marrow as platforms for delivering ion channels to the heart (Potapova et al., 2004) avoids allergic reactions but still requires safe and persistent expression of the transgene. In this setting, the cells would be thought of as a biologically inert vector that could deliver molecular/genetic information to adjoining myocardium. Experiments described below indicate that hMSCs provide an attractive platform for delivery pacemaker ion channels into the heart. Other cell types which may allow for packaging the pacemaker genetic material in vitro and delivering pacemaker ion channels in to the heart include, but are not limited to, any late-passage stem cells, connexin-expressing fibroblasts, cardiomyocytes, skeletal muscle cells, and endothelial cells.

Electroporation is a preferred in vitro method for genetically engineering cells such as hMSCs to overexpress I_{f} for in vivo delivery. Electroporation is a technique in which exposure of cells to a brief pulse of high voltage transiently opens pores in the cell membranes that allow macromolecules, such as DNA and proteins, to enter the cell. It has been demonstrated that electroporation can also be applied in vivo to deliver nucleic acids and proteins into muscle cells of live animals including rats, mice and rabbits (see U.S. Pat. No. 6,110,161), and the method has been used to deliver DNA directly into embryonic chick heart (Harrison et al., 1998) and into mammalian myocardium prior to transplantation (Wang et al., 2001c).

Other methods of introducing genes into cells for delivery into the heart include viral transfection using, for example, adenovirus, AAV, and lentivirus, liposome-mediated transfection (lipofection), transfection using a chemical transfection reagent, heat shock transfection, or microinjection. AAV, a small parvovirus associated with adenovirus, cannot replicate on its own and requires co-infection with adenovirus or herpesvirus in order to replicate. In the absence of helper virus, AAV enters a latent phase during which it stably integrates into the host cell genome. This latent phase makes AAV attractive for certain gene therapy applications involving transfer of genes of up to about 4.4 kb, as the gene inserted into AAV can persist in the host cell genome for a long period (Pfeifer and Verma, 2001). Lentivirus, a member of the retroviral family, provides a potentially interesting alternative (Amado and Chen, 1999; Trono, 2002). Unlike adenoviruses, electroporation and the use of lentiviral vectors allow persistent transgene expression without eliciting host immune responses.

Safety is a factor to be demonstrated especially with viral vectors. The absence of arrhythmias and neoplasia generated by viral vectors or cells should be demonstrated along with an absence of infection or engraftment at distant locations. Once safety and efficacy have been demonstrated, cost-effectiveness should also be considered. Even if the problems of expression and delivery are surmounted, long-term persistence of a cell-based pacemaker requires the absence of rejection if nonautologous cells are employed. In this regard, hMSCs could be obtained from an autologous source. However, evidence suggesting that these cells are immunoprivileged (Liechty et al., 2000) may reduce the need for autologous sources. The long-term extent of this privilege has not been tested, but no cellular or humoral rejection was evident six weeks following injection of hMSCs into canine hearts (Plotnikov et al., 2005b). Rejection remains a consideration for embryonic stem cells. Allogeneic solutions based on the immunoprivileged status of hMSCs would provide a more favorable model since off-the-shelf cells could be ready for implantation.

In different embodiments of the pacemaker systems and methods described herein, the nucleic acid is introduced directly into a cell of the heart by infection with a viral vector, plasmid transformation, cosmid transformation, electroporation, lipofection, transfection using a chemical transfection reagent, heat shock transfection, or microinjection. In other embodiments, the viral vector is an adenoviral, an AAV, or a retroviral vector. In yet other embodiments, the vector is administered onto or into the heart by injection or catheterization. In further embodiments, the vector is administered onto or into an atrium, a wall of a ventricle, a bundle branch of a ventricle, or the proximal left ventricular (LV) conducting system of the heart.

In certain embodiments, the nucleic acid is introduced into a cell so as to induce a current therein, which cell is administered to the heart. Preferably, the cell forms a functional syncytium with the heart and is a stem cell, a cardiomyocyte, a fibroblast or skeletal muscle cell engineered to express at least one cardiac connexin, or an endothelial cell. In certain embodiments, the stem cell is a MSC or a ESC that is substantially incapable of differentiation. In preferred embodiments, the stem cell is a hMSC or a hESC that is substantially incapable of differentiation. In further embodiments, the adult hMSCs that are substantially incapable of differentiation have been passaged at least 9 times, and in some embodiments preferably 9 to 12 times.

The nucleic acid may be introduced into the stem cell by electroporation, infection with a virus including, but not limited to, adenovirus, AAV or lentivirus, plasmid transformation, cosmid transformation, lipofection, transfection using a chemical transfection reagent, heat shock transfection, or microinjection.

Assuming the safety and persistence of transgene expression, a cell-based biological pacemaker also requires site-specific or focal delivery. Several methods to achieve focal delivery are feasible; for example, the use of catheters and needles, and/or growth on a matrix and a “glue.” Whatever approach is selected, the delivered cells should not disperse from the target site. Such dispersion could introduce unwanted electrical effects within the heart or in other organs. It is noteworthy that in a preliminary study involving injection of up to ˜10⁶ HCN2-transfected hMSCs into the LV subepicardium of six adult dogs, nests of hMSCs were consistently found adjacent to the injection site but not at a distance (Plotnikov et al., 2005b).

In various embodiments of the instant pacemaker systems and methods, the stem cell is administered onto or into the heart by injection, catheterization, surgical insertion, or surgical attachment. The delivery site is determined at the time of administration, based on the patient's pathology, to give the optimal activation and hemodynamic response. Thus, the chosen site could be the sinoatrial (SA) node, Bachmanns bundle, atrioventricular junctional region, bundle of His, left bundle branch, right bundle branch, Purkinje fibers, left or right atrial or ventricular muscle, the appropriate site being well known to one of ordinary skill in the art. The type of ion channel expressed in the heart may also be changed depending on the delivery site. In addition, different levels of expression of the ion channel gene may be desirable in different delivery sites. Such different levels of expression may be obtained by using different promoters to drive expression.

In another embodiment, the cell is locally administered by injection or catheterization directly onto or into the heart. In further embodiments, the cell is systemically administered by injection or catheterization into a coronary blood vessel or a blood vessel proximate to the heart. In still further embodiments, the cell is injected onto or into an area of an atrium or ventricle of the heart. In other embodiments, the cell is injected onto or into the left atrium, a wall of a ventricle, a bundle branch of a ventricle, or the proximal left ventricle conducting system of the heart.

Tandem System Comprising Biological and Electronic Pacemakers

The present invention encompasses a tandem pacemaker system for treating cardiac rhythm disorders comprising a combination of any of the biological pacemakers described herein with an electronic pacemaker. U.S. Provisional Application Nos. 60/701,312, filed Jul. 21, 2005, and 60/781,723, filed Mar. 14, 2006, and U.S. Ser. No. 11/490,997, filed Jul. 21, 2006, provide experimental data demonstrating, inter alia, that biological pacemakers based on expression of HCN genes or chimeras or mutants thereof operate seamlessly in tandem with electronic pacemakers to prevent heart rate from falling below a selected minimum beating rate. The tandem system also conserves the total number of electronic beats delivered, and provides a higher, more physiologic and catecholamine-responsive heart rate than is the case with an electronic pacemaker alone. The contents of U.S. Provisional Application Nos. 60/701,312, filed Jul. 21, 2005, and 60/781,723, filed Mar. 14, 2006, and U.S. Ser. No. 11/490,997, filed Jul. 21, 2006, are hereby incorporated herein by reference in their entirety.

Electronic pacemakers are known in the art. Exemplary electronic pacemakers are described in U.S. Pat. Nos. 5,983,138, 5,318,597 and 5,376,106; Hayes (2000); and Moses et al. (2000), the entire contents of all of which are incorporated herein by reference. The subject may have already been fitted with an electronic pacemaker or may be fitted with one simultaneously or after placement of the biological pacemaker. The appropriate site for the electronic pacemaker would be well known to a skilled practitioner, depending on the subject's condition and the placement of the biological pacemaker of the present invention. For example, if the subject had a functional sinoatrial node, but had a block between the sinoatrial node and the atrioventricular node, the biological pacemaker might preferably be administered to the atrioventricular node. Preferred insertion cites include, but are not limited to, the Bachmann's bundle, sinoatrial node, atrioventricular junctional region, His branch, left or right bundle branch, Purkinje fibers, left or right atrial muscle or ventricular muscle of the subject's heart.

In preferred embodiments of the present invention, the electronic pacemaker is programmed to produce its pacemaker signal on an “as-needed” basis, i.e., to sense the biologically generated beats and to discharge electrically when there has been failure of the biological pacemaker to fire and/or bypass bridge to conduct current for more than a preset time interval. At this point the electronic pacemaker will take over the pacemaker function until the biological pacemaker resumes activity. Accordingly, a determination should be made as to when the electronic pacemaker will produce its pacemaker signal. State of the art pacemakers have the ability to detect when the heart rate falls below a threshold level in response to which an electronic pacemaker signal should be produced. The threshold level may be a fixed number, but preferably it varies depending on patient activity such as physical activity or emotional status. When the patient is at rest or pursuing light activity the patient's baseline heart rate may be at 60-80 beats per minute (bpm) (individualized for each patient), for example. This baseline heart rate varies depending on the age and physical condition of the patient, with athletic patients typically having lower baseline heart rates. The electronic pacemaker can be programmed to produce a pacemaker signal when the patient's actual heart rate (including that induced by any biological pacemaker) falls below a certain threshold baseline heart rate, a certain differential, or other ways known to those skilled in the art. When the patient is at rest the baseline heart rate will be the resting heart rate. The baseline heart rate will likely change depending on the physical activity level or emotional state of the patient. For example, if the baseline heart rate is 80 bpm, the electronic pacemaker may be set to produce a pacemaker signal when the actual heart rate is detected to be about 64 bpm (i.e., 80% of 80 bpm).

The electronic component can also be programmed to intervene at times of exercise if the biological component fails, by intervening at a higher heart rate and then gradually slowing to a baseline rate. For example, if the heart rate increases to 120 bpm due to physical activity or emotional state, the threshold may increase to 96 bpm (80% of 120 bpm). The biological portion of this therapy brings into play the autonomic responsiveness and range of heart rates that characterize biological pacemakers and the baseline rates that function as a safety-net, characterizing the electronic pacemaker. The electronic pacemaker may be arranged to output pacemaker signals whenever there is a pause of an interval of X% (e.g., 20%) greater than the previous interval, as long as the previous interval was not due to an electronic pacemaker signal and was of a rate greater than some minimum rate (e.g., 50 bpm).

Accordingly, in an embodiment of the present pacemaker systems, the electronic pacemaker senses the heart beating rate and produces a pacemaker signal when the heart beating rate falls below a specified level. In a further embodiment, the specified level is a specified proportion of the beating rate experienced by the heart in a reference time interval. In a still further embodiment, the reference time interval is an immediately preceding time period of specified duration.

As described herein, implanted biological pacemakers were tested in tandem with electronic pacemakers in canine studies. The electronic-demand pacemaker was set at a predesignated escape rate and the frequency of electronically versus biologically initiated heartbeats was monitored. In this way, the electronic component measures the efficacy of the biological component of a tandem pacemaker unit. It is expected that such tandem biological-electronic pacemakers will not only meet the patient protection standards required in Phase 1 and 2 clinical trials but will also offer therapeutic advantages over purely electronic pacemakers. That is, the biological component of the tandem system will function to vary heart rate over the range demanded by a patient's changing exercise and emotional status, while the electronic component will provide a safety net if the biological component were to fail either partially or totally. In addition, by reducing the frequency of electronic beats that would normally be delivered over time by an electronic-only pacemaker, the tandem unit will extend the battery life of the electronic component. This could profoundly increase the interval between which power packs require replacement. Hence, the components of the tandem pacemaker system operate synergistically in maximizing the opportunity for safe and physiologic cardiac rhythm control.

Methods of Treatment with Tandem Pacemaker System

The tandem pacemaker concept raises several issues with respect to clinical applications. First, the system is redundant by design and would have two completely unrelated failure modes. Two independent implant sites and independent energy sources would provide a safety mechanism in the event of a loss of capture (e.g., due to myocardial infarction). Second, the electronic pacemaker would provide not only a baseline safety net, but an ongoing log of all heartbeats for review by clinicians, thus providing insight into a patient's evolving physiology and the performance of their tandem pacemaker system. Third, since the biologic pacemaker will be designed to perform the majority of cardiac pacing, the longevity of the electronic pacemaker could be dramatically improved. Alternatively longevity could be maintained while the electronic pacemaker could be further reduced in size. Finally, the biological component of a tandem system would provide true autonomic responsiveness, a goal that has eluded more than 50 years of electronic pacemaker research and development.

The present invention also provides a method of treating a subject afflicted with a cardiac rhythm disorder, which method comprises administering to a subject a tandem pacemaker system of the present invention. A biological pacemaker is provided to the subject's heart to generate an effective biological pacemaker current. An electronic pacemaker is also provided to the subject's heart to work in tandem with the biological pacemaker to treat the cardiac rhythm disorder. The electronic pacemaker may be provided before, simultaneously with, or after the biological pacemaker. The electronic and the biological pacemaker are provided to the area of the heart best situated to compensate/treat the cardiac rhythm disorder. For example the biological pacemaker may be administered to, but not limited to, the Bachmann's bundle, sinoatrial node, atrioventricular junctional region, His branch, left or right atrial or ventricular muscle, left or right bundle branch, or Purkinje fibers of the subject's heart. The biological pacemaker is as described above and preferably enhances beta-adrenergic responsiveness of the heart, decreases outward potassium current I_{K1}, and/or increases inward current I_{f}.

The electronic pacemaker works in tandem with the biological pacemaker as described above. For example, the electronic pacemaker is programmed to sense the subject's heart beating rate and to produce a pacemaker signal when the heart beating rate falls below a selected heart beating rate. In other embodiments, the selected beating rate is a selected proportion of the beating rate experienced by the heart in a reference time interval. In other embodiments, the reference time interval is an immediately preceding time period of selected duration. As such, the battery life of the electronic pacemaker is preserved or lasts longer as it does not need to “fire” or send pacemaking signals as often since in the tandem system the biological pacemaker preferably generates an effective pacemaking signal.

A cardiac rhythm disorder is any disorder that affects the heart beat rate and causes the heart rate to vary from a normal healthy heart rate. For example, the disorder may be, but is not limited to, a sinus node dysfunction, sinus bradycardia, marginal pacemaker activity, sick sinus syndrome, cardiac failure, tachyarrhythmia, sinus node reentry tachycardia, atrial tachycardia from an ectopic focus, atrial flutter, atrial fibrillation, or a bradyarrhythmia. In such situations, the biological pacemaker is preferably administered to the left or right atrial muscle, sinoatrial node or atrioventricular junctional region of the subject's heart.

In certain embodiment of the present methods for treating cardiac rhythm disorders, a pre-existing source of pacemaker activity in the heart is ablated, so as not to conflict with the biological pacemaker and/or the electronic pacemaker.

This invention further provides a method of inhibiting the onset of a cardiac rhythm disorder in a subject prone to such disorder comprising (a) inducing biological pacemaker activity in the subject's heart by functionally expressing in the heart at least one of (1) a nucleic acid encoding a HCN ion channel or a mutant or chimera thereof, (2) a nucleic acid encoding a MiRP1 beta subunit or a mutant thereof, and (3) a nucleic acid encoding both (i) a HCN ion channel or a mutant or chimera thereof and (ii) a MiRP1 beta subunit or a mutant thereof, at a level effective to induce a pacemaker activity in the heart; and (b) implanting an electronic pacemaker in the heart, so as to thereby inhibit the onset of the disorder in the subject. In certain embodiments, a biological pacemaker of the present invention is provided to a subject.

The present invention also provides a method of inducing in a cell a current capable of inducing biological pacemaker activity comprising administering to the heart any of the biological pacemakers described herein and thereby and functionally expressing in the heart a HCN ion channel or a mutant or chimera thereof, and/or a MiRP1 beta subunit or a mutant thereof, at a level effective to induce in the cell a current capable of inducing biological pacemaker activity, so as to thereby induce such current in the cell.

The invention disclosed herein also provides a method of increasing heart rate in a subject which comprises administering to the heart any of the biological pacemakers described herein and thereby expressing in the subject's heart a HCN ion channel or a mutant or chimera thereof, and/or a MiRP1 beta subunit or a mutant thereof, at a level effective to decrease the time constant of activation of the cell, so as to thereby increase heart rate in the subject.

The above-identified steps in the preceding method may also be used in methods of causing a contraction of a cell, shortening the time required to activate a cell, and changing the membrane potential of a cell.

Other Methods

The steps of the preceding method may also be used to preserve battery life of an electronic pacemaker implanted in a subject's heart, and to enhance the cardiac pacing function of an electronic pacemaker implanted in a subject's heart.

This invention further provides a method of monitoring cardiac signals with an electronic pacemaker having sensing capabilities implanted in a subject's heart comprising (a) selecting a site in or on the heart, (b) inducing biological pacemaker activity at the selected site by any of the methods described herein so as to enhance the natural pacemaker activity in the heart, (c) monitoring heart signals with the electronic pacemaker, and (d) storing the heart signals.

This invention also provides a method of enhancing the cardiac pacing function of an electronic pacemaker having sensing and demand pacing capabilities implanted in a subject's heart comprising (a) selecting a site in or on the heart, (b) inducing biological pacemaker activity at the selected site by any of the methods described herein so as to enhance the natural pacemaker activity in the heart, (c) monitoring heart signals with the electronic pacemaker, (d) determining when the heart should be paced based on the heart signals, and (e) selectively stimulating the heart with the electronic pacemaker when the natural pacemaker activity in tandem with the biological pacemaker activity fails to capture the heart.

This invention also provides a method of treating a subject afflicted with a cardiac rhythm disorder comprising administering to a region of the subject's heart any of the cells expressing a HCN polypeptide described herein, wherein expression of the HCN polypeptide in said region of the heart is effective to induce a pacemaker current in the heart and thereby treat the subject.

The invention also provides a method of inhibiting the onset of a cardiac rhythm disorder in a subject prone to such disorder comprising administering to a region of the subject's heart any of the cells expressing a HCN polypeptide described herein, wherein expression of the HCN polypeptide in the heart is effective to induce a pacemaker current in the heart and thereby inhibit the onset of the disorder in the subject. In preferred embodiments of the instant methods, the HCN polypeptide is a chimeric HCN polypeptide.

As used herein, “treating” a subject afflicted with a disorder shall mean causing the subject to experience a reduction, remission or regression of the disorder and/or its symptoms. In one embodiment, recurrence of the disorder and/or its symptoms is prevented. In a preferred embodiment, the subject is cured of the disorder and/or its symptoms.

“Inhibit” shall mean either lessening the likelihood of, or delaying, the disorder's onset, or preventing the onset of the disorder entirely. In a preferred embodiment, inhibiting the onset of a disorder means preventing its onset entirely.

“Inhibiting the onset” of a disorder shall mean either lessening the likelihood of, or delaying, the disorder's onset, or preventing the onset of the disorder entirely. In a preferred embodiment, inhibiting the onset of a disorder means preventing its onset entirely.

“Administering” shall mean delivering in a manner which is effected or performed using any of the various methods and delivery systems known to those skilled in the art. Administering can be performed, for example, pericardially, intracardially, subepicardially, transendocardially, via implant, via catheter, intracoronarily, endocardially, intravenously, intramuscularly, via thoracoscopy, subcutaneously, parenterally, topically, orally, intraperitoneally, intralymphatically, intralesionally, epidurally, or by in vivo electroporation. Administering can also be performed, for example, once, a plurality of times, and/or over one or more extended periods.

A “subject” shall mean any animal or artificially modified animal. Animals include, but are not limited to, humans, non-human primates, dogs, cats, cows, horses, sheep, pigs, rabbits, ferrets, rodents such as mice, rats and guinea pigs, and birds such as chickens and turkeys. Artificially modified animals include, but are not limited to, SCID mice with human immune systems. In a preferred embodiment, the subject is a human.

In an embodiment of any of the methods described herein for treating or inhibiting the onset of a cardiac rhythm disorder, a pre-existing source of pacemaker activity in the heart is ablated, for example by surgery or chemically. In another embodiment, the cell administered to the heart forms a functional syncytium with the heart. In other embodiments, the cell is administered to the region of the subject's heart by injection, catheterization, surgical insertion, or surgical attachment. In yet other embodiments, the cell is locally administered by injection or catheterization directly onto or into the heart. In further embodiments, the cell is systemically administered by injection or catheterization into at least one of a coronary blood vessel or other blood vessel proximate to the heart. In still further embodiments, the cell is administered to a region of an atrium or ventricle of the heart.

In certain embodiments of the instant methods, the disorder is a sinus node dysfunction, sinus bradycardia, marginal pacemaker function, sick sinus syndrome, tachyarrhythmia, sinus node reentry tachycardia, atrial tachycardia from an ectopic focus, atrial flutter, atrial fibrillation, bradyarrhythmia, or cardiac failure, and the cell is administered to the right or left atrial muscle, sinoatrial node, or atrioventricular junctional region of the subject's heart. In other embodiments, the disorder is a conduction block, complete atrioventricular block, incomplete atrioventricular block, or bundle branch block, and the cell is administered to a region of the subject's heart so as to compensate for the impaired conduction in the heart. This region may be a ventricular septum or free wall, atrioventricular junction, or bundle branch of the ventricle.

The present invention further provides a method of treating a subject afflicted with a cardiac rhythm disorder comprising transfecting a cell of the subject's heart with any of the nucleic acids expressing a HCN polypeptide described herein so as to functionally express the chimeric HCN polypeptide in the heart, wherein expression of the polypeptide is effective to induce a pacemaker current in the heart and thereby treat the subject.

The invention still further provides a method of inhibiting the onset of a cardiac rhythm disorder in a subject prone to such disorder comprising transfecting a cell of the subject's heart with any of the nucleic acids expressing a HCN polypeptide described herein so as to functionally express the chimeric HCN polypeptide in the heart, wherein expression of the polypeptide is effective to induce a pacemaker current in the heart and thereby inhibit the onset of the disorder in the subject. In certain embodiment of any of the treatment methods disclosed herein, a pre-existing source of pacemaker activity in the heart is ablated, for example by surgery or chemically.

In other embodiments, the cell of the heart is in an atrium or ventricle of the heart.

In certain embodiments, the disorder is a sinus node dysfunction, sinus bradycardia, marginal pacemaker function, sick sinus syndrome, tachyarrhythmia, sinus node reentry tachycardia, atrial tachycardia from an ectopic focus, atrial flutter, atrial fibrillation, bradyarrhythmia, or cardiac failure, and a cell in the right or left atrial muscle, sinoatrial node, or atrioventricular junctional region of the subject's heart is transfected. In other embodiments, the disorder is a conduction block, complete atrioventricular block, incomplete atrioventricular block, or bundle branch block, and a cell is transfected in a region of the subject's heart so as to compensate for the impaired conduction in the heart. This region may be a ventricular septum or free wall, atrioventricular junction, or bundle branch of the ventricle.

This invention also provides a method of producing any of the chimeric HCN polypeptides disclosed herein comprising (a) generating a recombinant nucleic acid by joining a nucleic acid encoding an amino terminal portion of a HCN polypeptide to a nucleic acid encoding an intramembranous portion of a HCN polypeptide and joining said nucleic acid encoding the intramembranous portion to a nucleic acid encoding a carboxy terminal portion of a HCN polypeptide, wherein the encoded portions of the HCN polypeptide are derived from more than one HCN isoform or mutant thereof, and (b) functionally expressing the recombinant nucleic acid in a cell so as to produce the chimeric HCN polypeptide.

The invention further provides a method of making any of the instant chimeric HCN polypeptides comprising splicing an amino terminus portion of an HCN channel to be contiguous with an intramembranous portion of a HCN channel to be contiguous with a carboxy terminus portion of a human HCN channel, wherein at least one of portions is derived from a HCN isoform which is different from the HCN isoform from which at least one of the other two portions is derived.

A major shortcoming of electronic pacemakers is their inadequate response to the demands of exercise or emotion. An added advantage of the methods of treating or inhibiting the onset of cardiac disorders disclosed herein is that the methods comprise enhancing beta-adrenergic responsiveness of the heart. These methods also comprises decreasing outward potassium current, I_{K1}, and increasing inward current, I_{f}.

The following Examples are presented to aid in understanding the invention, and are not intended, and should not be construed, to limit in any way the invention set forth in the claims which follow thereafter. These Examples do not include detailed descriptions of experimental methods that are well known to those of ordinary skill in the art, such as methods used in the construction of recombinant nucleic acid vectors, transfection of host cells with such recombinant vectors, and the functional expression of genes in transfected cells. Detailed descriptions of such conventional methods are provided in numerous publications, including Sambrook et al. (1989), the contents of which are hereby incorporated herein in their entirety.

### EXAMPLE 1

### Expression and Electrophysiological Characterization of HCN Channels in Cultured Cells

Isolation and Culture of Cardiomyocytes and Xenopus Laevis Oocytes

Adult rats were anesthetized with ketamine-xylazine before cardiectomy, and neonatal rats decapitated in accordance with the Institutional Animal Care and use Committee protocols of Columbia University. Newborn rat ventricular myocyte cultures were prepared as previously described (Protas and Robinson, 1999). Briefly, 1-2-day-old Wistar rats were euthanized, hearts were quickly removed and ventricles were dissociated using a standard trypsinization procedure. Myocytes were harvested, preplated to reduce fibroblast proliferation, cultured initially in serum-containing medium (except when being transfected with plasmids as described below), and then incubated in serum free medium (SFM) at 37° C., 5% CO₂ after 24 h. Action potential studies were conducted on 4-day-old monolayer cultures plated directly onto fibronectin-coated 9×22 mm glass coverslips. For voltage clamp experiments, 4-6 day old monolayer cultures were resuspended by brief (2-3 min) exposure to 0.25% trypsin, then replated onto fibronectin-coated coverslips and studied within 2-8 h.

Freshly isolated adult ventricular myocytes were prepared using the procedure described by Kuznetsov et al. (1995). This entailed a Langendorff perfusion of collagenase, followed by trimming away of the atria. The remaining tissue was minced and dissociated in additional collagenase solution. The isolated myocytes were suspended in a SFM then plated on 9×22 mm glass coverslips at 0.5-1×10³ cells/mm². Two to three hours later, after the myocytes had adhered to the coverslips, the adenoviral infection procedure was begun (see below).

For preparation of canine myocytes, adult dogs of either sex were killed using an approved protocol by an injection of sodium pentobarbital (80 mg kg⁻¹ body weight). Cardiomyocytes were isolated from the canine ventricle as previously described (Yu et al., 2000). A method of primary culture of canine cardiomyocytes was adapted from the procedure described for mouse cardiomyocytes (Zhou et al., 2000). The cardiomyocytes were plated at 0.5-1 (10⁴ cells cm⁻² in minimal essential medium (MEM) containing 2.5% fetal bovine serum (FBS) and 1% penicillin/streptomycin (PS) onto mouse laminin (10 μg ml⁻¹) precoated coverslips. After 1 h of culture in a 5% CO₂ incubator at 37° C., the medium was changed to FBS-free MEM. Stem cells were added after 24 h and coculture was maintained in Dulbecco's modified Eagle's medium (DMEM) with 5% FBS. Cell Tracker Green (Molecular Probes, Eugene, Oreg.) was used to distinguish hMSCs from HeLa cells in coculture in all experiments (Valiunas et al., 2000).

Oocytes were prepared from mature female Xenopus laevis in accordance with an approved protocol as previously described (Yu et al., 2004).

Expression of Wild-Type and Mutant HCN Channels in Cardiomyocytes and Oocytes

cDNAs encoding mouse HCN2 (mHCN2, GenBank AJ225122) or HCN4 (mHCN4, GenBank deposit in progress) were subcloned into the pCI mammalian expression vector (Promega, Madison, Wis.). The resulting plasmids (pCI-mHCN2 or pCI-mHCN4) were used for neonatal rat ventricular myocyte transfection, as indicated. A separate plasmid (pEGFP-CI; Clontech, Palo Alto, Calif.) expressing the gene of enhanced green fluorescent protein (EGFP) as a visual marker for successful DNA transfer was included in all transfection experiments. For transfection, 2 μg of pCI-mHCN and 1 pg of pEGFP-CI were first incubated in 200 μl of SFM containing 10 μl of lipofectin (Gibco Life Technologies, Rockville, Md.) at room temperature for 45 min. The mixture was then added to a 35-mm petri dish containing 106 cells suspended in 0.8 ml of SFM. After overnight incubation at 37° C. in a CO2 incubator, the medium containing the plasmids and lipofectin was discarded and the dish was refilled with 2 ml of fresh SFM. Patch clamp experiments were carried out on resuspended cells exhibiting detectable levels of GFP by fluorescence microscopy 3-5 days after transfection.

For increased expression efficiency, an adenoviral construct for mHCN2 was prepared. Gene delivery and transfer procedures followed previously published methods (Ng et al., 2000; He et al., 1998). A DNA fragment (between EcoRI and XbaI restriction sites) that included mHCN2 DNA downstream of the CMV promoter was obtained from plasmid pTR-mHCN2 (Santoro and Tibbs, 1999) and subcloned into the shuttle vector pDC516 (AdMax™; Microbix Biosystems, Toronto, Canada). The resulting pDC516-mHCN2 shuttle plasmid was co-transfected with a 35.5 kb El-deleted Ad genomic plasmid pBHGΔE1,3FLP (AdMax™) into El-complementing HEK293 cells. Successful recombination of the two vectors resulted in production of the adenovirus mHCN2 (AdmHCN2), which was subsequently plaque-purified, amplified in HEK293 cells, and harvested after CsCl-banding to achieve a titer of at least 10¹¹ ffu/ml.

An adenoviral construct of mouse mHCN2 (AdmHCN2) was also prepared as previously described (Qu et al., 2001). The mE324A point mutation was introduced into the mHCN2 sequence with the QuikChange® XL Site-Directed Mutagenesis Kit (Stratagene, La Jolla, Calif.) and packaged in the pDC515 shuttle vector (AdMaX™, Microbix Biosystems) to create pDC515mE324A. PDC515mE324A then was co-transfected with pBHGfrtΔE1,3FLP into El-complimenting HEK293 cells. The adenoviral construct AdmE324A was subsequently harvested and CsCl purified. For consistency with earlier studies (Qu et al., 2003), when preparing samples for in vivo injection, 3×10¹⁰ ffu of each adenovirus was mixed with an equal amount of a GFP-expressing adenovirus (AdGFP) in a total volume of 700 μl.

AdHCN2 infection of rat ventricular myocytes was carried out 2-3 h after the isolated cells were plated onto coverslips. The culture medium was removed from the dishes (35-mm) and the inoculum of 0.2-0.3 ml/dish was added containing AdHCN2. The value of m.o.i. (multiplicity of infection—the ratio of viral units to cells) was 15-100. The inoculum was dispersed over the cells every 20 min by gently tilting the dishes so that the cells were evenly exposed to the viral particles. The dishes were kept at 37° C. in a CO₂ incubator during the adsorption period of 2 h, then the inoculum was discarded and the dishes were washed and refilled with the appropriate culture medium. The dishes remained in the incubator for 24-48 h before electrophysiological experiments were conducted.

Adenoviral infection of the newborn ventricular myocytes was performed on cell monolayer cultures 4 days after initial plating. Cells were exposed to a virus-containing mix (m.o.i. 20, in 250 μl of culture medium) for 2 h, rinsed twice and incubated in SFM at 37° C., 5% CO₂ for 24-48 hours prior to the cells being resuspended as described above for electrophysiological study. In early experiments, AdGFP was employed but since >90% of cells exposed to AdmHCN2 in vitro were found to express the current (Qu et al., 2001), in later experiments cells were not co-infected with AdGFP to aid in the selection of infected cells.

For expression of HCN in Xenopus oocytes, oocytes were injected with 5 ng of cRNA made from mouse wild-type mHCN2 and mutant mHCN2 (E324A) plasmids. Injected oocytes were incubated at 18° C. for 24-48 h prior to electrophysiological analysis.

Electrophysiological Measurements in Cultured Cardiomyocytes and Oocytes

Voltage and current signals were recorded using patch clamp amplifiers (Axopatch 200). The current signals were digitized with a 16 bit A/D-converter (Digidata 1322A, Axon Instruments, Union City, Calif.) and stored with a personal computer. Data acquisition and analysis were performed with pCLAMP 8 software (Axon Instruments). Curve fitting and statistical analyses were performed using SigmaPlot and SigmaStat, respectively (SPSS, Chicago, Ill.).

The whole-cell patch clamp technique was employed to record mHCN2 current from cultured myocytes. Experiments were carried out on cells superfused at 35° C. The external solution contained (mM): NaCl, 140; NaOH, 2.3; MgCl₂, 1; KCl, 10; CaCl₂, 1; HEPES, 5; glucose, 10; pH 7.4. MnCl₂ (2 mM) and BaCl₂ (4 mM) were added to block other currents. The pipette solution contained (mM): aspartic acid, 130; KOH, 146; NaCl, 10; CaCl₂, 2; EGTA-KOH, 5; Mg-ATP, 2; HEPES-KOH, 10; pH 7.2.

To measure the HCN activation curve, a standard two-step protocol was employed. Hyperpolarizing steps from −25 to −135 mV for mHCN2 and from −5 or −15 to −135 mV for mE324A were applied from a holding potential of −10 mV, followed by a tail current step (to −125 or −135 mV). The duration of test steps was longer at less hyperpolarized potential for mHCN2 channels, to more closely approach steady-state activation at all voltages. The normalized plot of tail current versus test voltage was fit with a Boltzmann function and then the voltage of half maximum activation (V_{1/2}) and slope factor(s) were defined from the fitting. Activation kinetics were determined from the same traces, while deactivation kinetics were determined from traces recorded at each test potential after achieving full activation by a prepulse to −135 mV. Time constants were then obtained by fitting the early time course of activation or deactivation current traces with a monoexponential function; the initial delay and any late slow activation or deactivation phase were ignored (Qu et al., 2001; Altomare et al., 2001). Current densities are expressed as the value of the time-dependent component of current amplitude, measured at the end of the test potential and normalized to cell membrane capacitance. Records were not corrected for liquid junction potential, which was previously determined to be 9.8 mV under these conditions (Qu et al., 2001).

For measurements in Xenopus oocytes, oocytes were voltage-clamped using a two-microelectrode voltage clamp technique. The extracellular recording solution (OR2) contained (in mM): NaCl, 80; KCl, 2; MgCl₂, 1; and Na-HEPES, 5 (pH 7.6). For the recording of steady state activation of expressed Wt mHCN2, currents were elicited by 2-s long hyperpolarizing pulses between −30 mV and −160 mV with 10 mV increments, followed by a 1-s depolarizing pulse to +15 mV. The holding potential was −30 mV. As to the mHCN2 (E324A), currents were elicited by 3-s-long hyperpolarizing pulses between +20 mV and −130 mV with 10 mV increments, followed by a 1 second depolarizing pulse to +50 mV. The holding potential was +20 mV. To construct the current/voltage relationship for wildtype (Wt) mHCN2, the cell was held at '130 mV, the current was elicited by a 2-s hyperpolarizing voltage step to −140 mV to saturate activation, and followed by 2-s depolarizing voltage steps between '180 mV and +50 mV in 10 mV increments. For mHCN2 (E324A), the cell was held at +20 mV, current was elicited by a 1.5-s hyperpolarizing voltage step to −110 mV to saturate activation, and then followed by 1.5-s depolarizing voltage steps between −80 mV and +50 mV in 10 mV increments for the recording of tail currents. To record the current amplitudes for Wt mHCN2, the current was evoked by applying a 3-s hyperpolarizing voltage pulse to −120 mV from a holding potential of −30 mV. For mHCN2 (E324A), the current was evoked by applying a 3-s hyperpolarizing voltage pulse to −120 mV from a holding potential of +20 mV.

Data are presented as means ±SEM. Experimental data were compared using a Student's t-test or Chi-square test with Yates' correction, as appropriate. When making comparisons, matched cells from the same cultures were used, and data from at least 3 separate cultures were pooled for each comparison.

Pacemaker Currents induced by mHCN2 and mE324A in Cardiomyocytes

Previous experiments have shown that overexpression of HCN2 in neonatal rat myocytes in culture induced a pacemaker current which increased beating rate, and that mutations in the HCN2 pacemaker gene and/or the addition of appropriate accessory channel subunits altered the characteristics of the expressed current in ways that would be expected to further enhance the beating rate (U.S. Pat. No. 6,849,611; Qu et al., 2001; Qu et al., 2004). Infection with an Ad expressing HCN2 also significantly increased the spontaneous beating rate of monolayer cultures of synchronously beating (U.S. Pat. No. 6,849,611; Qu et al., 2001). Myocyte cultures were also infected with the HCN2 adenovirus and a second virus carrying either GFP or an HA-tagged form of MiRP1 which is the beta subunit for HCN2. The result was a significant increase in current magnitude and acceleration of activation and deactivation kinetics (Qu et al., 2004).

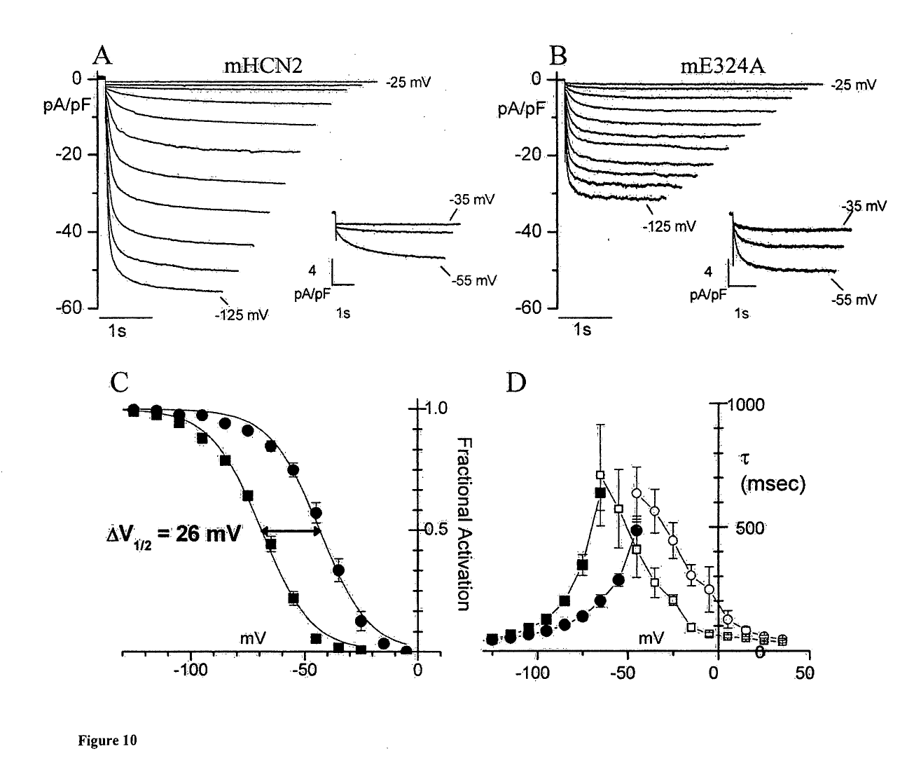

In the whole-cell voltage clamp experiments described herein, mHCN2- and mE324A-expressing myocytes both gave rise to an inward current in response to hyperpolarizing voltages. Representative normalized current traces obtained at test potentials ranging from −25 to −125 mV, from a holding potential of −10 mV, are shown in FIGS. 10A and B. It is apparent from the expanded currents in the insets that the activation threshold of mE324A channels is less negative than that of mHCN2 channels.

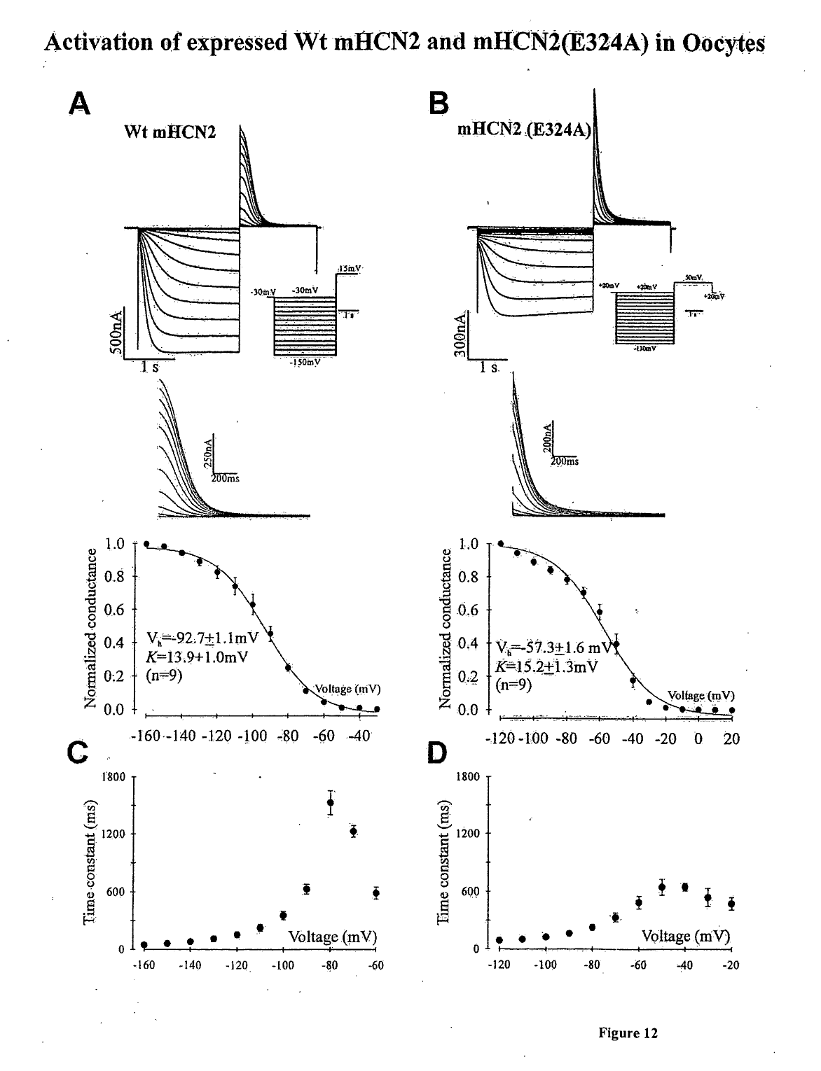

The difference in voltage dependence of activation between mHCN2 and mE324A is more evident from the mean current-voltage relationships shown in FIG. 10C. The curves were obtained from tail currents, as described above. The individual activation curves were each fit to the Boltzmann equation and the calculated midpoint (V_{1/2}) and slope factor (s) from all cells averaged and statistically compared. Mean parameters for mHCN2 (n=14) and mE324A (n=16) expressing cells, respectively, were: V_{1/2}=−66.1±1.5 mV and −46.9±1.2 mV (P<0.05) and s=10.7±0.5 mV and 9.6±0.4 mV (p>0.05). Thus, in agreement with data previously obtained in oocytes (Chen et al., 2001) and confirmed herein (see FIGS. 12-15), the mE324A mutation resulted in a positive shift of the activation curve relative to that of mHCN2 when both constructs were expressed in newborn myocytes.

The activation kinetics of mE324A channels appeared faster than those of mHCN2 (FIGS. 10A, B insets). To demonstrate this difference, time constants of activation and deactivation were measured at different voltages as described above, and averaged (FIG. 10D). These data show that the faster activation kinetics observed for mE324A channels were due to a positive shift of the voltage-dependence of gating kinetics. Both activation and deactivation voltage dependence shifted positively, so that at the positive voltages at which deactivation was measured, the deactivation was slower for mE324A than for mHCN2. Moreover, this shift is comparable to that in the current-voltage relationship. Indeed the relative peaks of the kinetic-voltage relations were consistent with the previously determined V_{1/2} values.

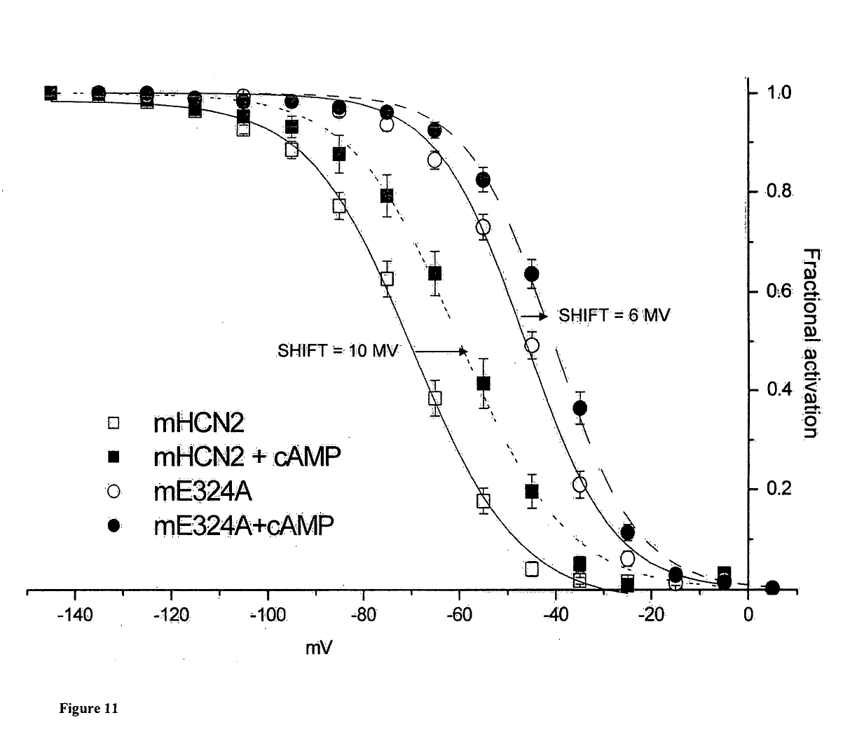

The positive shift in the activation relation and kinetics would be expected to result in more current being passed earlier in the cardiac cycle with mE324A in comparison to mHCN2. However, to be beneficial as a biological pacemaker, it also is necessary to preserve autonomic responsiveness. To assess this, mHCN2 and mE324A activation curves were compared in the absence and in the presence of cAMP in the pipette solution (FIG. 11). Both channels responded to the presence of saturating intracellular cAMP as detailed in the brief description of FIG. 11.

Whether the mutant channel expressed current as well as the wild-type was also investigated. The percentage of myocytes expressing mE324A current was significantly smaller than the percentage expressing mMCN2 (36.6% of 93 cells vs. 74.5% of 47 cells respectively, P<0.05) in 6 matched cell cultures. Moreover, in the cells that did express current the mE324A current density (measured at −135 mV) was about 2.5 times smaller than that of mHCN2 (21.0±3.5 pA/pF, n=12, vs. 53.5±8.7 pA/pF, n=10, respectively, P<0.05).

Currents induced by mHCN2 and mE324A in Xenopus Oocytes

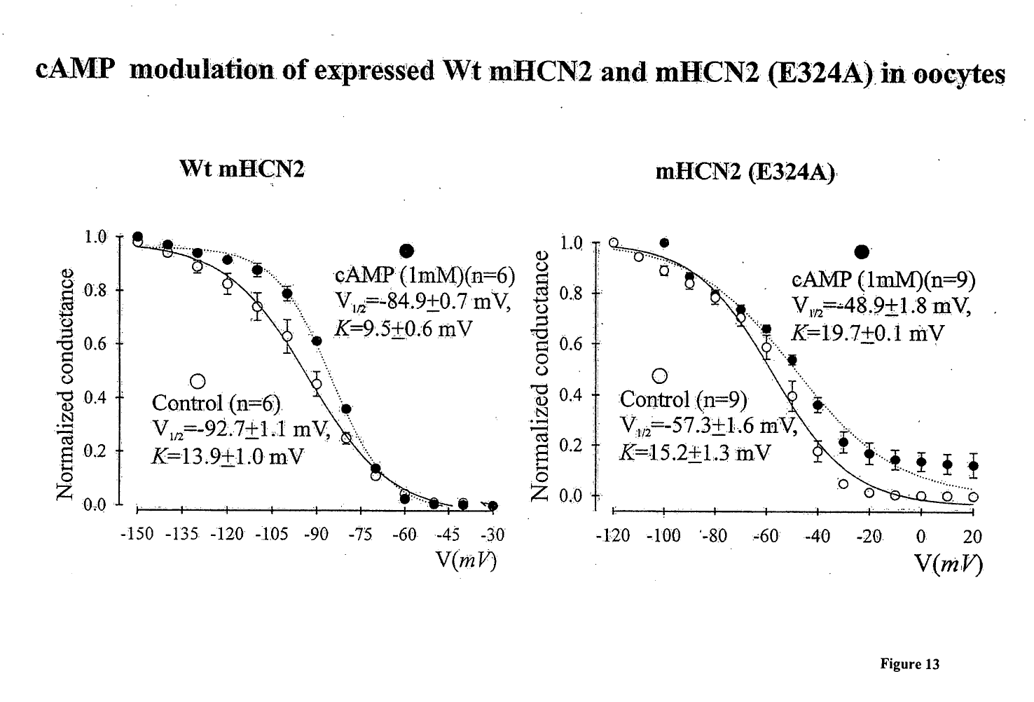

FIG. 12 shows activation properties and kinetics of the heterologously expressed current. In oocytes, the mHCN2 activates 35 mV more negatively than mE324A. This more positive activation is accompanied by both a shift in the voltage dependence of the kinetics of activation as well as more rapid kinetics at the midpoint of activation for mE324A. Both mHCN2 and mE324A responded to application of 8-Br-cAMP (1 mM) with a positive shift in activation (FIG. 13). For mHCN2, cAMP shifted the Vₕ by approximately 8 mV (Vₕ values were −92.7 mV±1.1 mV for control and −84.9 mV±0.7 mV for cAMP (n=6, P<0.01), and the corresponding slope (s) values were 13.9 mV±1.0 mV and 9.5 mV±0.6 mV (n=6, p>0.05). For mE324A, cAMP positively shifted the Vₕ by approximately 7 mV (Vₕ values were −57.3 mV±1.6 mV for control and −48.9 mV±1.8 mV for cAMP (n=9, P<0.01), and the corresponding slope (s) values were 15.2 mV±1.3 mV and 19.7 mV±0.1 mV (n=9, p>0.05).

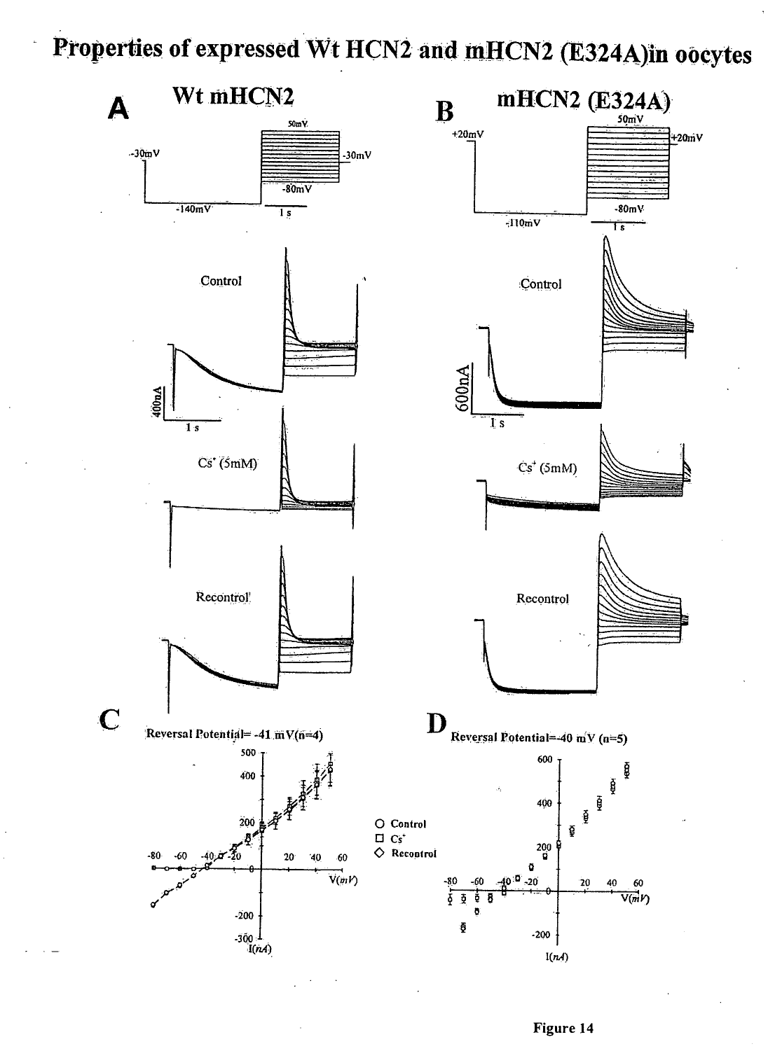

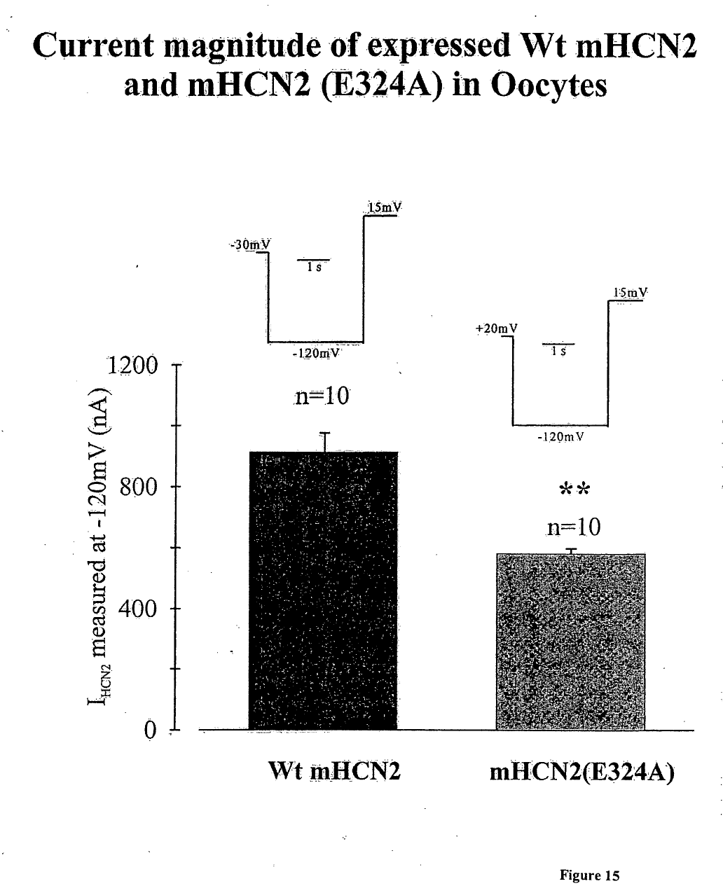

Both mHCN2 and mE324A generated inward currents blocked by 5 mM Cs⁺ with reversal potentials near −40 mV (FIG. 14). Finally, a single voltage pulse was applied near saturation (−120 mV) to compare the levels of expression of mHCN2 and mE324A. The HCN2 induced current was 912.7±63.7 nA, n=9, while the E324A induced current was 579.8±18.2 nA, n=9 (P<0.01). Thus, there was significantly reduced expression for those oocytes expressing mE324A (see FIG. 15).

### EXAMPLE 2

### Effects of Cellular Environment of Newborn and Adult Ventricular Myocytes on Gating and Excitability of HCN Channels

Voltage Dependence of HCN Isoforms in different Cell Types

Four members of the HCN gene family are currently known (Santoro et al., 1997; Ludwig et al., 1998; Santoro et al., 1998). Three of these (HCN1, HCN2 and HCN4) are present in the heart, but the relative message level of the three isoforms varies with region and age (Shi et al., 1999; Ishii et al., 1999; Ludwig et al., 1999). Sinus node and Purkinje fibers, in which I_{f} activates at less negative potentials, contain largely HCN1 and HCN4. Ventricles contain HCN2 and HCN4, with the ratio of mRNA of HCN2 relative to HCN4 being greater in the adult than newborn ventricle. This suggests that HCN2 is an inherently negatively activating isoform whose relative abundance determines the activation threshold in different regions of the heart or at different ages. However, heterologous expression studies do not support this simple explanation. While there is some variability between laboratories, when HCN2 and HCN4 have been expressed in mammalian cell lines activation voltages differed by less than 10 mV (Ludwig et al., 1999; Moosmang et al., 2001; Altomare et al., 2001). Thus, the intrinsic characteristics of the specific HCN isoform expressed does not seem, by itself, to be a sufficient explanation for the diverse voltage dependence of the native I_{f}, either regionally in the adult heart or developmentally in the ventricle.

An alternative hypothesis is that the cellular environment in which a particular isoform is expressed influences its voltage dependence. The cellular environment could include the presence or absence of beta subunits, cytoskeletal elements, kinases, phosphatases or other factors. Because of these potential differences, HCN2 and/or HCN4 voltage dependence might differ when expressed in myocytes rather than in a heterologous expression system. For the same reason, one or both of these isoforms may be sensitive to the maturational state of the myocyte, exhibiting distinct voltage dependence when expressed in newborn as compared to adult ventricular cells. Here, data is presented to address these issues.

Expression of HCN Isoforms in Cultured Cells

Adult and neonatal rat ventricular myocytes were isolated and cultured as described in Example 1. Mouse HCN2 and HCN4 were subcloned into the pCI and adenoviral vectors and expressed in these cultured myocytes also as described in Example 1.

Electrophysiological Measurements

The whole-cell voltage clamp technique was employed to record native I_{f} or expressed I_{HCN2} or I_{HCN4}. Action potentials were recorded in current clamp mode, again using a whole cell patch electrode. Experiments were carried out on cells superfused at 35° C. Extracellular solution contained (mM): NaCl, 140; NaOH, 2.3; MgCl₂, 1; KCl, 5.4; CaCl₂, 1.0; HEPES, 5; glucose, 10; pH 7.4. To record from myocytes expressing native currents (I_{f}), myocytes expressing HCN2 (I_{HCN2}) or HCN4 (I_{HCN4}), [K⁺]₀ was increased to 10 mM, and MnCl₂ (2 mM) and BaCl₂ (4 mM) added to the superfusate to eliminate calcium and inward rectifier (I_{K1}) currents. In some experiments CsCl (4 mM) was used extracellularly to identify the pacemaker current as the Cs-sensitive current. The patch pipette solution included (mM): aspartic acid, 130; KOH, 146; NaCl, 10; CaCl₂, 2; EGTA-KOH, 5; Mg-ATP, 2; HEPES-KOH, 10, pH 7.2. Where indicated, 10 μM cAMP was included in the pipette solution. A fast solution changing apparatus expedited the experimental protocols. The pipette resistance was typically 1-3 M. An Axopatch-200B amplifier and pClampS software (Axon Instruments) were used for acquisition and data analysis. The pacemaker current (I_{f}, I_{HCN2} or I_{HCN4}) was defined as the time-dependent component taken at the end of a hyperpolarizing step to voltages in the range of −35 to −145 mV, while the holding potential was −35 mV unless otherwise indicated. For I_{f} and I_{HCN2} measurements, the hyperpolarizing test pulses were 3 or 6 s long throughout the voltage range. To accurately record steady-state currents for the more slowly activating I_{HCN4}, the test voltages varied in length from 6 sec at −125 mV to as long as 60 s at −55 mV. When recording tail currents, the test pulses were followed by an 8-s voltage step to −125 mV. In all pacemaker current protocols, each episode ended with a pulse to −5 mV for 0.5 s to insure full deactivation.

The activation relation of the native or expressed current can be determined from the steady-state I-V relation. In this case, the reversal potential (V_{f}) was separately determined from the fully activated I-V relation (Accili et al., 1991) and used to generate the activation relation (y=I/(gₘₐₓ*(V−V_{f})), where gₘₐₓ is the maximal conductance). This method was used in the initial studies of expression with HCN2 or HCN4 plasmid in neonatal rat ventricular cells. Subsequent studies of I_{f} or I_{HCN2} employed tail current measurements. Tail current, after being plotted against the test voltage, gave the maximum conductance and activation-voltage relation. This relation was normalized by the maximum conductance and fitted with the Boltzmann function (y=1/(1+exp ((V−V_{1/2})/K))) to determine the voltage of half maximum activation (V_{1/2}) and slope factor (K). Tail currents were measured at a negative voltage (−125 mV) to avoid contamination by transient outward and other currents at less negative voltages.

The kinetics of activation were determined by a single exponential fit to the early time course of the current activated by hyperpolarizing pulses. Both the initial delay and any late slow activation were ignored. The kinetics of deactivation were determined by a single exponential fit of the time course of the current trace at each test voltage after maximal activation by a conditioning pulse to −125 mV. For both activation and deactivation, the length of the current trace being fit was at least three times as long as the measured time constant to insure accuracy.

All data are presented as mean ±S.E.M. Statistical significance was examined by f-test for paired and ANOVA for multiple comparisons, and determined at P<0.05.

Comparison of Neonatal Ventricular Myocytes expressing HCN2 and HCN4

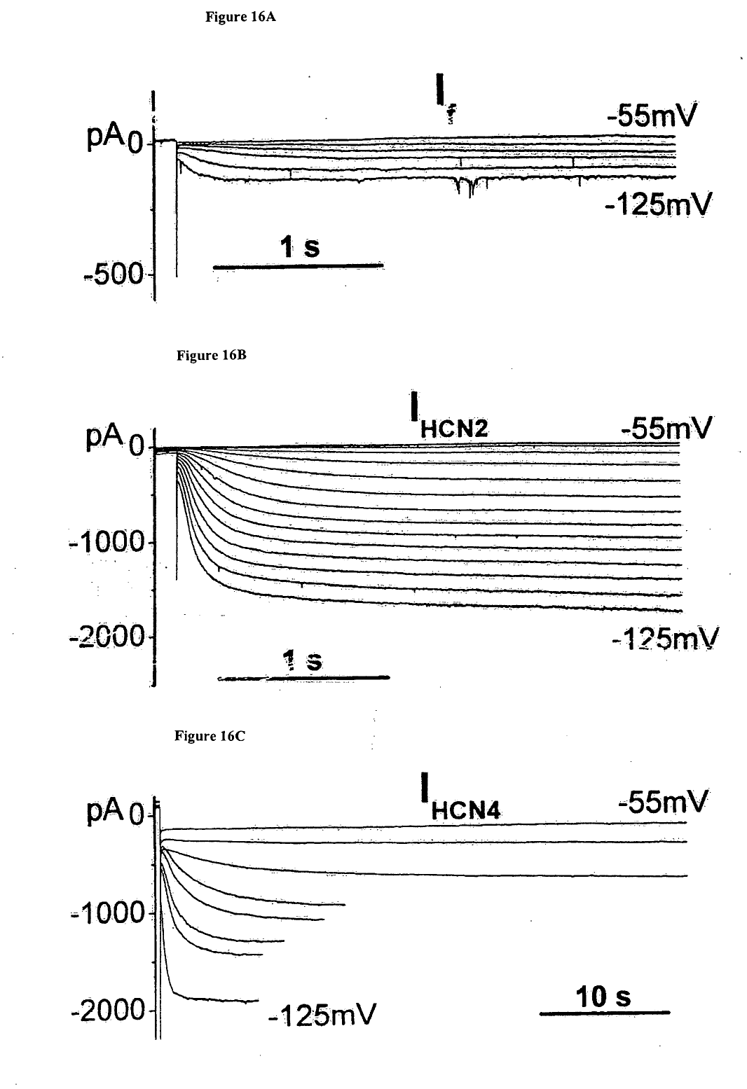

It has previously been reported that neonatal rat ventricle cells in culture exhibit a small I_{f} that activates with a threshold voltage around −70 mV (Robinson et al., 1997; Cerbai et al., 1999). FIG. 16A provides a representative family of current traces of the native I_{f} in a neonatal rat ventricle cell in culture. A time-dependent inward current component is apparent for voltage steps of −65 mV or more negative. Studies of message levels by RNase protection assay have indicated that both HCN2 and HCN4 are present in the newborn ventricle, with relative message levels of about 5:1 (Shi et al., 1999). Therefore, each of these isoforms were expressed separately in the neonatal ventricle cultures. As previously described, a lipofectin transfection method was employed and the HCN plasmids were co-transfected with pEGFP-Cl to aid in the identification of expressing cells. Expression efficiency was less than 5%, based on the number of visually detected fluorescent cells. More than 90% of fluorescent cells possessed an I_{f}-like current at least 10 times greater in magnitude than the native current. FIGS. 16B and C illustrates representative expressed current traces from myocytes transfected with HCN2 and HCN4, respectively. The current magnitude is such as to clearly distinguish the expressed current from the native current. Further, the slower kinetics of the expressed HCN4, compared to HCN2, is apparent (note different time scale in FIG. 16C). Slower HCN4 kinetics also have been reported in heterologous expression studies.

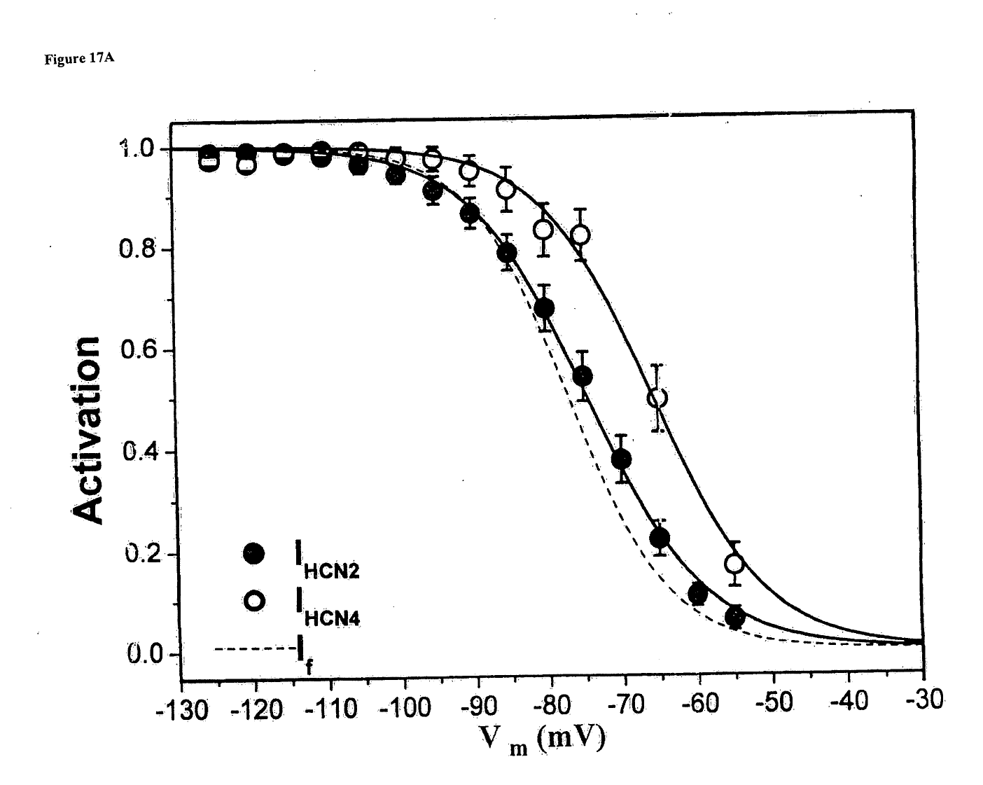

Current records such as those illustrated in FIG. 16 were used to determine the quasi steady-state I-V relation. For the native the current and for expressed HCN2, 3-s voltage steps were sufficient to approximate steady-state at most of the test voltages, although the current did not achieve steady-state at the less negative steps. Significantly longer pulses were required for adequate analysis of HCN4. Reversal potential in each case was separately determined, and used to convert the I-V relation to the corresponding activation relation of the native and expressed current. The average activation relations for native current (taken from Qu et al., 2001) and for expressed currents are shown in FIG. 17A. The experiments were conducted on 4-6 day old cultures that had been transfected the same day as cells were dissociated. Neonatal myocytes expressing HCN2 exhibited currents that activated at more negative voltages than those expressing HCN4, and this difference was statistically significant (V_{1/2} of −74.8±1.4 mV, n=17, and −66.3±2.0 mV, n=14, respectively; P=0.001). Slope factors (K) did not differ (7.7±0.7 mV and 6.7±0.7 mV; P=0.348). The 8.5 mV difference in midpoint of activation of HCN2 and HCN4, while statistically different, is considerably less than the 40 mV difference (based on threshold measurements) between adult and neonatal ventricle. This suggests that, while a developmental increase in the HCN2/HCN4 ratio might contribute to the age-dependent negative shift in activation of I_{f}, it cannot fully explain the shift. FIG. 17B compares the activation kinetics of the currents recorded from neonatal ventricular myocytes expressing HCN2 (n=6-9) and HCN4 (n=4-5). For most voltages, HCN4 activation kinetics are markedly slower than those of HCN2. Since HCN4 activates at less negative voltages than HCN2, this cannot be explained by a shift in the voltage dependence of activation. Rather, it represents a basic difference in the kinetics of the two isoforms, as has also been reported in heterologous expression experiments (Ludwig et al., 1999; Altomare et al., 2001). The native I_{f} (n=8) demonstrates activation kinetics intermediate between those of HCN2 and HCN4, but the small magnitude of the native current made it impractical to obtain reliable kinetic data at less negative voltages, where the behavior of the two expressed isoforms more markedly diverge.

Comparison of Neonatal and Adult Ventricular Myocytes Expressing HCN2

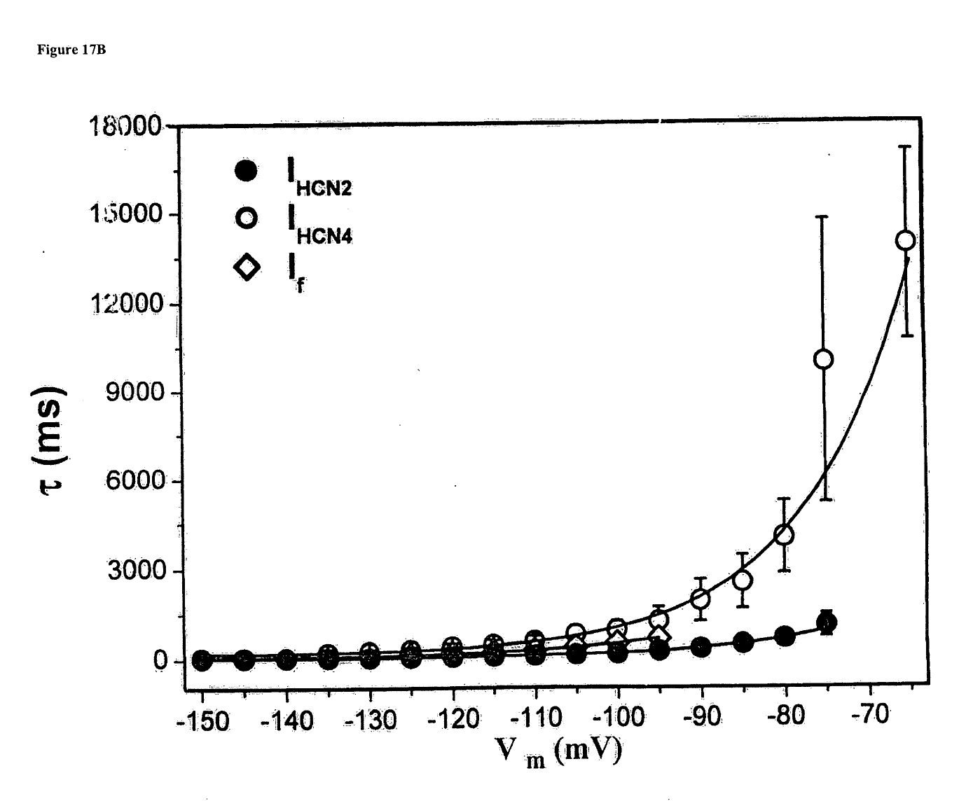

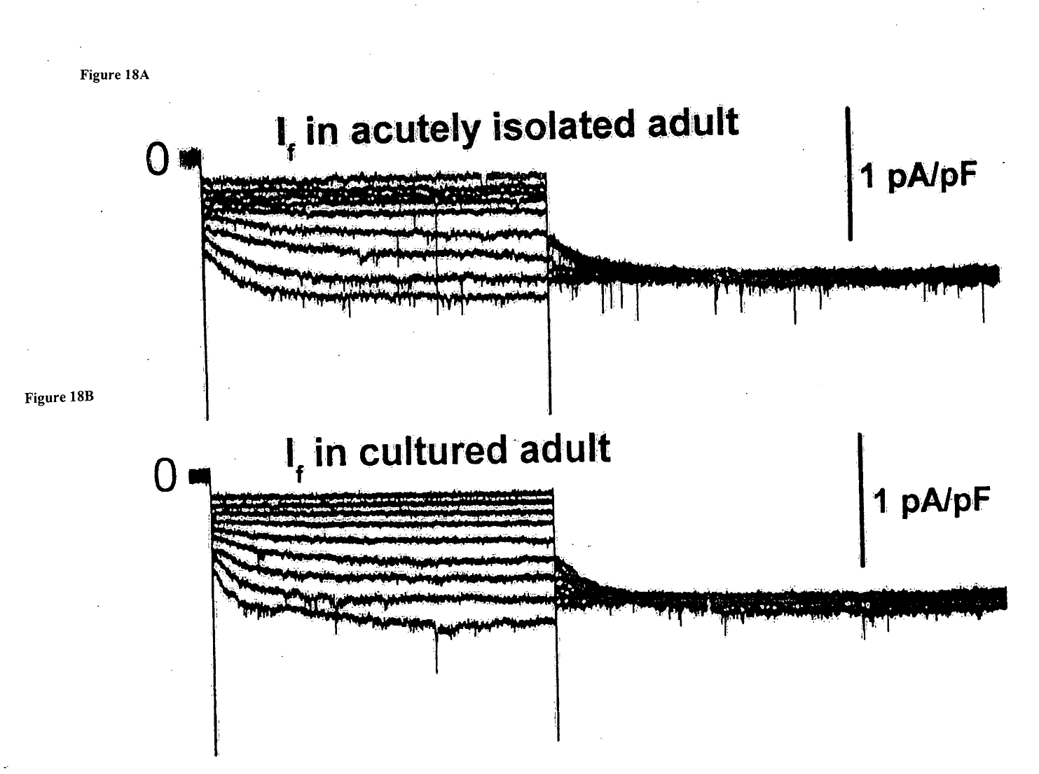

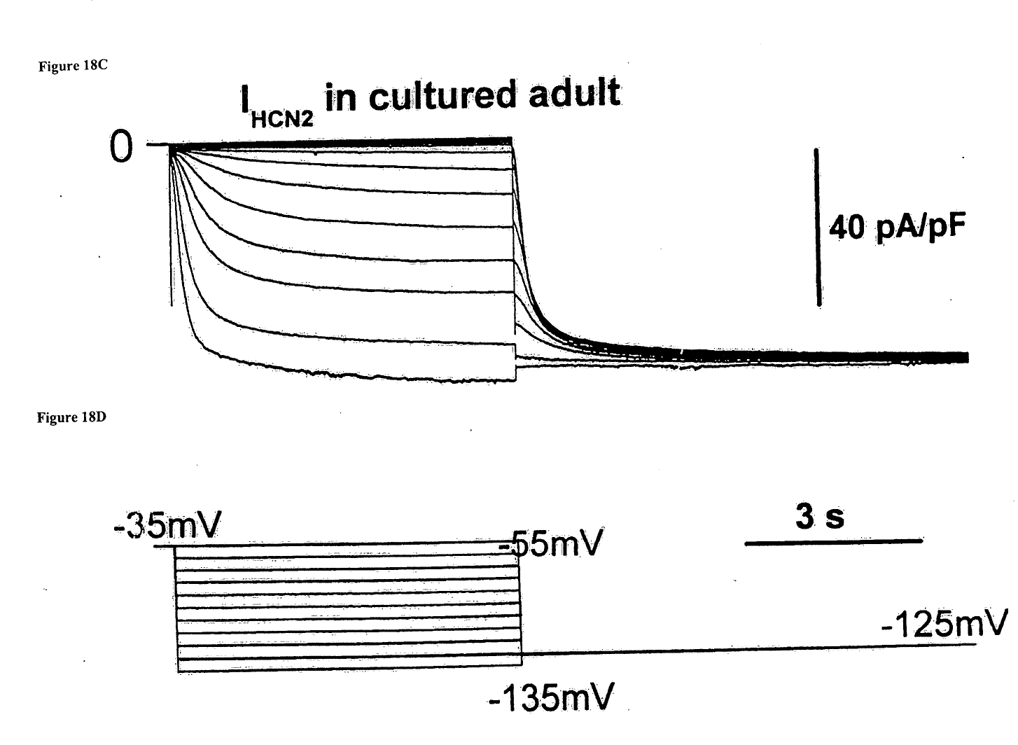

Since the preceding experiments suggested that an HCN4/HCN2 isoform switch was not likely to fully account for the differences in native I_{f} between neonatal and adult ventricle, the characteristics of HCN2 (the major ventricular HCN isoform, at the message level, at both ages (Shi et al., 1999) when expressed in adult versus neonatal ventricular myocytes was compared. This required maintaining adult ventricle cells in culture for 48 h. An earlier report indicated that longer culture conditions could result in a marked positive shift in the voltage dependence of activation of native current (Fares et al., 1998). Therefore, native current in acutely dissociated cells was first compared with that in cells maintained in culture in serum free medium for 2 days. A voltage clamp protocol that allowed direct construction of the activation relation without the need for a separate determination of the reversal potential, was employed. After a hyperpolarizing step to various test voltages, a second step to −125 mV generated a tail current, the amplitude of which was employed to determine the activation relation. FIGS. 18A and B provides representative current traces from acutely dissociated and cultured adult rat ventricle cells. In both cases, the cells were rod-shaped and quiescent and, as seen in the figure, in both cases the threshold voltage (i.e., first voltage step where a time dependent current is apparent) is more negative than was seen for the native current in the neonate (FIG. 16). The lipofectin transfection method, with its low efficiency, was inadequate for studies of HCN expression in adult myocytes. Therefore, an adenoviral construct (AdHCN2) that contained the mouse HCN2 sequence was prepared. Treatment of the adult cells with this adenoviral construct resulted in expression of high current levels (FIG. 18C, note different scale). In adult ventricular myocytes expressing HCN2, the recorded current activated with a more negative threshold than that previously observed in neonatal cells (FIG. 16B).

The HCN2 alpha subunit was employed because in neonatal myocytes it exhibits kinetics and cAMP sensitivity (Qu et al., 2001) that approximate the native sinus node pacemaker current. However, data suggest that native current in the sinus node is predominantly carried by the HCN4 alpha subunit, but HCN1 and HCN2 alpha subunits (Shi et al., 1999; 2000) and the MiRP1 beta subunit (Yu et al., 2001) are also present. Therefore, adenoviral constructs of these other alpha and beta subunits, alone or in combination, can be over-expressed in excitable cells in culture and employed in cell based rate assays. The present construct comprised HCN2 under the control of the CMV promoter which drives high level expression in mammalian cells, but constructs can also be prepared using regulatable promoters to provide greater control over the level of expression.

Neonatal rat ventricle cells were employed because they exhibit many of the other relevant currents of cardiac pacemaking. This includes the presence of T-type and L-type calcium currents and a low density of inward rectifier current. Further, they include pacemaker current, with an activation threshold at or near the physiologic voltage range (Qu et al., 2000). The native pacemaker current in these cells is small, but the fact that it activates at physiologically relevant voltages in the neonatal ventricle (compared to the adult ventricle, where it activates negative to the resting potential (Robinson et al., 1997) suggested that the over-expressed current also would activate in the physiologic voltage range. This prediction has been confirmed (Qu et al., 2001). In fact, both HCN2 and HCN4 are demonstrated to activate at physiologically relevant voltages when expressed in neonatal rat ventricle myocytes (FIGS. 16 and 17). These initial studies employed a low efficiency transfection method to over-express HCN2 or HCN4 in a small percentage of myocytes in culture. While this approach allowed characterization of the current, it did not affect spontaneous beating of the contiguous monoloayer culture since too few cells expressed the current at high density. However, infection of these cultures with an adenoviral construct of HCN2 allows over-expression of the current in >90% of the cells and thereby alter diastolic depolarization and beating rate of the entire culture.

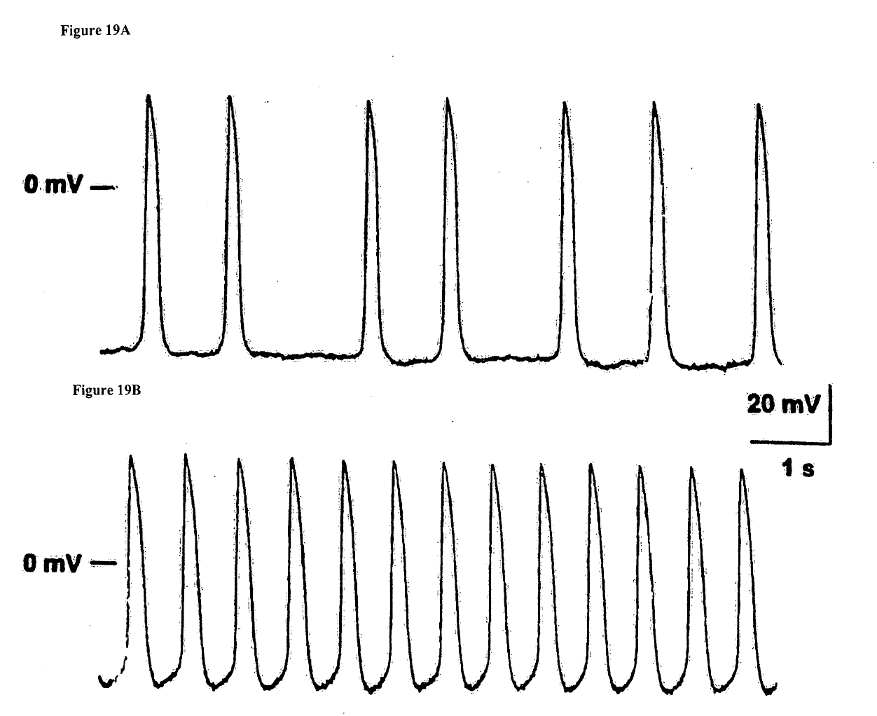

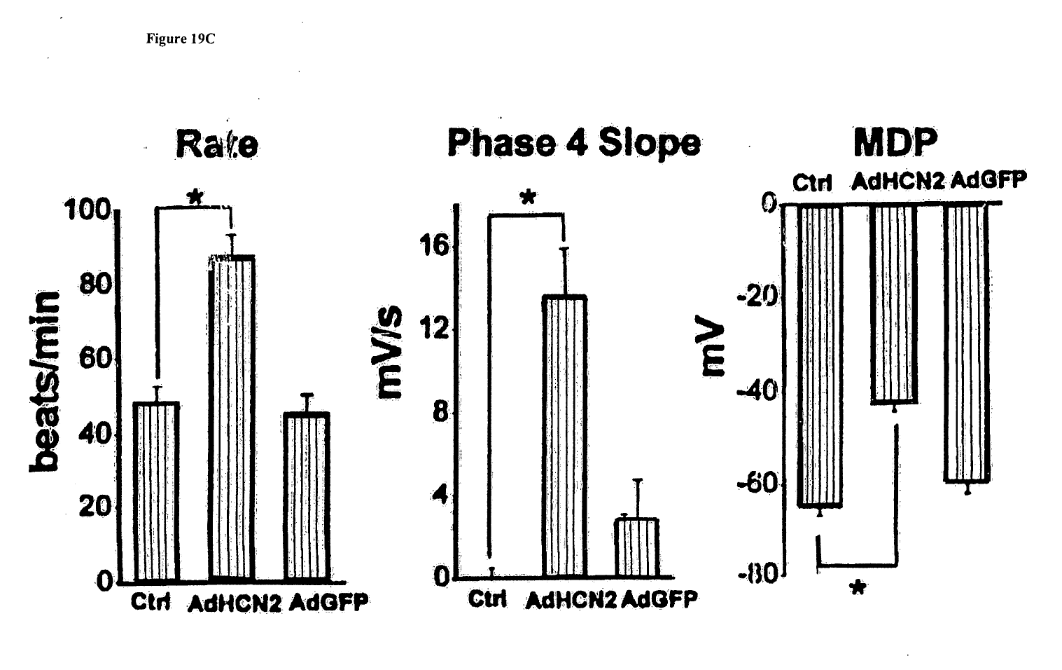

FIG. 19 (panel A) demonstrates that these cultures, when not over-expressing HCN2, beat spontaneously but lack the slow diastolic depolarization characteristic of the normal cardiac sinus node. Further, the cycle length is variable. In contrast, a culture over-expressing HCN2 beats at a faster rate, with a constant cycle length and a pronounced diastolic depolarization (panel B).

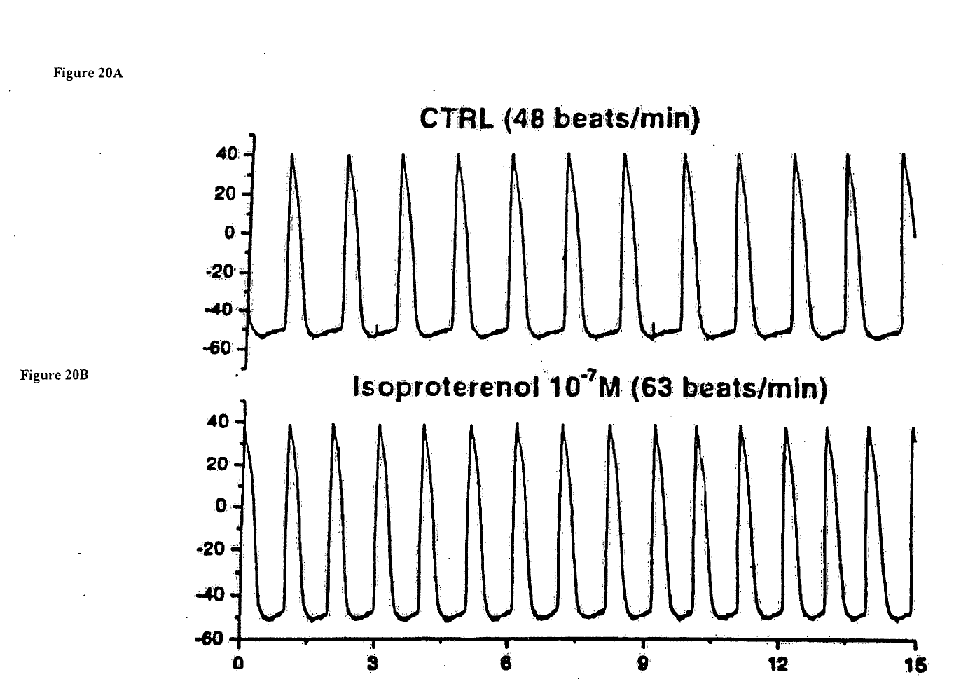

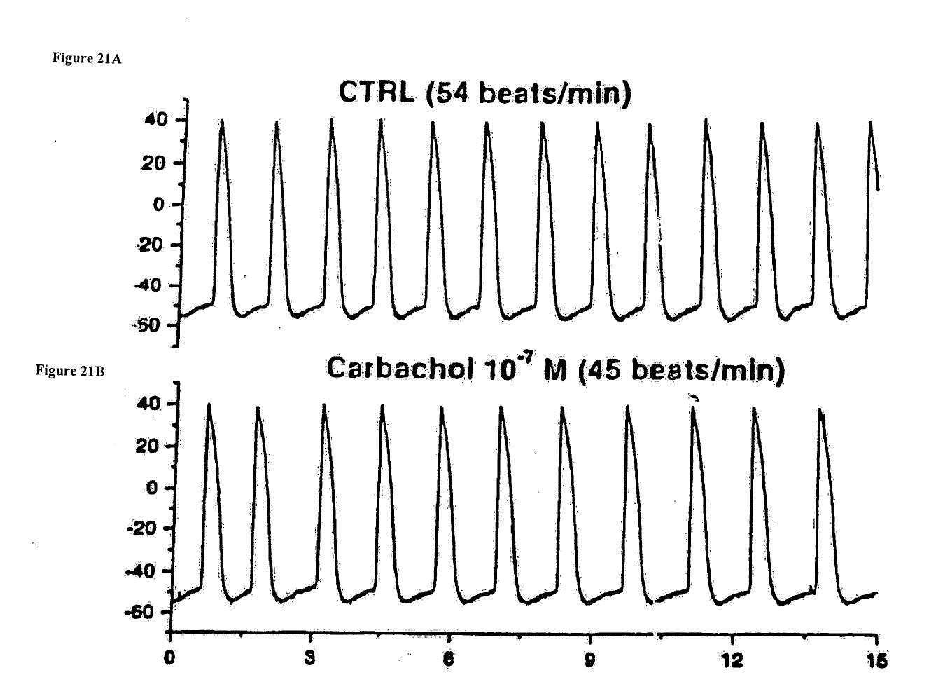

The normal cardiac pacemaker beats independently but is regulated by neurotransmitters released from sympathetic and parasympathetic neurons. The former release norepinephrine, which acts at beta-adrenergic receptors to increase cAMP concentration and increase heart rate. The latter release acetylcholine, which acts at muscarinic receptors to decrease cAMP concentration and decrease heart rate. FIG. 20 demonstrates that the beta-adrenergic agonist isoproterenol causes the predicted increase in heart rate in the HCN2 over-expressing cell culture. FIG. 21 demonstrates that the muscarinic agonist carbachol causes the predicted decrease in heart rate in the HCN2 over-expressing cell culture. FIG. 22 demonstrates that ZD-7288, a selective blocker of the pacemaker current that slows sinus rate, also slows the rate of the HCN2 over-expressing cell culture.

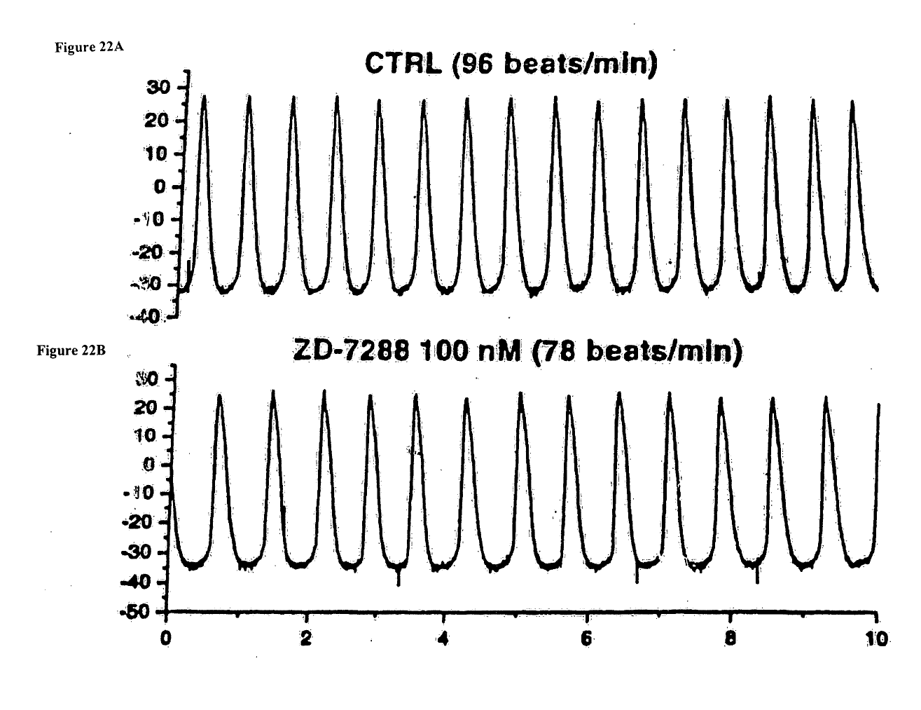

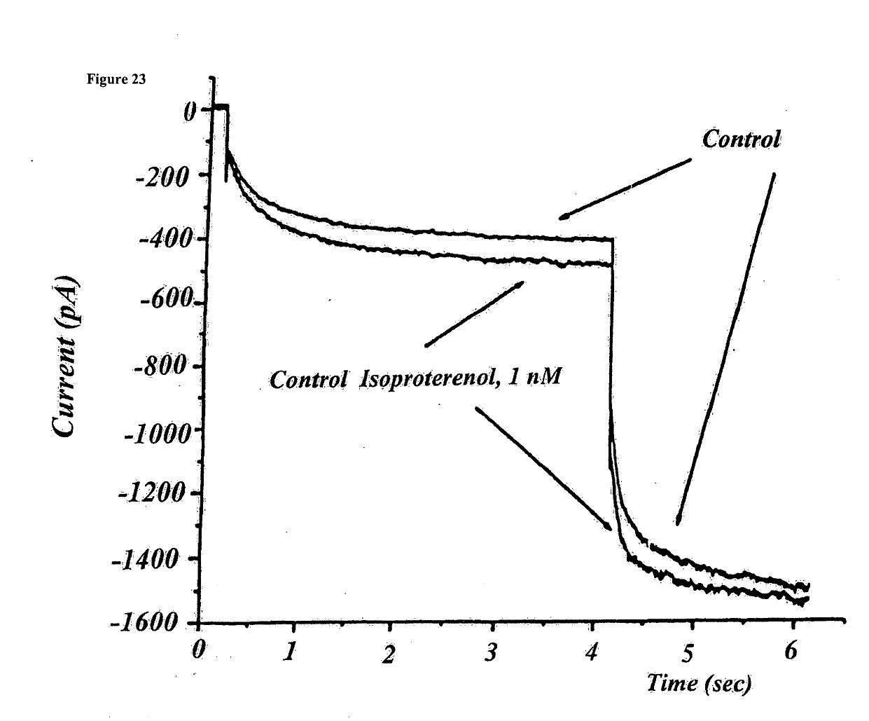

To further confirm that the over-expressed HCN2 channel responds similarly to the native pacemaker channel in sinus node and does not overwhelm the myocyte's natural signaling processes, the effect of a threshold concentration of isoproterenol on the over-expressed HCN2 in a neonatal ventricle myocyte was measured. In sinus node, the threshold concentration of isoproterenol on native pacemaker current was found to be approximately 1 nM (Zaza et al., 1996). The effect of isoproterenol is to shift the activation curve positive without increasing maximal current. This effect can be visualized by a two-step voltage protocol, with the first step to the midpoint of the activation curve and the second step to the maximum curve. FIG. 23 employs this two-step protocol to illustrate that this threshold concentration of isoproterenol shifts the activation curve of over-expressed HCN2 in a neonatal rat ventricle cell. The shift was approximately 5 mV, compatible with effects on native current.

Therefore, using adenoviral constructs to over-express pacemaker current alpha and beta subunits in neonatal rat ventricle cells results in cultures that beat spontaneously at a regular rate with a strong diastolic depolarization. Further, the rate of these modified cultures responds to drugs in a similar fashion as does the normal cardiac pacemaker in the sinus node. This provides a biologic basis for a high throughput rate assay that can be realized by growing the cells in an appropriate multiwell chamber and using calcium-sensitive or voltage-sensitive dyes to generate a convenient output signal to be detected by a fluorescence plate reader. Alternatively, the cell can be grown in a multiwell chamber that includes embedded recording electrodes and electrical activity measured directly as a read-out of the rate.

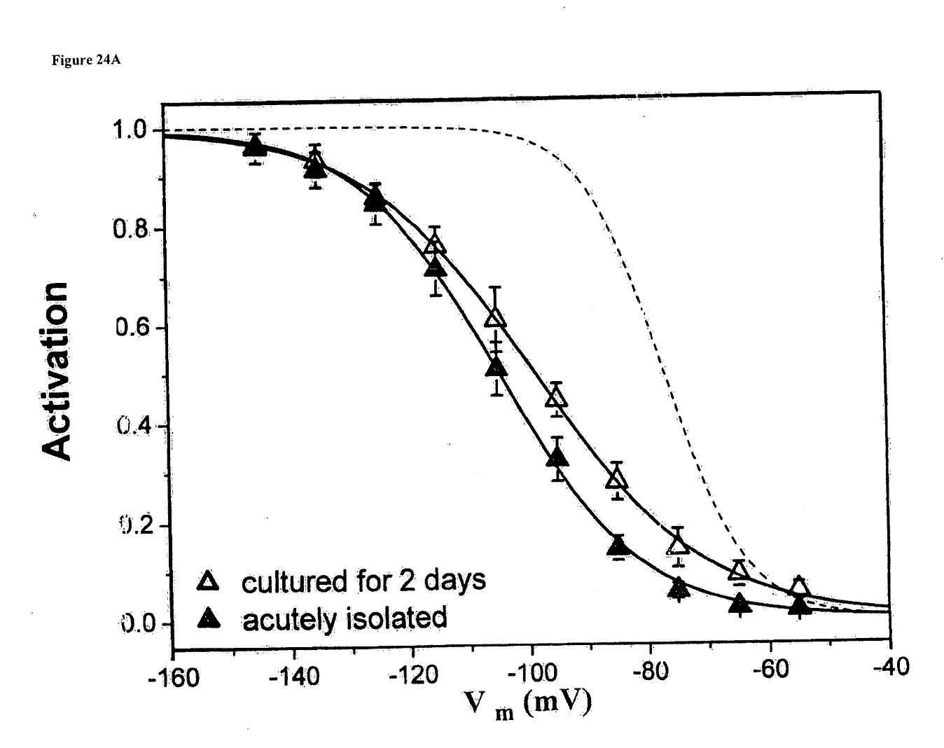

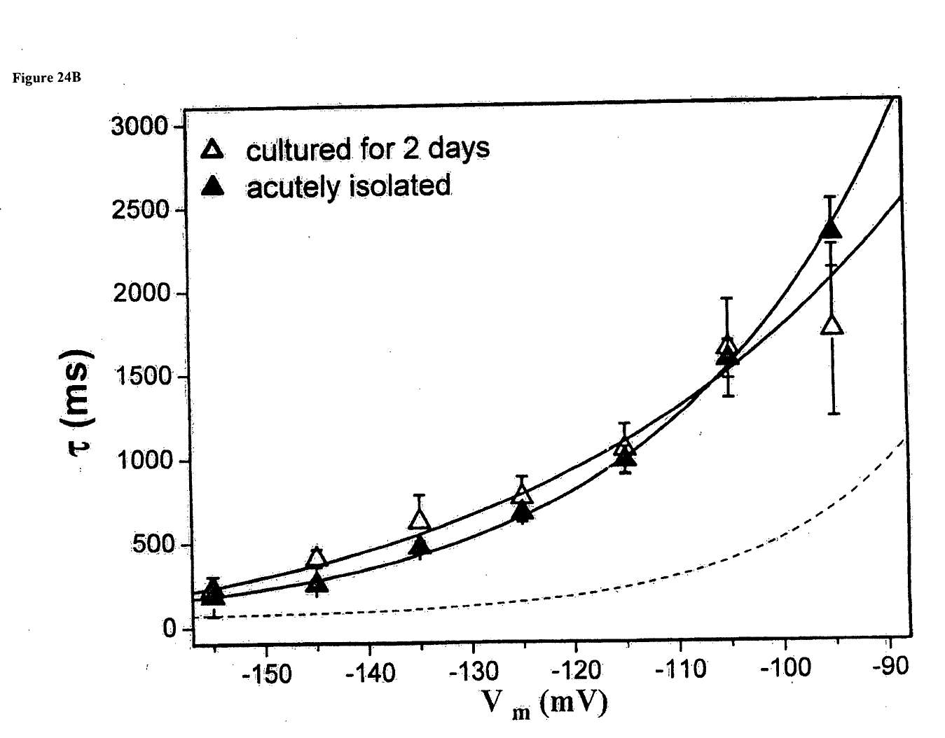

FIGS. 24A and B compares the activation relation and kinetics of native I_{f} in acutely dissociated and cultured adult ventricle cells. A 2-day culture period resulted in no significant difference in V_{1/2}, although the trend was toward a less negative midpoint after culture (−105.3±2.6 mV, n=12, vs. −98.7±1.8 mV, n=7, in acutely isolated vs. cultured cells; P=0.092), and there was also no significant difference in slope factor (10.9±1.2 vs. 14.4±1.9 mV). Activation kinetics also did not differ between acutely isolated and cultured adult ventricle. The neonatal data from FIG. 17 are superimposed (dashed lines) to illustrate the neonatal/adult difference in voltage dependence and kinetics of activation of the native I_{f}. Thus, short-term culture does not significantly alter I_{f}; any trend in voltage dependence is modest compared to the effect of development. The neonatal versus adult comparison confirms the earlier developmental study that reported an age-dependent difference in voltage dependence of activation (Robinson et al., 1997).

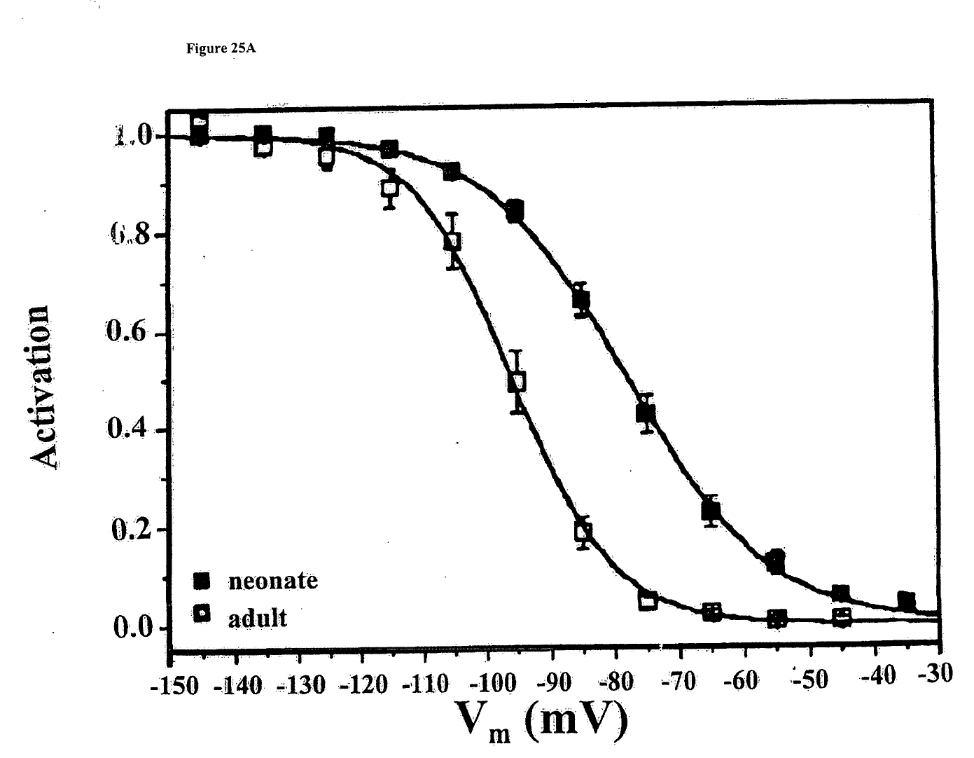

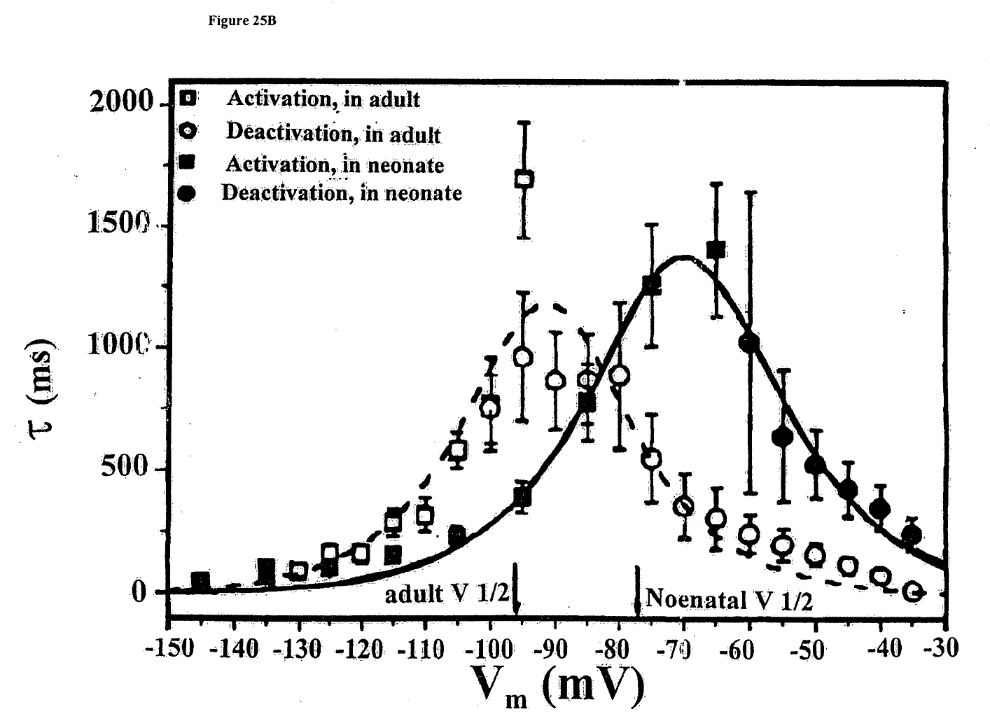

To compare the characteristics of HCN2 in neonatal and adult ventricular myocytes, the adenoviral construct was used with both preparations. The neonatal data are comparable to the earlier results using the lipofectin method (FIG. 17). FIG. 25A illustrates the average activation relations, from tail current measurements, obtained from myocytes expressing HCN2 in the two culture preparations. It is evident that, when the same protein is expressed in the neonatal and adult myocyte preparations, the resultant current activates at significantly more negative voltages in the adult cells. V_{1/2} values for HCN2 expressed in neonatal and adult myocytes were −77.6±1.6 mV (n=24) and −95.9±1.9 mV (n=13), respectively (P<0.001). In addition, the slope factor (K) also differed significantly (9.8±0.6 mV vs. 6.5±0.5 mV, P<0.001), reflecting a more shallow voltage dependence in the neonate. FIG. 25B provides data on the voltage dependence of activation/deactivation kinetics for the expressed HCN2. The data were well fit by a standard kinetic model, and exhibit little difference in the maximal value of activation time constant between the two cultures. However, the voltage dependence of the relation is shifted negative in the adult by an amount (21 mV) that is comparable to the shift in the activation relation (18 mV). Moreover, the relative peaks of the kinetic relations in the two culture preparations are consistent with the previously determined V_{1/2} values (arrows, FIG. 25B). Thus, the difference in the voltage dependence of activation kinetics of HCN2, when expressed in neonatal and adult myocytes, appears related to the voltage dependence of the steady-state activation relation.

Possible Basis for Difference Between Neonatal and Adult Myocytes Expressing HCN2

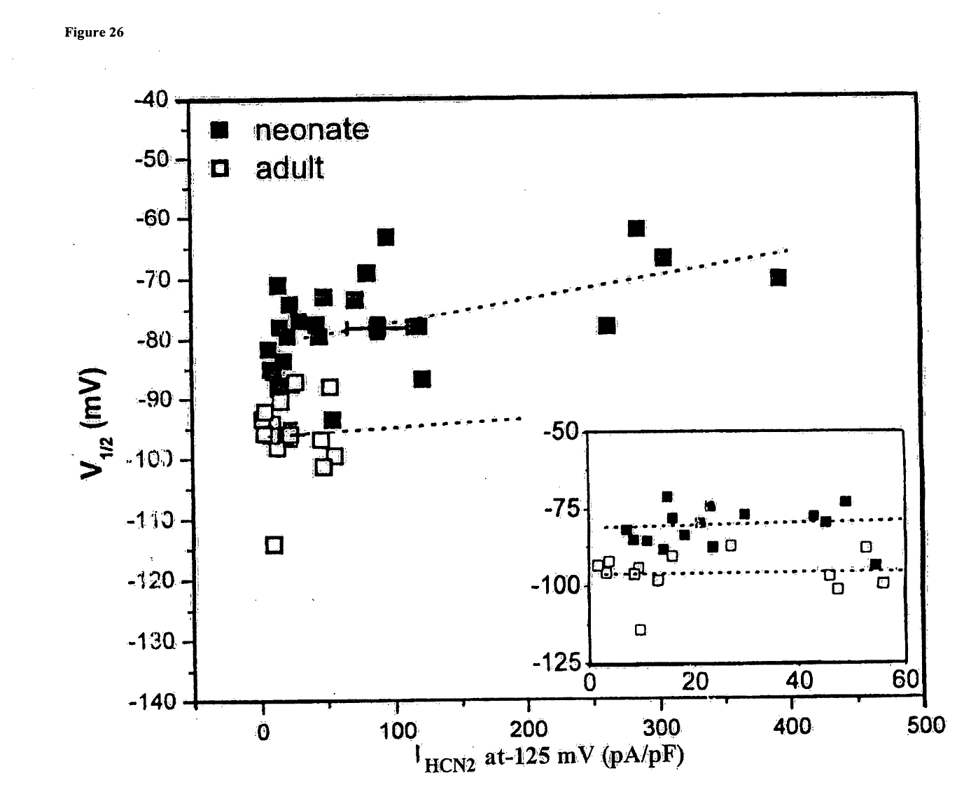

In heterologous expression of other currents, the biophysical characteristics of the expressed currents can sometimes depend on the current density achieved (Cui et al., 1994; Guillemare et al., 1992; Honore et al., 1992; Moran et al., 1992). To determine whether the difference in V_{1/2} of HCN2 between neonatal and adult was a result of this type of phenomenon, a linear regression analysis of the data was conducted (FIG. 26). The results indicate that, while there is some correlation of V_{1/2} with current density in the newborn, differences in expression level cannot explain the difference in HCN2 voltage dependence between neonatal and adult myocytes. The neonatal myocytes exhibited a wide range of current density for the expressed current, with a correlation coefficient for V_{1/2} of 0.51 (P=0.01); current density was less variable in the adult, with no correlation with activation midpoint (correlation coefficient 0.043, P=0.88). For current densities common to both preparations (i.e. <60 pA/pF, FIG. 26 inset) the expressed current in the neonatal myocytes demonstrated a significantly less negative V_{1/2} than in the adult myocytes (P<0.001).

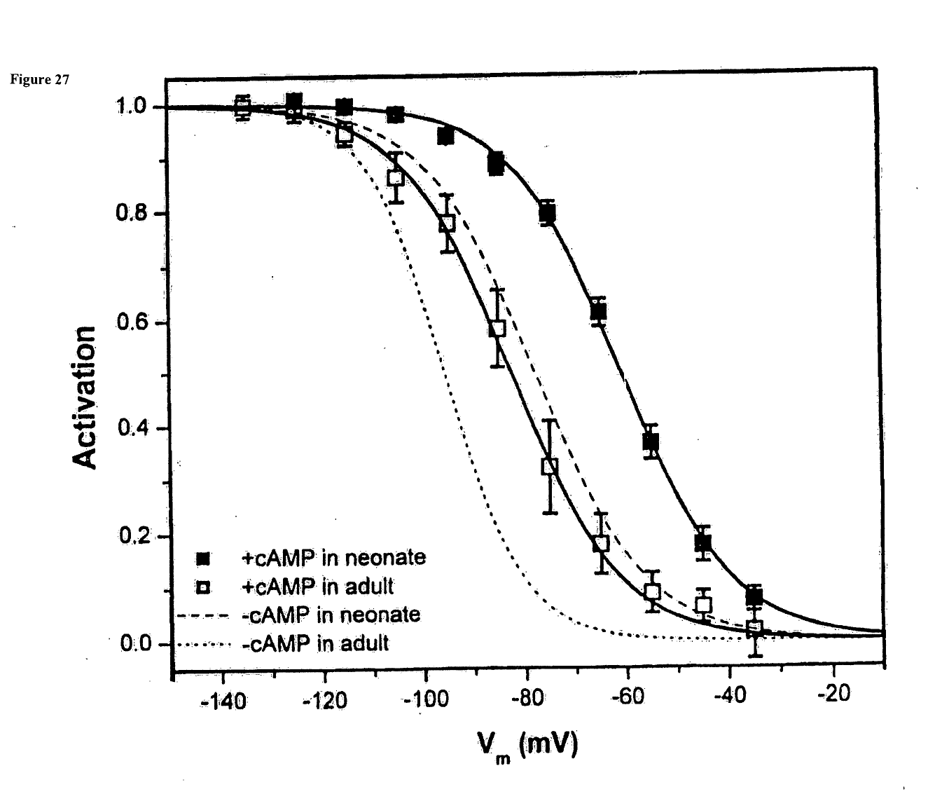

It is well known that both the native I_{f} and the expressed current respond to cAMP by a phosphorylation independent shift in the voltage dependence of activation (DiFrancesco et al., 1991; Kaupp et al., 2001), although phosphorylation-dependent mechanisms also have been reported (Chang et al., 2001; Yu et al., 1993; Accili et al., 1996). It is possible that the observed difference in activation of HCN2 in neonatal and adult myocytes simply reflected a different basal cAMP level within the two cells preparations. To test this, the experiments measuring the activation relation of the expressed current with AdHCN2 in neonatal and adult cells were repeated, but this time the experiments included 10 μM cAMP in the pipette solution to achieve a maximal positive shift of the current and eliminate any differences in intracellular cAMP levels. As seen in FIG. 27, the expressed current shifted positive by a comparable amount in both the neonatal and adult preparations (data in the absence of cAMP in the pipette are represented by the dashed and dotted lines), and the large difference in V_{1/2} values persisted. Thus, the age-dependent difference in the voltage dependence of activation of HCN2 does not arise from a difference in basal cAMP level between the two preparations.

Functional Effect of Overexpression of HCN2

The adenoviral construct of HCN2 resulted in expression of a large current in the majority of cells (at least 90% of cells patch clamped). Given the relatively positive activation of the expressed current in the neonatal cells, placing it within the physiologic range of voltages, it was next determined if overexpression of HCN2 resulted in a change in spontaneous rate of these cultures. These experiments were conducted using monolayer cultures of synchronously beating cells, with a whole cell patch electrode recording from one cell of the contiguous monolayer. The control (non-infected) cultures beat spontaneously, with a mean rate of 48.4±4.4 beats per min (bpm, n=17). There was little or no diastolic depolarization between action potentials and the cycle length tended to vary from beat to beat (FIG. 19A). The maximum diastolic potential (MDP) was −65.2−1.6 mV (n=17). In contrast, the cultures infected with AdHCN2 exhibited a more regular and faster rhythm (FIG. 19B), with mean rate of 88.0±5.4 bpm (n=16). Further, these cultures exhibited a marked diastolic depolarization and a less negative MDP (FIG. 19C). The differences in frequency, phase 4 slope, and MDP were statistically significant (P<0.05). Cultures infected with AdGFP (frequency: 45.8±4.7 bpm; MDP: ±59.5±2.4 mV; n=6) did not differ from uninfected control cultures, but did differ significantly from AdHCN2-infected cells.

The adult cultures did not beat spontaneously, either under control conditions or after infection with AdHCN2. This was not surprising, given the relatively negative activation relation of the expressed current in the adult cells. However, Ranjan et al. (1998) have proposed that native I_{f} in the adult mammalian ventricle contributes to anode break stimulation. If sufficiently strong, the hyperpolarizing stimulus activates I_{f}. The resultant inward tail current, combined with the voltage dependent block of I_{K1}, then causes the membrane potential to overshoot the resting potential in a depolarizing direction upon termination of the stimulus, leading to excitation. Therefore, the susceptibility to anode break excitation of control adult cultures and those infected with AdHCN2 was compared. Using a 20-ms stimulus, the maximal negative potential achieved for a threshold anodal stimulation was measured. Also measured was the I_{f} density at the end of a 2-s voltage step up to −125 mV in the same cells. FIG. 28 illustrates that the infected cells more readily exhibited anode break excitation. FIG. 28A illustrates representative control (left, with stimulus time course above) and infected (right) traces of anodal stimuli and resulting action potential upstrokes. The delay between the end of the anodal stimulus and the action potential threshold was not statistically different between control and infected cells (45±10 mV vs. 58±9 ms, P>0.05). FIG. 28B graphs the relation between maximal negative potential at threshold and I_{f} or I_{HCN2} density for control (unfilled symbol) and infected (filled symbol) cells. Control cells exhibited an inverse correlation between the maximal negative voltage required for anodal excitation and I_{f} density (FIG. 28B, inset), supporting the hypothesis that native I_{f} contributes to anode break excitation. In comparison, in infected cells it was sufficient to hyperpolarize the membrane to approximately −80 mV, i.e., the threshold for expressed HCN2 current. Anode break threshold was independent of expressed current density, indicating that the expressed current was large enough in all infected cells to generate a sufficient overshoot for achieving excitation at I_{HCN2} threshold. When required stimulus energy was calculated, as the integral of the area from start of the stimulus to threshold of the action potential, there was a significant difference between control and AdHCN2 infected cells (3140±279, n=10, vs. 2149±266 mV·ms, n=12; P<0.05). Neither the required stimulus energy nor the spontaneous rate of cells infected with AdGFP differed from those of control cells (data not shown), indicating that this was not simply an effect of the adenoviral infection. In addition, resting potential did not differ between control, AdHCN2- and AdGFP-infected myocytes (data not shown).

Factors Affecting Activation Voltage of HCN Channels in Neonatal and Adult Ventricles

This initial study investigated whether the distinct activation voltage of I_{f} in neonatal and adult ventricle was the result of a pronounced difference in the biophysical properties of the HCN2 and HCN4 isoforms when expressed in ventricular myocytes, or was due to an influence of the maturational state of the myocyte on an individual isoform, specifically HCN2. The results indicate that while HCN4, which is more prevalent in neonatal than adult ventricle, does activate at less negative voltages-than HCN2 when expressed in the neonatal ventricle, this isoform effect is modest. In comparison, when the HCN2 isoform is separately expressed in neonatal and adult ventricular myocytes, the midpoints of activation differ by 18 mV, compared to a difference of 22 mV in the midpoints of activation of the native I_{f} current in the neonate and adult ventricle in culture. Thus, the developmental difference in pacemaker current voltage dependence under these experimental conditions is largely accounted for by an effect of the myocyte maturational state on the HCN2 isoform rather than an HCN4/HCN2 isoform switch. Further, this difference in activation voltage results in a marked difference in the physiologic impact of expressed HCN2 current, due to the relative position of the current threshold with respect to the maximum diastolic potential as a function of age.

In investigating the question, the biophysical characteristics of mouse HCN2 and HCN4 expressed in neonatal rat ventricular myocytes, rather than in a heterologous mammalian expression system such as HEK293 cells, were first compared. As with prior heterologous expression studies, these data indicate that an inherent difference in the voltage dependence of HCN2 and HCN4, when expressed in myocytes, does not by itself account for the age-dependent difference in voltage dependence. At both ages, HCN2 is the dominant isoform based on RNase protection, although the relative ratio of HCN2/HCN4 message increases developmentally (Shi et al., 1999). The 9-mV negative shift of HCN2 activation, relative to HCN4 (−75 and −66 mV, respectively, using the lipofectin transfection method) in neonatal myocytes is far less than the developmental difference in native current activation. In addition, the kinetics of activation of the native I_{f} are faster in the neonate than adult, while the activation kinetics of HCN4 are slower than those of HCN2. Thus, a dominant contribution of HCN4 in the neonate, changing to a dominant contribution of HCN2 in the adult, is inadequate to explain the developmental difference in either activation voltage or activation kinetics.

It should be noted, however, that this does not preclude an isoform switch as a necessary or contributory component of the developmental change in voltage dependence. It could be that HCN4 would activate at markedly less negative voltages in adult as well as neonatal ventricle, i.e., that only HCN2 is sensitive to the maturational state of the myocyte. However, it seems unlikely given existing heterologous expression results concerning HCN4, which do not suggest that HCN4 is inherently positive. Admittedly, it is difficult to compare activation voltages between studies, since even with the same preparation considerable differences arise between laboratories as a result of variations in cell preparation and/or recording protocols. Still, it is interesting that HCN4 expression in the neonatal ventricle is much less negative than in any reported mammalian expression study. A midpoint of −66 mV was observed, whereas in other mammalian expression studies values ranging from −80 to −109 mV for this isoform have been reported (Ishii et al., 1999; Ludwig et al., 1999; Moosmang et al., 2001; Altomare et al., 2001). HCN2 in the neonatal ventricle also activates at less negative voltages than in other mammalian systems, with a midpoint of −78 mV (by tail measurement with adenoviral infection) in the present study, compared to values ranging from −83 to −97 mV (Ludwig et al., 1999; Moosmang et al., 2001; Altomare et al., 2001; Moroni et al., 2000). In those cases where activation voltage of HCN2 and HCN4 were measured in the same study, HCN2 activated ether slightly less negatively (Ludwig et al., 1999), equivalently (Moosmang et al., 2001) or slightly more negatively (Altomare et al., 2001) than HCN4. Thus, while the results largely agree with other studies that reported only a modest difference in activation voltage between HCN2 and HCN4, in general what was observed was less negative activation of both isoforms in the neonatal ventricle, compared to other mammalian expression systems. This suggests that perhaps the neonatal myocyte provides a unique environment, relative to alternative expression systems, allowing for less negative activation. However, at least one oocyte expression study (Santoro et al., 2000) reported HCN2 activation equivalent to that in the neonatal ventricle, with a midpoint of −78 mV, suggesting that other systems also are capable of expressing HCN2 with less negative voltage dependence (see also Example 1).

Whereas it is not clear whether it is the neonatal or the adult environment which is unique (or whether they are merely two distinct points on a continuum), it is clear that HCN2 exhibits markedly different voltage dependence when expressed in the two cell preparations, and that this parallels the developmental difference in native I_{f}. Under these experimental conditions, the midpoint of activation of native current in newborn and adult ventricle differed by approximately 22 mV, less than the previously reported difference in threshold value of approximately 40 mV (Robinson et al., 1997). A portion of the difference may result from the 48-h culture period, since acutely isolated adult myocytes had a midpoint value of activation that was 6 mV more negative. Although this was not a statistically significant difference, it is in keeping with an earlier study that found that extended culture under conditions that caused morphological dedifferentiation of adult myocytes resulted in a marked positive shift of activation voltage (Fares et al., 1998). In addition, the earlier developmental study specifically used adult epicardial myocytes (Robinson et al., 1997), while the present study used the whole ventricle of the adult heart to obtain a higher yield of viable cells for culture. A gradient of I_{f} activation, with epicardium more negative than endocardium, has been observed in the canine heart (Shi et al., 2000). If a similar gradient exists in adult rat ventricle, this also could contribute to the less negative adult values observed in the present study.

The actual midpoints of activation of native I_{f} in ventricle were −77 and −99 mV in neonate and adult, respectively, compared to values for HCN2 of −78 and −96 mV in these two preparations. Thus, HCN2 activation largely explains the voltage dependence of the native I_{f}. The difference in activation between neonatal and adult ventricle is not secondary to differences in cAMP levels, since saturating cAMP in the pipette shifts the voltage dependence of HCN2 by a comparable amount in the neonate and adult myocytes (17 and 14 mV, respectively). Beyond elimination of basal cAMP as a factor, the basis for the age-dependent difference in HCN2 voltage dependence when expressed in myocytes is unclear. The range of voltage dependence reported for I_{f} in different cardiac regions or as a function of age or disease is pronounced, and may reflect a combination of mechanisms. Studies of other channels have identified a number of factors that can alter the biophysical properties of native or expressed current, including beta subunits (Melman et al., 2001; Tinel et al., 2000), local membrane composition (Martens et al., 2000), cytoskeletal interactions (Chauhan VS, et al., 2000), phosphorylation/dephosphorylation (Chang et al., 1991; Yu et al., 1993; Walsh et al., 1991) and other post-translational modifications such as truncation (Gerhardstein et al., 2000). The extent to which any of these mechanisms contribute to the variation in voltage dependence of I_{f} or I_{HCN2} is unknown. In this context, it is interesting that when native I_{f} is studied in a cell free macro patch activation shifts markedly negative, but treatment of the intracellular face of the patch with pronase shifts activation back in the positive direction by 56 mV (Barbuti et al., 1999). In addition, when a large portion of the HCN2 C-terminal that includes the cyclic nucleotide binding domain is deleted, activation shifts positive by 24 mV (Wahler, 1992).

From these results one can speculate that interactions between cytoplasmic elements of the HCN protein contribute to more negative activation, but that these interactions are minimized in the intact cell, perhaps due to the presence of cytoskeletal elements, a beta subunit or other factors. The extent to which any of these factors actually contribute to the regional or developmental variation in activation voltage remains to be determined. However, if this reasoning is correct, then the factor(s) that contribute to the less negative activation of HCN2 in the neonate do not appear to be substrate limited, since no negative shift in activation voltage (and in fact a slight positive trend) at expression levels that were 2-3 orders of magnitude greater than that typical of native current in this preparation was observed.

The kinetic characteristics of the native current in neonate and adult ventricle also are largely explainable by HCN2, though perhaps not entirely. In the neonate, native current activates with kinetics that are intermediate between those of HCN2 and HCN4 expressed in these same cells. When the full activation/deactivation relation of expressed HCN2 is compared in neonate and adult, the difference is largely attributable to the difference in voltage dependence of activation. Thus, there does not appear to be an effect of maturational state of the myocyte directly on activation kinetics of expressed current, independent of the effect on voltage dependence of activation. However, native I_{f} kinetics in the adult appear slower than expressed HCN2 kinetics (compare FIGS. 24B and 25B).

Not surprisingly, expressing high levels of HCN2 in a neonatal culture results in a marked increase in spontaneous rate. This is accompanied by a less negative maximum diastolic potential and more pronounced phase 4 slope. The maximum diastolic potential in the HCN2 infected culture corresponds to the threshold voltage of the HCN2 current, indicating that even threshold levels of expressed current are sufficient to balance the contribution of I_{K1} (which is small in neonatal cultures). Expressing HCN2 in adult myocytes does not result in automaticity, either because of the more negative activation range in the adult cells or the greater I_{K1} density at this age. However, it does increase the susceptibility to anode break excitation. In HCN2 infected cultures of adult cells, the maximal negative voltage required during anodal stimulation in order to exhibit anode break excitation corresponds to the threshold voltage of the HCN2 current. Thus, the physiologic impact of overexpression of the HCN gene family in myocardium depends on the threshold voltage of the expressed current. This threshold voltage, and therefore the physiologic impact of HCN overexpression, to some extent depends on which isoform is expressed (i.e., HCN2 vs. HCN4 in neonate). However, this effect also is context dependent, with a distinct result depending on the maturational state of the target tissue. For the same reason, the effect is likely to depend on the cardiac region in which the channel is expressed and the disease state of the tissue, since native current is markedly affected by these factors. This has obvious implications for any future efforts to alter cardiac rhythm through the regional overexpression of selective HCN isoforms. It suggests rate can be enhanced by increasing current level, if the expressed current activates at an appropriate threshold voltage in the target tissue. As further insight into the mechanisms regulating the voltage dependence of this gene family is gained, it may be possible to control both the level of current and its activation voltage.

### EXAMPLE 3

### Effect of Molecular Composition of HCN Channels on Levels of Expression and Kinetics of the Channels

MiRP1: Beta Subunit of HCN Channel Enhances Expression and Speeds Kinetics

The HCN family of ion channel subunits has been identified as the molecular correlate of the currents I_{f} in heart, and Iₕ and I_{q} in neurons (Ludwig et al., 1998; Santoro et al., 1998; Santoro et al., 1999). However, a number of ion channels (including HCN channels) are heteromultimers of a large α-subunit and smaller β-subunits. The cardiac delayed rectifiers Iₖᵣ (Abbott et al., 1999) and I_{KS} (Sanguinetti et al., 1996) are examples of this basic principle. Their α-subunits derive from the ERG and KCNQ families respectively, but both also contain β subunits from a family of single transmembrane spanning proteins called minK and MiRPs (minK-related peptides).

MiRP1 enhances expression and speeds the kinetics of activation of the HCN family of channel subunits. RNase protection assays (RPAs) show that MiRP1 mRNA is prevalent in the primary cardiac pacemaking region, the sinoatrial node, and barely detectable in ventricle. Coimmunoprecipitation indicates that MiRP1 forms a complex with HCN1. Taken together, these results suggest that MiRP1 is a β subunit for the HCN family of ion channel protein subunits, and that it is likely to be an important regulator of cardiac pacemaker activity.

Heterologous Expression of HCN and MiRP1 Subunits in Xenopus Oocytes

cRNA encoding mouse HCN1 or HCN2, rat MiRP1 with or without an HA tag at the carboxy terminus, and rat minK were transcribed by using the mMessage mMachine kit (Ambion, Austin, Tex.). Xenopus laevis oocytes were isolated, injected with 2-5 ng (50-100 nl) of cRNA, and maintained in Barth medium at 18° C. for 1-2 days. For experiments using both HCN1 or HCN2 and MiRP1 or minK, the respective cRNAs were injected in 1:0.04-1 ratio.

Electrophysiologic studies on oocytes employed the two-microelectrode voltage clamp. The extracellular recording solution (OR2) contained: 80 mM NaCl, 2 mM KCI, 1 mM MgCl₂, and 5 mM Na-HEPES (pH 7.6). Group data are presented as means±SEM. Tests of statistical significance for midpoint and slope of activation curves were performed using unpaired Student's t-tests. P<0.05 is considered significant.

RNase Protection Assays

The procedures for the preparation of total RNA from rabbit heart tissues and the performance of the RNase protection assays was similar to those described previously (Dixon and McKinnon, 1994). Brain total RNA was obtained commercially from Clontech, and total RNA was isolated from left ventricle, right atrium and brain using SV Total RNA System (Promega). For each experiment 2 μg of total RNA was used. A cyclophilin probe was used in each experiment as an internal control over sample loss. RNA expression was quantified directly from dried RNase protection assay gels using a Storm phosphorimager (Molecular Dynamics), normalized to the cyclophilin signal in each lane. The MiRP1 signal consisted of two protected fragments in each rabbit tissue where MiRP1 was detected. The presence of two bands is likely the result of the dgenerate PCR primers, based on mouse and human sequences, used for the cloning of the RPA probes. The combined intensity of both bands was used in the quantification.

Protein Chemistry

For membrane protein preparation, all steps were performed on ice. 25 oocytes were washed with Ringer solution (96 mM NaCl, 1.8 mM CaCl₂, 5 mM Hepes (pH 7.4)) and lysed by vortexing with 1 ml of Lysis Buffer 1 (7.5 mM Na₂HPO₄ (pH 7.4), 1 mM EDTA) with protease inhibitors (aprotinin, leupeptine and pepstatin A, 5 μg/ml of each, and 1 mM PMSF). The lysate was centrifuged for 5 min at 150×g to remove yolk proteins and subsequently for 30 min at 14000×g. The membrane pellet was washed with Lysis Buffer 1 and resuspended in 1 ml of Lysis Buffer 2 (50 mM Tris-HCl (pH 7.5), 150 mM NaCl, 5 mM EDTA, 50 mM NaF, 50 mM Na pyrophosphate, 100 mM KH₂PO₄, 10 mM Na molybdate, 2 mM Na orthovanadate, 1% Triton® X-100, 0.5% NP40) with the same set of protease inhibitors as Lysis Buffer 1. Protein concentration of the membrane fractions was determined by the Lowry method.

For immunochemical analysis by western blotting, proteins associated with oocyte membrane fractions were separated on 10% SDS/PAGE (for HCN1) or on 16.5% Tricine-SDS/PAGE (for MiRP1 (Sclagger and von Jagow, 1987), and electroblotted to Hybond ECL™ nitrocellulose membranes (Amersham Pharmacia Biotech). Blocking and antibody incubations were done in PEST. The rabbit HCN1 antibodies (Quality Controlled Biochemicals) and the rat anti-HA high affinity antibodies (Roche Molecular Biochemicals) were used at 1:5000 and at 1:500 dilution, respectively. Secondary anti-rabbit (Kirkegaard Perry Laboratories, Maryland, USA) and anti-rat Ig-POD, Fab fragments (Boehringer Mannheim Biochemica) coupled to horseradish peroxidase were used at 1:10000 or 1:2000 dilution, respectively. The immnunoreactive protein bands were visualized using Lumi-Light^{PLUS} Western Blotting Substrate (Roche Molecular Biochemicals). The immunoprecipitation reactions were performed using 250 μg of membrane protein fractions and 10 μl of HCN1 antibodies cross-linked to protein A/G PLUS-Agarose (Santa Cruz Biotechnology, Inc.) with dimethylpimelimidate.

MiRP1 Enhances Expression and Conductance of HCN Channels Expressed in Oocytes

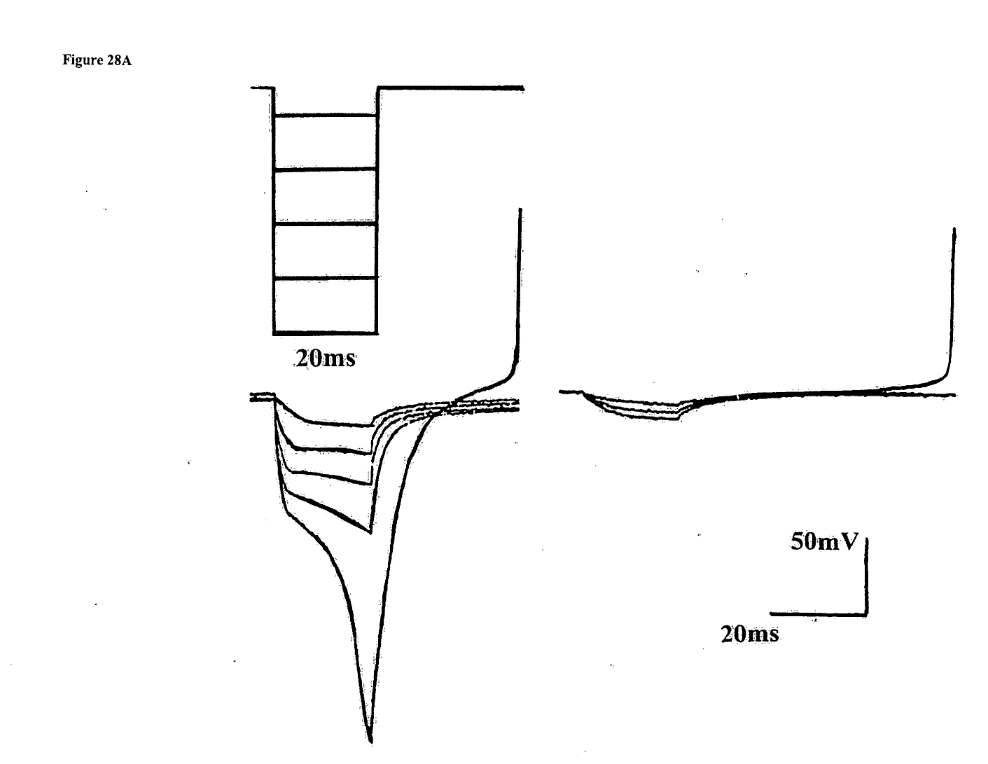

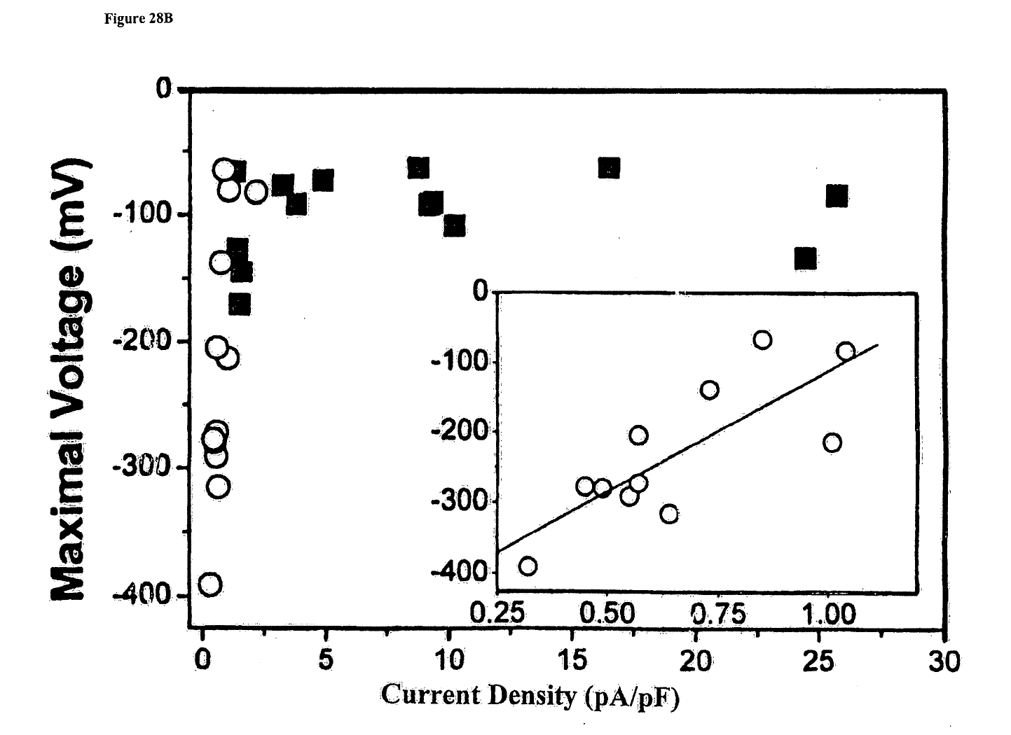

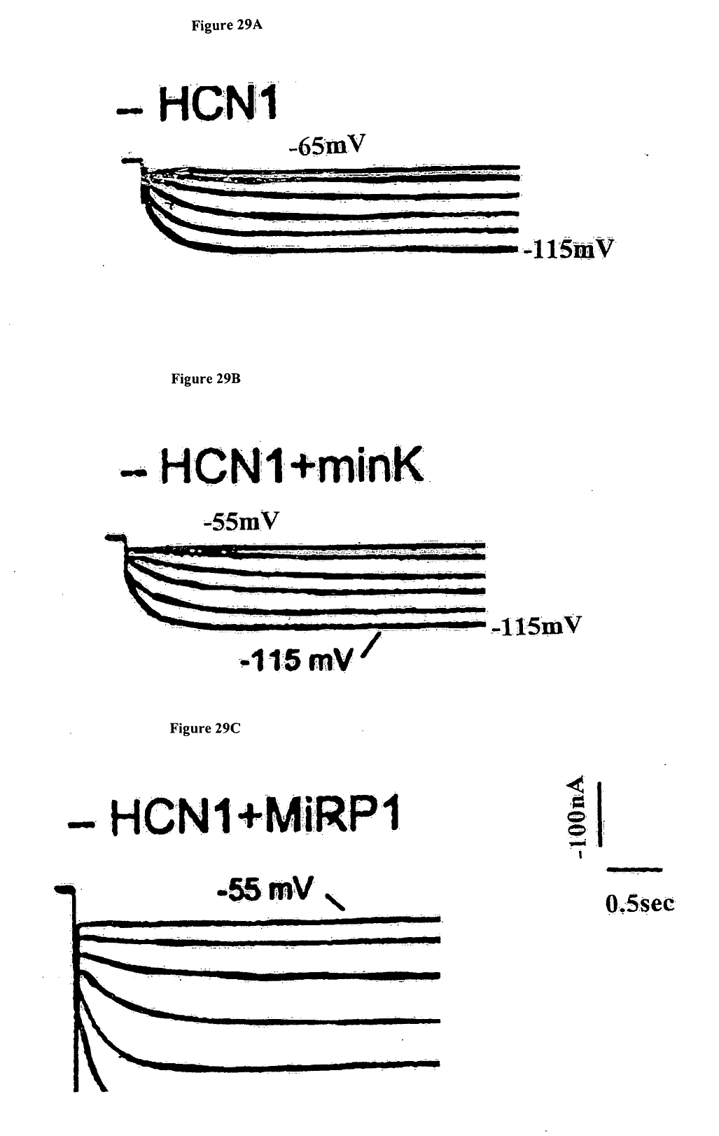

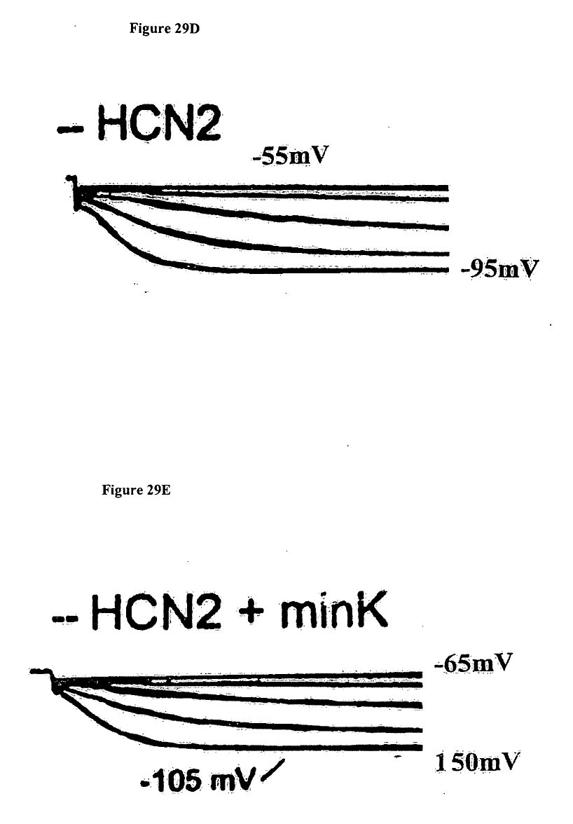

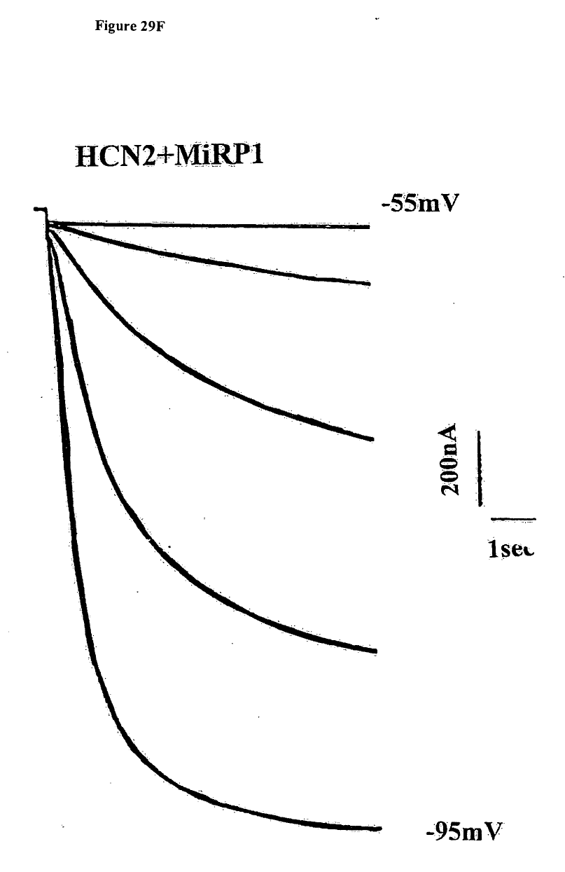

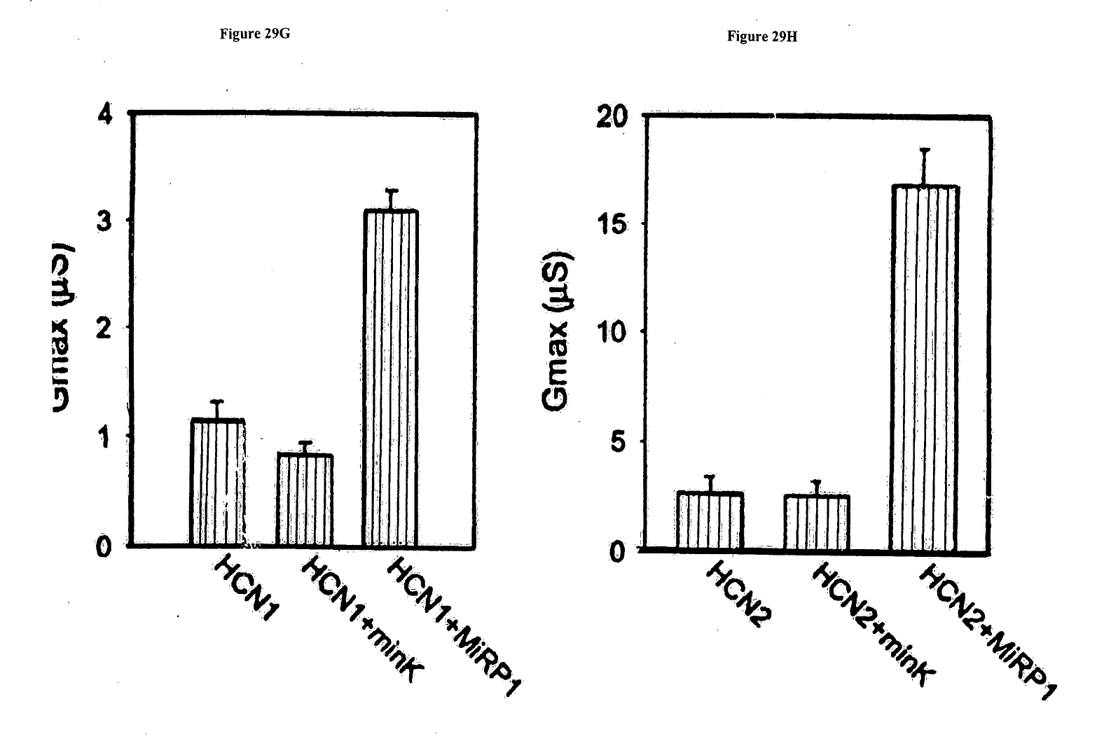

Xenopus oocytes were employed as a heterologous expression system and the expression of HCN1 and HCN2 individually and coexpressed with either minK (the minimal K channel protein, the first identified member of the single transmembrane spanning proteins family) or with MiRP1 was examined. The results are shown in FIG. 29. Both HCN1 (FIG. 29A) and HCN2 (FIG. 29D) express a small current when injected alone. Coexpression of either HCN1 (FIG. 29B) or HCN2 (FIG. 29E) with minK results in similar, low levels of current expression. However, a much larger current is observed when either HCN1 (FIG. 29C) or HCN2 (FIG. 29F) is coexpressed with MiRP1. Injection of MiRP1 by itself did not induce a current nor did injection with 100 nl of H₂O (not shown). The complete set of results for the expression studies of HCN1 and HCN2 with or without minK and MiRP1 are illustrated in FIGS. 29G and H. The maximal conductance is calculated by dividing the current onset at the most negative potential by the driving force (the reversal potential was measured in each oocyte). The results demonstrate an almost threefold enhancement of HCN1 conductance when HCN1 is coexpressed with MiRP1, whereas MiRP1 enhances expression of HCN2 by more than fivefold. Coexpression of either HCN1 or HCN2 with minK does not enhance HCN1 or HCN2 expression. Thus the enhancement of expression is specific for MiRP1.

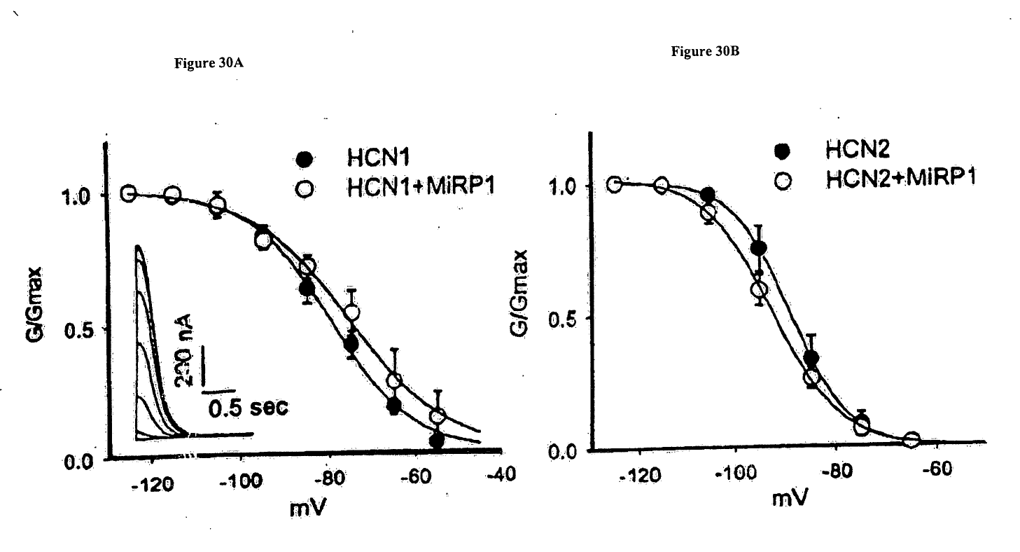

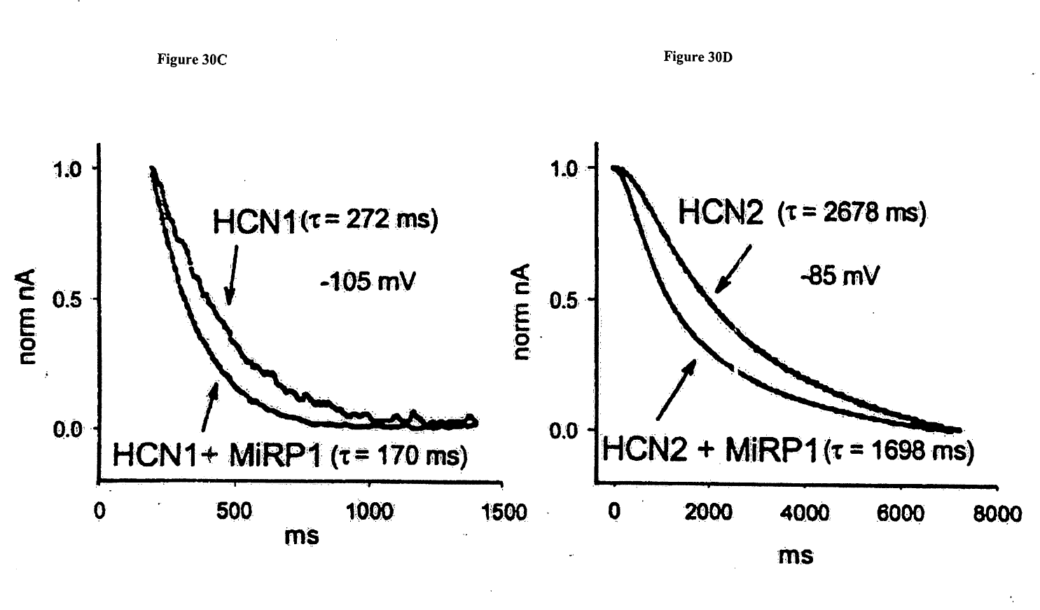

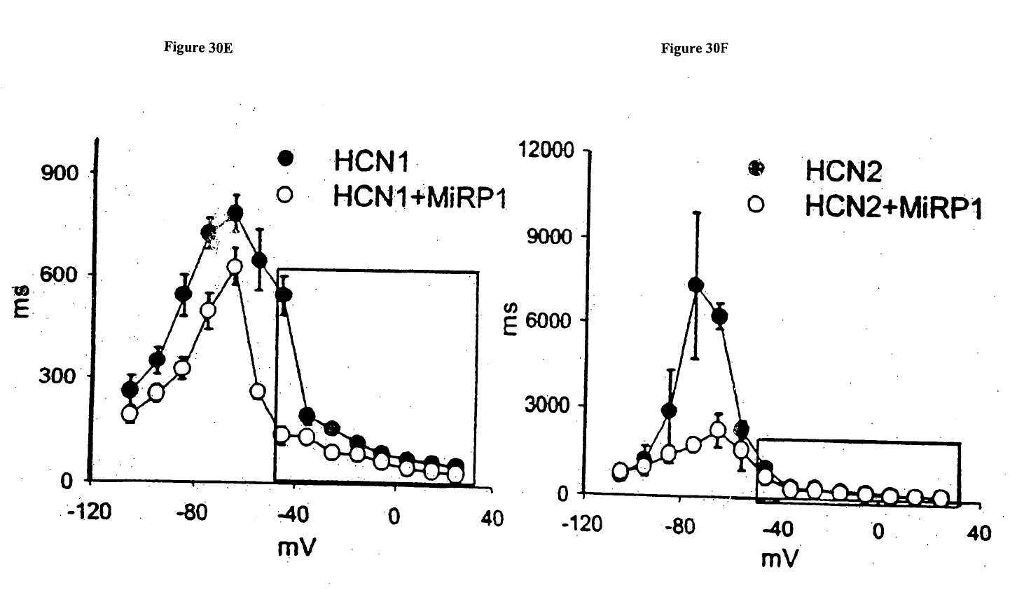

FIG. 30 shows the gating properties of MiRP1 coexpressed with either HCN1 or HCN2. Isochronal activation curves were constructed from tail currents recorded at −10 mV in response to 3-(for HCN1) or 8-s long (for HCN2) hyperpolarizing test pulses. The results demonstrate no significant difference in midpoint but statistically indicate a shallower slope for the activation of HCN channels coexpressed with MiRP1 (FIGS. 30A and B, see brief description of the figures for details).

FIGS. 30C-F show the kinetics of activation and deactivation. Raw data are shown for activation of both HCN1 (FIG. 30C) and HCN2 (FIG. 30D). As shown in the figures, MiRP1 decreases the time constant of activation. The average of all the results on activation and deactivation (indicated by the encircled box), provided in FIGS. 30E and F, indicates that coexpression with MiRP1 accelerates both processes.

The rectification properties of HCN1 or HCN2 expressed with or without MiRP1 were also studied. Coexpression of either HCN1 or HCN2 with MiRP1 did not alter the linearity of the fully activated current-voltage relationship (not shown).

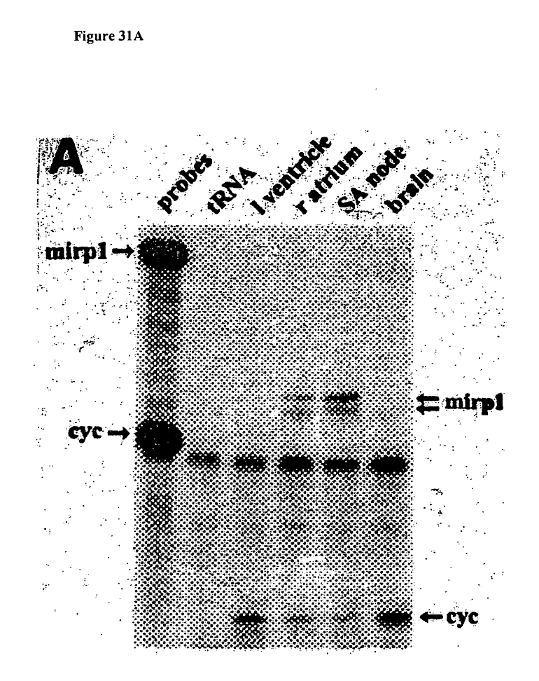

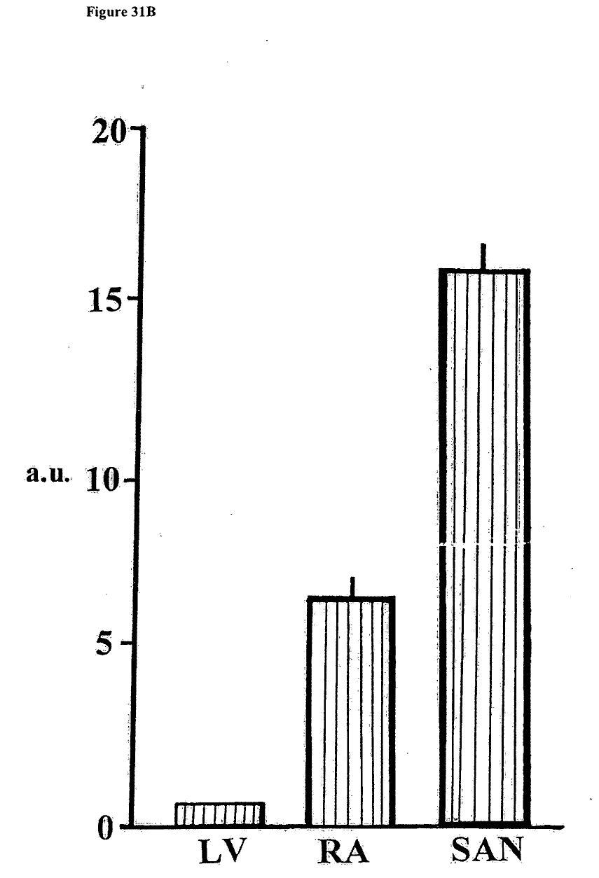

Previous studies examining the potential role of MiRP1 in generating Iₖᵣ employed Northern Blot analysis to demonstrate the presence of MiRP1 mRNA in whole rat heart (Abbott et al., 1999). If MiRP1 also regulates I_{f} current expression in vivo, mRNA for MiRP1 should be prominent in regions where I_{f} currents are large. RNase protection assays was employed to quantify the distribution of MiRP1 transcripts in SA node, right atrium and ventricle of the rabbit heart. As shown in FIG. 31, MiRP1 transcript levels are highest in the SA node, atrial levels are about 40% of those in SA node, while ventricular levels are barely detectable (<4% of level in SA node).

These data suggest that a complex probably exists between members of the HCN family and MiRP1, and indicates that MiRP1 may be a β-subunit for the HCN family. This hypothesis was further investigated using HCN1 for which antibodies are available.

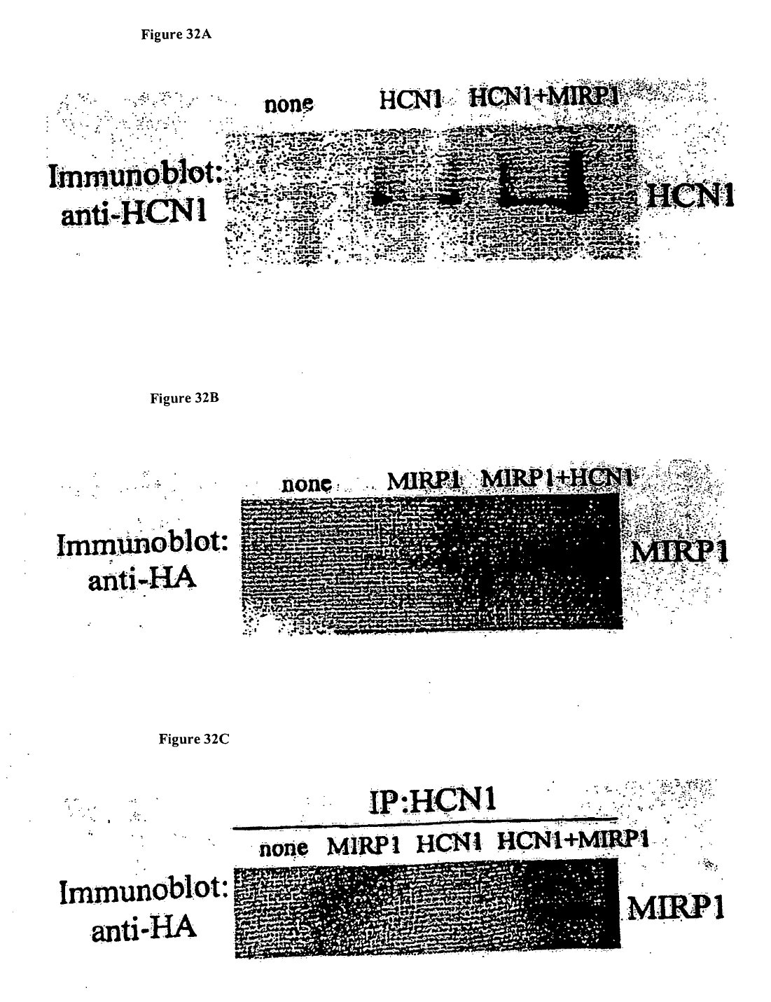

The HCN1 antibody recognizes a single polypeptide with an apparent molecular mass of 145 kDa (possibly glycosylated (Hansen et al., 1995). MiRP1, HA epitope-tagged at the carboxy-terminal end, was recognized by anti-HA high affinity antibodies as a 13.5 kDa band. Both proteins were localized in the membrane fraction, and protein expression was enhanced (about 2-fold) when they were co-expressed together (FIGS. 32A and B).

To test whether a complex of HCN1 and MiRP1 might exist in a heterologous expression system, coimmunoprecipitation experiments were performed using membrane fractions of oocytes injected with cRNAs encoding HCN1 alone, MiRP1 alone, or with both cRNAs. FIG. 32C shows the immunoprecipitation products tested by western blot analysis. The presence of MiRP1 in the anti-HCN1 immunoprecipitate only for oocytes injected by both HCN1 and MiRP1 cRNAs, together with its absence in oocytes injected by either one of these mRNAs, indicates that MiRP1 was pulled down by the anti-HCN1 antibody most likely because it was complexed with HCN1.

The results demonstrate that the proteins are colocalized as a complex in the membrane and enhance each other's expression. This strongly indicates that MiRP1 is a β-subunit for the HCN family of ion channels.

MiRP1 is a member of a family of single transmembrane spanning proteins that have been demonstrated to alter expression and serve as a , subunit of both KCNQ (minK) and ERG (MiRP1) family members (Abbott et al., 1999; Dixon and McKinnon, 1994). In these previous studies, as in this study, the minK family member altered gating and was demonstrated to be a beta subunit by co-immunoprecipitation.

In the present study, it has been shown that minK does not affect the properties of HCN1 and HCN2 channels expressed in Xenopus oocytes. MiRP1, on the other hand, dramatically enhances the current expression of both HCN subunits and hastens the kinetics of current activation and deactivation. A speeding up of deactivation kinetics is also seen when MiRP1 associates with HERG to form Iₖᵣ (Abbott et al., 1999). The data presented herein also show that MiRP1 and HCN1 probably form a complex in the membrane.

Pacemaker activity in the rabbit sinus node is generated by a net inward current of only a few pA (Vasalle et al., 2000). This net inward current is due to the balance of inward and outward currents more than an order of magnitude larger. Although the biophysical properties of each of the component currents is known, how this fine balance is achieved remains unknown. The results presented here show that a single beta subunit may control the expression of two important pacemaker currents, the outward Iₖᵣ, and the inward I_{f}. If this is the case, it is possible that MiRP1 serves as an important regulator of cardiac pacemaker rate.

### EXAMPLE 4

### Induction of Pacemaker Activity by Overexpression of HCN Channels in Heart In Situ

HCN2 Induces Pacemaker Current in Heart in situ

It was hypothesized that overexpression of I_{f} in either secondary pacemaker tissues of the cardiac specialized conducting system or in non-pacemaker cells of the myocardium could provide a nidus of pacemaker activity to drive the heart in a “demand” mode in the absence of dominant pacemaker function of the sinus node or failure of impulse propagation via the atrioventricular node. Attention was focused on HCN2 because its kinetics are more favorable than those of HCN4 and its cAMP responsiveness is greater than that of HCN1. Initial experiments were performed in neonatal rat myocytes in culture. These experiments indicated that not only could an overexpressed pacemaker current increase beating rate, but that mutations in the HCN2 pacemaker gene and/or the addition of appropriate accessory channel subunits could modify the characteristics of the expressed current in a manner that might be expected to further enhance the beating rate (U.S. Pat. No. 6,849,611; Qu et al., 2001; Qu et al., 2004; Chen et al., 2001b; Plotnikov et al., 2005a). These neonatal ventricular myocytes manifest a small endogenous pacemaker current and, when infected with an adenovirus carrying HCN2, express a markedly larger pacemaker current. When the spontaneous beating rate of monolayer cultures infected with an Ad expressing HCN2 and the green fluorescent protein (GFP) was compared with a virus incorporating GFP as a control and marker, the HCN2/GFP-expressing cultures beat significantly faster (Qu et al., 2001).

Based on the encouraging results and implications of the cell culture work, proof of concept was tested by injecting a small quantity of HCN2 and GFP genes in an adenoviral vector into canine left atrium (Qu et al., 2003). After recovery of the animals, the right vagus nerve was stimulated to induce sinoatrial slowing and/or block. In this setting, pacemaker activity originated in the left atrium and was pace-mapped to the site of adenoviral injection. Increasing the intensity of the vagal stimulation and adding left vagal stimulation as well caused cessation of biological pacemaker activity, implying parasympathetic responsiveness. The atrial myocytes were disaggregated from the site of injection, and overexpressed pacemaker current was demonstrated. In sum, the results indicate that such overexpressed pacemaker current could provide escape beats under circumstances of sinus slowing (Qu et al., 2003).

The next steps involved catheter injection of the same adenoviral HCN2/GFP construct into the canine proximal LV conducting system, under fluoroscopic control (Plotnikov et al., 2004). Animals so injected demonstrated idioventricular rhythms having rates of 50-60 bpm when sinus rhythm was suppressed by vagal stimulation. For the HCN2 group, the rhythms mapped to the site of injection. When bundle branch tissues were removed from the heart and studied with microelectrodes, automaticity in those injected with HCN2 was found to exceed that in control preparations, i.e., there was a significantly greater spontaneous rate generated by the HCN2 injected bundle branches than by those injected with either saline or virus carrying GFP alone (Plotnikov et al., 2004).

Biophysical Properties of Ion Currents as Predictors of Biological Pacemaker Function

The studies in neonatal rat myocytes (FIGS. 10 and 11) and in Xenopus oocytes (FIGS. 121-15) gave concordant results with regard to the function of mHCN2 and mE324A. That the mE324A mutation induced faster, more positive pacemaker current activation in these in vitro settings than did mHCN2 might be interpreted as suggesting the mutant channel would result in a faster pacemaker rate and/or a shorter escape interval after overdrive pacing than occurred in saline-injected or mHCN2 injected hearts. However, in situ both the saline- and mHCN2-injected hearts showed escape times equivalent to the mE324A-injected hearts. As for automatic rates per se, these were equivalent for mHCN2- and mE324A-injected hearts, and both were significantly faster than those injected with saline. In other words, for two important descriptors, rate attained and overdrive suppression, there was no clear discrimination between the effects of mHCN2 and mE324A in situ.

One explanation for this may be that the percent of myocytes expressing mE324A current was significantly less than that expressing mHCN2. Moreover there was a lesser current density in the E324A group. Thus, while a greater fraction of channels activate faster at a given voltage with mE324A compared to mHCN2, the total number of channels available or net current may be approximately equivalent at physiologically relevant voltages such as −55 mV (see insets in FIG. 10).

The extent to which biophysical results were predictive of those in situ is seen in the following: the biophysical data indicating that mE324A density is less than that of mHCN2, and that mE324A activation is positive to and faster than that of mHCN2, would suggest that for pacemaker rate there may be no advantage to either construct. The finding that the mE324A cAMP response is positive to that of mHCN2 would suggest that the magnitude or sensitivity of the mE324A response to epinephrine in situ might be greater than that for mHCN2. In fact, the studies in situ showed no rate advantage to either construct with a greater response to epinephrine of the mE324A mutant. Not only does this show concordance between biophysical finding and clinical implication, but it leads to the following hypotheses: first, as long as there is sufficient current density, a positive position of the activation curve and/or faster kinetics are more important than absolute current density in biological pacemaker functionality; and second, adrenergic responsiveness depends on the final position of the activation curve in the presence of cAMP more than the magnitude of the voltage shift.

### EXAMPLE 5

### Cell Therapy with Human Mesenchymal Stem Cells

Cell Cultures

Human mesenchymal stem cells (hMSCs; mesenchymal stem cells, human bone marrow; Poietics™) were purchased from Clonetics/BioWhittaker (Walkersville, Md., USA), cultured in mesenchymal stem cell (MCS) growth medium and used from passages 2-4. Isolated and purified hMSCs can be cultured for many passages (12) without losing their unique properties, i.e., normal karyotype and telomerase activity (van den Bos et al., 1997; Pittenger et al., 1999).

HeLa cells transfected with rat Cx40, rat Cx43 or mouse Cx45 were cocultured with hMSCs. Production, characterization and culture conditions of transfected HeLa cells have been previously described (Elfgang et al., 1995; Valiunas et al., 2000; 2002).

Anti-connexin Antibodies, Immunofluorescent Labeling, and Immunoblot Analysis

Commercially available mouse anticonnexin monoclonal and polyclonal antibodies (Chemicon International, Temecula, Calif.) of Cx40, Cx43 and Cx45 were used for immunostaining and immunoblots as described earlier (Laing and Beyer, 1995). Fluorescein-conjugated goat antimouse or antirabbit IgG (ICN Biomedicals, Inc., Costa Mesa, Calif.) was used as secondary antibody.

Electrophysiological Measurements across Gap Junctions

Glass coverslips with adherent cells were transferred to an experimental chamber perfused at room temperature (−22° C.) with bath solution containing (mM): NaCl, 150; KCl, 10; CaCl₂, 2; Hepes, 5 (pH 7.4); glucose, 5. The patch pipettes were filled with solution containing (mM): potassium aspartate, 120; NaCl, 10; MGATP, 3; Hepes, 5 (pH 7.2); EGTA, 10 (pCa ˜8); filtered through 0.22 μm pores. When filled, the resistance of the pipettes measured 1-2 MΩ. Experiments were carried out on cell pairs using a double voltage-clamp. This method permitted control of the membrane potential (Vₘ) and measurement of the associated junctional currents (Iⱼ).

Dye Flux Studies

Dye transfer through gap junction channels was investigated using cell pairs. Lucifer Yellow (LY; Molecular Probes) was dissolved in the pipette solution to reach a concentration of 2 mM. Fluorescent dye cell-to-cell spread was imaged using a 16 bit 64000 pixel grey scale digital CCD-camera (LYNXX 2000T, SpectraSource Instruments, Westlake Village, Calif.) (Valiunas et al., 2002). In experiments with heterologous pairs, LY was always injected into the cells which were tagged with Cell Tracker Green. The injected cell fluorescence intensity derived from LY is 10-1 5 times higher than the initial fluorescence from Cell Tracker Green.

Human MSCs Express Connexins

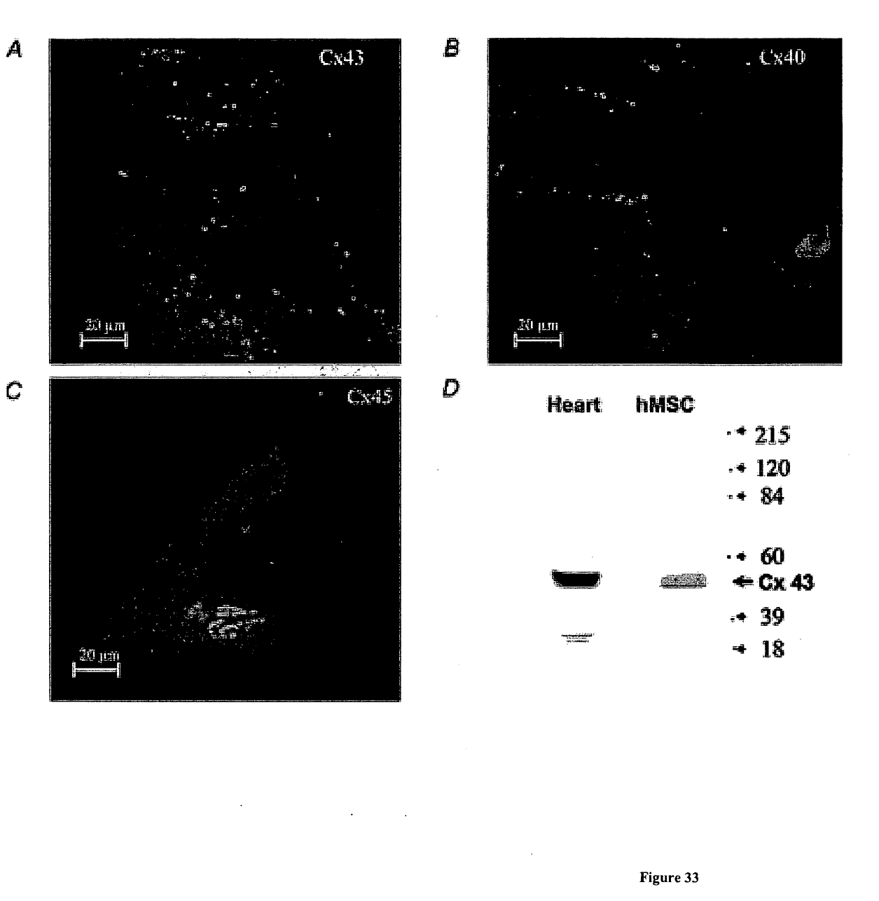

The connexins, Cx43 and Cx40, were immunolocalized, as evidenced by typical punctate staining, along regions of intimate cell-to-cell contact and within regions of the cytoplasm of the hMSCs grown in culture as monolayers (FIGS. 33A, B). Cx45 staining was also detected, but unlike that of Cx43 or Cx40, was not typical of connexin distribution in cells. Rather, it was characterized by fine granular cytoplasmic and reticular-like staining with no readily observed membrane-associated plaques (FIG. 33C). This does not exclude the possibility that Cx45 channels exist but does imply that their number relative to Cx43 and Cx40 homotypic, heterotypic and heteromeric channels is low. FIG. 33D illustrates Western blot analysis for canine ventricle myocytes and hMSCs with a Cx43 polyclonal antibody which adds further proof of Cx43 presence in hMSCs.

Gap Junctional Coupling between hMSCs and Various Cell Lines

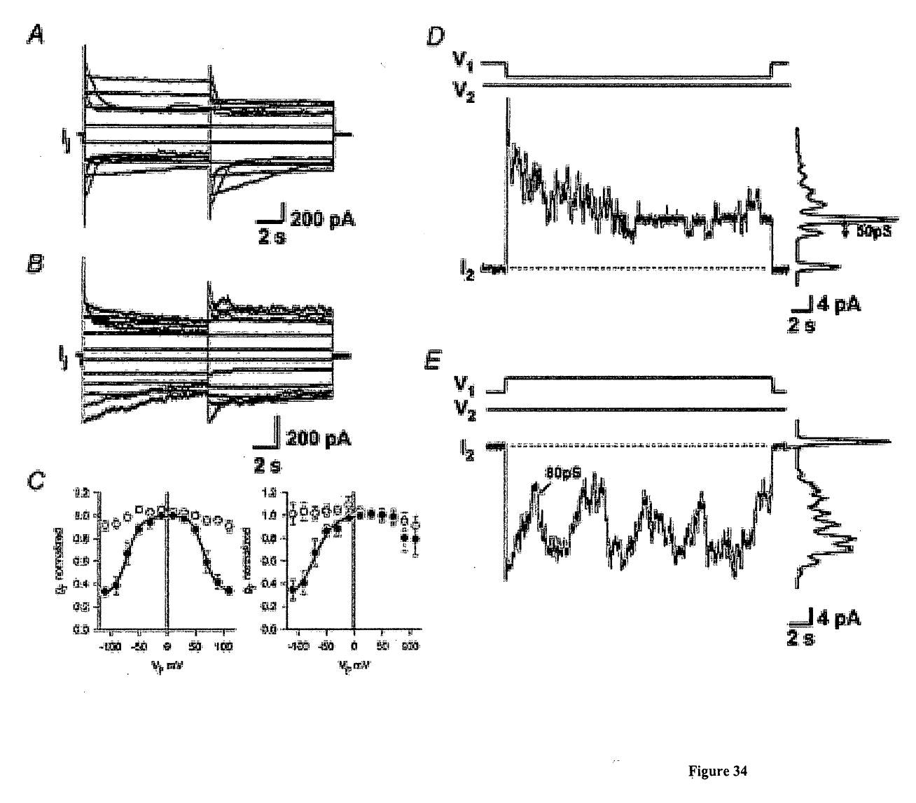

Gap junctional coupling among hMSCs is demonstrated in FIG. 34. Junctional currents recorded between hMSC pairs show quasi-symmetrical (FIG. 34A) and asymmetrical (FIG. 34B) voltage dependency arising in response to symmetrical 10-s transjunctional voltage steps (Vⱼ) of equal amplitude but opposite sign starting from ±10 mV to ±110 mV using increments of 20 mV. These behaviors are typically observed in cells which co-express Cx43 and Cx40 (Valiunas et al., 2001).

FIG. 34C summarizes the data obtained from hMSC pairs. The values of normalized instantaneous (g_{j,inst}, ∘) and steady state conductances (g_{j,ss}, ●) (determined at the beginning and at the end of each Vⱼ step, respectively) were plotted versus Vⱼ. The left panel shows a quasi-symmetrical relationship from five hMSC pairs. The continuous curves represent the best fit of data to the Boltzmann equation with the following parameters: half-deactivation voltage, V_{j,0}=−70/65 mV; minimum gⱼ, g_{j,min}=0.29/0.34; maximum gⱼ, g_{j,max}=0.99/1.00; gating charge, z=2.2/2.3 for negative/positive Vⱼ, respectively. Summarized plots from six asymmetrical cases are shown in the right panel. The g_{j,ss} declined in sigmoidal fashion at negative Vⱼ and showed a reduced voltage sensitivity to positive Vⱼ. Boltzman fitting for negative Vⱼ revealed the following values:
V_{j,0}=−72 mV, g_{i,min}=0.25, g_{j,max}=0.99, z=1.5.

FIGS. 34D and E illustrate typical multichannel recordings from a hMSC pair. Using 120 mM K aspartate as a pipette solution, channels were observed with unitary conductances of 28-80 pS range. Operation of channels with ˜50 pS conductance (see FIG. 29D) is consistent with previously published values (Valiunas et al., 1997; 2002) for Cx43 homotypic channels. This does not preclude the presence of other channel types, it merely suggests that Cx43 forms functional channels in hMSCs.

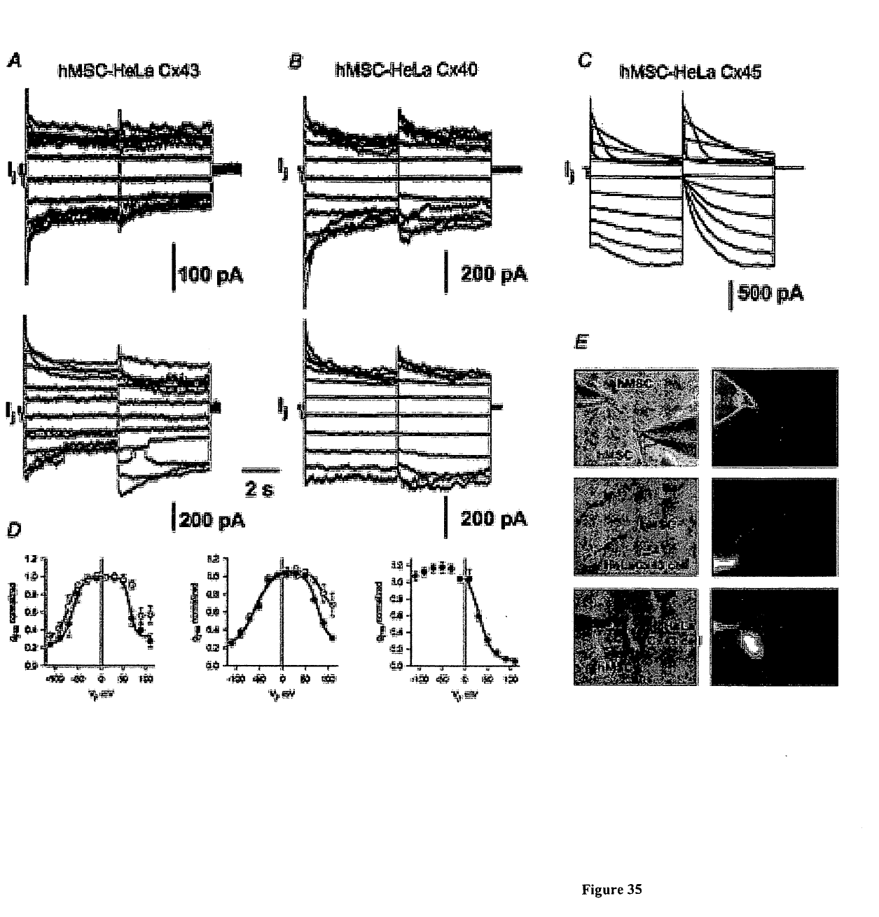

To further define the nature of the coupling, hMSCs were co-cultured with human HeLa cells stably transfected with Cx43, Cx40, and Cx45 (Elfgang et al., 1995) and it was found that hMSCs were able to couple to all these transfectants. FIG. 35A shows an example of junctional currents recorded between an hMSC and HeLaCx43 cell pairs that manifested symmetrically and asymmetrically voltage dependent currents in response to a series (from ±10 mV to ±110 mV) of symmetrical transjunctional voltage steps (Vⱼ). The quasi-symmetric record suggests that the dominant functional channel is homotypic Cx43 while the asymmetric record suggests the activity of another connexin in the hMSC (presumably Cx40 as shown by immunohistochemistry; see FIG. 33) that could be either a heterotypic or heteromeric form or both. These records are similar to those published for transfected cells: heterotypic and mixed (heteromeric) forms of Cx40 and Cx43 (Valiunas et al., 2000; 2001). Co-culture of hMSCs with HeLa cells transfected with Cx40 (FIG. 35B) also revealed symmetric and asymmetric voltage dependent junctional currents consistent with the co-expression of Cx43 and Cx40 in the hMSCs similar to the data for Cx43 HeLa-hMSC pairs. HeLa cells transfected with Cx45 coupled to hMSCs always produced asymmetric junctional currents with pronounced voltage gating when Cx45 (HeLa) side was negative (FIG. 35C). This is consistent with the dominant channel forms in the hMSC being Cx43 and Cx40 as both produce asymmetric currents when they form heterotypic channels with Cx45 (Valiunas et al., 2000; 2001). This does not exclude Cx45 as a functioning channel in hMSCs but it does indicate that Cx45 is a minor contributor to cell to cell coupling in hMSCs. The lack of visualized plaques in the immunostaining for Cx45 (FIG. 28) further supports this interpretation.

The summarized plots of g_{j,ss} versus Vⱼ from pairs between hMSC and transfected HeLa cells are shown in FIG. 35D. The left panel shows the results from hMSC-HeLaCx43 pairs. For symmetrical data (●, four preparations), Boltzmann fits (continuous lines) yielded the following parameters: V_{j,0}=−61/65 mV, g_{j,min}=0.24/0.33, g_{j,max}=0.99/0.99, z=2.4/3.8 for negative/positive Vⱼ. For asymmetrical data (∘, three preparations), the Boltzmann fit (dashed line) at negative Vⱼ values revealed the following parameter values: V_{j,0}=−70 mV, g_{j,min}=0.31, g_{j,max}=1.00, z=2.2. The middle panel shows data from hMSC-HeLaCx40 pairs including three symmetrical (●) and two asymmetrical (∘) g_{j,ss}-Vⱼ relationships. The continuous lines correspond to a Boltzmann fit to symmetrical data (V_{j,0}=−57/76 mV, g_{j,min}=0.22/0.29, g_{j,max}=1.1/1.0, z=1.4/2.3; negative/positive Vⱼ) and the dashed line is a fit to the asymmetrical data (V_{j,0}=−57/85 mV, g_{j,min}=0.22/0.65, g_{j,max}=1.1/1.0, z=1.3/2.2; negative/positive Vⱼ). The data from the six complete experiments from hMSC-HeLaCx45 cell pairs are shown on the right panel. The g_{j,ss} plot versus Vⱼ was strongly asymmetrical and the best fit of the data to the Boltzmann equation at positive Vⱼ values revealed following parameter values: V_{j,0}=31 mV, g_{j,min}=0.07, g_{j,max}=1.2, z=1.8.

FIG. 35E shows Lucifer Yellow transfer from an hMSC to an hMSC (upper panel), from a HeLaCx43 to an hMSC (middle panel), and from an hMSC to a HeLaCx43 (bottom panel). The junctional conductance of the cell pairs was simultaneously measured by methods described earlier (Valiunas et al., 2002) and revealed conductances of ˜13, ˜16 and —18 nS, respectively. The transfer of Lucifer Yellow was similar to that previously reported for homotypic Cx43 or co-expressed Cx43 and Cx40 in HeLa cells (Valiunas et al., 2002). Cell Tracker Green (Molecular Probes) was always used in one of the two populations of cells to allow heterologous pairs to be identified (Valiunas et al., 2000). Lucifer Yellow was always delivered to the cell containing cell tracker. The fluorescence intensity generated by the Cell Tracker Green was 10-15 times less than fluorescence intensity produced by the concentration of Lucifer Yellow delivered to the source cell.

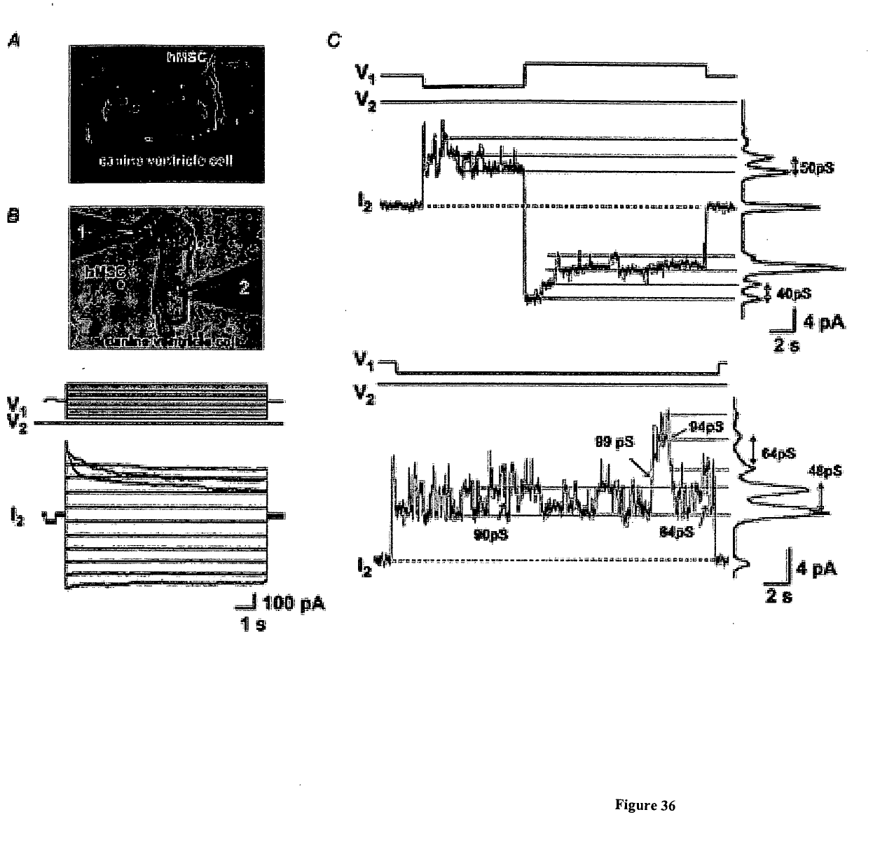

Human MSCs were also co-cultured with adult canine ventricular myocytes as shown in FIG. 36. Immunostaining for Cx43 was detected between the rod-shaped ventricular myocytes and hMSCs as shown in FIG. 36A. The hMSCs couple electrically with cardiac myocytes. Both macroscopic (FIG. 36B) and multichannel (FIG. 36C) records were obtained. Junctional currents in FIG. 36B are asymmetrical while those in FIG. 36C show unitary events of the size range typically resulting from the operation of homotypic Cx43 or heterotypic Cx43-Cx40 or homotypic Cx40 channels (Valiunas et al., 2000; 2001). Heteromeric forms are also possible whose conductances are the same or similar to homotypic or heterotypic forms.

The studies of cell pairs have demonstrated effective coupling of hMSC to other hMSC (13.8±2.4 nS, n=14), to HeLaCx43 (7.9±2.1 nS, n=7), to HeLaCx40 (4.6±2.6 nS, n=5), to HeLaCx45 (11±2.6 nS, n=5), and to ventricular myocytes (1.5±1.3 nS, n=4).

Use of hMSCs as a Delivery Platform for Biological Pacemaking

Human MSCs are viewed as a favorable platform candidate for delivering biological pacemakers into the heart partly on the basis, suggested by Liechty et al. (2000), that they might be immunoprivileged and as such would hopefully not give rise to a rejection response. This is important because in the tradeoff between biological and electronic pacemakers, any need for immunosuppression using the former approach would be a detriment to cell therapy approaches and clinically undesirable.

Human MSCs are obtained readily commercially or from the bone marrow, and are identified by the presence of CD44 and CD29 surface markers, as well as by the absence of other markers that are specific for hematopoietic progenitor cells. Using a gene chip analysis, it was determined that the hMSCs do not carry message for HCN isoforms. Importantly, they also do have a significant message level for the gap junctional protein, connexin43. The latter observation is critical because the theory behind platform therapy is that the hMSC would be loaded with the gene of interest, e.g., HCN2, and implanted into myocardium (Rosen et al., 2004). However, having a cell loaded with a signal would not work unless the cell formed functional connections with its neighbors. The philosophy underlying the use of hMSCs as a delivery platform is summarized in FIG. 2. In brief, in the normal sinus node, hyperpolarization of the membrane initiates inward (I_{f}) current which generates phase 4 depolarization and an automatic rhythm. The changes in membrane potential result in current flow via the low resistance gap junctions such that the action potential propagates from one cell to the next. Use of the hMSC as a platform involves loading it with the gene of interest, e.g., HCN2, preferably via electroporation, thereby avoiding any viral component of the process (Rosen et al., 2004; Rosen, 2005; Cohen et al., 2005; Potapova et al., 2004). The hMSC would have to be coupled effectively to the adjacent myocyte. If this occurred, then the high negative membrane potential of coupled myocytes would hyperpolarize the hMSC, opening the HCN channel and permitting inward current to flow. This current, in turn, would propagate though the low resistance gap junctions, depolarize a coupled myocyte and bring it to threshold potential, resulting in an action potential that would then propagate further in the conducting system. In other words, the hMSC and the myocyte each would have to carry an essential piece of machinery: the myocyte would bring the ionic components that generate an action potential, the hMSC would carry the pacemaker current, and—if gap junctions were present—the two separate structural entities would function as a single, seamless physiologic unit.

The key question then is whether gap junctions are formed between hMSCs and myocytes. The answer is affirmative, as the experimental data disclosed above show. FIG. 33 shows that connexins 43 and 40 are clearly demonstrable in hMSCs. In addition, hMSCs form functional gap junction channels with cell lines expressing Cx43, Cx40 or Cx45 as well as with canine ventricular cardiomyocytes (see also Valiunas et al., 2004, the entire contents of which are hereby incorporated by reference). Lucifer Yellow passage between an hMSC and another hMSC or a HeLaCx43 cell (see FIG. 35E) is yet another indicator of robust gap junction-mediated coupling. The transfer of Lucifer Yellow between hMSCs and HeLa cells transfected with Cx43 is similar to that of homotypic Cx43 or coexpressed Cx43 and Cx40. It excludes homotypic Cx40 as a dominating channel type as Cx40 is some 5 times less permeable to Lucifer Yellow than Cx43 (Valiunas et al., 2002). Moreover, injection of current into an hMSC in close proximity to a myocyte results in current flow to the myocyte (FIG. 36), further indicative of the establishment of functional gap junctions.

These data suggest that MSCs should readily integrate into electrical syncytia of many tissues, promoting repair or serving as the substrate for a therapeutic delivery system. In particular, the data support the possibility of using hMSCs as a therapeutic substrate for repair of cardiac tissue. Other syncytia such as vascular smooth muscle or endothelial cells should also be able to couple to the hMSCs because of the ubiquity of Cx43 and Cx40 (Wang et al., 2001 a). Thus, they may also be amenable to hMSCs-based therapeutics. For example, hMSCs can be transfected to express ion channels which then can influence the surrounding syncytial tissue. Alternatively, the hMSCs can be transfected to express genes that produce small therapeutic molecules capable of permeating gap junctions and influencing recipient cells. Further, for short term therapy, small molecules can be directly loaded into hMSCs for delivery to recipient cells. The success of such approaches is dependent on gap junction channels as the final conduit for delivery of the therapeutic agent to the recipient cells. The feasibility of the first approach has been demonstrated herein by delivering HCN2-transfected hMSCs to the canine heart where they generate a spontaneous rhythm.

Another question concerned the autonomic responsiveness of the hMSCs. As shown by Potapova et al. (2004), the addition of isoproterenol to hMSCs loaded with HCN2 resulted in a shift in activation such that increased current flowed at more positive potentials. The result, as would be expected for native HCN2, should be an increased pacemaker rate. Potapova et al. (2004) also investigated the response of I_{f} expressed by hMSCs to acetylcholine. Acetylcholine alone had no effect on current, but in the presence of isoproterenol antagonized the beta-adrenergic effect of the latter. This is entirely consistent with the physiologic phenomenon of accentuated antagonism.

Human MSCs loaded with HCN2 were also site-specifically injected into the hearts of dogs in which vagal stimulation was used to terminate sinoatrial pacemaker function and/or atrioventricular conduction (Potapova et al., 2004). This resulted in spontaneous pacemaker function that was pace-mapped to the site of injection. Moreover, tissues removed from the site showed gap junctional formation between myocyte and hMSC elements. Finally, the stem cells stained positively for vimentin, indicating that they were mesenchymal, and positively for human CD44 antigen, indicating that they were hMSCs of human origin (Potapova et al., 2004).

In a preliminary study, Plotnikov et al. (2005b) followed the function of hMSC-based biological pacemaking through six weeks post-implantation and found that the rate generated is stable. Equally importantly, staining for immune globulin and for canine lymphocytes was used to determine if rejection of the hMSCs was occurring. Using 2-week and 6-week time points, there was no evidence for humoral or cellular rejection. This is consistent with the earlier work of Liechty et al. (2000) suggesting that hMSCs may be immunoprivileged. If more detailed investigation demonstrates this to be the case, then it would abrogate any need for immunosuppression.

Overall, therefore, hMSCs appear to provide a very attractive platform for delivering pacemaker ion channels to the heart for several reasons: they can be obtained in relatively large numbers through standard clinical interventions; they are easily expanded in culture; preliminary evidence suggests they are capable of long-term transgene expression; and their administration can be autologous or via banked stores (as they are immunoprivileged). Whereas hMSCs might in theory be differentiated in vitro into cardiac-like cells capable of spontaneous activity, the genetic engineering approach described herein does not depend on differentiation along a specific lineage. Moreover, this ex vivo transfection method allows evaluation of DNA integration and engineering of the cell carriers with fail-safe death mechanisms. Accordingly, adult hMSCs are a preferred ion channel delivery platform to be employed in methods for treating subjects afflicted with cardiac rhythm disorders comprising the induction of biological pacemaker activity in the subject's heart, and in making kits for use in such methods.

It is important to emphasize the conceptual and practical differences between the design of (1) gene therapy, and (2) stem cell therapy as described herein. Whereas both have one endpoint in common—the delivery of a biological pacemaker—gene therapy uses specific HCN isoforms to engineer a cardiac myocyte into a pacemaker cell, whereas hMSC therapy uses stem cells as a platform to carry specific HCN and/or MiRP1 isoforms to a heart whose myocytes retain their original function. Gene therapy makes use of preexisting homeotypic cell-cell coupling among myocytes to facilitate propagation of the pacemaker impulses from those myocytes in which pacemaker current is overexpressed to those that retain their original function. In contrast, stem cells depend on heterotypic coupling of cells with somewhat dissimilar populations of connexins to deliver pacemaker current alone from a stem cell to a myocyte whose function is left unchanged. Importantly, and unlike sinus node cells, HCN2-transfected hMSCs are not excitable, because they lack the other currents necessary to generate an action potential. However, when transfected, these cells generate a depolarizing current, which spreads to coupled myocytes, driving myocytes to threshold. In effect, the myocyte acts like a trip wire whose hyperpolarization turns on pacemaker current in the stem cell and whose depolarization turns off the current. The data presented herein suggest that as long as the hMSCs contain the pacemaker gene and couple to cardiac myocytes via gap junctions, they will function as a cardiac pacemaker in an analogous manner to the normal primary pacemaker the sinoatrial node.

Mass of Biological Pacemaker Required for Normal Pacemaker Function

A biological pacemaker needs an optimal size (in terms of cell mass) and an optimal cell-to-cell coupling for long-term normal function. It was fortuitous in the early studies that the HCN constructs used, and the number of transfected hMSCs administered to the canine heart in situ, coupled to surrounding myocytes and functioned as well as they did to generate significant, easily measurable pacemaker activity. A mathematical model has subsequently been used to identify the appropriate hMSC numbers and coupling ratios needed to optimize function.

The mathematical model was used to reconstruct an in vivo stem cell injection using quantum dot nanoparticles (QD). Approximately 120,000 QD-containing hMSCs were injected into rat LV free wall (at z=4.9 mm), and the animal was terminated 1 h after injection. Transverse 10-μm sections were cut and visualized for QD fluorescence at 655 nm with phase contrast overlay to show tissue borders. QD were found within the delivered hMSCs and single QD⁺-cells were visualized in the myocardium at higher resolutions. QD⁺-regions from 230 serial 10-μm transverse sections were identified and used to reconstruct the 3D distribution of QD clusters in the heart. A biological pacemaker was then mathematically modeled taking into account the properties of I_{f} in a stem cell, the effects of cell geometry on the propagation of an action potential, the number of stem cells, the resting-voltage-induced reductions of I_{f}, and the requirements for propagation of an action potential. The radius of a hMSC was assumed to be 7 μm, which meant that the radius of a cluster of 10⁵ stem cells is 0.03 cm, and 0.07 cm for 10⁶ stem cells.

The model indicated that: 10⁵ or more stem cells would generate a muscle action potential; the characteristic input resistance of muscle saturates at about 0.03 cm; because of voltage-dependent reductions in I_{f}, current leaving the stem cell cluster saturates at about 0.03 cm and thus the pacemaker potential in muscle saturates at about 0.03 cm. It was concluded that self sustaining propagation of an action potential in muscle is essentially guaranteed if a shell of cells of radius of about 0.03 cm or larger reaches threshold. This implies that if 1,000,000 stem cells are injected, only 10% need to survive to create a biological pacemaker. These conclusions are consistent with the experimental results on the induction of pacemaker activity in heart tissue in situ disclosed herein.

### EXAMPLE 6

### Use of Chimeric HCN Channels for Biological Pacemaking

Chimeric HCN Channel Constructs

Because the I_{f} pacemaker current flows only at diastolic potentials and should not affect action-potential duration, many recent studies on biological pacemakers have targeted I_{f} as the molecular target. However, it has not previously been suggested or demonstrated that the molecular structure of the HCN channel may be manipulated to produce chimeras with preferred properties for biological pacemaking and treating cardiac rhythm disorders. As described below, portions of different HCN isoforms exhibiting desirable characteristics may be recombined into a chimeric channel having superior functionality compared to the Wt HCN channels from which the chimera is derived.

For constructing HCN chimeras, the HCN genes are first subcloned into expression vectors. For example, mammalian genes encoding HCN1-4 (Santoro et al., 1998; Ludwig et al., 1998; 1999; Ishii et al., 1999) are subcloned into vectors such as pGH19 (Santoro et al., 2000) and pGHE (Chen et al., 2001b). Deletion and chimeric mutants are then made by a PCR/subcloning strategy, and the sequences of the resulting mutant HCN constructs are verified by DNA sequencing.

HCN channels can be characterized as having three main portions, a hydrophilic, cytoplasmic N-terminal portion (region 1), a six-membered, S1-S6 core membrane-spanning (intramembranous) portion (region 2) comprising mainly hydrophobic amino acids, and a hydrophilic, cytoplasmic C-terminal portion (region 3). The boundaries of these portions can readily be determined by one of ordinary skill in the art based on the primary structure of the protein and the known hydrophilicity or hydrophobicity of the constituent amino acids. For example, in mHCN1, the C-terminal portion is D390-L910. The C-terminal portion of mHCN2 is D443-L863. Polynucleotide sequences encoding the entire N-terminal domain, the core transmembrane domain, or the C-terminal domain from any of HCN1, HCN2, HCN3 and HCN4, can be interchanged. The different chimeras so constructed are identified using the nomenclature HCNXYZ, where X, Y, or Z is a number (either 1, 2, 3 or 4) that refers to the identity of the N-terminal domain, core transmembrane domains, or C-terminal domain, respectively.

Thus, for example, in the mHCN112 chimera (see FIG. 1), the N-terminal and the intramembranous portions are from mHCN1 whereas the C-terminal amino acids D390-L910 of mHCN1 are substituted by the carboxy-terminal amino acids D443-L863 of mHCN2. Conversely, in mHCN221, the carboxy-terminal amino acids D443-L863 of mHCN2 are substituted by the carboxy-terminal amino acids D390-L910 of mHCN1. In mHCN211, the amino terminal amino acids M1-S128 of mHCN1 are substituted the amino terminal amino acids M1-S181 of mHCN2. Conversely, in mHCN122, amino acids M1-S181 of mHCN2 are substituted by M1-S128 of mHCN1. In mHCN121, the S1-S6 transmembrane domain amino acids D129-L389 of mHCN1 are substituted by the transmembrane domain amino acids D182-L442 of mHCN2. Conversely, in mHCN212 (FIG. 1), amino acids D182-L442 of HCN2 (i.e., the intramembrane portion) are substituted by D129-L389 of mHCN1 (see Wang et al., 2001b). For preparing human chimeric HCN channels, the same principles are applied mutatis mutandis, employing domains from human HCN channels. For example, hHCN112 has an amino terminal domain and an intramembrane domain from hHCN1, and a carboxy terminal domain from hHCN2.

Expression of these HCN chimeras is readily observable in Xenopus oocytes. For example, cRNA can be transcribed from NheI-linearized DNA (for HCN1 and mutants based on the HCN1 background) or SphI-linearized DNA (for HCN2 and mutants based on the HCN2 background) using a T7 RNA polymerase (Message Machine; Ambion, Austin, Tex.). 50 ng of cRNA is injected into Xenopus oocytes as described previously (Goulding et al., 1992).

Chimeric HCN Channels enhances Biological Pacemaking

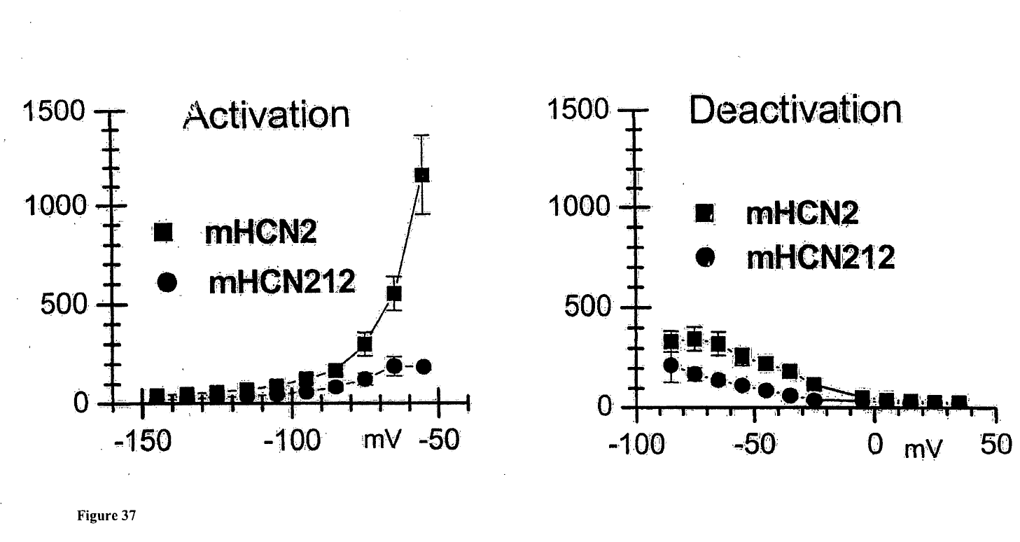

Experiments were performed to compare the gating kinetics of HCN2 and chimeric HCN212 channels when expressed in neonatal rat ventricular myocytes. FIG. 37 shows the results obtained using mHCN2 and a chimeric channel (mHCN212) created by substituting D182-L442 of murine HCN2 with D129-L389 of murine HCN1. Analysis of the activation and deactivation kinetics reveals that mHCN212 exhibits faster kinetics at all voltages compared to mHCN2.

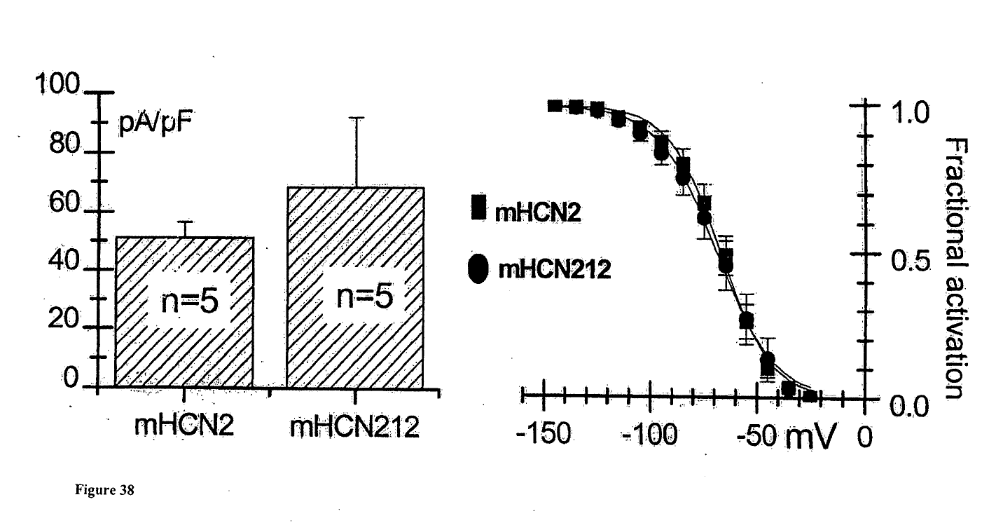

A comparison of expression efficiency of HCN2 and chimeric HCN212 channels in neonatal rat ventricular myocytes is shown in FIG. 38. The results indicate that the expression of the chimeric channel is at least as good as that of the wild-type channel. Moreover, analysis of the voltage dependence of activation indicates no difference in voltage dependence of HCN2 and HCN212 channels when expressed in myocytes.

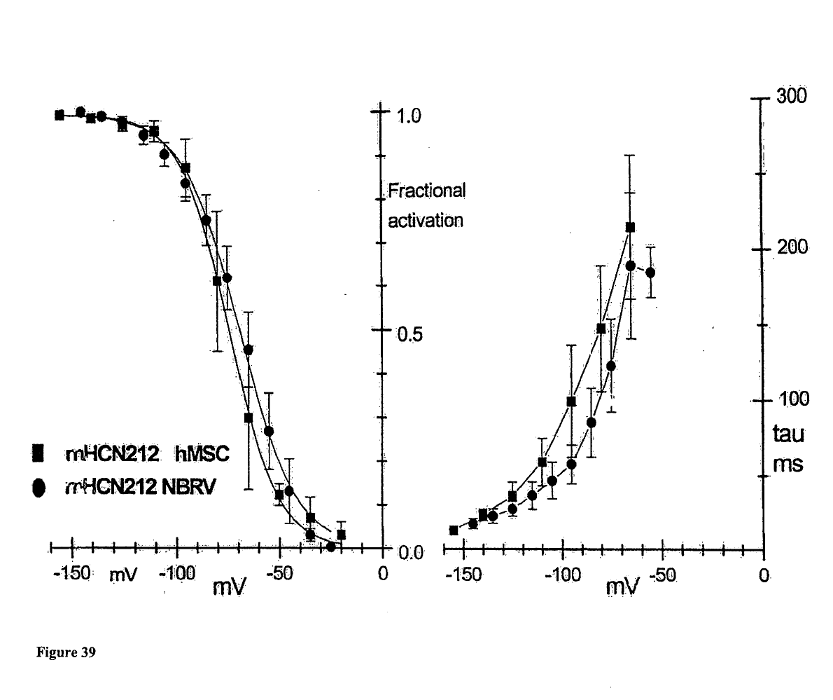

Murine HCN212 was expressed in neonatal rat ventricular myocytes and human adult mesenchymal stem cells and the expressed current subsequently studied in culture. There is no significant difference in the voltage dependence of activation or the kinetics of activation when the chimeric mHCN212 channel is expressed in the two different cell types (see FIG. 39).

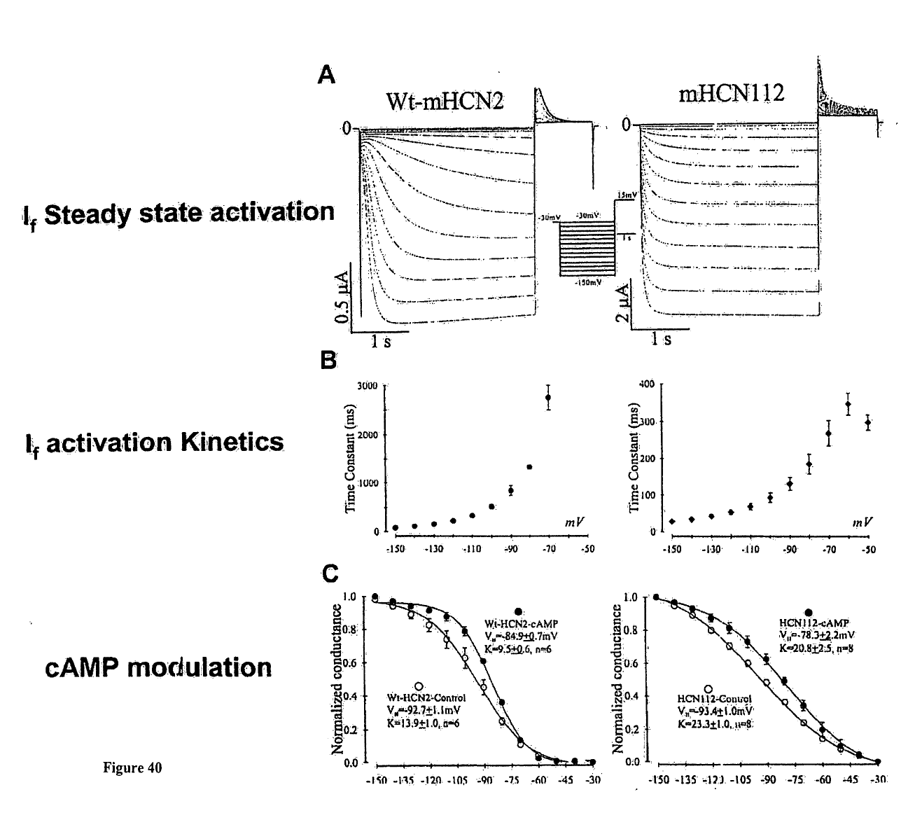

FIG. 40 shows the steady state activation curve, activation kinetics and cAMP modulation of wildtype mHCN2 and mHCN112 in oocytes. The data illustrate that the chimeric HCN112 channel achieves significantly faster kinetics than HCN2 while preserving a strong cAMP response.

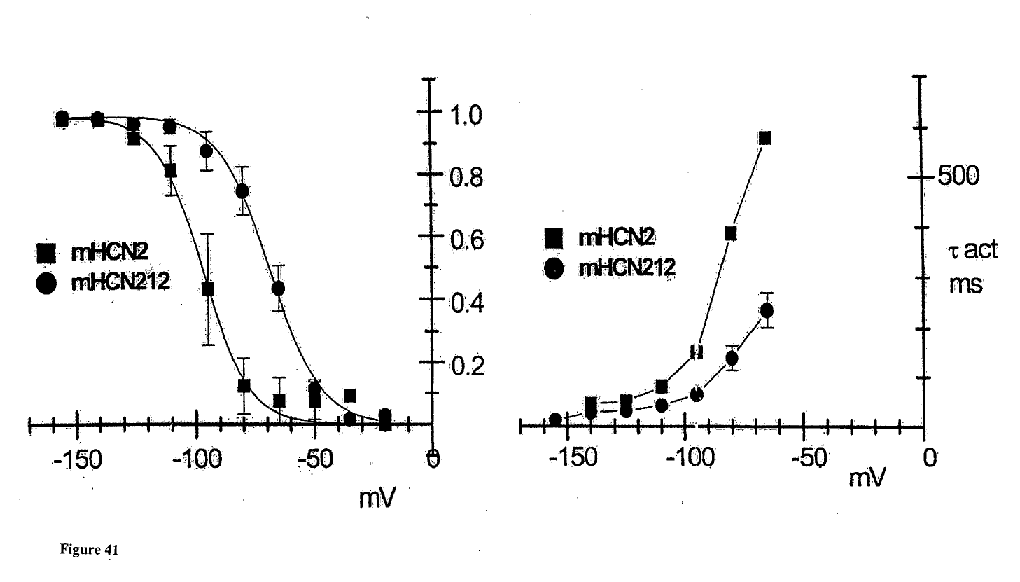

A comparison of the gating characteristics of mHCN2 and chimeric mHCN212 channels expressed in adult hMSCs (FIG. 41) shows that the voltage dependence of activation is shifted significantly positive, and the kinetics of activation at any measured voltage are significantly faster, for mHCN212 compared to HCN2.

These data suggest that the HCN212 chimera has significant advantages over the wild-type HCN2 channel in inducing pacemaker activity for therapeutic applications. Importantly, the positive shift and faster kinetics would be expected to result in more current at shorter times for any specific voltage, and in particular, for voltages in the diastolic potential range of cardiac cells (−50 to −90 mV).

Thus, manipulations can be employed to create chimeric HCN channels that have suppressed or enhanced activities compared to the native HCN channels from which they were derived, which allows selection of channels with different characteristics optimized for treating cardiac conditions. For example, the activation curves of the HCN channel current may be shifted to more positive or more negative potentials; the hyperpolarization gating may be enhanced or suppressed; the sensitivity of the channel to cyclic nucleotides may be increased or decreased; and differences in basal gating may be introduced. More particularly, the data provide evidence that a pacemaker channel with fast kinetics and good responsiveness to cAMP (and hence altered responsiveness to autonomic stimulation) can be obtained by, for example, selection of HCN1 components. Slower kinetics may also be obtained by, for example, selection of HCN4 components in the chimera. The creation of HCN chimeras exhibiting characteristics that are beneficial for treating heart disorders has not previously been reported.

### EXAMPLE 7

### Pacemaking by Tandem Biological and Electronic Pacemakers In Situ

Implantation of Tandem Biological and Electronic Pacemakers in Dogs

Experiments involving animals were performed using protocols approved by the Columbia University Institutional Animal Care and Use Committee and conform to the Guide for the Care and Use of Laboratory Animals (NIH Publication No. 85-23, revised 1996).

Adult mongrel dogs weighing 22-25 kg were anesthetized with propofol 6 mg/kg IV and inhalational isoflurane (1.5%-2.5%). Using a steerable catheter, saline (n=5), AdmHCN2 (n=6) or AdmE324A (n=4) were injected into the left bundle branch (LBB) as described previously (Plotnikov et al., 2004). In 2 additional dogs AdmE324A was injected into the LV septal myocardium as an internal control. Complete AV block was induced via radiofrequency ablation and each site of injection was paced via catheter electrode to distinguish electrocardiographically the origin of the idioventricular rhythm during the follow up period.

An electronic pacemaker (Discovery II, Flextend lead; Guidant, Indianapolis, Ind.) was implanted and set at VVI 45 bpm. ECG, 24 hour Holter monitoring, pacemaker log record check, and overdrive pacing at 80 bpm were performed daily for 14 days. To evaluate beta-adrenergic responsiveness, on day 14, epinephrine (1.0, 1.5 and 2.0 μg/kg/min for up to 10 min each) was infused to an endpoint of a 50% increase in idioventricular rate or ventricular arrhythmia (single ventricular premature beats having a morphology other than that of the dominant idioventricular rhythm or ventricular tachycardia), whichever occurred first. If none of the above responses was observed within 10 min after onset of the maximal dose of 2 μg/kg/min, the infusion was terminated.

Data are presented as means ±SEM. In the in situ experiments, the 5 saline-injected dogs and the 2 injected into the myocardium (rather than the LBB) with AdmE324A showed no electrophysiologic differences and were combined into one control group for subsequent analysis. One-way ANOVA was used to evaluate the effect of an implanted construct on electrophysiological parameters. Subsequent analysis was performed using Bonferroni's test where equal variances were assumed and the Games-Howell test where variances were unequal. A two-way contingency table analysis was conducted to evaluate whether epinephrine had different effects across three groups. Data were analyzed using SPSS for Windows software (SPSS, Inc.). P<0.05 was considered to be significant.

Operation of Tandem Biological and Electronic Pacemakers in situ

In a preliminary experiment, the possibility that injecting an adenovirus carrying the E324A mutant might provide an effective alternative to HCN2 was tested in vivo. It was found that E324A-infected dogs manifested basal rates that did not differ significantly from those of HCN2-infected animals, while their catecholamine-responsiveness was greater (Plotnikov et al., 2005a).

In the present experiments, adenoviral vectors carrying the HCN2 and E234A-HCN2 genes, respectively, were then used to generate pacemaking activity in vivo in tandem with implanted electronic pacemakers, and the performance of the tandem pacemakers was compared with that of an electronic pacemaker used alone. Six dogs received injections of an adenoviral vector incorporating the HCN2 gene in 0.6 ml of saline into the left bundle branch (LBB) via a steerable catheter. The HCN2 virus had been characterized in neonatal rat myocytes as follows: midpoint of activation=−69.3 mV (n=5); at −65 mV activation τ=639±72 ms (n=5); expressed current at −135 mV=53.5±8.3 pA/pF (n=10). Four dogs were injected with an adenoviral vector incorporating the mutant E324A gene in the LBB, and two additional dogs were injected into the LV septal myocardium as an internal control. As another control, five dogs received 0.6 ml of saline injected into the LBB.

Complete AV block was induced via radiofrequency ablation, and electronic pacemakers were implanted into the right ventricular apical endocardium and set a VV1 45 bpm. ECG and 24-h monitoring were performed daily for 14 days. Beta-adrenergic responsiveness was also evaluated as described above.

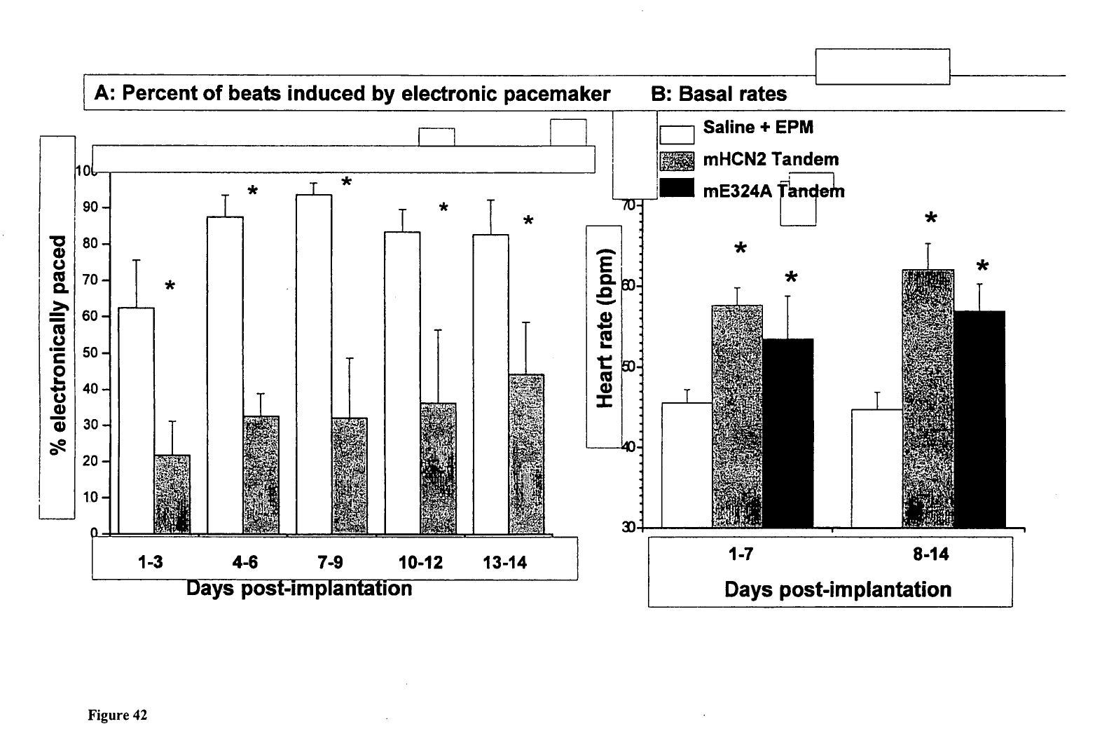

The electronic pacemaker triggered 83±5% of all beats in controls, contrasting (P<0.05) with 26±6% in the mHCN2 and 36±7% in the mE324A groups (for the latter two, P>0.05). A temporal analysis of the electronically paced beats for the tandem HCN2-electronic versus the electronic-only pacemaker is shown in FIG. 42A. It is noteworthy that a significantly lower number of beats was initiated electronically in the HCN2 group throughout the study period. Results for E324A (not shown) did not differ significantly from HCN2.

Escape time was evaluated daily by performing three 30-s periods of ventricular overdrive pacing at 80 bpm followed by an abrupt cessation of pacing. The average time between the final electronically paced beat and the first intrinsic beat was then determined. Escape times ranged from 1-5 s across all three groups and incorporated a wide variability, such that no significant differences were seen. Hence no advantage accrued to any group with regard to escape intervals. There was a different result with regard to basal heart rates throughout the 14-day period, however. As shown in FIG. 42B, average heart rate in saline controls was that determined by the rate of the electronic pacemaker (45 bpm). This was significantly slower throughout the study than that of mHCN2 or mE324A-injected dogs, which groups did not differ from one another.

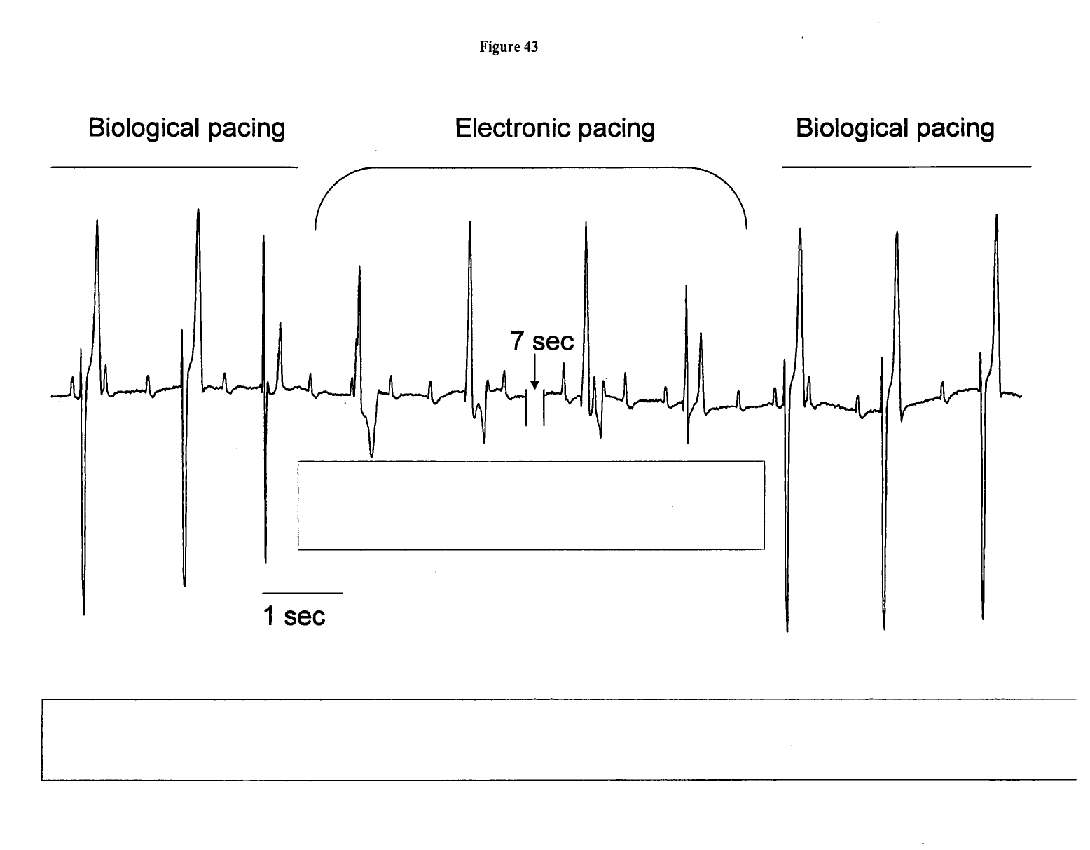

An example of the interrelationship between the biological and the electronic components of the tandem pacemaker is shown in FIG. 43. It is evident that as the biological component slows, the electronic takes over, and that as the biological component speeds in rate, the electronic ceases to fire.

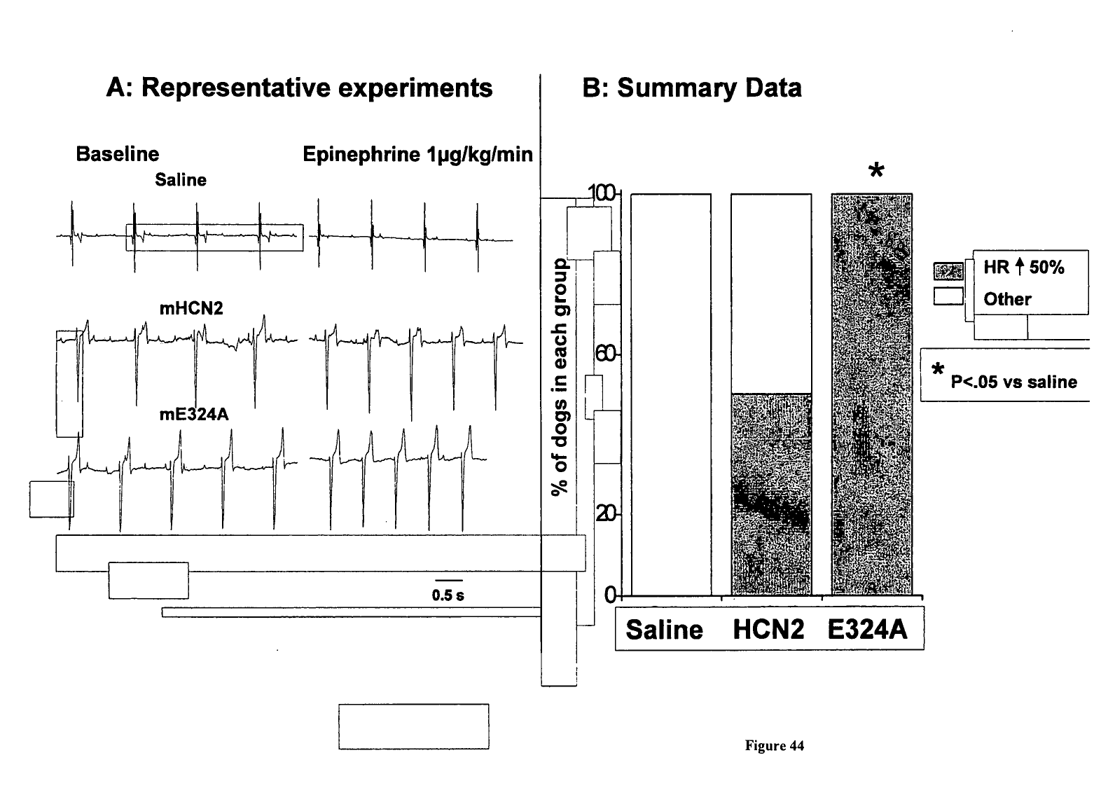

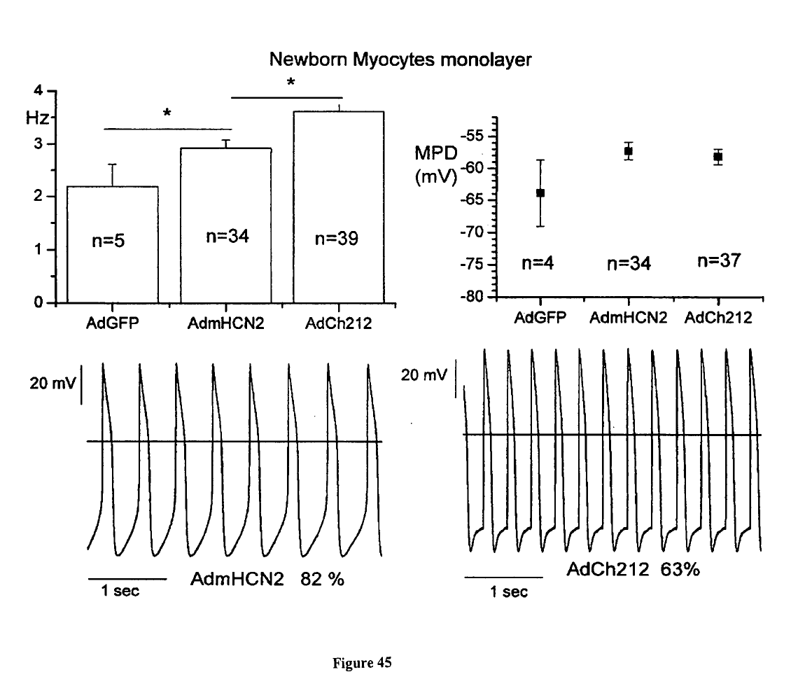

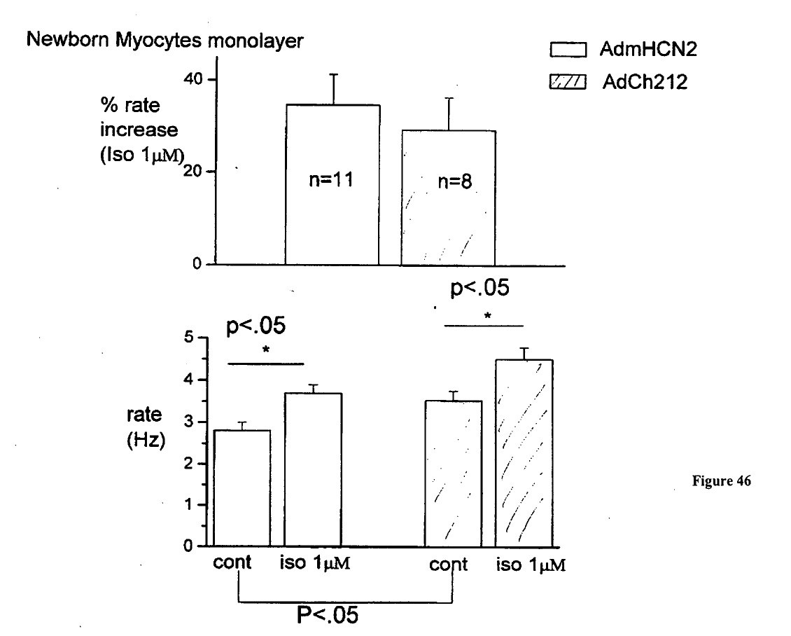

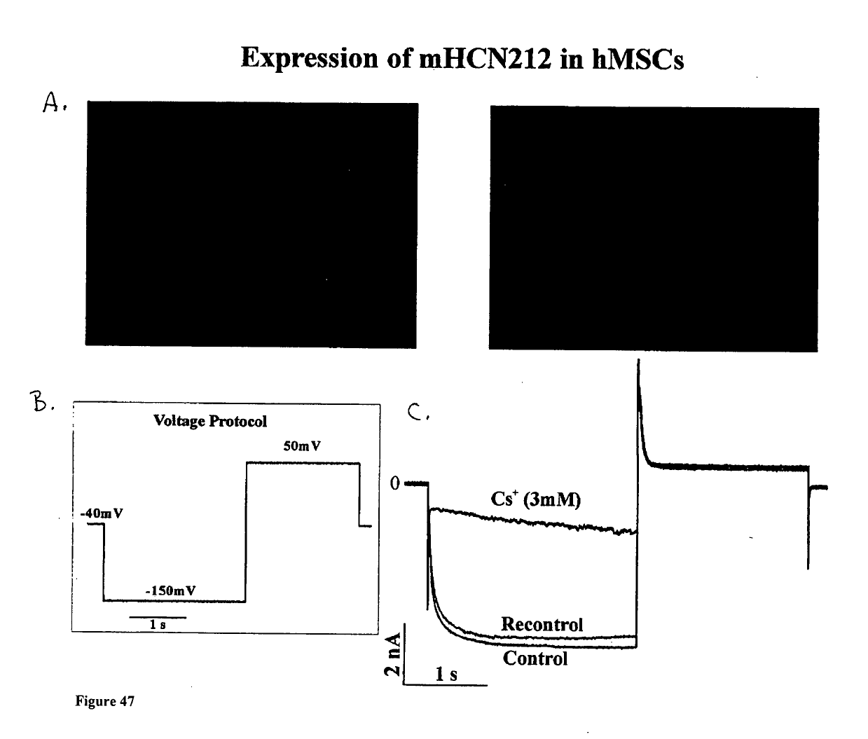

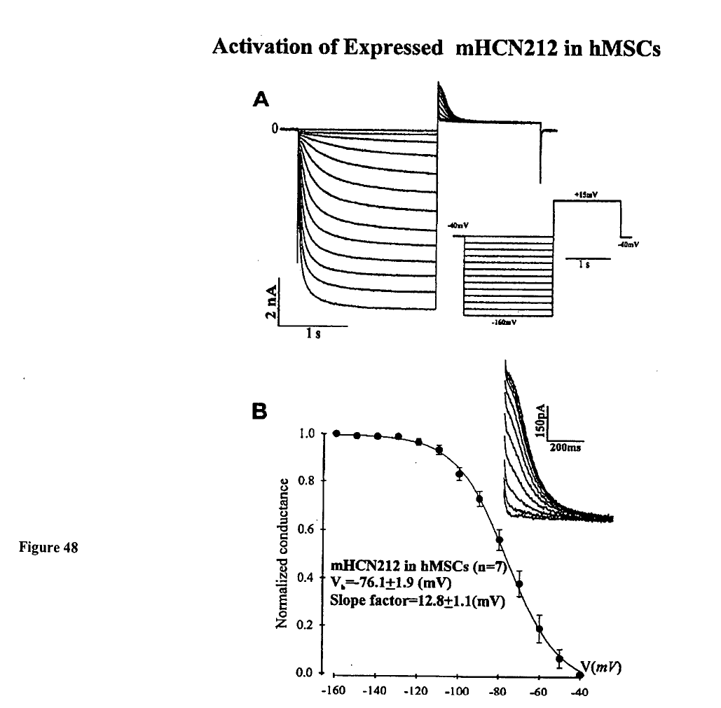

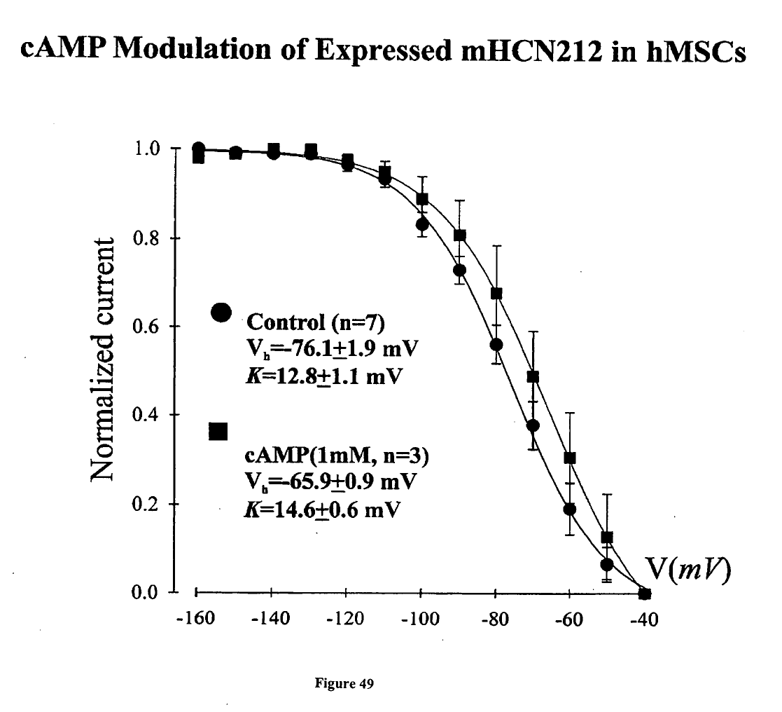

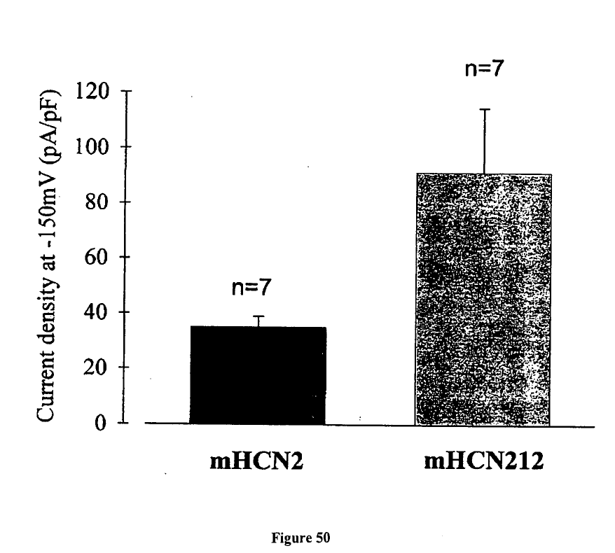

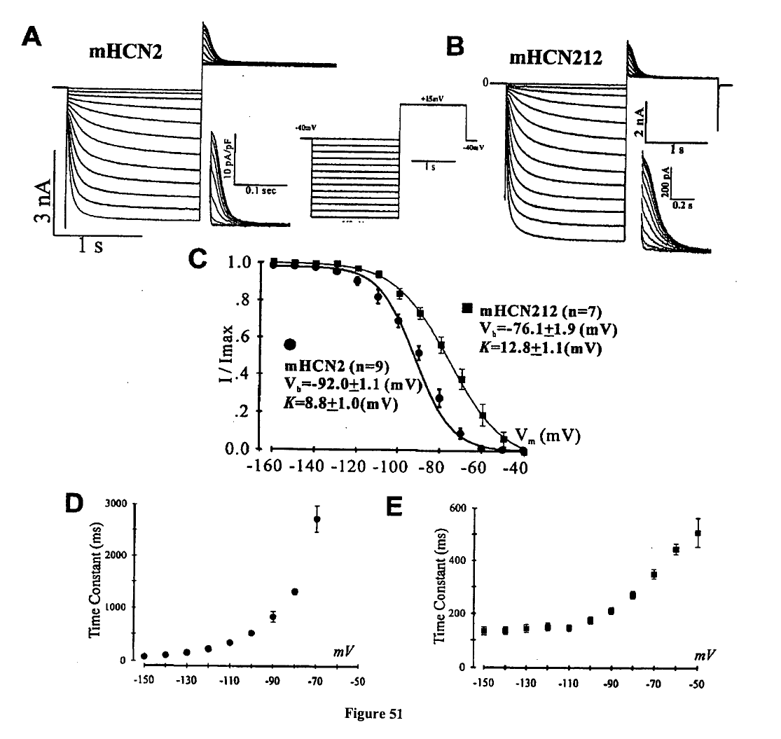

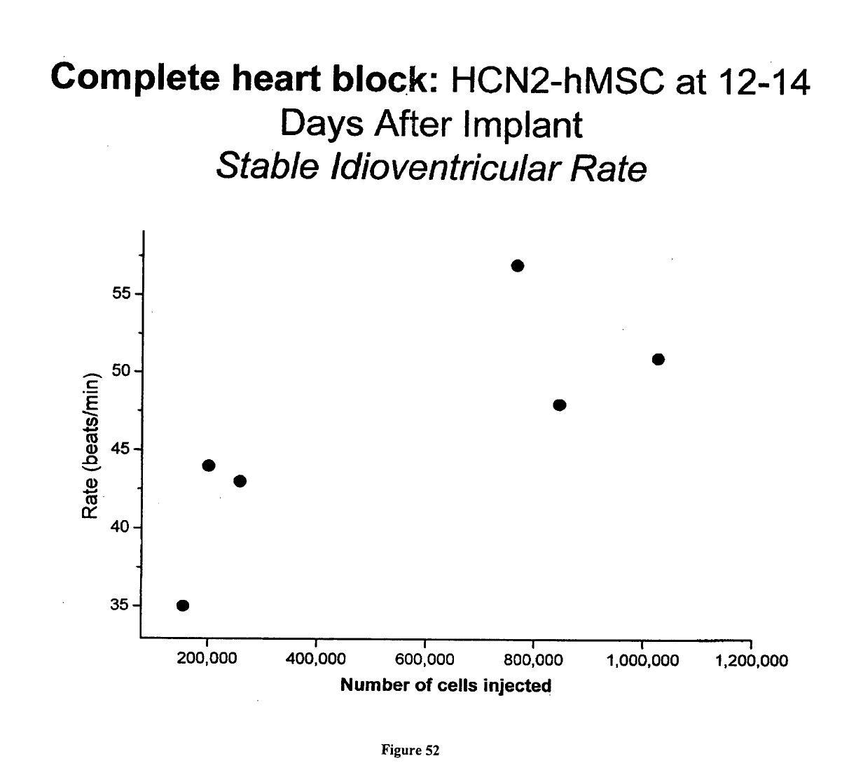

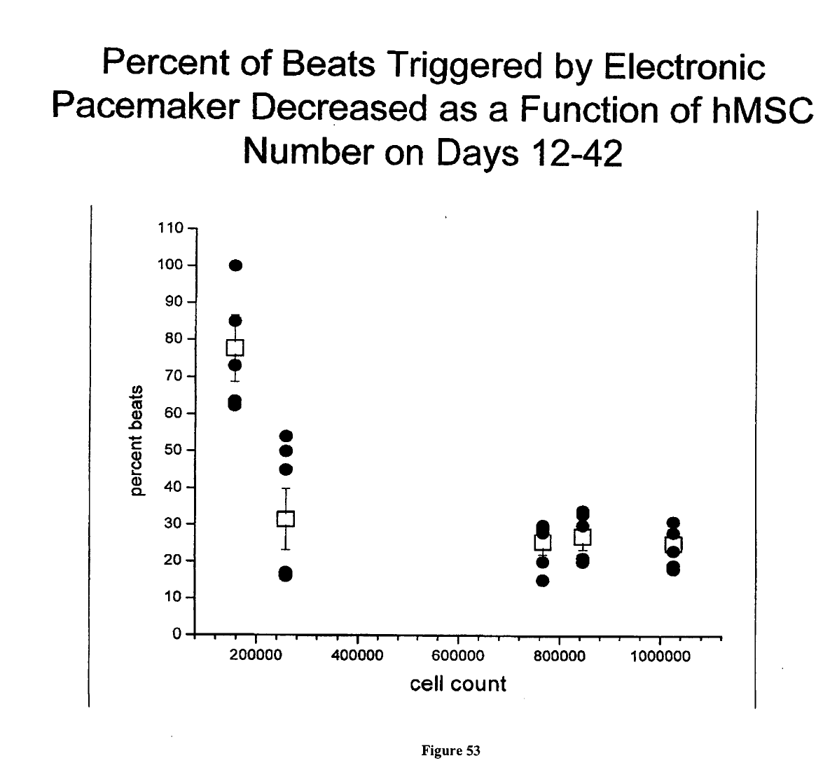

FIG. 44 demonstrates the response to epinephrine in terminal experiments. Panel A shows representative ECGs for all three groups prior to and during infusion of epinephrine, 1 μg/kg/min. Control rates were 42, 44 and 52 bpm for the saline, mHCN2 and mE324A groups, respectively. With epinephrine, rates increased to 44, 60 and 81 bpm. Panel B summarizes the rate changes occurring at all doses of epinephrine. As can be seen, in the saline group all dogs showed less than a 50% increase in rate and/or ventricular premature depolarizations throughout the range of epinephrine concentrations administered. One-half of the mHCN2 group generated a 50% or more increase in heart rate, of which 33% required the highest dose of epinephrine to achieve this increase. The remainder had less than a 50% increase in heart rate or the occurrence of ventricular premature depolarizations. Finally, the mE324A group manifested greater than a 50% increase in heart rate at the lowest dose of epinephrine given. Hence there was far greater epinephrine sensitivity in the mE324A group than in either of the others.

Tandem Therapy as an alternative to either Electronic or Biological Pacemaking

The experimental data presented above demonstrate, inter alia, that biological pacemakers based on expression of mHCN2 and mE234A genes operate seamlessly in tandem with electronic pacemakers to prevent heart rate from falling below a selected minimum beating rate (FIG. 42); there is conservation of total number of electronic beats delivered (FIG. 43); and there is provision of a higher, more physiologic and catecholamine-responsive heart rate than is the case with an electronic pacemaker alone (FIG. 44). Although an adenoviral vector was used to introduce the pacemaker genes into canine hearts, data presented herein also indicate that hMSCs can provide an effective platform for delivery of ion channel currents into the heart. Factors favoring the use of hMSCs include their demonstrated ability to form gap junctions with a variety of cell types, including cardiomyocytes (FIGS. 33-36); their ability to generate in heart tissue pacemaker activity that appears to be stable, at least over a 6-week period (Plotnikov et al., 2005b); and evidence of no humoral or cellular rejection after six weeks (Plotnikov et al., 2005b), which if confirmed over the longer term, would abrogate any need for immunosuppression in hMSC-mediated therapy. Data were also provided indicating that HCN channel domains can be recombined to produce chimeric HCN channels that exhibit desirable gating characteristics for use in treating cardiac conditions.

The data provided herein confirm the feasibility of engineering a biologic pacemaker to meet the demands placed on modern day electronic pacemakers, specifically to provide a physiologic basal heart rate and a means to elevate heart rate during times of increased demand. mHCN2, mE324A and chimeric HCN channels provide biologic pacemakers with different characteristics; yet they demonstrate the principle that biologic pacemakers, like their electronic counterparts, can be tuned for basal heart rate and catecholamine responsiveness.

The strengths and weaknesses of electronic pacemakers have been previously considered (Rosen et al., 2004; Rosen, 2005; Cohen et al., 2005): clearly they are the state of the art as life-saving devices for treating a number of cardiac arrhythmias and are being used increasingly for cardiac failure. These advantages more than outweigh their disadvantages (see Background). Because electronic pacemakers represent a highly successful form of medical palliation, they will not easily be replaced, but the fact that they are not completely physiologic does make them a target for improvement and ultimately replacement. However, the therapy that replaces them should be more long-lasting, have less potential for inflicting damage, and be more physiologic. It is with this in mind that biological pacemakers are being developed. It has been suggested that biological pacemakers should have the potential to (1) create a lifelong, stable physiologic rhythm without need of replacement; (2) compete effectively with electronic pacemakers in satisfying the demand for a safe baseline rhythm, coupled with autonomic responsiveness to facilitate responsiveness to the demands of exercise and emotion; (3) be implanted at sites adjusted from one patient to another such that propagation through an optimal pathway of activation occurs and efficiency of contraction is optimized; (4) confer no risk of inflammation, neoplasia or rejection; (5) have no arrhythmogenic potential. In other words, they should represent not palliation, but cure (Rosen et al., 2004; Rosen, 2005).

There are two reasons to consider the use of tandem therapy as opposed to therapy based on biological or electronic pacemakers alone: one associated with clinical trials, and the other associated with more widespread clinical use. After the appropriate safety and efficacy preclinical testing is completed, a study of tandem pacemaking in patients in complete heart block and atrial fibrillation would be a reasonable starting point for a combined phase 1/phase 2 trial. Such a population has need of pacemaker therapy and is not a candidate for AV sequential electronic pacing. The state of the art therapy for such patients—a demand form of electronic ventricular pacing—would be indicated and a biological implant could be made as well. Moreover, the electronic component set at a sufficiently low rate would ensure a “safety net” in case the biological component failed. However, even if phase 1 and phase 2 trials provide evidence of safety and efficacy of the biological pacemaker there is a need to understand how long a biological pacemaker will last. And in the first generation of patients to receive them, this should likely be a lifelong question, during which there must be continued electronic backup.

With respect to broader clinical application of the tandem pacemaker concept there are several issues to consider. First, the system is redundant by design and would have two completely unrelated failure modes. Two independent implant sites and independent energy sources would provide a safety mechanism in the event of a loss of capture (e.g., due to myocardial infarction). Second, the electronic pacemaker would provide not only a baseline safety net, but an ongoing log of all heartbeats for review by clinicians, thus providing insight into a patient's evolving physiology and the performance of their tandem pacemaker system. Third, since the biologic pacemaker will be designed to perform the majority of cardiac pacing, the longevity of the electronic pacemaker could be dramatically improved. Alternatively longevity could be maintained while the electronic pacemaker could be further reduced in size. Finally, the biological component of a tandem system would provide true autonomic responsiveness, a goal that has eluded more than 40 years of electronic pacemaker research and development.

### REFERENCES

U.S. Pat. No. 5,318,597, issued Jun. 7, 1994 to Hauck J A et al.

U.S. Pat. No. 5,376,106, issued Dec. 27,;1994 to Stahmann J E et al.

U.S. Pat. No. 5,983,138, issued Nov. 9, 1999 to Kramer A P.

U.S. Pat. No. 6,110,161, issued Aug. 29, 2000 to Mathiesen I et al.

U.S. Pat. No. 6,783,979, issued Aug. 31, 2004 to Rosen M R et al.

U.S. Pat. No. 6,849,611, issued Feb. 1, 2005 to Rosen M R et al.

U.S. Ser. No. 10/745,943, filed Dec. 24, 2003 by Rosen M R et al.

U.S. Ser. No. 10/342,506, filed Jan. 15, 2003 by Rosen M R et al., published Jul. 15, 2004 as U.S. Application Publication No. 2004/0137621 A1.

U.S. Provisional Application No. 60/532,363, filed Dec. 24, 2003 by Brink P R et al.

U.S. Provisional Application No. 60/701,312, filed Jul. 21, 2005 by Brink P R et al.

U.S. Provisional Application No. 60/704,210, filed Jul. 29, 2005 by Brink P R et al.

U.S. Provisional Application No. 60/715,934, filed Sep. 9, 2005 by Rosen M R et al.

PCT International Publication No. WO 2005/062857, published Jul. 14, 2005.

Abraham W T, Hayes D L (2003) Cardiac resynchronization therapy for heart failure. Circulation 108: 2596-2603.

Abbott G W, et al.: MiRP1 forms Iₖᵣ potassium channels with HERG and is associated with cardiac arrhythmia. Cell. Vol. 97, No. 2, Apr. 16, 1999, pages 175-187.

Accili E A, et al.: Differential control of the hyperpolarization-activated current (I_{f}) by intracellular cAMP and phosphatase inhibition. J. Physiol. Vol. 491, 1996, pages 115.

Altomare C, et al.: Allosteric voltage-dependent gating of HCN channels. Biophys. J. Vol. 80, 2001, pages 241a.

Altomare C, Bucchi A, Camatini E, Baruscotti M, Viscomi C, Moroni A, DiFrancesco D (2001) Integrated allosteric model of voltage gating of HCN channels. J Gen Physiol 117: 519-532.

Amado R G, Chen I S (1999) Lentiviral Vectors—the promise of gene therapy within reach? Science 285: 674-676.

Ausubel F M, Brent R, Kingston R E, Moore D D, Seidman J G, Smith J A, Struhl K, eds (2004) Current Protocols in Molecular Biology, John Wiley & Sons.

Barbuti A, et al.: Action of internal pronase on the f-channel kinetics in the rabbit SA node. J. Physiol. Vol. 520, No. 3, Nov. 1, 1999, pages 737-744.

Biel M, Schneider A, Wahl C (2002) Cardiac HCN channels: Structure, function, and modulation. Trends Cardiovasc Med 12: 202-216.

Cerbai E, et al.: Influence of postnatal-development on I_{f} occurrence and properties in neonatal rat ventricular myocytes. Cardiovasc. Res. Vol. 42, No. 2, May 1999, pages 416-423.

Chang F, et al.: Effects of protein kinase inhibitors on canine Purkinje fibre pacemaker depolarization and the pacemaker current I_{f}. J. Physiol. Vol. 440, 1991, pages 367-384.

Chauhan V S, et al.: Abnormal cardiac Na(+) channel properties and QT heart rate adaptation in neonatal ankyrin(B) knockout mice. Circ. Res. Vol. 86, No. 4, Mar. 3, 2000, pages 441-447.

Chen J, Mitcheson J S, Tristani-Firouzi M, Lin M, & Sanguinetti M C (2001a) The S4-S5 linker couples voltage sensing and activation of pacemaker channels. Proc Natl Acad Sci USA 98: 11277-11282.

Chen S, Wang J, Siegelbaum S A (2001b) Properties of hyperpolarization-activated pacemaker current defined by coassembly of HCN1 and HCN2 subunits and basal modulation by cyclic nucleotide. J Gen Physiol 117: 491-504.

Cho H C, Kashiwakura Y, Azene E, Marban E (2005) Conversion of non-excitable cells to self-contained biological pacemakers. Circulation (Abstract) 112: II-307.

Clapham D E (1998) Not so funny anymore: pacing channels are cloned. Neuron 21: 5-7.

Cleland J G, Daubert J C, Erdmann E, Freemantle N, Gras D, Kappenberger L, Tavazzi L, Cardiac Resynchronization-Heart Failure (CARE-HF) Study Investigators (2005) The effect of cardiac resynchronization on morbidity and mortality in heart failure. N Engl J Med 352:1539-1549.

Cohen I S, Brink, P R, Robinson R B, Rosen M R (2005) The why, what, how and when of biological pacemakers. Nat Clin Pract Cardiovasc Med 2: 374-375.

Cui J, et al.: Gating of IsK expressed in Xenopus oocytes depends on the amount of mRNA injected. Gen. Physiol. Vol. 104, No. 1, July 1994, pages 87-105.

Decher N, Chen J, Sanguinetti M C (2004) Voltage-dependent gating of hyperpolarization-activated, cyclic nucleotide-gated pacemaker channels: molecular coupling between the S4-S5 and C-linkers. J Biol Chem 279: 13859-13865.

DiFrancesco D (1993) Pacemaker mechanisms in cardiac tissue. Annu Rev Physiol 55: 455-472.

DiFrancesco D, et al.: Direct activation of cardiac pacemaker channels by intracellular cyclic AMP. Nature. Vol. 351, No. 6322, May 9, 1991, pages 145-147.

Dixon J E and McKinnon D: Quantitative analysis of potassium channel expression in atrial and ventricular muscle of rats. Circ. Res. Vol. 75, No. 2, August 1994, pages 252-260.

Edelberg J M, Aird W C, Rosenberg R D (1998) Enhancement of murine cardiac chronotropy by the molecular transfer of the human β-adrenergic receptor cDNA. J Clin Invest 101: 337-343.

Edelberg J M, Huang D T, Josephson M E, Rosenberg R D (2001) Molecular enhancement of porcine cardiac chronotropy. Heart 86: 559-562.

Elfgang C, Eckert R, Lichtenberg-Frate H, Butterweck A, Traub O, Klein R A, Hulser D F, Willecke K. (1995) Specific permeability and selective formation of gap junction channels in connexin-transfected HeLa cells. J Cell Biol 129: 805-817.

Fares N, et al.: Characterization of a hyperpolarization-activated current in dedifferentiated adult rat ventricular cells in rimary culture. J. Physiol. Vol. 506, No. 1, Jan. 1, 1998, pages 73-82.

Ferrer T, Rupp J, Piper D R, Tristani-Firouzi M (2006) The S4-S5 linker directly couples voltage sensor movement to the activation gate in the human ether-á-go-go-related gene (HERG) K⁺ channel. J Biol Chem 281:12858-12864.

Freudenberger R S, Wilson A C, Lawrence-Nelson J, Hare J M, Kostis J B; Myocardial Infarction Data Acquisition System Study Group (MIDAS 9) (2005) Permanent pacing is a risk factor for the development of heart failure. Am J Cardiol 95: 671-674.

Gerhardstein B L, et al.: Proteolytic processing of the C terminus of the alpha (1C) subunit of L-type calcium channels and role of a proline-rich domain in membrane tethering of proteolytic fragments. J Biol. Chem. Vol. 275, No. 12, Mar. 24, 2000, pages 8556-8563.

Goulding E H, Ngai J, Kramer R H, Colicos S, Axel R, Siegelbaum S A, Chess A. (1992) Molecular cloning and single-channel properties of the cyclic nucleotide-gated channel from catfish olfactory neurons. Neuron 8: 45-58.

Guillemare E, et al.: Effects of the level of mRNA expression on biophysical properties, sensitivity to neurotoxins, and regulation of the brain delayed rectifier K+ channels Kvl.2. Biochemistry. Vol. 31, No. 49, Dec. 15, 1992, pages 12463-12468.

Hansen J E, et al.: Prediction of O-glycosylation of mammalian proteins: Specificity patterns of UDP-GalNAc:polypeptide N-acetylgalactosaminyltransferase. Biochem. J. Vol. 308, No. 3, Jun. 15, 1995, pages 801-813.

Harrison R L, Byrne B J, Tung 1 (1998) Electroporation-mediated gene transfer in cardiac tissue. FEBS Lett 435: 1-5.

Hayes D L (2000) Pacemaker timing cycles and electrocardiography, Chapter 6 of Cardiac Pacing and Defibrillation, pp. 201-223, Mayo Foundation.

He T C, Zhou S, da Costa L T, Yu J, Kinzler K W, Vogelstein B (1998) Simplified system for generating recombinant adenoviruses. Proc Natl Acad Sci USA 95: 2509-2514.

Honore E, et al.: Different types of K+ channel current are generated by different levels of a single mRNA. EMBO J. Vol. 11, No. 7, July 1992, pages 2465-2471.

Ishii T M, Takano M, Xie L H, Noma A, Ohmori H (1999) Molecular characterization of the hyperpolarization-activated cation channel in rabbit heart sinoatrial node. J Biol Chem 274: 12835-12839.

Kaupp U B, et al.: Molecular diversity of pacemaker ion channels. Annu, Rev. Physiol. Vol. 63, 2001, pages 235-257.

Kehat, I, Khimovich L, Caspi O, Gepstein A, Shofti R, Arbel G, Huber I, Satin J, Iskovitz-Eldor J, Gepstein L (2004) Electromechanical integration of cardiomyocytes derived from human embryonic stem cells. Nature Biotech 22: 1282-1289.

Kuznetsov V Pak E, Robinson R B, Steinberg S F (1995) β-2-adrenergic receptor actions in neonatal and adult rat ventricular myocytes. Circ Res 76: 40-52.

Laing J G and Beyer E C (1995) The gap junction protein connexin43 is degraded via the ubiquitin proteasome pathway. J Biol Chem 270: 26399-26403.

Larsson H P (2002) The Search Is on for the Voltage Sensor-to-gate Coupling. J Gen Physiol 120: 475-481.

Liechty K W, MacKenzie T C, Shaaban A F, Radu A, Moseley A M, Deans R, Marshak D R, Flake A W (2000) Human mesenchymal stem cells engraft and demonstrate site-specific differentiation after in utero transplantation in sheep. Nat Med 6: 1282-1286.

Lu Y, Mahaut-Smith M P, Huang C L, Vandenberg J I (2003) Mutant MiRP1 subunits modulate HERG K+ channel gating: a mechanism for pro-arrhythmia in long QT syndrome type 6. J Physiol 551: 253-62.

Ludwig A, Zong X, Jeglitsch M, Hofmann F, Biel M (1998) A family of hyperpolarization-activated mammalian cation channels. Nature 393: 587-591.

Ludwig A, Zong X, Stieber J, Hullin R, Hofmnann F, Biel M (1999) Two pacemaker channels from human heart with profoundly different activation kinetics. EMBO J 18: 2323-2329.

Macri V, Accili E A (2004) Structural elements of instantaneous and slow gating in hyperpolarization-activated cyclic nucleotide-gated channels. J Biol Chem 279: 16832-16846.

Martens J R, et al.: Differential targeting of Shaker-like potassium channels to lipid rafts. BiolChem. Vol. 275, No. 11, Mar. 17, 2000, pages 7443-7446.

Melman Y F, et al.: Structural determinants of KvLQT1 control by the KCNE family of proteins. J Biol Chem. Vol. 276, No. 9, Mar. 2, 2001, pages 6439-6444.

Miake J, Marban E, Nuss H B (2003) Functional role of inward rectifier current in heart probed by Kir2.1 overexpression and dominant-negative-suppression. J Clin Invest 111: 1529-1536.

Miake J, Marban E, Nuss H B (2002) Gene therapy: biological pacemaker created by gene transfer. Gene therapy: biological pacemaker created by gene transfer. Nature 419: 132-133.

Mitcheson J S, Chen J, Sanguinetti M C (2000) Trapping of a methanesulfonanilide by closure of the HERG potassium channel activation gate. J Gen Physiol 115: 229-40.

Monteggia L M, Eisch A J, Tang M D, Kaczmarek L K, Nestler E J (2000) Cloning and localization of the hyperpolarization-activated cyclic nucleotide-gated channel family in rat brain. Brain Res Mol Brain Res 81: 129-139.

Moosmang S, et al.: Cellular expression and functional characterization of four hyperpolarization-activated pacemaker channels in cardiac and neuronal tissues. Eur. J. Biochem. Vol. 268, No. 6, March 2001, pages 1646-1652.

Moran O, et al.: Level of expression controls modes of gating of a K+ channel. FEBS Lett. Vol. 302, No. 1, May 4, 1992, pages 21-25.

Moroni A, et al.: Kinetic and ionic properties of the human HCN2 pacemaker channel. Pflugers Arch. Vol. 439, No. 5, March 2000, pages 618-626.

Moses H W, Moulton K P, Miller B D and Schneider J A (2000) Types of pacemakers and hemodynamics of pacing, Chapter 5 of A Practical Guide to Cardiac Pacing-Fifth Edition, pp. 78-84, Cippincott Williams & Wilkins, Philadelphia.

Ng P, Parks R J, Cummings D T, Evelegh C M, Graham F L (2000) An enhanced system for construction of adenoviral vectors by the two-plasmid rescue method. Hum Gene Ther 11: 693-699.

Orlic D, Kajstura J, Chimenti S, Jakoniuk I, Anderson S M, Li B, Pickel J, McKay R, Nadal-Ginard B, Bodine D M, Leri A, Anversa P (2001) Bone marrow cells regenerate infarcted myocardium. Nature 410: 701-705.

Pape H C (1996) Queer current and pacemaker: the hyperpolarization-activated cation current in neurons. Annu Rev Physiol 58: 299-327.

Perin E C, Geng Y J, Willerson J T (2003) Adult stem cell therapy in perspective. Circulation 107: 935-938.

Pfeifer A, Verma I M (2001) Gene therapy: promises and problems. Annu Rev Genomics Hum Genet @: 177-211.

Piper D R, Hinz W A, Tallurri C K, Sanguinetti M C, Tristani-Firouzi M (2005) Regional specificity of human ether-a-go-go-related gene channel activation and inactivation gating. J Biol Chem 2005 February 25;280(8):7206-17.

Pittenger M F, Mackay A M, Beck S C, Jaiswal R K, Douglas R, Mosca J D, Moorman M A, Simonetti D W, Craig S & Marshak D R (1999) Multilineage potential of adult human mesenchymal stem cells. Science 284: 143-147.

Plotnikov A N, Shlapakova I N, Kryukova Y, Bucchi A, Pan Z, Danilo P Jr, Brink P R, Cohen I S, Robinson R B, Rosen M R (2005a) Comparison of mHCN2 and mHCN2-E324A genes as biological pacemakers. Circulation 112: II-126 (Abstract).

Plotnikov A N, Shlapakova I N, Szabolcs M J, Danilo P, Jr., Lu Z, Potapova I, Lorell B H, Brink P R, Robinson R B, Cohen I S, Rosen M R (2005b) Adult human mesenchymal stem cells carrying HCN2 gene perform biological pacemaker function with no overt rejection for 6 weeks in canine heart. Circulation 112: II-221 (Abstract).

Plotnikov A N, Sosunov E A, Qu J, Shlapakova I N, Anyukhovsky E P, Liu L, Janse M J, Brink P R, Cohen I S, Robinson R B, Danilo P Jr, Rosen M R (2004) A biological pacemaker implanted in the canine left bundle branch provides ventricular escape rhythms having physiologically acceptable rates. Circulation 109: 506-512.

Potapova I, Plotnikov A, Lu Z, Danilo P Jr, Valiunas V, Qu J, Doronin S, Zuckerman J, Shlapakova I N, Gao J, Pan Z, Herron A J, Robinson R B, Brink P R, Rosen M R, Cohen I S (2004) Human mesenchynal stem cells as a gene delivery system to create cardiac pacemakers. Circ Res 94: 952-959.

Protas L, Robinson R B (1999) Neuropeptide Y contributes to innervation-dependent increase in I(Ca, L) via ventricular Y2 receptors. Am J Physiol 277: H940-946.

Qu J, Barbuti A, Protas L, Santoro B, Cohen I S, Robinson R B (2001) HCN2 overexpression in newborn and adult ventricular myocytes: distinct effects on gating and excitability. Circ Res 89: E8-14.

Qu J, Kryukova Y, Potapova I A, Doronin S V, Larsen M, Krishnamurthy G, Cohen I S, Robinson R B (2004) MiRP1 modulates HCN2 channel expression and gating in cardiac myocytes. J Biol Chem 279: 43497-43502.

Qu J, Plotnikov A N, Danilo P Jr, Shlapakova I, Cohen I S, Robinson R B, Rosen M R (2003) Expression and function of a biological pacemaker in canine heart. Circulation 107: 1106-1109.

Qu J, et al.: Sympathetic innervation alters activation of pacemaker current (I_{f}) in rat ventricles. J. Physiol. Vol. 526, No. 3, Aug. 1, 2000, pages 561-569.

Ranjan R, et al.: Mechanism of anode break stimulation in the heart. Biophys. J. Vol. 74, No. 4, April 1998, pages 1850-1863.

Robinson R B, et al.: Developmental change in the voltage dependence of the pacemaker current, I_{f}, in rat ventricle cells. Pflugers Arch. Vol. 433, 1991, pages 533-535.

Robinson R B, Siegelbaum S A (2003) Hyperpolarization-activated cation currents: from molecules to physiological function. Annu Rev Physiol 65: 453-480.

Rosen M R, Brink, P R, Cohen I S, Robinson R B (2004) Genes, stem cells and biological pacemakers. Cardiovasc Res 64: 12-23.

Rosen M (2005) Biological pacemaking: In our lifetime? Heart Rhythm 2: 418-428.

Sambrook J, Fritsch E F and Maniatis T (1989) Molecular Cloning: A Laboratory Manual, 2^{nd} ed., Cold Spring Harbor Laboratory Press, New York.

Sanguinetti M C, et al.: Coassembly of KvLGQT1 and minK (I_{SK}) proteins to form cardiac I_{SK} potassium channels. Nature. Vol. 384, No. 6604, Nov. 7, 1996, pages 80-83.

Santoro B, et al.: Interactive cloning with the SH3 domain of N-src identifies a new brain specific ion channel protein, with homology to Eag and cyclic nucleaotide-gated channels. Proc. Natl. Sci. USA. Vol. 94, No. 26, Dec. 23, 1997, pages 14815-14820.

Santoro B, Chen S, Luthi A, Pavlidis P, Shumyatsky G P, Tibbs G R, Siegelbaum S A (2000) Molecular and functional heterogeneity of hyperpolarization-activated pacemaker channels in the mouse CNS. J Neurosci 20: 5264-5275.

Santoro B, Liu D T, Yao H, Bartsch D, Kandel E R, Siegelbaum S A, Tibbs G R (1998) Identification of a gene encoding a hyperpolarization-activated pacemaker channel of brain. Cell 93(5): 717-729.

Santoro B, Tibbs G R (1999) The HCN Gene Family: Molecular Basis of the Hyperpolarization-Activated Pacemaker Channels. Ann NY Acad Sci 868: 741-764.

Sclagger H and von Jagow G: Tricine-sodium dodecyl sulfate-polyacrylamide gel electrophoresis for separation of proteins in the range from 1 to 100 Kda. Analytical Biochem. Vol. 166, No. 2, Nov. 1, 1987, pages 368-379.

Shi W, et al.: The distribution and prevalence of HCN isoforms in the canine heart and their relation to the voltage dependence of I_{f}. Biophys. J. Vol. 78, 2000, pages 353A.

Shi W, et al.: Distribution and Prevalence of hyperpolarization-activated cation channel (HCN) mRNA Expression in Cardiac Tissues. Circ. Res. Vol. 85, No. 1, Jul. 9, 1999, pages el-e6.

Stieber J, Stockl G, Herrmann S, Hassfurth B, Hofmann F (2005) Functional expression of the human HCN3 channel. J Biol Chem 280: 34635-34643.

Strauer B E, Brehm M, Zeus T, Kostering M, Hernandez A, Sorg R V, Kogler G, Wemet P (2002) Repair of infarcted myocardium by autologous intracoronary mononuclear bone marrow cell transplantation in humans. Circulation 106; 1913-1918.

Tinel N, et al.: KCNE2 confers background current characteristics to the cardiac KCNQ1 potassium channel. EMBO J. Vol. 19, No. 23, Dec. 1, 2000, pages 6326-6330.

Trono D (2002) Lentiviral Vectors. Springer-Verlag: New York/Berlin/Heidelberg.

Tsang S Y, Lesso H, Li R A (2004) Dissecting the structural and functional roles of the S3-S4 linker of pacemaker (hyperpolarization-activated cyclic nucleotide-modulated) channels by systematic length alterations. J Biol Chem 279: 43752-43759.

Tse H F, Xue T, Lau C P, Siu C W, Wang K, Zhang Q Y, Tomaselli G F, Akar F G, Li R A (2006) Bioartificial sinus node constructed via in vivo gene transfer of an engineered pacemaker HCN channel reduces the dependence on electronic pacemaker in a sick-sinus syndrome model. Published online in Circulation on Aug. 21, 2006.

Valiunas V, Beyer E C, Brink P R (2002) Cardiac gap junction channels show quantitative differences in selectivity. Circ Res 91: 104-111.

Valiunas V, Bukauskas F F, Weingart R (1997) Conductances and selective permeability of connexin43 gap junction channels examined in neonatal rat heart cells. Circ Res 80: 708-719.

Valiunas V, Doronin S, Valiuniene L, Potapova I, Zuckerman J, Walcott B, Robinson R B, Rosen M R, Brink P R, Cohen I S (2004) Human mesenchymal stem cells make cardiac connexins and form functional gap junctions. J Physiol 555: 617-26.

Valiunas V, Gemel J, Brink P R, Beyer E C (2001) Gap junction channels formed by coexpressed connexin40 and connexin43. Am J Physiol Heart Circ Physiol 281: H1675-H1689.

Valiunas V, Weingart R, Brink P R (2000) Formation of heterotypic gap junction channels by connexins 40 and 43. Circ Res 86: E42 -E49.

van den Bos C, Mosca J D, Winkles J, Kerrigan L, Burgess W H & Marshak D R (1997) Human mesenchymal stem cells respond to fibroblast growth factors. Hum Cell 10: 45-50.

Vassalle M, et al.: Pacemaker channels and cardiac automaticity In “Cardiac Electrophysiology. From Cell to Bedside”, Eds. Zipes D and Jalife W B Saunders Co. Philadelphia, Pa., 2000, pages 94-103.

Vemana S, Pandey S, Larsson H P (2004) S4 movement in a mammalian HCN channel. J Gen Physiol 123: 21-32.

Wahler G M: Developmental increases in the inwardly rectifying potassium current of rat ventricular myocytes. Am. J. Physiol. Vol. 262, No. 5 Pt. 1, May 1992, pages C1266.

Walsh K B, et al.: Disctinct voltage-dependent regulation of heart-delayed I_{K} by protein kinases A and C. Am. J. Physiol. Vol. 261, No. 6 Pt. 1, December 1991, pages C1081-C1090.

Wang H Z, Day N, Valcic M, Hsieh K, Serels S, Brink P R, Christ G J (2001a) Intercellular communication in cultured human vascular smooth muscle cells. Am J Physiol Cell Physiol 281: C75-C88.

Wang J, Chen S, Siegelbaum S A (2001b) Regulation of hyperpolarization-activated HCN channel gating and cAMP modulation due to interactions of COOH terminus and core transmembrane regions. J Gen Physiol 118: 237-250.

Wang Y, Bai Y, Price C, Boros P, Qin L, Bielinska A U, Kukowska-Latallo J F, Baker J R Jr, Bromberg J S (2001c) Combination of electroporation and DNA/dendrimer complexes enhances gene transfer into murine cardiac transplants. Am J Transplant 1: 334-338.

Xue T, Cho H C, Akar F G, Tsang S Y, Jones S P, Marban E, Tomaselli G F, Li R A (2005) Functional integration of electrically active cardiac derivatives from genetically engineered human embryonic stem cells with quiescent recipient ventricular cardiomyocytes. Circulation 111: 11-20.

Yu H, Gao J, Wang H, Wymore R, Steinberg S, McKinnon D, Rosen M R & Cohen I S (2000) Effects of the renin-angiotensin system on the current I(to) in epicardial and endocardial ventricular myocytes from the canine heart. Circ Res 86: 1062-1068.

Yu H, Lu Z, Pan Z, Cohen I S (2004) Tyrosine kinase inhibition differentially regulates heterologously expressed HCN channels. Pflugers Archiv. 447: 392-400.

Yu H, et al.: Phosphatase inhibition by calyculin A increases I_{f} in canine Purkinje fibers and myocytes. Pflugers Arch. Vol. 422, No. 6, March 1993, pages 614-616.

Yu, H., Wu, J., Potapova, I. Wymore, R. T., Holmes, B., Zuckerman, J., Pan, Z., Wang, H., W., Robinson, R. B., El-Maghrabi, R., Benjamin, W., Dixon, J., McKinnon, D., Cohen, I. S., & Wymore, R. (2001) MinK-related protein 1: A beta subunit for the HCN ion channel subunit family enhances expression and speeds activation. Circ Res 88: E84-87.

Zhou Y Y, Wang S Q, Zhu W Z, Chruscinski A, Kobilka B K, Ziman B, Wang S, Lakatta E G, Cheng H & Xiao R P (2000) Culture and adenoviral infection of adult mouse cardiac myocytes: methods for cellular genetic physiology. Am J Physiol Heart Circ Physiol 279: H429-H436.

Zaza, A., Robinson, R. B., & DiFrancesco, D. (1996). Basal responses of the L type Ca²⁺ and hyperpolarization-activated currents to autonomic agonists in the rabbit sino-atrial node. Journal of Physiology (London) 491, 347-355.

What is claimed:

1. A chimeric hyperpolarization-activated, cyclic nucleotide-gated (HCN) polypeptide comprising portions derived from more than one HCN channel isoform.

2. The HCN polypeptide of claim 1, wherein the portions are an amino terminal portion, an intramembranous portion, and a carboxy terminal portion.

3. The chimeric HCN polypeptide of claim 2, wherein the portions are derived from human HCN isoforms.

4. The chimeric HCN polypeptide of claim 2, wherein at least one portion of the HCN chimera is derived from an animal species which is different from the animal species from which at least one of the other two portions is derived.

5. The chimeric HCN polypeptide of claim 2, wherein the intramembranous portion is derived from an HCN1 channel.

6. The chimeric HCN polypeptide of claim 5, wherein the intramembranous portion is D140-L400 of hHCN1 having the sequence set forth in SEQ ID NO:______.

7. The chimeric HCN polypeptide of claim 5, wherein the intramembranous portion is D129-L389 of mHCN1 having the sequence set forth in SEQ ID NO:______.

8. The chimeric HCN polypeptide of claim 2, wherein the amino terminal portion is derived from HCN2, HCN3 or HCN4 and the carboxy terminal portion is derived from HCN2, HCN3 or HCN4.

9. The chimeric HCN polypeptide of claim 2, wherein the amino terminal portion is derived from HCN2 and the carboxy terminal portion is derived from HCN2.

10. The chimeric HCN polypeptide of claim 1, wherein the polypeptide provides an improved characteristic, as compared to a wild-type HCN channel, selected from the group consisting of faster kinetics, more positive activation, increased expression, increased stability, enhanced cAMP responsiveness, and enhanced neurohumoral response.

11. The chimeric HCN polypeptide of claim 1, wherein the polypeptide comprises mHCN112, mHCN212, mHCN312, mHCN412, mHCN114, mHCN214, mHCN314, mHCN414, hHCN112, hHCN212, hHCN312, hHCN412, hHCN114, hHCN214, hHCN314, or hHCN414.

12. The chimeric HCN polypeptide of claim 11, wherein the polypeptide is hHCN212 having the sequence set forth in SEQ ID NO:______.

13. The chimeric HCN polypeptide of claim 11, wherein the polypeptide is mHCN212 having the sequence set forth in SEQ ID NO:______.

14. The chimeric HCN polypeptide of claim 1, wherein at least one portion of the polypeptide is derived from a HCN channel containing a mutation which provides an improved characteristic, as compared to a portion from a wild-type HCN channel, selected from the group consisting of faster kinetics, more positive activation, increased expression, increased stability, enhanced cAMP responsiveness, and enhanced neurohumoral response.

15. The chimeric HCN polypeptide of claim 14, wherein the mutant HCN channel contains a mutation in a region of the channel selected from the group consisting of the S4 voltage sensor, the S4-S5 linker, S5, S6 and S5-S6 linker, the C-linker, and the CNBD.

16. The chimeric HCN polypeptide of claim 14, wherein the mutant portion is derived from mHCN2 having the sequence set forth in SEQ ID NO:______ and comprises E324A-mHCN2, Y331A-mHCN2, R339A-mHCN2, or Y331A,E324A-mHCN2.

17. The chimeric HCN polypeptide of claim 16, wherein the mutant portion comprises E324A-mHCN2.

18. A nucleic acid encoding the chimeric HCN polypeptide of claim 12.

19. A pharmaceutical composition comprising the nucleic acid of claim 18 and a pharmaceutically acceptable carrier.

20. A vector comprising the nucleic acid of claim 18.

21. The vector of claim 20, which is a plasmid, cosmid, or viral vector.

22. A pharmaceutical composition comprising the vector of claim 18 and a pharmaceutically acceptable carrier.

23. A cell comprising the nucleic acid of claim 18, wherein the cell expresses the chimeric HCN polypeptide.

24. The cell of claim 23, which expresses the chimeric HCN polypeptide at a level effective to induce a pacemaker current in the cell.

25. The cell of claim 23, which is a stem cell, a cardiomyocyte, a fibroblast or skeletal muscle cell engineered to express at least one cardiac connexin, or an endothelial cell.

26. The cell of claim 25, wherein the stem cell is an adult mesenchymal stem cell or an embryonic stem cell.

27. The cell of claim 26, wherein the stem cell is a human adult mesenchymal stem cell.

28. The cell of claim 23, which further expresses at least one cardiac connexin.

29. The cell of claim 28, wherein the at least one cardiac connexin is Cx43, Cx40, or Cx45.

30. A pharmaceutical composition comprising the cell of claim 27 and a pharmaceutically acceptable carrier.

31. A method of treating a subject afflicted with a cardiac rhythm disorder comprising administering the cell of claim 27 to a region of the subject's heart, wherein expression of the chimeric HCN polypeptide in said region of the heart is effective to induce a pacemaker current in the heart and thereby treat the subject.

32. The method of claim 31, wherein a pre-existing source of pacemaker activity in the heart is ablated.

33. The method of claim 32, wherein the cell forms a functional syncytium with the heart.

34. The method of claim 32, wherein the cell is administered to the region of the heart by injection, catheterization, surgical insertion, or surgical attachment.

35. The method of claim 4, wherein the cell is locally administered by injection or catheterization directly onto or into the heart tissue.

36. The method of claim 34, wherein the cell is administered by injection or catheterization into at least one of a coronary blood vessel or other blood vessel proximate to the heart.

37. The method of claim 31, wherein the cell is administered to a region of an atrium or ventricle of the heart.

38. The method of claim 37, wherein the disorder is a sinus node dysfunction, sinus bradycardia, marginal pacemaker function, sick sinus syndrome, tachyarrhythmia, sinus node reentry tachycardia, atrial tachycardia from an ectopic focus, atrial flutter, atrial fibrillation, bradyarrhythmia, or cardiac failure, and the cell is administered to the right or left atrial muscle, sinoatrial node, or atrioventricular junctional region of the subject's heart.

39. The method of claim 37, wherein the disorder is a conduction block, complete atrioventricular block, incomplete atrioventricular block, or bundle branch block, and the cell is administered to a region of the subject's heart so as to compensate for the impaired conduction in the heart.

40. The method of claim 39, wherein the cell is administered to a ventricular septum or free wall, atrioventricular junction, or bundle branch of the ventricle.

41. A method of inhibiting the onset of a cardiac rhythm disorder in a subject prone to such disorder comprising administering the cell of claim 27 to a region of the subject's heart, wherein expression of the chimeric HCN polypeptide in the heart is effective to induce a pacemaker current in the heart and thereby inhibit the onset of the disorder in the subject.

42. A method of treating a subject afflicted with a cardiac rhythm disorder comprising transfecting a cell of the subject's heart with the nucleic acid of claim 18 so as to functionally express the chimeric HCN polypeptide in the heart, wherein expression of said polypeptide is effective to induce a pacemaker current in the heart and thereby treat the subject.

43. The method of claim 42, wherein a pre-existing source of pacemaker activity in the heart is ablated.

44. The method of claim 42, wherein the cell of the heart is in an atrium or ventricle of the heart.

45. The method of claim 42, wherein the disorder is a sinus node dysfunction, sinus bradycardia, marginal pacemaker function, sick sinus syndrome, tachyarrhythmia, sinus node reentry tachycardia, atrial tachycardia from an ectopic focus, atrial flutter, atrial fibrillation, bradyarrhythmia, or cardiac failure, and a cell in the right or left atrial muscle, sinoatrial node, or atrioventricular junctional region of the subject's heart is transfected.

46. The method of claim 47, wherein the disorder is a conduction block, complete atrioventricular block, incomplete atrioventricular block, or bundle branch block, and a cell is transfected in a region of the subject's heart so as to compensate for the impaired conduction in the heart.

47. The method of claim 46, wherein a cell in a ventricular septum or free wall, atrioventricular junction, or bundle branch of the ventricle is transfected.

48. A method of inhibiting the onset of a cardiac rhythm disorder in a subject prone to such disorder comprising transfecting a cell of the subject's heart with the nucleic acid of claim 18 so as to functionally express the chimeric HCN polypeptide in the heart, wherein expression of said polypeptide is effective to induce a pacemaker current in the heart and thereby inhibit the onset of the disorder in the subject.

49. A method of producing the chimeric HCN polypeptide of claim 2 comprising (a) generating a recombinant nucleic acid by joining a nucleic acid encoding an amino terminal portion of a HCN polypeptide to a nucleic acid encoding an intramembranous portion of a HCN polypeptide and joining said nucleic acid encoding the intramembranous portion to a nucleic acid encoding a carboxy terminal portion of a HCN polypeptide, wherein the encoded portions of the HCN polypeptide are derived from more than one HCN isoform or mutant thereof, and (b) functionally expressing said recombinant nucleic acid in a cell so as to produce the chimeric HCN polypeptide.
